SEQUENCE LISTING (559 sequences)

SEQ ID NO: 1 - protein, 15 aa, Homo sapiens
APGFPGPRGPPGPQG

SEQ ID NO: 2 - protein, 15 aa, Homo sapiens
PAGFAGPPGADGQPG

SEQ ID NO: 3 - protein, 15 aa, Homo sapiens
GIAGFKGDQGPKGET

SEQ ID NO: 4 - protein, 9 aa, Homo sapiens
HLVEALYLV

SEQ ID NO: 5 - protein, 9 aa, Homo sapiens
LNIDLLWSV

SEQ ID NO: 6 - protein, 9 aa, Homo sapiens
VLFGLGFAI

SEQ ID NO: 7 - protein, 9 aa, Homo sapiens
VYLKTNLFL

SEQ ID NO: 8 - protein, 9 aa, Homo sapiens
VYLKTNVFL

SEQ ID NO: 9 - protein, 13 aa, Homo sapiens
QHLQKDYRAYYTF

SEQ ID NO: 10 - protein, 13 aa, Homo sapiens
YTFLNFMSNVGDP

SEQ ID NO: 11 - protein, 9 aa, Homo sapiens
KYNKANAFL

SEQ ID NO: 12 - protein, 9 aa, Homo sapiens
KYNIANVFL

SEQ ID NO: 13 - protein, 9 aa, Homo sapiens
KYNKANVFL

SEQ ID NO: 14 - protein, 9 aa, Homo sapiens
FQDENYLYL

SEQ ID NO: 15 - protein, 9 aa, Homo sapiens
LYLVCGERG

SEQ ID NO: 16 - protein, 9 aa, Homo sapiens
LYLVCGERI

SEQ ID NO: 17 - protein, 15 aa, Homo sapiens
SLQPLALEGSLQKRG

SEQ ID NO: 18 - protein, 15 aa, Homo sapiens
SLQPLALEGSLQSRG

SEQ ID NO: 19 - protein, 13 aa, Homo sapiens
NFFRMVISNPAAT

SEQ ID NO: 20 - protein, 13 aa, Homo sapiens
NFIRMVISNPAAT

SEQ ID NO: 21 - protein, 13 aa, Homo sapiens
FFYTPKTRREAED

SEQ ID NO: 22 - protein, 9 aa, Homo sapiens
KYQAVTTTL

SEQ ID NO: 23 - protein, 9 aa, Homo sapiens
KYCLITIFL

SEQ ID NO: 24 - protein, 9 aa, Homo sapiens
ALWMRLLPL

SEQ ID NO: 25 - protein, 9 aa, Homo sapiens
LWMRLLPLL

SEQ ID NO: 26 - protein, 9 aa, Homo sapiens
RLLPLLALL

SEQ ID NO: 27 - protein, 10 aa, Homo sapiens
HLCGSHLVEA

SEQ ID NO: 28 - protein, 9 aa, Homo sapiens
HLVEALYLV

SEQ ID NO: 29 - protein, 9 aa, Homo sapiens
ALYLVCGER

SEQ ID NO: 30 - protein, 10 aa, Homo sapiens
LYLVCGERGF

SEQ ID NO: 31 - protein, 9 aa, Homo sapiens
LVCGERGFF

SEQ ID NO: 32 - protein, 10 aa, Homo sapiens
VCGERGFFYT

SEQ ID NO: 33 - protein, 8 aa, Homo sapiens
GERGFFYT

SEQ ID NO: 34 - protein, 9 aa, Homo sapiens
ERGFFYTPK

SEQ ID NO: 35 - protein, 9 aa, Homo sapiens
FYTPKTRRE

SEQ ID NO: 36 - protein, 11 aa, Homo sapiens
TPKTRREAEDL

SEQ ID NO: 37 - protein, 9 aa, Homo sapiens
SLQPLALEG

SEQ ID NO: 38 - protein, 9 aa, Homo sapiens
ALEGSLQKR

SEQ ID NO: 39 - protein, 10 aa, Homo sapiens
SLQKRGIVEQ

SEQ ID NO: 40 - protein, 10 aa, Homo sapiens
GIVEQCCTSI

SEQ ID NO: 41 - protein, 9 aa, Homo sapiens
IVEQCCTSI

SEQ ID NO: 42 - protein, 9 aa, Homo sapiens
SLYQLENYC

SEQ ID NO: 43 - protein, 21 aa, Homo sapiens
MEVGWYRSPFSRVVHLYRNGK

SEQ ID NO: 44 - protein, 15 aa, Homo sapiens
IGPRHPIRALVGDEV

SEQ ID NO: 45 - protein, 20 aa, Homo sapiens
EVGWYRSPFSRVVHLYRNGK

SEQ ID NO: 46 - protein, 9 aa, Homo sapiens
KVEDPFYWV

SEQ ID NO: 47 - protein, 10 aa, Homo sapiens
RTFDPHFLRV

SEQ ID NO: 48 - protein, 9 aa, Homo sapiens
FLRVPCWKI

SEQ ID NO: 49 - protein, 10 aa, Homo sapiens
KITLFVIVPV

SEQ ID NO: 50 - protein, 9 aa, Homo sapiens
VLGPLVALI

SEQ ID NO: 51 - protein, 9 aa, Homo sapiens
TLFVIVPVL

SEQ ID NO: 52 - protein, 10 aa, Homo sapiens
RLAGQFLEEL

SEQ ID NO: 53 - protein, 13 aa, Homo sapiens
TCFFRDHSYQEEA

SEQ ID NO: 54 - protein, 13 aa, Homo sapiens
TCFFRDHSYQSEA

SEQ ID NO: 55 - protein, 15 aa, Homo sapiens
ALIICYNWLHRRLAG

SEQ ID NO: 56 - protein, 15 aa, Homo sapiens
LVLLAVLPVLLLQIT

SEQ ID NO: 57 - protein, 15 aa, Homo sapiens
FLRVPCWKITLFVIV

SEQ ID NO: 58 - protein, 15 aa, Homo sapiens
RHPIRALVGDEVELP

SEQ ID NO: 59 - protein, 15 aa, Homo sapiens
RVPCWKITLFVIVPV

SEQ ID NO: 60 - protein, 9 aa, Homo sapiens
SLLLELEEV

SEQ ID NO: 61 - protein, 9 aa, Homo sapiens
LMWAKIGPV

SEQ ID NO: 62 - protein, 9 aa, Homo sapiens
VLFSSDFRI

SEQ ID NO: 63 - protein, 9 aa, Homo sapiens
LMWAKIGPV

SEQ ID NO: 64 - protein, 9 aa, Homo sapiens
VLFSSDFRI

SEQ ID NO: 65 - protein, 9 aa, Homo sapiens
SLSRFSWGA

SEQ ID NO: 66 - protein, 20 aa, Homo sapiens
KYLATASTMDHARHGFLPRH

SEQ ID NO: 67 - protein, 17 aa, Homo sapiens
ENPVVHFFKNIVTPRTP

SEQ ID NO: 68 - protein, 19 aa, Homo sapiens
LSRFSWGAEGQRPGFGYGG

SEQ ID NO: 69 - protein, 25 aa, Homo sapiens
AQGTLSKIFKLGGRDSRSGSPMARR

SEQ ID NO: 70 - protein, 13 aa, Homo sapiens
NPVVHFFKNIVTP

SEQ ID NO: 71 - protein, 13 aa, Homo sapiens
VHFFKNIVTPRTP

SEQ ID NO: 72 - protein, 13 aa, Homo sapiens
PVVHFFKNIVTPR

SEQ ID NO: 73 - protein, 18 aa, Homo sapiens
YIYFNTWTTCQSIAFPSK

SEQ ID NO: 74 - protein, 15 aa, Homo sapiens
GAVRQIFGDYKTTIC

SEQ ID NO: 75 - protein, 15 aa, Homo sapiens
NYQDYEYLINVIHAF

SEQ ID NO: 76 - protein, 9 aa, Homo sapiens
FLYGALLLA

SEQ ID NO: 77 - protein, 15 aa, Homo sapiens
ATLVSLLTFMIAATY

SEQ ID NO: 78 - protein, 15 aa, Homo sapiens
TLVSLLTFMIAATYN

SEQ ID NO: 79 - protein, 15 aa, Homo sapiens
AEGFYTTGAVRQIFG

SEQ ID NO: 80 - protein, 16 aa, Homo sapiens
HCLGKWLGHPDKFVGI

SEQ ID NO: 81 - protein, 12 aa, Homo sapiens
QLQPFPQPELPY

SEQ ID NO: 82 - protein, 11 aa, Homo sapiens
PQPELPYPQPE

SEQ ID NO: 83 - protein, 13 aa, Homo sapiens
SGEGSFQPSQQNP

SEQ ID NO: 84 - protein, 14 aa, Homo sapiens
GDLIAEVETDKATV

SEQ ID NO: 85 - protein, 14 aa, Homo sapiens
GDLLAEIETDKATI

SEQ ID NO: 86 - protein, 15 aa, Homo sapiens
GDLLAEIETDKATIG

SEQ ID NO: 87 - protein, 15 aa, Homo sapiens
AQWLAEFRKYLEKPI

SEQ ID NO: 88 - protein, 15 aa, Homo sapiens
RLLLQLLGSPGRRYY

SEQ ID NO: 89 - protein, 15 aa, Homo sapiens
GRVFVSPLAKKLAVE

SEQ ID NO: 90 - protein, 15 aa, Homo sapiens
DIPISNIRRVIAQRL

SEQ ID NO: 91 - protein, 15 aa, Homo sapiens
AQWLAEFRKYLEKPI

SEQ ID NO: 92 - protein, 15 aa, Homo sapiens
SPGRRYYSLPPHQKV

SEQ ID NO: 93 - protein, 15 aa, Homo sapiens
GRVFVSPLAKKLAVE

SEQ ID NO: 94 - protein, 15 aa, Homo sapiens
ETIANDVVSLATKAR

SEQ ID NO: 95 - protein, 14 aa, Homo sapiens
GEIRTMNNFLDREQ

SEQ ID NO: 96 - protein, 14 aa, Homo sapiens
GEIRTMNNFLDREI

SEQ ID NO: 97 - protein, 15 aa, Homo sapiens
KREWIKFAAACREGE

SEQ ID NO: 98 - protein, 15 aa, Homo sapiens
MFIINRNTGEIRTMN

SEQ ID NO: 99 - protein, 15 aa, Homo sapiens
SQYKLKASAISVTVL

SEQ ID NO: 100 - protein, 15 aa, Homo sapiens
WSFFRVVAMLFIFLV

SEQ ID NO: 101 - protein, 15 aa, Homo sapiens
SKIAFKIIRQEPSDS

SEQ ID NO: 102 - protein, 15 aa, Homo sapiens
TNVGILKVVKPLDYE

SEQ ID NO: 103 - protein, 15 aa, Homo sapiens
MDWSFFRVVAMLFIF

SEQ ID NO: 104 - protein, 15 aa, Homo sapiens
KNGTIKWHSIRRQKR

SEQ ID NO: 105 - protein, 15 aa, Homo sapiens
RTNVGILKVVKPLDY

SEQ ID NO: 106 - protein, 15 aa, Homo sapiens
FGIFVVDKNTGDINI

SEQ ID NO: 107 - protein, 15 aa, Homo sapiens
CECNIKVKDVNDNFP

SEQ ID NO: 108 - protein, 15 aa, Homo sapiens
NKAEFHQSVISRYRV

SEQ ID NO: 109 - protein, 15 aa, Homo sapiens
DSTFIVNKTITAEVL

SEQ ID NO: 110 - protein, 15 aa, Homo sapiens
SITTLNATSALLRAQ

SEQ ID NO: 111 - protein, 15 aa, Homo sapiens
ILSSELLRFQVTDLD

SEQ ID NO: 112 - protein, 15 aa, Homo sapiens
EGILKVVKALDYEQL

SEQ ID NO: 113 - protein, 15 aa, Homo sapiens
STPVTIQVINVREGI

SEQ ID NO: 114 - protein, 15 aa, Homo sapiens
AIFVVVILVHGELRI

SEQ ID NO: 115 - protein, 15 aa, Homo sapiens
RTNEGILKVVKALDY

SEQ ID NO: 116 - protein, 15 aa, Homo sapiens
DSKTAEIKFVKNMNR

SEQ ID NO: 117 - protein, 15 aa, Homo sapiens
GAGILYLVTPPSVVG

SEQ ID NO: 118 - protein, 15 aa, Homo sapiens
RSQVETDDLILKPGV

SEQ ID NO: 119 - protein, 14 aa, Homo sapiens
EKPLPVDMVLISLC

SEQ ID NO: 120 - protein, 15 aa, Homo sapiens
GAGILYLVTPPSVVG

SEQ ID NO: 121 - protein, 15 aa, Homo sapiens
TAEFLAMLIFVLLSL

SEQ ID NO: 122 - protein, 15 aa, Homo sapiens
LRQMRTVTPIRMQGG

SEQ ID NO: 123 - protein, 15 aa, Homo sapiens
AVNIVGYSNAQGVDY

SEQ ID NO: 124 - protein, 15 aa, Homo sapiens
RFGISNYCQIYPPNV

SEQ ID NO: 125 - protein, 15 aa, Homo sapiens
PCIIHRGKPFQLEAV

SEQ ID NO: 126 - protein, 15 aa, Homo sapiens
TVKVMGDDGVLACAI

SEQ ID NO: 127 - protein, 15 aa, Homo sapiens
EAINQGYMHADAYPF

SEQ ID NO: 128 - protein, 15 aa, Homo sapiens
KDLTYTFLRDFEQYL

SEQ ID NO: 129 - protein, 15 aa, Homo sapiens
RQLRTLVNEAINQGY

SEQ ID NO: 130 - protein, 15 aa, Homo sapiens
MDKIRYRLVYNRQNT

SEQ ID NO: 131 - protein, 15 aa, Homo sapiens
LNQRKIYLKTNVYLK

SEQ ID NO: 132 - protein, 15 aa, Homo sapiens
EYILYLQGIELGYWK

SEQ ID NO: 133 - protein, 15 aa, Homo sapiens
TCATLLIHQGVAITT

SEQ ID NO: 134 - protein, 15 aa, Homo sapiens
AKHMRQLRTLVNEAI

SEQ ID NO: 135 - protein, 15 aa, Homo sapiens
IRYRLVYNRQNTLNR

SEQ ID NO: 136 - protein, 15 aa, Homo sapiens
ENFIRINGKRWLYFK

SEQ ID NO: 137 - protein, 15 aa, Homo sapiens
TGAAATYAIDSIADA

SEQ ID NO: 138 - protein, 15 aa, Homo sapiens
NATFSMDQLKFGDTI

SEQ ID NO: 139 - protein, 15 aa, Homo sapiens
DRTVVSSIGAYKLIQ

SEQ ID NO: 140 - protein, 15 aa, Homo sapiens
MVVQHNLRAMNSNRM

SEQ ID NO: 141 - protein, 15 aa, Homo sapiens
KMRKQIRGLSQASLN

SEQ ID NO: 142 - protein, 15 aa, Homo sapiens
GAYKLIQKELGLASS

SEQ ID NO: 143 - protein, 15 aa, Homo sapiens
QHNLRAMNSNRMLGI

SEQ ID NO: 144 - protein, 15 aa, Homo sapiens
YKLIQKELGLASSIG

SEQ ID NO: 145 - protein, 15 aa, Homo sapiens
HNIQVADDARFVLNA

SEQ ID NO: 146 - protein, 15 aa, Homo sapiens
ADDARFVLNAGKKKF

SEQ ID NO: 147 - protein, 15 aa, Homo sapiens
GCISYALVSHTAKGS

SEQ ID NO: 148 - protein, 15 aa, Homo sapiens
ADDIVKMLNDPALNR

SEQ ID NO: 149 - protein, 15 aa, Homo sapiens
LPVTVTLDIITAPLQ

SEQ ID NO: 150 - protein, 15 aa, Homo sapiens
SGCISYALVSHTAKG

SEQ ID NO: 151 - protein, 15 aa, Homo sapiens
DIVKMLNDPALNRHN

SEQ ID NO: 152 - protein, 15 aa, Homo sapiens
ARFVLNAGKKKFTGT

SEQ ID NO: 153 - protein, 24 aa, Homo sapiens
TYTEHAKRKTVTAMDVVYALKRQG

SEQ ID NO: 154 - protein, 15 aa, Homo sapiens
EHAKRKTVTAMDVVY

SEQ ID NO: 155 - protein, 15 aa, Homo sapiens
AKRKTVTAMDVVYAL

SEQ ID NO: 156 - protein, 15 aa, Homo sapiens
HAKRKTVTAMDVVYA

SEQ ID NO: 157 - protein, 15 aa, Homo sapiens
RKTVTAMDVVYALKR

SEQ ID NO: 158 - protein, 14 aa, Homo sapiens
TVTAMDVVYALKRQ

SEQ ID NO: 159 - protein, 15 aa, Homo sapiens
PKKGSKKAVTKAQKK

SEQ ID NO: 160 - protein, 15 aa, Homo sapiens
KAVTKAQKKDGKKRK

SEQ ID NO: 161 - protein, 21 aa, Homo sapiens
STDHPKYSDMIVAAIQAEKNR

SEQ ID NO: 162 - protein, 15 aa, Homo sapiens
KYSDMIVAAIQAEKN

SEQ ID NO: 163 - protein, 15 aa, Homo sapiens
SQEYSGSVANEANVY

SEQ ID NO: 164 - protein, 15 aa, Homo sapiens
SHSLPYESSISTALE

SEQ ID NO: 165 - protein, 15 aa, Homo sapiens
TGAYSNASSTESASY

SEQ ID NO: 166 - protein, 15 aa, Homo sapiens
SFLSQEYSGSVANEA

SEQ ID NO: 167 - protein, 20 aa, Homo sapiens
KTTKQSFDLSVKAQYKKNKH

SEQ ID NO: 168 - protein, 15 aa, Homo sapiens
KTTKQSFDLSVKAQY

SEQ ID NO: 169 - protein, 15 aa, Homo sapiens
TTKQSFDLSVKAQYK

SEQ ID NO: 170 - protein, 15 aa, Homo sapiens
GALVPGGVADAAAAY

SEQ ID NO: 171 - protein, 15 aa, Homo sapiens
AAQFGLVGAAGLGGL

SEQ ID NO: 172 - protein, 15 aa, Homo sapiens
APRPGVLLLLLSILH

SEQ ID NO: 173 - protein, 15 aa, Homo sapiens
GGVADAAAAYKAAKA

SEQ ID NO: 174 - protein, 15 aa, Homo sapiens
VLLLLLSILHPSRPG

SEQ ID NO: 175 - protein, 15 aa, Homo sapiens
AAKAAQFGLVGAAGL

SEQ ID NO: 176 - protein, 15 aa, Homo sapiens
VAAKAQLRAAAGLGA

SEQ ID NO: 177 - protein, 15 aa, Homo sapiens
KSAAKVAAKAQLRAA

SEQ ID NO: 178 - protein, 15 aa, Homo sapiens
AAQFGLVGAAGLGGL

SEQ ID NO: 179 - protein, 15 aa, Homo sapiens
KAQLRAAAGLGAGIP

SEQ ID NO: 180 - protein, 15 aa, Homo sapiens
VPGALAAAKAAKYGA

SEQ ID NO: 181 - protein, 15 aa, Homo sapiens
RPTMDGDPDTPKPVS

SEQ ID NO: 182 - protein, 15 aa, Homo sapiens
SYKEAVLRAIDGINQ

SEQ ID NO: 183 - protein, 15 aa, Homo sapiens
NQRSSDANLYRLLDL

SEQ ID NO: 184 - protein, 15 aa, Homo sapiens
KRIVQRIKDFLRNLV

SEQ ID NO: 185 - protein, 15 aa, Homo sapiens
EFKRIVQRIKDFLRN

SEQ ID NO: 186 - protein, 15 aa, Homo sapiens
RIVQRIKDFLRNLVP

SEQ ID NO: 187 - protein, 15 aa, Homo sapiens
RFALLGDFFRKSKEK

SEQ ID NO: 188 - protein, 15 aa, Homo sapiens
QRIKDFLRNLVPRTE

SEQ ID NO: 189 - protein, 15 aa, Homo sapiens
DGRYVLNGHWVVSPP

SEQ ID NO: 190 - protein, 15 aa, Homo sapiens
THVVYTRDTGPQETL

SEQ ID NO: 191 - protein, 15 aa, Homo sapiens
RLLHYCGSDFVFQAR

SEQ ID NO: 192 - protein, 15 aa, Homo sapiens
HDLLLQVLLQEPNPG

SEQ ID NO: 193 - protein, 15 aa, Homo sapiens
ETRYEVRIQLVYKNR

SEQ ID NO: 194 - protein, 15 aa, Homo sapiens
HRDYLMAVQRLVSPD

SEQ ID NO: 195 - protein, 15 aa, Homo sapiens
EGHAFYHSFGRVLDG

SEQ ID NO: 196 - protein, 15 aa, Homo sapiens
RNHLALMGGDGRYVL

SEQ ID NO: 197 - protein, 15 aa, Homo sapiens
NPGIEFEFWLPRERY

SEQ ID NO: 198 - protein, 15 aa, Homo sapiens
VQRVFRDAGAFAGYW

SEQ ID NO: 199 - protein, 15 aa, Homo sapiens
QLVYKNRSPLRAREY

SEQ ID NO: 200 - protein, 15 aa, Homo sapiens
RRFEYDDPRFLRLLD

SEQ ID NO: 201 - protein, 15 aa, Homo sapiens
TPVVVLNGLAAVREA

SEQ ID NO: 202 - protein, 15 aa, Homo sapiens
ENLRIVVADLFSAGM

SEQ ID NO: 203 - protein, 20 aa, Homo sapiens
TLAWGLLLMILHPDVQRRVQ

SEQ ID NO: 204 - protein, 20 aa, Homo sapiens
TTLITNLSSVLKDEAVWEKP

SEQ ID NO: 205 - protein, 15 aa, Homo sapiens
DPRFLRLLDLAQEGL

SEQ ID NO: 206 - protein, 15 aa, Homo sapiens
RMELFLFFTSLLQHF

SEQ ID NO: 207 - protein, 15 aa, Homo sapiens
DLFSAGMVTTSTTLA

SEQ ID NO: 208 - protein, 15 aa, Homo sapiens
ELFLFFTSLLQHFSF

SEQ ID NO: 209 - protein, 15 aa, Homo sapiens
DQLRRRFGDVFSLQL

SEQ ID NO: 210 - protein, 15 aa, Homo sapiens
EQRRFSVSTLRNLGL

SEQ ID NO: 211 - protein, 20 aa, Homo sapiens
RRFEYDDPRFLRLLDLAQEG

SEQ ID NO: 212 - protein, 20 aa, Homo sapiens
AGMVTTSTTLAWGLLLMILH

SEQ ID NO: 213 - protein, 21 aa, Homo sapiens
AGMVTTSTTLAWGLLLMILHP

SEQ ID NO: 214 - protein, 15 aa, Homo sapiens
QRRFSVSTLRNLGLG

SEQ ID NO: 215 - protein, 15 aa, Homo sapiens
ESGFLREVLNAVPVL

SEQ ID NO: 216 - protein, 15 aa, Homo sapiens
GKVLRFQKAFLTQLD

SEQ ID NO: 217 - protein, 15 aa, Homo sapiens
DPRFLRLLDLAQEGL

SEQ ID NO: 218 - protein, 15 aa, Homo sapiens
GKVLRFQKAFLTQLD

SEQ ID NO: 219 - protein, 15 aa, Homo sapiens
ENLRIVVADLFSAGM

SEQ ID NO: 220 - protein, 15 aa, Homo sapiens
DIEVQGFRIPKGTTL

SEQ ID NO: 221 - protein, 15 aa, Homo sapiens
KPEAFLPFSAGRRAC

SEQ ID NO: 222 - protein, 15 aa, Homo sapiens
YKKLLKERKEMFSYL

SEQ ID NO: 223 - protein, 15 aa, Homo sapiens
DELRTDLKAVEAKVQ

SEQ ID NO: 224 - protein, 15 aa, Homo sapiens
NKITNSLVLDIIKLA

SEQ ID NO: 225 - protein, 14 aa, Homo sapiens
NRLDRCLKAVRKER

SEQ ID NO: 226 - protein, 12 aa, Homo sapiens
LIQQGARVGRID

SEQ ID NO: 227 - protein, 15 aa, Homo sapiens
SPSLDVLITLLSLGS

SEQ ID NO: 228 - protein, 15 aa, Homo sapiens
DQKSCFKSMITAGFE

SEQ ID NO: 229 - protein, 15 aa, Homo sapiens
YTFRGFMSHTNNYPC

SEQ ID NO: 230 - protein, 15 aa, Homo sapiens
YNERLLHTPHNPISL

SEQ ID NO: 231 - protein, 15 aa, Homo sapiens
DCILCIHSTTSCFAP

SEQ ID NO: 232 - protein, 15 aa, Homo sapiens
SSLLNKITNSLVLDI

SEQ ID NO: 233 - protein, 15 aa, Homo sapiens
GSSLLNKITNSLVLD

SEQ ID NO: 234 - protein, 15 aa, Homo sapiens
NDSFIQEISKMYPGR

SEQ ID NO: 235 - protein, 15 aa, Homo sapiens
KEMFSYLSNQIKKLS

SEQ ID NO: 236 - protein, 15 aa, Homo sapiens
STLELFLHELAIMDS

SEQ ID NO: 237 - protein, 15 aa, Homo sapiens
NSLVLDIIKLAGVHT

SEQ ID NO: 238 - protein, 15 aa, Homo sapiens
SKCMHLIQQGARVGR

SEQ ID NO: 239 - protein, 15 aa, Homo sapiens
RSHEHLIRLLLEKGK

SEQ ID NO: 240 - protein, 15 aa, Homo sapiens
RRHYRFIHGIGRSGD

SEQ ID NO: 241 - protein, 15 aa, Homo sapiens
SNGYKKLLKERKEMF

SEQ ID NO: 242 - protein, 20 aa, Homo sapiens
VVSSHYSRRFTPEIAKRPKV

SEQ ID NO: 243 - protein, 16 aa, Homo sapiens
QFFMSDKPLHLAVSLN

SEQ ID NO: 244 - protein, 15 aa, Homo sapiens
QFFMSDKPLHLAVSL

SEQ ID NO: 245 - protein, 15 aa, Homo sapiens
DVIGLTFRRDLYFSR

SEQ ID NO: 246 - protein, 15 aa, Homo sapiens
NVVLYSSDYYVKPVA

SEQ ID NO: 247 - protein, 15 aa, Homo sapiens
SSVRLLIRKVQHAPL

SEQ ID NO: 248 - protein, 15 aa, Homo sapiens
EVPFRLMHPQPEDPA

SEQ ID NO: 249 - protein, 15 aa, Homo sapiens
KKIKAFVEQVANVVL

SEQ ID NO: 250 - protein, 15 aa, Homo sapiens
STPTKLQESLLKKLG

SEQ ID NO: 251 - protein, 15 aa, Homo sapiens
KKVYVTLTCAFRYGQ

SEQ ID NO: 252 - protein, 15 aa, Homo sapiens
KTLTLLPLLANNRER

SEQ ID NO: 253 - protein, 16 aa, Homo sapiens
NRERRGIALDGKIKHE

SEQ ID NO: 254 - protein, 15 aa, Homo sapiens
EAAWQFFMSDKPLHL

SEQ ID NO: 255 - protein, 15 aa, Homo sapiens
QFFMSDKPLHLAVSL

SEQ ID NO: 256 - protein, 15 aa, Homo sapiens
TFIQFKKDLKESMKC

SEQ ID NO: 257 - protein, 15 aa, Homo sapiens
DTGALDVIRNFTLDM

SEQ ID NO: 258 - protein, 15 aa, Homo sapiens
EVIHLIEEHRLVREH

SEQ ID NO: 259 - protein, 15 aa, Homo sapiens
REYRKKMDIPAKLIV

SEQ ID NO: 260 - protein, 15 aa, Homo sapiens
EEILKALDAAFYKTF

SEQ ID NO: 261 - protein, 15 aa, Homo sapiens
KRFLLAVDVSASMNQ

SEQ ID NO: 262 - protein, 15 aa, Homo sapiens
TDMTLQQVLMAMSQI

SEQ ID NO: 263 - protein, 15 aa, Homo sapiens
EVWKALLQEMPLTAL

SEQ ID NO: 264 - protein, 15 aa, Homo sapiens
SHKDLLRLSHLKPSS

SEQ ID NO: 265 - protein, 15 aa, Homo sapiens
YKTFKTVEPTGKRFL

SEQ ID NO: 266 - protein, 15 aa, Homo sapiens
ETEKLLKYLEAVEKV

SEQ ID NO: 267 - protein, 15 aa, Homo sapiens
RIHPFHILIALETYK

SEQ ID NO: 268 - protein, 15 aa, Homo sapiens
EKDSYVVAFSDEMVP

SEQ ID NO: 269 - protein, 15 aa, Homo sapiens
TPADVFIVFTDNETF

SEQ ID NO: 270 - protein, 15 aa, Homo sapiens
QKLGLENAEALIRLI

SEQ ID NO: 271 - protein, 15 aa, Homo sapiens
KLLKKARIHPFHILI

SEQ ID NO: 272 - protein, 15 aa, Homo sapiens
DDQTCREDLHILFSN

SEQ ID NO: 273 - protein, 15 aa, Homo sapiens
TDEYKNDVKNRSVYI

SEQ ID NO: 274 - protein, 15 aa, Homo sapiens
SIFVVFDSIESAKKF

SEQ ID NO: 275 - protein, 15 aa, Homo sapiens
TDLLILFKDDYFAKK

SEQ ID NO: 276 - protein, 15 aa, Homo sapiens
HQIEYYFGDFNLPRD

SEQ ID NO: 277 - protein, 15 aa, Homo sapiens
KEQIKLDEGWVPLEI

SEQ ID NO: 278 - protein, 15 aa, Homo sapiens
DKGQVLNIQMRRTLH

SEQ ID NO: 279 - protein, 15 aa, Homo sapiens
EIMIKFNRLNRLTTD

SEQ ID NO: 280 - protein, 15 aa, Homo sapiens
RDKFLKEQIKLDEGW

SEQ ID NO: 281 - protein, 15 aa, Homo sapiens
SIFVVFDSIESAKKF

SEQ ID NO: 282 - protein, 15 aa, Homo sapiens
SEDKTKIRRSPSKPL

SEQ ID NO: 283 - protein, 15 aa, Homo sapiens
QVLNIQMRRTLHKAF

SEQ ID NO: 284 - protein, 15 aa, Homo sapiens
RNKEVTWEVLEGEVE

SEQ ID NO: 285 - protein, 15 aa, Homo sapiens
NRLTTDFNVIVEALS

SEQ ID NO: 286 - protein, 15 aa, Homo sapiens
KGSIFVVFDSIESAK

SEQ ID NO: 287 - protein, 15 aa, Homo sapiens
KTKIRRSPSKPLPEV

SEQ ID NO: 288 - protein, 15 aa, Homo sapiens
IFVVFDSIESAKKFV

SEQ ID NO: 289 - protein, 15 aa, Homo sapiens
KERIANFKIEPPGLF

SEQ ID NO: 290 - protein, 15 aa, Homo sapiens
QGSIKYIMLNPSSRI

SEQ ID NO: 291 - protein, 15 aa, Homo sapiens
QREKFAWAIDMADED

SEQ ID NO: 292 - protein, 15 aa, Homo sapiens
IQGSIKYIMLNPSSR

SEQ ID NO: 293 - protein, 15 aa, Homo sapiens
YNASITLQQQLKELT

SEQ ID NO: 294 - protein, 15 aa, Homo sapiens
EQLMKLEVQATDREE

SEQ ID NO: 295 - protein, 15 aa, Homo sapiens
SQYFKAQTEARKQMS

SEQ ID NO: 296 - protein, 15 aa, Homo sapiens
KERIANFKIEPPGLF

SEQ ID NO: 297 - protein, 15 aa, Homo sapiens
IQGSIKYIMLNPSSR

SEQ ID NO: 298 - protein, 15 aa, Homo sapiens
YIMLNPSSRIKGEKD

SEQ ID NO: 299 - protein, 15 aa, Homo sapiens
KILSYNRANRAVAIL

SEQ ID NO: 300 - protein, 15 aa, Homo sapiens
KLIEDEFIIDESDQS

SEQ ID NO: 301 - protein, 15 aa, Homo sapiens
KVYKTLDTPFFSTGK

SEQ ID NO: 302 - protein, 15 aa, Homo sapiens
QDILVFYVNFGDLLC

SEQ ID NO: 303 - protein, 15 aa, Homo sapiens
KVMLKKIEIDNKVSD

SEQ ID NO: 304 - protein, 15 aa, Homo sapiens
EDNIMTAQNVPLKPQ

SEQ ID NO: 305 - protein, 15 aa, Homo sapiens
TREIILMDLVRPQDT

SEQ ID NO: 306 - protein, 15 aa, Homo sapiens
TSVSQNVIPSSAQKR

SEQ ID NO: 307 - protein, 15 aa, Homo sapiens
RIRDSEYEIQRQAKK

SEQ ID NO: 308 - protein, 15 aa, Homo sapiens
TYQFFVKHGELKVYK

SEQ ID NO: 309 - protein, 15 aa, Homo sapiens
DEEFYLSVGSPSVLL

SEQ ID NO: 310 - protein, 15 aa, Homo sapiens
REIILMDLVRPQDTY

SEQ ID NO: 311 - protein, 15 aa, Homo sapiens
YQFFVKHGELKVYKT

SEQ ID NO: 312 - protein, 15 aa, Homo sapiens
HDGYSLDGPEEIECT

SEQ ID NO: 313 - protein, 15 aa, Homo sapiens
KCSYTEDAQCIDGTI

SEQ ID NO: 314 - protein, 15 aa, Homo sapiens
GYSLDGPEEIECTKL

SEQ ID NO: 315 - protein, 15 aa, Homo sapiens
KVSFFCKNKEKKCSY

SEQ ID NO: 316 - protein, 15 aa, Homo sapiens
DLPFSTVVPLKTFYE

SEQ ID NO: 317 - protein, 15 aa, Homo sapiens
ECLPQHAMFGNDTIT

SEQ ID NO: 318 - protein, 15 aa, Homo sapiens
CKVPVKKATVVYQGE

SEQ ID NO: 319 - protein, 15 aa, Homo sapiens
KVSFFCKNKEKKCSY

SEQ ID NO: 320 - protein, 15 aa, Homo sapiens
YSCKPGYVSRGGMRK

SEQ ID NO: 321 - protein, 15 aa, Homo sapiens
KKATVVYQGERVKIQ

SEQ ID NO: 322 - protein, 15 aa, Homo sapiens
KVSFFCKNKEKKCSY

SEQ ID NO: 323 - protein, 15 aa, Homo sapiens
EVPKCFKEHSSLAFW

SEQ ID NO: 324 - protein, 15 aa, Homo sapiens
VPKCFKEHSSLAFWK

SEQ ID NO: 325 - protein, 15 aa, Homo sapiens
KCFKEHSSLAFWKTD

SEQ ID NO: 326 - protein, 15 aa, Homo sapiens
QPFMFRLDNRYQPME

SEQ ID NO: 327 - protein, 15 aa, Homo sapiens
RIKNQADCIPFFRSC

SEQ ID NO: 328 - protein, 15 aa, Homo sapiens
SSLRCMVDLGPCWAG

SEQ ID NO: 329 - protein, 15 aa, Homo sapiens
QQRQALAQISLPRII

SEQ ID NO: 330 - protein, 15 aa, Homo sapiens
RSLMFMQWGQLLDHD

SEQ ID NO: 331 - protein, 15 aa, Homo sapiens
QEARKIVGAMVQIIT

SEQ ID NO: 332 - protein, 15 aa, Homo sapiens
DNRYQPMEPNPRVPL

SEQ ID NO: 333 - protein, 15 aa, Homo sapiens
ELLSYFKQPVAATRT

SEQ ID NO: 334 - protein, 15 aa, Homo sapiens
QLGTVLRNLKLARKL

SEQ ID NO: 335 - protein, 15 aa, Homo sapiens
YLPLVLGPTAMRKYL

SEQ ID NO: 336 - protein, 15 aa, Homo sapiens
LGTVLRNLKLARKLM

SEQ ID NO: 337 - protein, 15 aa, Homo sapiens
KNNIFMSNSYPRDFV

SEQ ID NO: 338 - protein, 15 aa, Homo sapiens
SLQMRGNPGSHFCGG

SEQ ID NO: 339 - protein, 15 aa, Homo sapiens
TRVALYVDWIRSTLR

SEQ ID NO: 340 - protein, 15 aa, Homo sapiens
TLIHPSFVLTAAHCL

SEQ ID NO: 341 - protein, 15 aa, Homo sapiens
NDVLLIQLSSPANLS

SEQ ID NO: 342 - protein, 15 aa, Homo sapiens
DPPAQVLQELNVTVV

SEQ ID NO: 343 - protein, 15 aa, Homo sapiens
HCLRDIPQRLVNVVL

SEQ ID NO: 344 - protein, 15 aa, Homo sapiens
DWIRSTLRRVEAKGR

SEQ ID NO: 345 - protein, 15 aa, Homo sapiens
TLIHPSFVLTAAHCL

SEQ ID NO: 346 - protein, 15 aa, Homo sapiens
RPHNICTFVPRRKAG

SEQ ID NO: 347 - protein, 15 aa, Homo sapiens
HPSFVLTAAHCLRDI

SEQ ID NO: 348 - protein, 15 aa, Homo sapiens
DVLLIQLSSPANLSA

SEQ ID NO: 349 - protein, 15 aa, Homo sapiens
YVDWIRSTLRRVEAK

SEQ ID NO: 350 - protein, 15 aa, Homo sapiens
EYVTLKKMREIIGWP

SEQ ID NO: 351 - protein, 15 aa, Homo sapiens
NFFRMVISNPAATHQ

SEQ ID NO: 352 - protein, 15 aa, Homo sapiens
DSVILIKCDERGKMI

SEQ ID NO: 353 - protein, 15 aa, Homo sapiens
KISVSLPLSLSQSVC

SEQ ID NO: 354 - protein, 14 aa, Homo sapiens
QLSKDTSVLTFTFC

SEQ ID NO: 355 - protein, 17 aa, Homo sapiens
CSDAHPGDSSGDSSGLN

SEQ ID NO: 356 - protein, 15 aa, Homo sapiens
RGEVRQFTLRHWLKV

SEQ ID NO: 357 - protein, 13 aa, Homo sapiens
GDYLNDEALWNKC

SEQ ID NO: 358 - protein, 15 aa, Homo sapiens
GKVIDDNDHLSQEIC

SEQ ID NO: 359 - protein, 14 aa, Homo sapiens
LMANSTWGYPFHDG

SEQ ID NO: 360 - protein, 14 aa, Homo sapiens
LNVVPWNLTLFSIL

SEQ ID NO: 361 - protein, 12 aa, Homo sapiens
THSFTAFKRHVC

SEQ ID NO: 362 - protein, 13 aa, Homo sapiens
NLSLPPSLSLSIC

SEQ ID NO: 363 - protein, 16 aa, Homo sapiens
ERPSSVLTIYDIGIQC

SEQ ID NO: 364 - protein, 15 aa, Homo sapiens
CYQQYTNLQERPSSV

SEQ ID NO: 365 - protein, 15 aa, Homo sapiens [sequence omitted]

SEQ ID NO: 366 - protein, 17 aa, Homo sapiens
CSRKKRADKKENGTKLL

SEQ ID NO: 367 - protein, 9 aa, Homo sapiens
FLLVLGFII

SEQ ID NO: 368 - protein, 9 aa, Homo sapiens
VLPSVAMFL

SEQ ID NO: 369 - protein, 9 aa, Homo sapiens
LVLGFIIAL

SEQ ID NO: 370 - protein, 9 aa, Homo sapiens
KVVTSSFVV

SEQ ID NO: 371 - protein, 9 aa, Homo sapiens
LVPGTKFYI

SEQ ID NO: 372 - protein, 9 aa, Homo sapiens
LLPIRTLPL

SEQ ID NO: 373 - protein, 9 aa, Homo sapiens
YLVKKGTAT

SEQ ID NO: 374 - protein, 9 aa, Homo sapiens
SLFAETIWV

SEQ ID NO: 375 - protein, 9 aa, Homo sapiens
MLIAMYFYT

SEQ ID NO: 376 - protein, 9 aa, Homo sapiens
LMWTLPVML

SEQ ID NO: 377 - protein, 9 aa, Homo sapiens
MLIVYIFEC

SEQ ID NO: 378 - protein, 9 aa, Homo sapiens
YIFECASCI

SEQ ID NO: 379 - protein, 9 aa, Homo sapiens
LVLMLIVYI

SEQ ID NO: 380 - protein, 9 aa, Homo sapiens
ALCRRRSMV

SEQ ID NO: 381 - protein, 9 aa, Homo sapiens
LLSGLSLFA

SEQ ID NO: 382 - protein, 9 aa, Homo sapiens
FLLVVGLIV

SEQ ID NO: 383 - protein, 9 aa, Homo sapiens
LVVGLIVAL

SEQ ID NO: 384 - protein, 9 aa, Homo sapiens
KVVKSDFVV

SEQ ID NO: 385 - protein, 9 aa, Homo sapiens
TLPVQTLPL

SEQ ID NO: 386 - protein, 9 aa, Homo sapiens
DLHVISNDV

SEQ ID NO: 387 - protein, 9 aa, Homo sapiens
VLVHPQWVL

SEQ ID NO: 388 - protein, 9 aa, Homo sapiens
FLRPGDDSS

SEQ ID NO: 389 - protein, 9 aa, Homo sapiens
ALGTTCYAS

SEQ ID NO: 390 - protein, 9 aa, Homo sapiens
KLQCVDLHV

SEQ ID NO: 391 - protein, 9 aa, Homo sapiens
ELAHYDVLL

SEQ ID NO: 392 - protein, 9 aa, Homo sapiens
NLNGAGDPL

SEQ ID NO: 393 - protein, 9 aa, Homo sapiens
TLRVDCTPL

SEQ ID NO: 394 - protein, 9 aa, Homo sapiens
MMNDQLMFL

SEQ ID NO: 395 - protein, 9 aa, Homo sapiens
ALFDIESKV

SEQ ID NO: 396 - protein, 9 aa, Homo sapiens
LLHETDSAV

SEQ ID NO: 397 - protein, 9 aa, Homo sapiens
VLAKELKFV

SEQ ID NO: 398 - protein, 9 aa, Homo sapiens
ILLWQPIPV

SEQ ID NO: 399 - protein, 9 aa, Homo sapiens
DLFGIWSKV

SEQ ID NO: 400 - protein, 9 aa, Homo sapiens
PLERFAELV

SEQ ID NO: 401 - protein, 9 aa, Homo sapiens
KQGNFNAWV

SEQ ID NO: 402 - protein, 9 aa, Homo sapiens
NLLRRMWVT

SEQ ID NO: 403 - protein, 9 aa, Homo sapiens
NLFETPILA

SEQ ID NO: 404 - protein, 9 aa, Homo sapiens
NLFETPVEA

SEQ ID NO: 405 - protein, 9 aa, Homo sapiens
GLQHWVPEL

SEQ ID NO: 406 - protein, 9 aa, Homo sapiens
VQFVASYKV

SEQ ID NO: 407 - protein, 9 aa, Homo sapiens
RLLAALCGA

SEQ ID NO: 408 - protein, 9 aa, Homo sapiens
LLLLTVLTV

SEQ ID NO: 409 - protein, 9 aa, Homo sapiens
LLLTVLTVV

SEQ ID NO: 410 - protein, 9 aa, Homo sapiens
FLSFHISNL

SEQ ID NO: 411 - protein, 9 aa, Homo sapiens
LLVLVCVLV

SEQ ID NO: 412 - protein, 9 aa, Homo sapiens
ALLVLVCVL

SEQ ID NO: 413 - protein, 9 aa, Homo sapiens
SLSYTNPAV

SEQ ID NO: 414 - protein, 9 aa, Homo sapiens
NLTISDVSV

SEQ ID NO: 415 - protein, 9 aa, Homo sapiens
ALASTAPPV

SEQ ID NO: 416 - protein, 9 aa, Homo sapiens
AILCWTFWV

SEQ ID NO: 417 - protein, 9 aa, Homo sapiens
FILMFIVYA

SEQ ID NO: 418 - protein, 9 aa, Homo sapiens
LTAECIFFV

SEQ ID NO: 419 - protein, 9 aa, Homo sapiens
MLQDNCCGV

SEQ ID NO: 420 - protein, 9 aa, Homo sapiens
ILCWTFWVL

SEQ ID NO: 421 - protein, 9 aa, Homo sapiens
KILLAYFIL

SEQ ID NO: 422 - protein, 9 aa, Homo sapiens
FVGICLFCL

SEQ ID NO: 423 - protein, 9 aa, Homo sapiens
VLLSVAMFL

SEQ ID NO: 424 - protein, 9 aa, Homo sapiens
LLSVAMFLL

SEQ ID NO: 425 - protein, 9 aa, Homo sapiens
ILGSLPFFL

SEQ ID NO: 426 - protein, 9 aa, Homo sapiens
ILNAYLVRV

SEQ ID NO: 427 - protein, 9 aa, Homo sapiens
FLLVGFAGA

SEQ ID NO: 428 - protein, 9 aa, Homo sapiens
NLQPQLASV

SEQ ID NO: 429 - protein, 9 aa, Homo sapiens
CMFDSKEAL

SEQ ID NO: 430 - protein, 9 aa, Homo sapiens
YLYVLVDSA

SEQ ID NO: 431 - protein, 9 aa, Homo sapiens
YMDGTMSQV

SEQ ID NO: 432 - protein, 9 aa, Homo sapiens
KMARFSYSV

SEQ ID NO: 433 - protein, 9 aa, Homo sapiens
GLVMDEHLV

SEQ ID NO: 434 - protein, 9 aa, Homo sapiens
FLPGCDGLV

SEQ ID NO: 435 - protein, 9 aa, Homo sapiens
CMLGSFCAC

SEQ ID NO: 436 - protein, 9 aa, Homo sapiens
YLAFRDDSI

SEQ ID NO: 437 - protein, 9 aa, Homo sapiens
WLPKKCSLC

SEQ ID NO: 438 - protein, 9 aa, Homo sapiens
CLNGGTCML

SEQ ID NO: 439 - protein, 9 aa, Homo sapiens
MLVGICLSI

SEQ ID NO: 440 - protein, 9 aa, Homo sapiens
FELGLVAGL

SEQ ID NO: 441 - protein, 9 aa, Homo sapiens
KMVRFSYSV

SEQ ID NO: 442 - protein, 9 aa, Homo sapiens
CLNEGTCML

SEQ ID NO: 443 - protein, 9 aa, Homo sapiens
MLAGICLSI

SEQ ID NO: 444 - protein, 9 aa, Homo sapiens
RLLFFLLFL

SEQ ID NO: 445 - protein, 9 aa, Homo sapiens
TLAYLIFCL

SEQ ID NO: 446 - protein, 9 aa, Homo sapiens
LLFLTPMEV

SEQ ID NO: 447 - protein, 9 aa, Homo sapiens
KLMSPKLYV

SEQ ID NO: 448 - protein, 9 aa, Homo sapiens
LLFFLLFLV

SEQ ID NO: 449 - protein, 9 aa, Homo sapiens
SLFLGILSV

SEQ ID NO: 450 - protein, 9 aa, Homo sapiens
AISGMILSI

SEQ ID NO: 451 - protein, 9 aa, Homo sapiens
FIRAHTPYI

SEQ ID NO: 452 - protein, 9 aa, Homo sapiens
SLNFIRAHT

SEQ ID NO: 453 - protein, 9 aa, Homo sapiens
LKMESLNFI

SEQ ID NO: 454 - protein, 9 aa, Homo sapiens
SHFLKMESL

SEQ ID NO: 455 - protein, 9 aa, Homo sapiens
YLFLGILSV

SEQ ID NO: 456 - protein, 24 aa, Encephalomyocarditis virus
GIFNAHYAGYFADLLIHDIETNPG

SEQ ID NO: 457 - protein, 25 aa, Encephalomyocarditis virus
GIFNAHYAGYFADLLIHDIETNPGP

SEQ ID NO: 458 - protein, 25 aa, Encephalomyocarditis virus
RIFNAHYAGYFADLLIHDIETNPGP

SEQ ID NO: 459 - protein, 25 aa, Mengo virus
HVFETHYAGYFSDLLIHDVETNPGP

SEQ ID NO: 460 - protein, 25 aa, Theiler's encephalomyelitis virus
KAVRGYHADYYKQRLIHDVEMNPGP

SEQ ID NO: 461 - protein, 25 aa, Theiler's encephalomyelitis virus
RAVRAYHADYYKQRLIHDVEMNPGP

SEQ ID NO: 462 - protein, 25 aa, Theiler's encephalomyelitis virus
KAVRGYHADYYRQRLIHDVETNPGP

SEQ ID NO: 463 - protein, 25 aa, unidentified
  REGION 1..25: note = Description of Unknown:Theiler's-like virus
KHVREYHAAYYKQRLMHDVETNPGP

SEQ ID NO: 464 - protein, 26 aa, Ljungan virus
MHSDEMDFAGGKFLNQCGDVETNPGP

SEQ ID NO: 465 - protein, 26 aa, Ljungan virus
MHNDEMDYSGGKFLNQCGDVESNPGP

SEQ ID NO: 466 - protein, 26 aa, Ljungan virus
MHSDEMDFAGGKFLNQCGDVETNPGP

SEQ ID NO: 467 - protein, 26 aa, Ljungan virus
YHDKDMDYAGGKFLNQCGDVETNPGP

SEQ ID NO: 468 - protein, 19 aa, Foot-and-mouth disease virus
LLNFDLLKLAGDVESNPGP

SEQ ID NO: 469 - protein, 19 aa, Foot-and-mouth disease virus
LLNFDLLKLAGDVESNPGP

SEQ ID NO: 470 - protein, 19 aa, Foot-and-mouth disease virus
LLNFDLLKLAGDVESNPGP

SEQ ID NO: 471 - protein, 19 aa, Foot-and-mouth disease virus
LLNFDLLKLAGDMESNPGP

SEQ ID NO: 472 - protein, 19 aa, Foot-and-mouth disease virus
LTNFDLLKLAGDVESNPGP

SEQ ID NO: 473 - protein, 19 aa, Foot-and-mouth disease virus
LLNFDLLKLAGDVESNPGP

SEQ ID NO: 474 - protein, 19 aa, Foot-and-mouth disease virus
LLSFDLLKLAGDVESNPGP

SEQ ID NO: 475 - protein, 19 aa, Foot-and-mouth disease virus
MCNFDLLKLAGDVESNPGP

SEQ ID NO: 476 - protein, 19 aa, Foot-and-mouth disease virus
LLNFDLLKLAGDVESNPGP

SEQ ID NO: 477 - protein, 19 aa, Equine rhinitis A virus
CTNYSLLKLAGDVESNPGP

SEQ ID NO: 478 - protein, 20 aa, Equine rhinitis B virus
GATNFSLLKLAGDVELNPGP

SEQ ID NO: 479 - protein, 20 aa, Human endogenous retrovirus
GATNFDLLKLAGDVESNPGP

SEQ ID NO: 480 - protein, 22 aa, Porcine teschovirus 1
GPGATNFSLLKQAGDVEENPGP

SEQ ID NO: 481 - protein, 22 aa, Porcine teschovirus 2
GPGATNFSLLKQAGDVEENPGP

SEQ ID NO: 482 - protein, 22 aa, Porcine teschovirus 3
GPGASSFSLLKQAGDVEENPGP

SEQ ID NO: 483 - protein, 22 aa, Porcine teschovirus 4
GPGASNFSLLKQAGDVEENPGP

SEQ ID NO: 484 - protein, 22 aa, Porcine teschovirus 5
GPGAANFSLLRQAGDVEENPGP

SEQ ID NO: 485 - protein, 22 aa, Porcine teschovirus 6
GPGATNFSLLKQAGDVEENPGP

SEQ ID NO: 486 - protein, 22 aa, Porcine teschovirus 7
GPGATNFSLLKQAGDVEENPGP

SEQ ID NO: 487 - protein, 22 aa, Porcine teschovirus 8
GPGATNFSLLKQAGDIEENPGP

SEQ ID NO: 488 - protein, 22 aa, Porcine teschovirus 9
GPGATNFSLLKQAGDVEENPGP

SEQ ID NO: 489 - protein, 22 aa, Porcine teschovirus 10
GPGATNFSLLKQAGDVEENPGP

SEQ ID NO: 490 - protein, 22 aa, Porcine teschovirus 11
GPGATNFSLLKRAGDVEENPGP

SEQ ID NO: 491 - protein, 20 aa, Cricket paralysis virus
FLRKRTQLLMSGDVESNPGP

SEQ ID NO: 492 - protein, 20 aa, Drosophila C virus
EAARQMLLLLSGDVETNPGP

SEQ ID NO: 493 - protein, 20 aa, Acute bee paralysis virus
GSWTDILLLLSGDVETNPGP

SEQ ID NO: 494 - protein, 20 aa, Acute bee paralysis virus
GSWTDILLLLSGDVETNPGP

SEQ ID NO: 495 - protein, 20 aa, Acute bee paralysis virus
GSWTDILLLWSGDVETNPGP

SEQ ID NO: 496 - protein, 20 aa, Infectious flacherie virus
TRAEIEDELIRAGIESNPGP

SEQ ID NO: 497 - protein, 20 aa, Tomato aspermy virus
RAEGRGSLLTCGDVEENPGP

SEQ ID NO: 498 - protein, 20 aa, Equine encephalosis virus
QGAGRGSLVTCGDVEENPGP

SEQ ID NO: 499 - protein, 20 aa, Acyrthosiphon pisum virus
NYPMPEALQKIIDLESNPPP

SEQ ID NO: 500 - protein, 20 aa, Kashmir bee virus
GTWESVLNLLAGDIELNPGP

SEQ ID NO: 501 - protein, 20 aa, Perina nuda picorna-like virus
AQGWVPDLTVDGDVESNPGP

SEQ ID NO: 502 - protein, 20 aa, Perina nuda picorna-like virus
IGGGQKDLTQDGDIESNPGP

SEQ ID NO: 503 - protein, 20 aa, Ectropis obliqua picorna-like virus
AQGWAPDLTQDGDVESNPGP

SEQ ID NO: 504 - protein, 20 aa, Ectropis obliqua picorna-like virus
IGGGQRDLTQDGDIESNPGP

SEQ ID NO: 505 - protein, 19 aa, Providence virus
VGDRGSLLTCGDVESNPGP

SEQ ID NO: 506 - protein, 19 aa, Providence virus
SGGRGSLLTAGDVEKNPGP

SEQ ID NO: 507 - protein, 19 aa, Providence virus
GDPIEDLTDDGDIEKNPGP

SEQ ID NO: 508 - protein, 20 aa, Bovine rotavirus
SKFQIDRILISGDIELNPGP

SEQ ID NO: 509 - protein, 20 aa, Porcine rotavirus
AKFQIDKILISGDVELNPGP

SEQ ID NO: 510 - protein, 20 aa, Human rotavirus
SKFQIDKILISGDIELNPGP

SEQ ID NO: 511 - protein, 20 aa, Bombyx mori
FRSNYDLLKLCGDIESNPGP

SEQ ID NO: 512 - protein, 20 aa, Lymantria dispar
FRSNYDLLKLCGDVESNPGP

SEQ ID NO: 513 - protein, 20 aa, Dendrolimus punctatus
FRSNYDLLKLCGDVESNPGP

SEQ ID NO: 514 - protein, 20 aa, Trypanosoma brucei
SSIIRTKMLVSGDVEENPGP

SEQ ID NO: 515 - protein, 20 aa, Trypanosoma brucei
SSIIRTKMLLSGDVEENPGP

SEQ ID NO: 516 - protein, 20 aa, Trypanosoma brucei
SSIIRTKMLLSGDVEENPGP

SEQ ID NO: 517 - protein, 20 aa, Trypanosoma brucei
SSIIRTKILLSGDVEENPGP

SEQ ID NO: 518 - protein, 20 aa, Trypanosoma cruzi
CDAQRQKLLLSGDIEQNPGP

SEQ ID NO: 519 - protein, 21 aa, Thermotoga maritima
  MOD_RES 21: note = Any amino acid
YIPDFGGFLVKADSEFNPGPX

SEQ ID NO: 520 - protein, 21 aa, Bordetella bronchiseptica
VHCAGRGGPVRLLDKEGNPGP

SEQ ID NO: 521 - protein, 21 aa, Mus musculus
  MOD_RES 21: note = Any amino acid
DLELETVGSHQADAETNPGPX

SEQ ID NO: 522 - protein, 21 aa, Drosophila melanogaster
  MOD_RES 21: note = Any amino acid
TAADKIQGSWKMDTEGNPGPX

SEQ ID NO: 523 - protein, 20 aa, Aspergillus nidulans
PITNRPRNSGLIDTEINPGP

SEQ ID NO: 524 - DNA, 583 bp, Encephalomyocarditis virus
  source 1..583: mol_type = unassigned DNA
cccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccac

SEQ ID NO: 525 - DNA, 366 bp, synthetic construct
  misc_feature 1..366: note = Description of Artificial Sequence: Syntheticpolynucleotide
  source 1..366: mol_type = other DNA
ctgggcggtcaacaagtgcgggcctggctcagcgcgggggggcgcggagaccgcgaggcgaccgggagcggctgggttcccggctgcgcgcccttcggccaggccgggagccgcgccagtcggagcccccggcccagcgtggtccgcctccctctgggcgtccacctgcccggagtactgccagcgggcatgaccgacccaccaggggcgccgccgccggcgctcgcaggccgcggatgaagaagaaaacccggcgccgctcgacccggagcgaggagttgacccggagcgaggagttgaccctgagtgaggaagcgacctggagtgaagaggcgacccagagtgaggaggcgacccagggcgaag

SEQ ID NO: 526 - DNA, 92 bp, synthetic construct
  misc_feature 1..92: note = Description of Artificial Sequence: Syntheticoligonucleotide
  source 1..92: mol_type = other DNA
aaaagaaggaaaaagaaggaaaagaaggaaaaagaaggctgcaggcggctgcagaaaagaaggaaaaagaaggaaaagaaggaaaaagaagg

SEQ ID NO: 527 - protein, 611 aa, synthetic construct
  REGION 1..611: note = Description of Artificial Sequence: Syntheticpolypeptide
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAATNSGGSNYKLTFGKGTLLTVNPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSSGSGATNFSLLKQAGDVEENPGPMSNQVLCCVVLCFLGANTVDGGITQSPKYLFRKEGQNVTLSCEQNLNHDAMYWYRQDPGQGLRLIYYSQIVNDFQKGDIAEGYSVSREKKESFPLTVTSAQKNPTAFYLCASGGRVYQPQHFGDGTRLSILEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 528 - protein, 281 aa, synthetic construct
  REGION 1..281: note = Description of Artificial Sequence: Syntheticpolypeptide
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAATNSGGSNYKLTFGKGTLLTVNPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 529 - protein, 308 aa, synthetic construct
  REGION 1..308: note = Description of Artificial Sequence: Syntheticpolypeptide
MSNQVLCCVVLCFLGANTVDGGITQSPKYLFRKEGQNVTLSCEQNLNHDAMYWYRQDPGQGLRLIYYSQIVNDFQKGDIAEGYSVSREKKESFPLTVTSAQKNPTAFYLCASGGRVYQPQHFGDGTRLSILEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 530 - protein, 255 aa, synthetic construct
  REGION 1..255: note = Description of Artificial Sequence: Syntheticpolypeptide
DQQVKQNSPSLSVQEGRISILNCDYTNSMFDYFLWYKKYPAEGPTFLISISSIKDKNEDGRFTVFLNKSAKHLSLHIVPSQPGDSAVYFCAATNSGGSNYKLTFGKGTLLTVNPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 531 - protein, 289 aa, synthetic construct
  REGION 1..289: note = Description of Artificial Sequence: Syntheticpolypeptide
DGGITQSPKYLFRKEGQNVTLSCEQNLNHDAMYWYRQDPGQGLRLIYYSQIVNDFQKGDIAEGYSVSREKKESFPLTVTSAQKNPTAFYLCASGGRVYQPQHFGDGTRLSILEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 532 - DNA, 1622 bp, synthetic construct
  misc_feature 1..1622: note = Description of Artificial Sequence: Syntheticpolynucleotide
  source 1..1622: mol_type = other DNA
atggccatgctcctgggggcatcagtgctgattctgtggcttcagccagactgggtaaacagtcaacagaagaatgatgaccagcaagttaagcaaaattcaccatccctgagcgtccaggaaggaagaatttctattctgaactgtgactatactaacagcatgtttgattatttcctatggtacaaaaaataccctgctgaaggtcctacattcctgatatctataagttccattaaggataaaaatgaagatggaagattcactgtcttcttaaacaaaagtgccaagcacctctctctgcacattgtgccctcccagcctggagactctgcagtgtacttctgtgcagcaacaaatagtggaggtagcaactataaactgacatttggaaaaggaactctcttaaccgtgaatccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagcggctccggagccacgaacttctctctgttaaagcaagcaggagacgtggaagaaaaccccggtcccatgagcaaccaggtgctctgctgtgtggtcctttgtttcctgggagcaaacaccgtggatggtggaatcactcagtccccaaagtacctgttcagaaaggaaggacagaatgtgaccctgagttgtgaacagaatttgaaccacgatgccatgtactggtaccgacaggacccagggcaagggctgagattgatctactactcacagatagtaaatgactttcagaaaggagatatagctgaagggtacagcgtctctcgggagaagaaggaatcctttcctctcactgtgacatcggcccaaaagaacccgacagctttctatctctgtgccagtgggggacgggtctatcagccccagcattttggtgatgggactcgactctccatcctagaggacctgaacaaggtgttcccacccgaggtcgctgtgtttgagccatcagaagcagagatctcccacacccaaaaggccacactggtgtgcctggccacaggcttcttccctgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatga

SEQ ID NO: 533 - protein, 603 aa, synthetic construct
  REGION 1..603: note = Description of Artificial Sequence: Syntheticpolypeptide
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAATNSGGSNYKLTFGKGTLLTVNPNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSSGSGATNFSLLKQAGDVEENPGPMSNQVLCCVVLCFLGANTVDGGITQSPKYLFRKEGQNVTLSCEQNLNHDAMYWYRQDPGQGLRLIYYSQIVNDFQKGDIAEGYSVSREKKESFPLTVTSAQKNPTAFYLCASGGRVYQPQHFGDGTRLSILEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 534 - protein, 277 aa, synthetic construct
  REGION 1..277: note = Description of Artificial Sequence: Syntheticpolypeptide
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAATNSGGSNYKLTFGKGTLLTVNPNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 535 - protein, 304 aa, synthetic construct
  REGION 1..304: note = Description of Artificial Sequence: Syntheticpolypeptide
MSNQVLCCVVLCFLGANTVDGGITQSPKYLFRKEGQNVTLSCEQNLNHDAMYWYRQDPGQGLRLIYYSQIVNDFQKGDIAEGYSVSREKKESFPLTVTSAQKNPTAFYLCASGGRVYQPQHFGDGTRLSILEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 536 - protein, 251 aa, synthetic construct
  REGION 1..251: note = Description of Artificial Sequence: Syntheticpolypeptide
DQQVKQNSPSLSVQEGRISILNCDYTNSMFDYFLWYKKYPAEGPTFLISISSIKDKNEDGRFTVFLNKSAKHLSLHIVPSQPGDSAVYFCAATNSGGSNYKLTFGKGTLLTVNPNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 537 - protein, 285 aa, synthetic construct
  REGION 1..285: note = Description of Artificial Sequence: Syntheticpolypeptide
DGGITQSPKYLFRKEGQNVTLSCEQNLNHDAMYWYRQDPGQGLRLIYYSQIVNDFQKGDIAEGYSVSREKKESFPLTVTSAQKNPTAFYLCASGGRVYQPQHFGDGTRLSILEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 538 - protein, 605 aa, synthetic construct
  REGION 1..605: note = Description of Artificial Sequence: Syntheticpolypeptide
METLLGVSLVILWLQLARVNSQQGEEDPQALSIQEGENATMNCSYKTSINNLQWYRQNSGRGLVHLILIRSNEREKHSGRLRVTLDTSKKSSSLLITASRAADTASYFCATGRMDSSYKLIFGSGTRLLVRPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSSGSGATNFSLLKQAGDVEENPGPMDSWTFCCVSLCILVAKHTDAGVIQSPRHEVTEMGQEVTLRCKPISGHNSLFWYRQTMMRGLELLIYFNNNVPIDDSGMPEDRFSAKMPNASFSTLKIQPSEPRDSAVYFCASSEQLSGNTIYFGEGSWLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 539 - protein, 273 aa, synthetic construct
  REGION 1..273: note = Description of Artificial Sequence: Syntheticpolypeptide
METLLGVSLVILWLQLARVNSQQGEEDPQALSIQEGENATMNCSYKTSINNLQWYRQNSGRGLVHLILIRSNEREKHSGRLRVTLDTSKKSSSLLITASRAADTASYFCATGRMDSSYKLIFGSGTRLLVRPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 540 - protein, 310 aa, synthetic construct
  REGION 1..310: note = Description of Artificial Sequence: Syntheticpolypeptide
MDSWTFCCVSLCILVAKHTDAGVIQSPRHEVTEMGQEVTLRCKPISGHNSLFWYRQTMMRGLELLIYFNNNVPIDDSGMPEDRFSAKMPNASFSTLKIQPSEPRDSAVYFCASSEQLSGNTIYFGEGSWLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 541 - protein, 252 aa, synthetic construct
  REGION 1..252: note = Description of Artificial Sequence: Syntheticpolypeptide
QQGEEDPQALSIQEGENATMNCSYKTSINNLQWYRQNSGRGLVHLILIRSNEREKHSGRLRVTLDTSKKSSSLLITASRAADTASYFCATGRMDSSYKLIFGSGTRLLVRPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 542 - protein, 289 aa, synthetic construct
  REGION 1..289: note = Description of Artificial Sequence: Syntheticpolypeptide
GVIQSPRHEVTEMGQEVTLRCKPISGHNSLFWYRQTMMRGLELLIYFNNNVPIDDSGMPEDRFSAKMPNASFSTLKIQPSEPRDSAVYFCASSEQLSGNTIYFGEGSWLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 543 - protein, 593 aa, synthetic construct
  REGION 1..593: note = Description of Artificial Sequence: Syntheticpolypeptide
MHSLHVSLVFLWLQLGGVSSQEKVQQSPESLTVPEGAMASLNCTISDSASQSIWWYQQNPGKGPKALISIFSNGNKKEGRLTVYLNRASLHVSLHIRDSHPSDSAVYLCAASLAGSWQLIFGSGTQLTVMPDIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSSATNFSLLKQAGDVEENPGPMGSIFLSCLAVCLLVAGPVDPKIIQKPKYLVAVTGSEKILICEQYLGHNAMYWYRQSAKKPLEFMFSYSYQKLMDNQTASSRFQPQSSKKNHLDLQITALKPDDSATYFCASSQGGTTNSDYTFGSGTRLLVIEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKRKNS

SEQ ID NO: 544 - protein, 268 aa, synthetic construct
  REGION 1..268: note = Description of Artificial Sequence: Syntheticpolypeptide
MHSLHVSLVFLWLQLGGVSSQEKVQQSPESLTVPEGAMASLNCTISDSASQSIWWYQQNPGKGPKALISIFSNGNKKEGRLTVYLNRASLHVSLHIRDSHPSDSAVYLCAASLAGSWQLIFGSGTQLTVMPDIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 545 - protein, 306 aa, synthetic construct
  REGION 1..306: note = Description of Artificial Sequence: Syntheticpolypeptide
MGSIFLSCLAVCLLVAGPVDPKIIQKPKYLVAVTGSEKILICEQYLGHNAMYWYRQSAKKPLEFMFSYSYQKLMDNQTASSRFQPQSSKKNHLDLQITALKPDDSATYFCASSQGGTTNSDYTFGSGTRLLVIEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKRKNS

SEQ ID NO: 546 - protein, 243 aa, synthetic construct
  REGION 1..243: note = Description of Artificial Sequence: Syntheticpolypeptide
QSPESLTVPEGAMASLNCTISDSASQSIWWYQQNPGKGPKALISIFSNGNKKEGRLTVYLNRASLHVSLHIRDSHPSDSAVYLCAASLAGSWQLIFGSGTQLTVMPDIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 547 - protein, 285 aa, synthetic construct
  REGION 1..285: note = Description of Artificial Sequence: Syntheticpolypeptide
KIIQKPKYLVAVTGSEKILICEQYLGHNAMYWYRQSAKKPLEFMFSYSYQKLMDNQTASSRFQPQSSKKNHLDLQITALKPDDSATYFCASSQGGTTNSDYTFGSGTRLLVIEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKRKNS

SEQ ID NO: 548 - DNA, 1782 bp, synthetic construct
  misc_feature 1..1782: note = Description of Artificial Sequence: Syntheticpolynucleotide
  source 1..1782: mol_type = other DNA
atgcattccttacatgtttcactagtgttcctctggcttcaactaggtggggtgagcagccaggagaaggtacagcagagcccagaatctctcacagtcccagagggagccatggcctccctcaactgcactatcagcgacagtgcttctcagtccatctggtggtaccaacagaatcctgggaaaggccccaaagcactaatatccatattctctaatggcaacaagaaagaaggcagattgacagtttacctcaatagagccagcctgcatgtttccctgcacatcagagactcccatcccagtgactccgccgtctacctctgtgcagcgagccttgcgggcagctggcaactcatctttggatctggaacccaactgacagttatgcctgacatccagaacccagaacctgctgtgtaccagttaaaagatcctcggtctcaggacagcaccctctgcctgttcaccgactttgactcccaaatcaatgtgccgaaaaccatggaatctggaacgttcatcactgacaaaactgtgctggacatgaaagctatggattccaagagcaatggggccattgcctggagcaaccagacaagcttcacctgccaagatatcttcaaagagaccaacgccacctaccccagttcagacgttccctgtgatgccacgttgaccgagaaaagctttgaaacagatatgaacctaaactttcaaaacctgtcagttatgggactccgaatcctcctgctgaaagtagccggatttaacctgctcatgacgctgaggctgtggtccagtgccacgaacttctctctgttaaagcaagcaggagacgtggaagaaaaccccggtcccatgggctccattttcctcagttgcctggccgtttgtctcctggtggcaggtccagtcgacccgaaaattatccagaaaccaaaatatctggtggcagtcacagggagcgaaaaaatcctgatatgcgaacagtatctaggccacaatgctatgtattggtatagacaaagtgctaagaagcctctagagttcatgttttcctacagctatcaaaaacttatggacaatcagactgcctcaagtcgcttccaacctcaaagttcaaagaaaaaccatttagaccttcagatcacagctctaaagcctgatgactcggccacatacttctgtgccagcagccaaggggggacaacaaactccgactacaccttcggctcagggaccaggcttttggtaatagaggatctgagaaatgtgactccacccaaggtctccttgtttgagccatcaaaagcagagattgcaaacaaacaaaaggctaccctcgtgtgcttggccaggggcttcttccctgaccacgtggagctgagctggtgggtgaatggcaaggaggtccacagtggggtcagcacggaccctcaggcctacaaggagagcaattatagctactgcctgagcagccgcctgagggtctctgctaccttctggcacaatcctcgcaaccacttccgctgccaagtgcagttccatgggctttcagaggaggacaagtggccagagggctcacccaaacctgtcacacagaacatcagtgcagaggcctggggccgagcagactgtggaatcacttcagcatcctatcatcagggggttctgtctgcaaccatcctctatgagatcctactggggaaggccaccctatatgctgtgctggtcagtggcctggtgctgatggccatggtcaagagaaaaaattcctga

SEQ ID NO: 549 - protein, 458 aa, Homo sapiens
MNRGVPFRHLLLVLQLALLPAATQGKKVVLGKKGDTVELTCTASQKKSIQFHWKNSNQIKILGNQGSFLTKGPSKLNDRADSRRSLWDQGNFPLIIKNLKIEDSDTYICEVEDQKEEVQLLVFGLTANSGTHLLQGQSLTLTLESPPGSSPSVQCRSPRGKNIQGGKTLSVSQLELQDSGTWTCTVLQNQKKVEFKIDIVVLAFQKASSIVYKKEGEQVEFSFPLAFTVEKLTGSGELWWQAERASSSKSWITFDLKNKEVSVKRVTQDPKLQMGKKLPLHLTLPQALPQYAGSGNLTLALEAKTGKLHQEVNLVVMRATQLQKNLTCEVWGPTSPKLMLSLKLENKEAKVSKREKAVWVLNPEAGMWQCLLSDSGQVLLESNIKVLPTWSTPVQPMALIVLGGVAGLLLFIGLGIFFCVRCRHRRRQAERMSQIKRLLSEKKTCQCPHRFQKTCSPI

SEQ ID NO: 550 - DNA, 1661 bp, Homo sapiens
  source 1..1661: mol_type = genomic DNA
atgaaccggggagtcccttttaggcacttgcttctggtgctgcaactggcgctcctcccagcagccactcagggaaagaaagtggtgctgggcaaaaaaggggatacagtggaactgacctgtacagcttcccagaagaagagcatacaattccactggaaaaactccaaccagataaagattctgggaaatcagggctccttcttaactaaaggtccatccaagctgaatgatcgcgctgactcaagaagaagcctttgggaccaaggaaactttcccctgatcatcaagaatcttaagatagaagactcagatacttacatctgtgaagtggaggaccagaaggaggaggtgcaattgctagtgttcggattgactgccaactctggtacccacctgcttcaggggcagagcctgaccctgaccttggagagcccccctggtagtagcccctcagtgcaatgtaggagtccaaggggtaaaaacatacagggggggaagaccctctccgtgtctcagctggagctccaggatagtggcacctggacgtgcactgtcttgcagaaccagaagaaggtggagttcaaaatagacatcgtggtgctagctttccagaaggcctccagcatagtctataagaaagagggggaacaggtggagttctccttcccactcgcctttacagttgaaaagctgacgggcagtggcgagctgtggtggcaggcggagagggcttcctcctccaagtcttggatcacctttgacctgaagaacaaggaagtgtctgtaaaacgggttacccaggaccctaagctccagatgggcaagaagctcccgctccacctcaccctgccccaggccttgcctcagtatgctggctctggaaacctcaccctggcccttgaagcgaaaacaggaaagttgcatcaggaagtgaacctggtggtgatgagagccactcagctccagaaaaatttgacctgtgaggtgtggggacccacctcccctaagctgatgctgagcttgaaactggagaacaaggaggcaaaggtctcgaagcgggagaaggcggtgtgggtgctgaaccctgaggcggggatgtggcagtgtctgctgagtgactcgggacaggtcctgctggaatccaacatcaaggttctgcccacatggtccaccccggtgcagccaatggccctgattgtgctggggggcgtcgccggcctcctgcttttcattgggctaggcatcttcttctgtgtcaggtgccggcaccgaaggcgccaagcagagcggatgtctcagatcaagagactcctcagtgagaagaagacctgccagtgccctcaccggtttcagaagacatgtagccccatttgagtcgacaagggcgaattaattcagatcttacgtagctagcggatcccaattgctcgagcgggatcaattccgccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctgg

SEQ ID NO: 551 - protein, 457 aa, Mus sp.
MCRAISLRRLLLLLLQLSQLLAVTQGKTLVLGKEGESAELPCESSQKKITVFTWKFSDQRKILGQHGKGVLIRGGSPSQFDRFDSKKGAWEKGSFPLIINKLKMEDSQTYICELENRKEEVELWVFKVTFSPGTSLLQGQSLTLTLDSNSKVSNPLTECKHKKGKVVSGSKVLSMSNLRVQDSDFWNCTVTLDQKKNWFGMTLSVLGFQSTAITAYKSEGESAEFSFPLNFAEENGWGELMWKAEKDSFFQPWISFSIKNKEVSVQKSTKDLKLQLKETLPLTLKIPQVSLQFAGSGNLTLTLDKGTLHQEVNLVVMKVAQLNNTLTCEVMGPTSPKMRLTLKQENQEARVSEEQKVVQVVAPETGLWQCLLSEGDKVKMDSRIQVLSRGVNQTVFLACVLGGSFGFLGFLGLCILCCVRCRHQQRQAARMSQIKRLLSEKKTCQCPHRMQKSHNLI

SEQ ID NO: 552 - DNA, 1620 bp, Mus sp.
  source 1..1620: mol_type = genomic DNA
atgtgccgagccatctctcttaggcgcttgctgctgctgctgctgcagctgtcacaactcctagctgtcactcaagggaagacgctggtgctggggaaggaaggggaatcagcagaactgccctgcgagagttcccagaagaagatcacagtcttcacctggaagttctctgaccagaggaagattctggggcagcatggcaaaggtgtattaattagaggaggttcgccttcgcagtttgatcgttttgattccaaaaaaggggcatgggagaaaggatcgtttcctctcatcatcaataaacttaagatggaagactctcagacttatatctgtgagctggagaacaggaaagaggaggtggagttgtgggtgttcaaagtgaccttcagtccgggtaccagcctgttgcaagggcagagcctgaccctgaccttggatagcaactctaaggtctctaaccccttgacagagtgcaaacacaaaaagggtaaagttgtcagtggttccaaagttctctccatgtccaacctaagggttcaggacagcgacttctggaactgcaccgtgaccctggaccagaaaaagaactggttcggcatgacactctcagtgctgggttttcagagcacagctatcacggcctataagagtgagggagagtcagcggagttctccttcccactcaactttgcagaggaaaacgggtggggagagctgatgtggaaggcagagaaggattctttcttccagccctggatctccttctccataaagaacaaagaggtgtccgtacaaaagtccaccaaagacctcaagctccagctgaaggaaacgctcccactcaccctcaagataccccaggtctcgcttcagtttgctggttctggcaacctgactctgactctggacaaagggacactgcatcaggaagtgaacctggtggtgatgaaagtggctcagctcaacaatactttgacctgtgaggtgatgggacctacctctcccaagatgagactgaccctgaagcaggagaaccaggaggccagggtctctgaggagcagaaagtagttcaagtggtggcccctgagacagggctgtggcagtgtctactgagtgaaggtgataaggtcaagatggactccaggatccaggttttatccagaggggtgaaccagacagtgttcctggcttgcgtgctgggtggctccttcggctttctgggtttccttgggctctgcatcctctgctgtgtcaggtgccggcaccaacagcgccaggcagcacgaatgtctcagatcaagaggctcctcagtgagaagaagacctgccagtgcccccaccggatgcagaagagccataatctcatctgaagcggccgcgtcgactcgagcgggatcaattccgccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctgg

SEQ ID NO: 553 - protein, 773 aa, synthetic construct
  REGION 1..773: note = Description of Artificial Sequence: Syntheticpolypeptide
MEHSGILASLILIAVLPQGSPFKIQVTEYEDKVFVTCNTSVMHLDGTVEGWFAKNKTLNLGKGVLDPRGIYLCNGTEQLAKVVSSVQVHYRMCQNCVELDSGTMAGVIFIDLIATLLLALGIYCFAGHETGRPSGAAEVQALLKNEQLYQPLRDREDTQYSRLGGNWPRNKKSGPVKQTLNFDLLKLAGDVESNPGPMEQRKGLAGLFLVISLLQGTVAQTNKAKNLVQVDGSRGDGSVLLTCGLTDKTIKWLKDGSIISPLNATKNTWNLGNNAKDPRGTYQCQGAKETSNPLQVYYRMCENCIELNIGTISGFIFAEVISIFFLALGVYLIAGQDGVRQSRASDKQTLLQNEQLYQPLKDREYDQYSHLQGNQLRKKRSEGRGSLLTCGDVEENPGPMRWNTFWGILCLSLLAVGTCQDDAENIEYKVSISGTSVELTCPLDSDENLKWEKNGQELPQKHDKHLVLQDFSEVEDSGYYVCYTPASNKNTYLYLKARVCEYCVEVDLTAVAIIIIVDICITLGLLMVIYYWSKNRKAKAKPVTRGTGAGSRPRGQNKERPPPVPNPDYEPIRKGQRDLYSGLNQRAVGSATNFSLLKQAGDVEENPGPMKWKVSVLACILHVRFPGAEAQSFGLLDPKLCYLLDGILFIYGVIITALYLRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPR

SEQ ID NO: 554 - DNA, 2322 bp, synthetic construct
  misc_feature 1..2322: note = Description of Artificial Sequence: Syntheticpolynucleotide
  source 1..2322: mol_type = other DNA
atggaacacagcgggattctggctagtctgatactgattgctgttctcccccaagggagccccttcaagatacaagtgaccgaatatgaggacaaagtatttgtgacctgcaataccagcgtcatgcatctagatggaacggtggaaggatggtttgcaaagaataaaacactcaacttgggcaaaggcgttctggacccacgagggatatatctgtgtaatgggacagagcagctggcaaaggtggtgtcttctgtgcaagtccattaccgaatgtgccagaactgtgtggagctagactcgggcaccatggctggtgtcatcttcattgacctcatcgcaactctgctcctggctttgggcatctactgctttgcaggacatgagaccggaaggccttctggggctgctgaggttcaagcactgctgaagaatgagcagctgtatcagcctcttcgagatcgtgaagatacccagtacagccgtcttggagggaactggccccggaacaagaaatccggaccggtgaaacagactttgaattttgaccttctcaagttggcgggagacgtggagtccaacccagggcccatggagcagaggaagggtctggctggcctcttcctggtgatctctcttcttcaaggcactgtagcccagacaaataaagcaaagaatttggtacaagtggatggcagccgaggagacggttctgtacttctgacttgtggcttgactgacaagactatcaagtggcttaaagacgggagcataataagtcctctaaatgcaactaaaaacacatggaatctgggcaacaatgccaaagaccctcgaggcacgtatcagtgtcaaggagcaaaggagacgtcaaaccccctgcaagtgtattacagaatgtgtgaaaactgcattgagctaaacataggcaccatatccggctttatcttcgctgaggtcatcagcatcttcttccttgctcttggtgtatatctcattgcgggacaggatggagttcgccagtcaagagcttcagacaagcagactctgttgcaaaatgaacagctgtaccagcccctcaaggaccgggaatatgaccagtacagccatctccaaggaaaccaactgaggaagaagagatctgagggcagaggaagtctgctaacatgcggtgacgtcgaggagaatcctggcccaatgcggtggaacactttctggggcatcctgtgcctcagcctcctagctgttggcacttgccaggacgatgccgagaacattgaatacaaagtctccatctcaggaaccagtgtagagttgacgtgccctctagacagtgacgagaacttaaaatgggaaaaaaatggccaagagctgcctcagaagcatgataagcacctggtgctccaggatttctcggaagtcgaggacagtggctactacgtctgctacacaccagcctcaaataaaaacacgtacttgtacctgaaagctcgagtgtgtgagtactgtgtggaggtggacctgacagcagtagccataatcatcattgttgacatctgtatcactctgggcttgctgatggtcatttattactggagcaagaataggaaggccaaggccaagcctgtgacccgaggaaccggtgctggtagcaggcccagagggcaaaacaaggagcggccaccacctgttcccaacccagactatgagcccatccgcaaaggccagcgggacctgtattctggcctgaatcagagagcagtcggatccgccacgaacttctctctgttaaagcaagcaggagacgtggaagaaaaccccggtcccatgaagtggaaagtgtctgttctcgcctgcatcctccacgtgcggttcccaggagcagaggcacagagctttggtctgctggaccccaaactctgctacttgctagatggaatcctcttcatctacggagtcatcatcacagccctgtacctgagagcaaaattcagcaggagtgcagagactgctgccaacctgcaggaccccaaccagctctacaatgagctcaatctagggcgaagagaggaatatgacgtcttggagaagaagcgggctcgggacccagagatgggaggcaaacagcagaggaggaggaacccccaggaaggcgtatacaatgcactgcagaaagacaagatggcagaagcctacagtgagatcggcacaaaaggcgagaggcggagaggcaaggggcacgatggcctttaccagggtctcagcactgccaccaaggacacctatgatgccctgcatatgcagaccctggcccctcgctaa

SEQ ID NO: 555 - protein, 550 aa, unidentified
  REGION 1..550: note = Description of Unknown:luciferase protein sequence
MEDAKNIKKGPAPFYPLEDGTAGEQLHKAMKRYALVPGTIAFTDAHIEVNITYAEYFEMSVRLAEAMKRYGLNTNHRIVVCSENSLQFFMPVLGALFIGVAVAPANDIYNERELLNSMNISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKGGKSKL

SEQ ID NO: 556 - DNA, 1904 bp, unidentified
  misc_feature 1..1904: note = Description of Unknown:luciferase sequence
  source 1..1904: mol_type = unassigned DNA
aaaggaggaaaaactgtttcatacagaaggcgtggaggaaaaactgtttcatacagaaggcgtggaggaaaaactgtttcatacagaaggcgtattttgacacccccataatatttttccagaattaacagtataaattgcatctcttgttcaagagttccctatcactctctttaatcactactcacagtaacctcaactcctgccacaggtaccgagctcaagtttgtacaaaaaagcaggctgccaccatggaagacgccaaaaacataaagaaaggcccggcgccattctatccgctagaggatggaaccgctggagagcaactgcataaggctatgaagagatacgccctggttcctggaacaattgcttttacagatgcacatatcgaggtgaacatcacgtacgcggaatacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgaacatttcgcagcctaccgtagtgtttgtttccaaaaaggggttgcaaaaaattttgaacgtgcaaaaaaaattaccaataatccagaaaattattatcatggattctaaaacggattaccagggatttcagtcgatgtacacgttcgtcacatctcatctacctcccggttttaatgaatacgattttgtaccagagtcctttgatcgtgacaaaacaattgcactgataatgaactcctctggatctactgggttacctaagggtgtggcccttccgcatagaactgcctgcgtcagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttttacgatcccttcaggattacaaaattcaaagtgcgttgctagtaccaaccctattttcattcttcgccaaaagcactctgattgacaaatacgatttatctaatttacacgaaattgcttctgggggcgcacctctttcgaaagaagtcggggaagcggttgcaaaacgcttccatcttccagggatacgacaaggatatgggctcactgagactacatcagctattctgattacacccgagggggatgataaaccgggcgcggtcggtaaagttgttccattttttgaagcgaaggttgtggatctggataccgggaaaacgctgggcgttaatcagagaggcgaattatgtgtcagaggacctatgattatgtccggttatgtaaacaatccggaagcgaccaacgccttgattgacaaggatggatggctacattctggagacatagcttactgggacgaagacgaacacttcttcatagttgaccgcttgaagtctttaattaaatacaaaggataccaggtggcccccgctgaattggagtcgatattgttacaacaccccaacatcttcgacgcgggcgtggcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagtccaaattgtaa

SEQ ID NO: 557 - DNA, 876 bp, synthetic construct
  misc_feature 1..876: note = Description of Artificial Sequence: Syntheticpolynucleotide
  source 1..876: mol_type = other DNA
ctgggagcaaacaccgtggatggtggaatcactcagtccccaaagtacctgttcagaaaggaaggacagaatgtgaccctgagttgtgaacagaatttgaaccacgatgccatgtactggtaccgacaggacccagggcaagggctgagattgatctactactcacagatagtaaatgactttcagaaaggagatatagctgaagggtacagcgtctctcgggagaagaaggaatcctttcctctcactgtgacatcggcccaaaagaacccgacagctttctatctctgtgccagtgggggacgggtctatcagccccagcattttggtgatgggactcgactctccatcctagaggatctgagaaatgtgactccacccaaggtctccttgtttgagccatcaaaagcagagattgcaaacaaacaaaaggctaccctcgtgtgcttggccaggggcttcttccctgaccacgtggagctgagctggtgggtgaatggcaaggaggtccacagtggggtcagcacggaccctcaggcctacaaggagagcaattatagctactgcctgagcagccgcctgagggtctctgctaccttctggcacaatcctcgcaaccacttccgctgccaagtgcagttccatgggctttcagaggaggacaagtggccagagggctcacccaaacctgtcacacagaacatcagtgcagaggcctggggccgagcagactgtgggattacctcagcatcctatcaacaaggggtcttgtctgccaccatcctctatgagatcctgctagggaaagccaccctgtatgctgtgcttgtcagtacactggtggtgatggctatggtcaaaagaaagaattcatga

SEQ ID NO: 558 - protein, 7 aa, synthetic construct
  REGION 1..7: note = Description of Artificial Sequence: Syntheticpeptide
  MOD_RES 1: note = Val or Ile
  MOD_RES 3: note = Any amino acid
XEXNPGP

SEQ ID NO: 559 - protein, 6 aa, synthetic construct
  REGION 1..6: note = Description of Artificial Sequence: Synthetic6xHis tag
HHHHHH

(19) United States
(12) Patent Application Publication
(10) Pub. No.: US 2024/0201171 A1
(43) Pub. Date: Jun. 20, 2024

(54) ASSAY TO MEASURE THE POTENCY OF RECEPTOR-LIGAND INTERACTIONS IN NANOMEDICINES

(71) Applicant: UTI Limited Partnership, Calgary (CA)

(72) Inventor: Pedro SANTAMARIA, Calgary (CA)

(21) Appl. No.: 18/459,960

(22) Filed: Sep. 1, 2023

Related U.S. Application Data
(62) Division of application No. 16/603,180, filed on Oct. 4, 2019, now abandoned.
(60) Provisional application No. 62/483,298, filed on Apr. 7, 2017.

(51) Int. Cl. G01N 33/50; A61K 39/00; A61K 47/69; C07K 14/725; C07K 14/74; C12N 5/0783; G01N 33/569

(52) U.S. Cl. CPC G01N 33/505; A61K 39/0008; A61K 47/6929; C07K 14/7051; C07K 14/70539; C12N 5/0636; G01N 33/56977; A61K 2039/55516; A61K 2039/605

(57) ABSTRACT

Described herein, is an isolated cell comprising a recombinant T cell receptor (TCR) and a TCR-pathway-dependent reporter, wherein the recombinant T cell receptor is specific for a disease-relevant antigen bound to an MHC molecule. Also described are methods of use for the isolated cell as an assay to determine the function or potency of a peptide-major histocompatibility complex (pMHC) coupled to a nanoparticle (pMHC-NP) that can be used as a medicine for treating an autoimmune disease or cancer.

### CROSS-REFERENCE TO RELATED APPLICATIONS

This application is a divisional of U.S. application Ser. No. 16/603,180, filed on Oct. 4, 2019, which is a national stage entry under 35 U.S.C. § 371 of International Application No. PCT/IB2018/000510, filed on Apr. 5, 2018, which claims the benefit of priority of U.S. Provisional Application Ser. No. 62/483,298 filed on Apr. 7, 2017, all of which are incorporated by reference herein in their entireties.

### INCORPORATION OF SEQUENCE LISTING

The sequence listing that is contained in the file named “PTI0009-515D1-US.xml,” which was created Sep. 1, 2023, and which has a file size of 510 kilobytes as measured in Microsoft Windows operating system, is filed electronically herewith and incorporated herein by reference.

### BACKGROUND

Autoimmune diseases such as type 1 diabetes (T1D), multiple sclerosis and rheumatoid arthritis result from chronic autoimmune responses involving T cells and B cells recognizing numerous antigenic epitopes on incompletely defined lists of autoantigens (Santamaria, P. (2010) Immunity 32:437-445; Babbe, H. et al. (2000) J. Exp. Med. 192:393-404; Firestein, G. S. (2003) Nature 423:356-361). Eliminating or suppressing all polyclonal autoreactive T-cell specificities (known and unknown) in each individual autoimmune disorder without compromising systemic immunity is not currently possible.

It was recently discovered that nanoparticles coupled with antigen-major histocompatibility complex (pMHC) molecules can trigger re-programming and expansion of type 1 regulatory T (TR1) cells in vivo. See PCT application No. PCT/IB2016/000691. However, no high-throughput method exists to measure the biological and expansive potency of the pMHC-coupled nanoparticles in vitro or pMHC complexes uncoupled to a nanoparticle, given the need for stable antigen-specific T-cell clones, the highly variable inter-experimental variability associated with the use of primary cells, and the poor quantitative inter-experimental reproducibility of read-outs distal to the antigen receptor-triggering event. In addition, the methods in the art (e.g., semi-quantitative proximal TCR signaling events as measured via western blotting) cannot closely mimic the complex relationship between pMHC density on nanoparticles and biological activity over a concentration range. As a result, these methods cannot faithfully predict whether a particular preparation is of sufficient quality to yield optimal biological responses. Therefore, there is a need in the art to develop in vitro methods for measuring the agonistic or expansive potency of a pMHC. This disclosure satisfies this need and provides related advantages as well.

### SUMMARY

Autoimmune diseases such as type 1 diabetes (T1D), multiple sclerosis and rheumatoid arthritis result from chronic autoimmune responses involving T cells and B cells recognizing numerous antigenic epitopes on incompletely defined lists of autoantigens (Santamaria, P. (2010) Immunity 32:437-445; Babbe, H. et al. (2000) J. Exp. Med. 192:393-404; Firestein, G. S. (2003) Nature 423:356-361). Eliminating or suppressing all polyclonal autoreactive T-cell specificities (known and unknown) in each individual autoimmune disorder without compromising systemic immunity is not currently possible.

Adoptive transfer of polyclonal FOXP3⁺CD4⁺CD25⁺ regulatory T (T_{reg}) cells expanded ex vivo has been proposed as an alternative therapeutic approach (Sakaguchi, S. et al. (2006) Immunol. Rev. 212:8-27). The potential for bystander immunosuppression, the lack of effective strategies for expanding antigen-specific T_{reg} cells in vitro, and the lineage instability of FOXP3⁺ T_{reg} cells have hindered the clinical translation of this approach (Zhou, X. et al. (2009) Nature Immunol. 10:1000-1007; Komatsu, N. et al. (2014) Nature Med. 20:62-68; Bailey-Bucktrout, S. L. et al. (2013) Immunity 39:949-962). T_{R}1 FOXP3⁻CD4⁺CD25⁻ T cells, which produce the cytokines IL-10 and IL-21 and express the surface markers CD49b and LAG-3 and the transcription factor c-Maf 8, constitute another regulatory T-cell subset recently exploited for the treatment of human inflammatory diseases (McLarnon, A. (2012) Nature Rev. Gastroenterol. Hepatol. 9:559; Desreumaux, P. et al. (2012) Gastroenterology 143:1207-1217; Roncarolo, M. G. et al. (2011) Immunol. Rev. 241:145-163). However, as with FOXP3+ T_{reg} cells, there are no pharmacological approaches that can expand autoantigen- or disease-specific T_{R}1-like cells in vivo.

Applicant has previously shown that systemic delivery of nanoparticles coated with autoimmune-disease-relevant (U.S. Pat. No. 8,354,110), gastrointestinal-relevant (WO 2013/144811), or cancer or tumor-relevant (U.S. Pat. No. 9,511,151) peptides bound to major histocompatibility complex molecules trigger the generation and expansion of antigen-specific regulatory cells in different mouse models, including mice humanized with lymphocytes from patients, leading to resolution of established autoimmune phenomena (see also WO 2016/198932 and Clemente-Casares, X. et al. (2016) “Expanding antigen-specific regulatory networks to treat autoimmunity,” Nature 530:434-440). However, no high-throughput method exists to measure the biological and expansive potency of the pMHC-coupled nanoparticles in vitro or pMHC complexes uncoupled to a nanoparticle, given the need for stable antigen-specific T cell clones, the technical challenges and highly variable inter-experimental variability associated with the use of primary cells, and the poor quantitative inter-experimental reproducibility of read-outs distal to the antigen receptor-triggering event.

The data presented here provide unexpected results in that primary TCR-MHC peptide interactions are accurately modeled in vitro by a cell line transduced/transfected with a pathway dependent reporter and the receptor complex (TCR plus CD4 or CD8 co-receptor) that responds with its natural ligand (peptide MHC class II or peptide MHC class I molecules). See FIG. 1 I vs J. The methods and compositions described in this disclosure are generally applicable to measuring the potency of a nanomedicine comprising either a ligand or receptor interacting with a cell expressing its cognate receptor or ligand. For example, the methods and compositions described herein can be used to design nanomedicines that comprise a nanoparticle described herein and a ligand for a receptor that can be deployed to modify and reprogram in vivo cellular responses. For example, beta-cell function is positively influenced by binding to E, P, and N-cadhereins. The methods and compositions described herein allow development and testing of compositions of matter that comprise a nanoparticle and E, P, or N-cadherein. Such compositions can be mated with an appropriate cell line, such as a Min6 cell line (glucose responsive beta-cell line), with a beta-cell reporter that can be chosen for its response to glucose.

In specific embodiments, this disclosure provides compositions and methods to measure the agonistic or antagonistic activity or “potency” of a pMHC complex that is optionally bound to a nanoparticle. In one aspect, provided is an isolated cell transduced with one or more polynucleotides encoding: a recombinant T cell receptor (TCR); a TCR-pathway-dependent reporter; and a co-receptor that binds a class I or a class II major histocompatibility complex (MHC) ligand. In a further aspect, the cells express a TCR-associated multi-subunit CD3 chain signaling complex. In a yet further embodiment, the cells are transduced with one or more polynucleotides that encode receptors or ligands for one or more of a co-stimulatory molecule and/or a cytokine.

Non-limiting examples of MHC ligands are selected from the group of receptors to bind: a classical MHC class I protein, a non-classical MHC class I protein, a classical MHC class II protein, a non-classical MHC class II protein, an MHC dimer (Fc fusions), a MHC tetramers, or a polymeric form of an MHC protein. In one aspect the polynucleotide encodes a MHC class I co-receptor such as CD8. In another aspect, the polynucleotide encodes a MHC class II co-receptor such as CD4. The polynucleotides are optionally operatively linked to regulatory elements that drive expression of the polynucleotides and further optionally, an enhancer element.

In one aspect, the polynucleotide(s) encoding the T cell receptor encodes TCRα and/or TCRβ that optionally contains regulatory elements operatively linked to the TCRα and/or TCRβ encoding polynucleotide(s). These polynucleotides can optionally further comprise a ribosome skipping sequence. In one aspect, the ribosome skipping sequence comprises, or yet further consists essentially of, or alternatively consists of a 2A ribosome skipping sequence. Non-limiting examples of the 2A ribosome skipping sequence comprise, or alternatively consist essentially of, or yet further consist of an F2A, aT2A or a P2A ribosome skipping sequence, or a combination thereof.

In a further aspect, the TCR-pathway-dependent reporter is a reporter of TCR activation or TCR pathway activation that may optionally provide one or more measurements of gene expression, activity, protein localization, protein modification, or protein-protein interaction. In a further aspect, the TCR-pathway-dependent reporter comprises, or alternatively consists essentially of, or yet further consists of a protein selected from the group of a luciferase, a beta lactamase, CAT, SEAP, or a fluorescent protein. In a yet further aspect, the TCR-pathway-dependent reporter comprises a nuclear factor of activated T cells (NFAT) transcription factor-binding DNA sequence or promoter.

In another embodiment, the cell has been transduced with a polynucleotide encoding a TCR-associated multi-subunit CD3 chain signaling complex that is optionally operatively coupled to regulatory sequences for expression of the CD3 signaling complex on the cells surface, e.g., promoters and/or enhancers. In one embodiment, the cell does not endogenously express a CD3 signaling complex.

The cells are useful to determine the activation potential of any antigen, examples of such include without limitation an autoimmune or cancer-relevant antigen that are optionally coupled to the MHC (pMHC). The pMHC are optionally coupled to a nanoparticle core or other carrier. In one aspect, the pMHC is complexed to a nanoparticle core, optionally via a linker to the core or via a coating on the core. The number of pMHC per nanoparticle core can vary, e.g., from about 10:1 to about 1000:1, and ranges in between 10:1 to about 1000:1. The nanoparticle core can optionally further comprise a plurality of co-stimulatory molecules and/or cytokines as is appropriate.

In a particular aspect, the pMHC, the cytokine and/or the co-stimulatory molecule is complexed to the nanoparticle core via a coating on the core. The coating can be, for example, a polymer, optionally a polyethylene glycol (PEG) coating, and the number of pMHC, the cytokine and/or the co-stimulatory molecule, per core can be measured by “density” or the number of pMHC per surface area of the nanoparticle core coated with the polymer. Any density can be measured, for example, from about 0.025 pMHC/100 nm² to about 100 pMHC/100 nm² per surface area of the nanoparticle core, and ranges in between 0.025 pMHC/100 nm² to about 100 pMHC/100 nm² per surface area of the nanoparticle core.

Any appropriate eukaryotic cell can be transduced with polynucleotides encoding the requisite elements; non-limiting examples of such include JurMA, Jurkat, BW5147, HuT-78, CEM, or Molt-4. The cells can be of any appropriate species, animal, mammalian, e.g., human. In a further aspect when the cell is to be transduced with a polynucleotide encoding the CD3 chain signaling complex, the cell does not endogenously express the CD3 chain signaling complex.

Further provided is a population of the cells identified herein, wherein in one aspect is substantially homogeneous. Methods for culturing the cells and populations of cells are further provided herein.

The disclosure also provides methods to prepare the isolated cells as described herein. In one aspect, the method comprises, or alternatively consists essentially of, or yet further consists of, transducing an isolated cell with one or more polynucleotides encoding: a recombinant T cell receptor (TCR); and a TCR-pathway-dependent reporter; and a co-receptor that binds class I or class II major histocompatibility complex (MHC) ligand. In one embodiment, the method comprises, or alternatively consists essentially of, or yet further consists of, transducing an isolated cell with a polynucleotide encoding TCR-associated multi-subunit CD3 chain signaling complex. The method further comprises culturing the cells under conditions that favor expression of the one or more polynucleotides encoding a recombinant T cell receptor (TCR), a TCR-pathway-dependent reporter and a receptor that binds class I or class II major histocompatibility complex (MHC) ligands. In another embodiment, the method further comprises culturing the cells under conditions that favor expression of a TCR-associated multi-subunit CD3 chain signaling complex.

In a further embodiment, the methods further comprise, or alternatively consist essentially of, or yet further consist of, transducing a cell with one or more polynucleotides that express receptors or ligands for one or more of: a plurality of co-stimulatory molecules, a plurality of co-stimulatory antibodies, a plurality of inhibitory receptor-blocking antibodies, and/or a plurality of cytokines.

Cells that express the receptors and the expression products of transduced polynucleotides can be identified by any appropriate method known in the art using for example, detectably labeled ligands and/or antibodies that bind the expression products by methods known in the art such as flow cytometry.

Upon transduction of the cells, the cells are grown under conditions that favor expression of the polynucleotides and for the production of a population of cells.

The cells and cell populations are useful in an in vitro method of measuring the agonistic or antagonistic activity of a composition comprising an antigen-MHC complex (pMHC) (optionally bound to a nanoparticle core) and optionally a co-stimulatory molecule and/or a cytokine, by contacting the composition with an isolated cell as described herein that favors binding of the receptors to their ligands, and then detecting any TCR pathway-dependent reporter signal produced by the reporter. As is apparent to the skilled artisan, the composition and cell are selected for probable interaction, e.g., the composition contains a pMHC class II specific TCR molecule and the cell expresses a MHC class II co-receptor, e.g., CD4.

In one aspect, after contacting of the cell to the composition, any reporter signal produced by the cells or population is quantified. The measured response can be catalogued and then compared with the quantified signal with a pre-determined and/or post-determined measurement of agonistic or antagonistic activity to monitor effectiveness of a therapy against other therapies or compositions. When the composition comprises a co-stimulatory molecule and the cells and cell populations express the appropriate receptors, the measured response can be catalogued and then compared with the quantified signal with a pre-determined and/or post-determined measurement of antagonistic activity to monitor effectiveness of a therapy against other therapies or compositions.

Thus, certain aspects of the disclosure relate to a combination comprising at least an isolated transduced cell or transduced cell population as described herein, an isolated complex, wherein the isolated complex comprises, or alternatively consists essentially of, or yet further consists of, nanoparticle cores coupled to a plurality of pMHC complexes, wherein the nanoparticle cores optionally further comprise, or further consist thereof, or alternatively further consist essentially of one or more co-stimulatory molecules and/or one or more cytokines coupled to the nanoparticle core.

For these compositions containing a plurality of the complexes, the pMHC complexes on each nanoparticle core are the same or different from each other; and/or the MHC of the pMHC complexes on each nanoparticle core are the same or different from each other; and/or the cytokines on each nanoparticle core are the same or different from each other; and/or the costimulatory molecules on each nanoparticle core are the same or different from each other; and/or the diameters of the nanoparticle cores are the same or different from each other; and/or the valency of the pMHC complexes on each nanoparticle core are the same or different from each other; and/or the density of the pMHC complexes on each nanoparticle core are the same or different from each other; and/or the valency or density of the co-stimulatory molecules on each nanoparticle core are the same or different from each other; and/or the valency or density of the cytokines on each nanoparticle core are the same or different from each other.

In one aspect, described herein, is a composition comprising: (a) at least one cell comprising (i) a recombinant T cell receptor (TCR) comprising a TCR alpha chain and a TCR beta chain; and (ii) a T cell receptor-pathway-dependent reporter, wherein the recombinant TCR is specific for a disease-relevant antigen bound to a major histocompatibility (MHC) molecule; and (b) a nanomedicine, comprising a disease-relevant antigen bound to an MHC molecule coupled to a nanoparticle. In certain embodiments, the T cell receptor-pathway-dependent reporter is actively transcribed. In certain embodiments, the disease-relevant antigen bound to the MHC molecule is coupled to the nanoparticle at a ratio of 10:1 or greater. In certain embodiments, the nanoparticle has a diameter of between 1 nanometer and 100 nanometers. In certain embodiments, the nanoparticle comprises a metal core. In certain embodiments, the disease-relevant antigen is an autoimmune or inflammatory disease-relevant antigen. In certain embodiments, the autoimmune or inflammatory disease-relevant antigen is selected from the list consisting of an asthma or allergic asthma antigen, a diabetes mellitus Type I antigen, a multiple sclerosis antigen, a peripheral neuropathy antigen, a primary biliary cirrhosis antigen, a neuromyelitis optica spectrum disorder antigen, a stiff-person syndrome antigen, an autoimmune encephalitis antigen, a pemphigus vulgaris antigen, a pemphigus foliaceus antigen, a psoriasis antigen, a Sjogren's disease/syndrome antigen, an inflammatory bowel disease antigen, an arthritis or rheumatoid arthritis antigen, a systemic lupus erythematosus antigen, a scleroderma antigen, an ANCA-associated vasculitis antigen, a Goodpasture syndrome antigen, a Kawasaki's disease antigen, a celiac disease, an autoimmune cardiomyopathy antigen, a myasthenia gravis antigen, an autoimmune uveitis antigen, a Grave's disease antigen, an anti-phospholipid syndrome antigen, an autoimmune hepatitis antigen, a sclerosing cholangitis antigen, a primary sclerosing cholangitis antigen, chronic obstructive pulmonary disease antigen, or a uveitis relevant antigen, and combinations thereof. In certain embodiments, the T cell receptor-pathway-dependent reporter activates transcription of a gene selected from the group consisting of a luciferase gene, a beta lactamase gene, a chloramphenicol acetyltransferase (CAT) gene, a secreted embryonic alkaline phosphatase (SEAP) gene, a fluorescent protein gene, and combinations thereof. In certain embodiments, the T cell receptor-pathway-dependent reporter consists of a polynucleotide sequence selected from the list comprises a nuclear factor of activated T cells (NFAT) transcription factor-binding DNA sequence or promoter, an NF-κB transcription factor-binding DNA sequence or promoter, an AP1 transcription factor-binding DNA sequence or promoter, an IL-2 transcription factor-binding DNA sequence or promoter, and combinations thereof. In certain embodiments, the at least one cell is selected from JurMA, Jurkat, BW5147, HuT-78, CEM, or Molt-4. In certain embodiments, the disease-relevant antigen is a polypeptide consisting of any one of SEQ ID Nos: 1 to 352 and combinations thereof. In certain embodiments, the disease-relevant antigen is a polypeptide consisting of any one of SEQ ID NOs: 353 to 455 and combinations thereof. In certain embodiments, the TCR alpha chain and TCR beta chain are translated as a single polypeptide. In certain embodiments, the TCR alpha chain and TCR beta chain of the single polypeptide are separated by a ribosome skipping sequence. In certain embodiments, the ribosome skipping sequence is set forth in any one of SEQ ID NOs: 456 to 523. In certain embodiments, the single polypeptide comprises an amino acid sequence at least 80%, 90%, 95%, or 100% identical to any one of SEQ ID NOs: 527, 533, or 538. In certain embodiments, the TCR alpha chain and TCR beta chain are translated as separate polypeptides. In certain embodiments, the TCR alpha chain and the TCR beta chain, wherein the TCR alpha chain comprises an amino acid sequence at least 80%, 90%, 95%, or 100% identical to any one of SEQ ID NOs: 528, 530, 534, 536 539, 541, and the TCR beta chain comprises an amino acid sequence at least 80%, 90%, 95%, or 100% identical to any one of SEQ ID NOs: 529, 531, 535, 537, 540, or 542. In certain embodiments, the TCR alpha chain and TCR beta chain are expressed at the surface of the cell. In certain embodiments, the cell comprises at least one exogenous polynucleotide encoding the TCR alpha chain and the TCR beta chain. In certain embodiments, the at least one exogenous polynucleotide comprises an IRES nucleic acid sequence. In certain embodiments, the IRES nucleic acid sequence is set forth in any one of SEQ ID NOs: 524 to 526. In certain embodiments, the polynucleotide comprises a nucleic acid sequence at least 80%, 90%, 95%, or 100% homologous to that set forth in any one of SEQ ID NOs: 532 or 557. In certain embodiments, the composition is for in vitro use in determining a potency or activity of a nanomedicine. In certain embodiments, the nanomedicine is for use in a human individual.

In another aspect, described herein, is a cell comprising a recombinant T cell receptor (TCR) and a T cell receptor-pathway-dependent reporter, wherein the recombinant T cell receptor is specific for a disease-relevant antigen bound to a major histocompatibility molecule. In certain embodiments, the T cell receptor-pathway-dependent reporter is actively transcribed. In certain embodiments, the disease-relevant antigen is an autoimmune or inflammatory disease-relevant antigen. In certain embodiments, the autoimmune or inflammatory disease-relevant antigen is selected from the list consisting of an asthma or allergic asthma antigen, a diabetes mellitus Type I antigen, a multiple sclerosis antigen, a peripheral neuropathy antigen, a primary biliary cirrhosis antigen, a neuromyelitis optica spectrum disorder antigen, a stiff-person syndrome antigen, an autoimmune encephalitis antigen, a pemphigus vulgaris antigen, a pemphigus foliaceus antigen, a psoriasis antigen, a Sjogren's disease/syndrome antigen, an inflammatory bowel disease antigen, an arthritis or rheumatoid arthritis antigen, a systemic lupus erythematosus antigen, a scleroderma antigen, an ANCA-associated vasculitis antigen, a Goodpasture syndrome antigen, a Kawasaki's disease antigen, a celiac disease, an autoimmune cardiomyopathy antigen, a myasthenia gravis antigen, an autoimmune uveitis antigen, a Grave's disease antigen, an anti-phospholipid syndrome antigen, an autoimmune hepatitis antigen, a sclerosing cholangitis antigen, a primary sclerosing cholangitis antigen, chronic obstructive pulmonary disease antigen, or a uveitis relevant antigen, and combinations thereof. In certain embodiments, the T cell receptor-pathway-dependent reporter activates transcription of a gene selected from the group consisting of a luciferase gene, a beta lactamase gene, a chloramphenicol acetyltransferase (CAT) gene, a secreted embryonic alkaline phosphatase (SEAP) gene, a fluorescent protein gene, and combinations thereof. In certain embodiments, the T cell receptor-pathway-dependent reporter comprises a polynucleotide sequence selected from the list consisting of a nuclear factor of activated T cells (NFAT) transcription factor-binding DNA sequence or promoter, an NF-κB transcription factor-binding DNA sequence or promoter, an AP1 transcription factor-binding DNA sequence or promoter, an IL-2 transcription factor-binding DNA sequence or promoter, and combinations thereof. In certain embodiments, the cell is selected from JurMA, Jurkat, BW5147, HuT-78, CEM, or Molt-4. In certain embodiments, the disease-relevant antigen is a polypeptide consisting of any one of SEQ ID NOs: 1 to 352 and combinations thereof. In certain embodiments, the disease-relevant antigen is a polypeptide consisting of any one of SEQ ID NOs: 353 to 455 and combinations thereof. In certain embodiments, the TCR alpha chain and TCR beta chain are translated as a single polypeptide. In certain embodiments, the TCR alpha chain and TCR beta chain of the single polypeptide are separated by a ribosome skipping sequence. In certain embodiments, the ribosome skipping sequence is set forth in any one of SEQ ID NOs: 456 to 523. In certain embodiments, the single polypeptide comprises an amino acid sequence at least 80%, 90%, 95%, or 100% identical to any one of SEQ ID NOs: 527, 533, or 538. In certain embodiments, the TCR alpha chain and TCR beta chain are translated as separate polypeptides. In certain embodiments, the TCR alpha chain and the TCR beta chain, wherein the TCR alpha chain comprises an amino acid sequence at least 80%, 90%, 95%, or 100% identical to any one of SEQ ID NOs: 528, 530, 534, 536 539, 541, and the TCR beta chain comprises an amino acid sequence at least 80%, 90%, 95%, or 100% identical to any one of SEQ ID NOs: 529, 531, 535, 537, 540, or 542. In certain embodiments, the TCR alpha chain and TCR beta chain are expressed at the surface of the cell. In certain embodiments, the cell comprises at least one exogenous polynucleotide encoding the TCR alpha chain and the TCR beta chain. In certain embodiments, the at least one exogenous polynucleotide comprises an IRES nucleic acid sequence. In certain embodiments, the IRES nucleic acid sequence is set forth in any one of SEQ ID NOs: 524 to 526. In certain embodiments, the at least one exogenous polynucleotide comprises a nucleic acid sequence at least 80%, 90%, 95%, or 100% homologous to that set forth in any one of SEQ ID NOs: 532 or 557. In certain embodiments the cell is a population of cells. In certain embodiments, the cell or the population of cells are for in vitro use in determining a potency or activity of a nanomedicine. In certain embodiments, the nanomedicine is for use in a human individual.

In another aspect described herein is an in vitro method of measuring agonistic activity of a nanomedicine comprising a disease-relevant antigen bound to an MHC molecule coupled to a nanoparticle, the method comprising: (a) contacting the nanomedicine with the cell or population of cells described herein; and (b) detecting a signal produced by the T cell receptor-pathway-dependent reporter. In certain embodiments, the nanomedicine comprises a plurality of nanoparticles. In certain embodiments, the plurality of nanoparticles comprise a plurality of nanoparticles comprising a plurality of disease-relevant antigens bound to an MHC molecule coupled to the nanoparticle. In certain embodiments, the disease-relevant antigen is an autoimmune or inflammatory disease-relevant antigen. In certain embodiments, the autoimmune or inflammatory disease-relevant antigen is selected from the list consisting of a diabetes mellitus Type I antigen, an asthma or allergic asthma antigen, a multiple sclerosis antigen, a peripheral neuropathy antigen, a primary biliary cirrhosis antigen, a neuromyelitis optica spectrum disorder antigen, a stiff-person syndrome antigen, an autoimmune encephalitis antigen, a pemphigus vulgaris antigen, a pemphigus foliaceus antigen, a psoriasis antigen, a Sjogren's disease/syndrome antigen, an inflammatory bowel disease antigen, an arthritis or rheumatoid arthritis antigen, a systemic lupus erythematosus antigen, a scleroderma antigen, an ANCA-associated vasculitis antigen, a Goodpasture syndrome antigen, a Kawasaki's disease antigen, a celiac disease, an autoimmune cardiomyopathy antigen, a myasthenia gravis antigen, an autoimmune uveitis antigen, a Grave's disease antigen, an anti-phospholipid syndrome antigen, an autoimmune hepatitis antigen, a sclerosing cholangitis antigen, a primary sclerosing cholangitis antigen, chronic obstructive pulmonary disease antigen, or a uveitis relevant antigen, and combinations thereof. In certain embodiments, the plurality of nanoparticles comprise a plurality of nanoparticles with a diameter from 1 nanometer to about 100 nanometers. In certain embodiments, the method further comprises quantifying the T cell receptor-pathway-dependent reporter signal. In certain embodiments, the quantitation comprises determining a concentration of the nanomedicine that initiates a response that is about 50% of a maximal response, wherein the maximal response is the response initiated at the highest concentration of nanomedicine contacted with the cell or population of cells when a plurality of concentrations of the nanomedicine are contacted with the cell or population of cells. In certain embodiments, the plurality of the concentrations of the nanomedicine are contacted with the cell or population of cells in the same assay. In certain embodiments, the quantitation comprises determining a concentration of the nanomedicine that initiates a response that is at least about 200%, of a negative control; wherein the negative control comprises a nanomedicine that does not specifically interact with the recombinant T cell receptor (TCR) of the cell or the population of cells. In certain embodiments, the signal is produced by an enzyme. In certain embodiments, the enzyme is luciferase or peroxidase. In certain embodiments, the signal is a fluorescent signal. In certain embodiments, the method is utilized as a quality control step in a manufacturing process.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1A to FIG. 1J show effects of NP size and pMHC valency on T-cell agonistic activity and TCR signaling. FIG. 1A shows the production of IFNγ by 8.3-CD8⁺ T-cells in response to NRP-V7/Kd-SFP, as a function of pMHC valency and NP numbers. FIG. 1B shows the agonistic properties of the NRP-V7/Kd-SFPs from FIG. 1A, as a function of pMHC concentration in the assay. FIG. 1C and FIG. 1D show the production of IFNγ by 8.3-CD8⁺ T-cells in response to PF conjugated with two different NRP-V7/Kd valencies, as a function of pMHC-NP (FIG. 1C) or pMHC concentration in the assay (FIG. 1D). FIG. 1E and FIG. 1F shows the comparison of the agonistic properties of small (SFP) vs. larger (PF) NPs coated with low NRP-V7/Kd valencies, as a function of pMHC-NP (FIG. 1E) or pMHC concentration (FIG. 1F). Data in FIG. 1A to FIG. 1F correspond to the average+SEM values of IFNγ secretion in triplicate wells (error bars were usually smaller than the size of the symbols used to display the data) and each panel corresponds to one representative out of at least three independent experiments. Negative controls involved the use of unconjugated or Cys-conjugated NPs at the high concentration of NPs (i.e., 50×10¹¹ NPs/mL in a), yielding zero IFNγ values. FIG. 1G shows the comparison of the agonistic properties of PF NPs conjugated with 10 different BDC2.5mi/IA^{g7} valencies on BDC2.5-CD4⁺ T-cells, as a function of pMHC-NP (top) or pMHC concentration (bottom). Data shown correspond to one experiment. Data for 5 and 10 μg of pMHC were repeated twice more with similar results. As a negative control, Applicant used Cys-conjugated NPs at a concentration of iron equivalent to that of 10 μg pMHC/mL of the 10 pMHC/NP preparation (95×10¹¹ NP/mL), yielding zero IFNγ values. FIG. 1H shows the relationship between BDC2.5mi/IA^{g7} valency and density (bottom and top horizontal axis, respectively) on PF NPs (grouped according to sub-threshold, threshold, minimal optimal, and supra-threshold densities) and agonistic activity on BDC2.5-CD4⁺ T-cells at 10 μg/mL (left) and 5 μg/mL (right) (concentrations of pMHC yielding near-maximal agonistic activity). P values between sub-threshold/threshold vs. minimal optimal valency/suprathreshold valencies were calculated via Mann-Whitney U. FIG. 1I shows luciferase activity (average+SEM of triplicates) in BDC2.5-TCR/mCDA/NFAT-luciferase-expressing JurMA cells in response to stimulation for various periods of time with BDC2.5mi/IA^{g7}-PF-M (12.5 μg/mL), soluble anti-hCD3ε mAb (10 μg/mL) and PMA/ionomycin. RLU (relative light units). As a negative control, Applicant used Cys-conjugated NPs at a concentration of iron equivalent to that of 5 μg pMHC/mL of the 10 pMHC/NP preparation (45.5×10¹¹ NP/mL), yielding 1.05 RLUs. Data shown are representative of at least three independent experiments per stimulation condition. P values between conditions were calculated via two-way ANOVA. FIG. 1J shows the relationship between BDC2.5mi/IA^{g7} valency and density (bottom and top horizontal axis, respectively) on PF NPs (grouped according to sub-threshold, threshold, minimal optimal, and supra-threshold densities) and agonistic activity on BDC2.5-TCR/mCD4/NFAT-luciferase-expressing JurMA cells at 5 μg/mL. P values were calculated via Mann-Whitney U.

FIG. 2A and FIG. 2B show the schematic representation of pMHC-NP binding to cognate-T-cells. FIG. 2A, Top Panel: schematic representation of a TCR nanocluster, composed of 16 units spanning 140 nm (assuming a 4 nm globular size of TCRαβ and 5 nm spacing), binding to 4 densely coated pMHC-NPs (carrying pMHC monomers spaced by 4 nm each). The bottom panel cartoon illustrates how these 4 pMHC-NPs interact with TCR islands (left) or nanoclusters (right), as viewed from the NP's perspective. FIG. 2B illustrates pMHC-NPs coated at supra-threshold, threshold and infra-threshold valencies (left) and their relative abilities to elicit TCR signaling in the clusters, taking into account overall binding avidity, pMHC-TCR association and dissociation rates, and both the kinetic proofreading and cooperative TCR signaling models. pMHC-NPs capable of ligating contiguous TCR heterodimers in these clusters are efficient in eliciting TCR signaling. These models explain why small NPs coated with closely apposed pMHCs have optimal immunological properties.

FIG. 3A to FIG. 3G show sustained binding and clustering of pMHC-NPs on cognate T-cells as a function of pMHC density. FIG. 3A and FIG. 3B show 2D TEM images of BDC2.5mi-CD4⁺ (FIG. 3A) or 8.3-CD8⁺ T-cells (FIG. 3B) incubated with BDC2.5mi/IA^{g7}- or NRP-V7/K^{d}-PF-M, respectively, coated at supra-threshold pMHC densities (46 pMHCs/NP). The two right panels in FIG. 3A and the four right panels in FIG. 3B show the presence of NPs in intracellular vesicles after 3 hr incubation at 37° C. FIG. 3C shows 2D TEM images of BDC2.5mi-CD4⁺ and 8.3-CD8⁺ T-cells incubated with non-cognate NRP-V7/K^{d}-PF-M and BDC2.5mi/IA^{g7}-PF-M, respectively. FIG. 3D, Left panel: 3D image: super-resolution microscopy of 8.3-CD8⁺ T-cells incubated with NRP-V7/K^{d}-PF-M-Alexa-647 at 4° C. for 30 min. Middle and Right panels: 2D images: T-cells incubated at 4° C. for 30 min and at 4° C. for 30 min followed by 37° C. for 1 hr. The histogram plot shows that the NP clusters increase in diameter with incubation time and temperature (179.1±4.6 nm to 401.7±4.2 nm; n=100 clusters/condition; P values calculated by Mann-Whitney U). Light gray: NRP-V7/K^{d}-PF-MAlexa-647; Dark gray: DAPI. Bar: 1 μm. FIG. 3E and FIG. 3F show 2D TEM images of BDC2.5mi-CD4⁺ T-cells incubated with BDC2.5mi/IA^{g7}-PF-M preparations carrying sub-threshold 10 pMHCs/NP; (FIG. 3E) or threshold (24 pMHCs/NP; (FIG. 3F) pMHC valencies. Four left panels in FIG. 3E and FIG. 3F show absence (e) or presence (f) of microclusters on the T-cell membrane. Two right panels on FIG. 3E and FIG. 3F show presence of intracellular vesicles. FIG. 3G shows average size of microclusters in cells cultured in the presence of pMHC-NPs coated at (59.5±6.5 nm), (271.2±17.3 nm) and (370±21.3 nm); (n=50-60 clusters on 9-15 cells/condition). P values were calculated by Mann-Whitney U. The experiments described in this figure are repeatable and can be reproduced with consistent results.

FIG. 4A and FIG. 4B show the sustained clustering of pMHC-NPs on cognate T-cell with scanning electron microscopy (SEM). FIG. 4A shows 3D SEM images of 8.3-CD8⁺ T-cells in the absence (left) or presence (right) of NRP-V7/K^{d}-PF-M. Magnification, 100,000×; Bar: 500 nm. Black dashed lines correspond to representative pMHC-NP clusters. FIG. 4B shows EDS spectral analysis. Three representative cluster-containing (a-c) and cluster-free membrane areas (d-f) shown in an enlarged SEM image were analyzed via EDS and data plotted as histograms. P value was obtained with Mann-Whitney U test.

FIG. 5 shows the results inter-assay variability of a potency assay.

FIG. 6A to FIG. 6D show results using a potency assay to determine the effect of serum and anti-pMHC-NP component antibodies on the ability of pMHC to stimulate a T cell line. pMHC-NP were either pre-incubated with human serum as shown in FIG. 6C and FIG. 6D, or without, as shown in FIG. 6A or FIG. 6B; and subsequently incubated with the indicated antibody or rabbit hyper immune (H.I) serum. Each antibody was incubated with the pMHC and cell as indicated at dilutions (from left to right) of 1:10, 1:100, and 1:1000 for serum in FIG. 6B and FIG. 6D, and molar ratios (from left to right) of Ab:pMHC of 1:1, 1:4, and 1:16. Bars indicate standard deviation.

FIG. 7A to FIG. 7D show flow cytometry of GFP labeled JURMA cells expressing a TCR specific for DR complexed with the IGRP₁₃₋₂₅ polypeptide. FIG. 7A shows cell line by itself; FIG. 7B shows cell line incubated with PE labeled DR3 IGRP₁₃₋₂₅ made by standard leucine zipper dimerization technology; FIG. 7C shows cell line incubated with PE labeled DR3 IGRP₁₃₋₂₅ made using knob-in-hole and cys-trap dimerization technology, lacking a leucine zipper; FIG. 7D shows cell line incubated with irrelevant PE labeled MHC class II heterodimers.

FIG. 8A and FIG. 8B show stimulation of JURMA cells expressing a TCR specific for DR complexed with the IGRP₁₃₋₂₅ polypeptide conjugated to a nanoparticle.

### DETAILED DESCRIPTION

In one aspect, described herein, is a composition comprising: (a) at least one cell comprising (i) a recombinant T cell receptor (TCR) comprising a TCR alpha chain and a TCR beta chain; and (ii) a T cell receptor-pathway-dependent reporter, wherein the recombinant TCR is specific for a disease-relevant antigen bound to a major histocompatibility (MHC) molecule; and (b) a nanomedicine, comprising a disease-relevant antigen bound to an MHC molecule coupled to a nanoparticle.

In another aspect, described herein, is a cell comprising a recombinant T cell receptor (TCR) and a T cell receptor-pathway-dependent reporter, wherein the recombinant T cell receptor is specific for a disease-relevant antigen bound to a major histocompatibility molecule.

In another aspect described herein is an in vitro method of measuring agonistic activity of a nanomedicine comprising a disease-relevant antigen bound to an MHC molecule coupled to a nanoparticle, the method comprising: (a) contacting the nanomedicine with the cell or population of cells described herein; and (b) detecting a signal produced by the T cell receptor-pathway-dependent reporter.

Throughout and within this disclosure, reference is made to technical and patent literature to more fully describe the state of the art to which this disclosure relates. Some publications are identified by an Arabic number and the full bibliographic information for the publication is found in the reference section, immediately preceding the claims. All publications are incorporated by reference herein to more fully describe the state of the art to which this disclosure pertains.

It is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

It must be noted that as used herein and in the appended claims, the singular forms “a”, “an”, and “the” include plural referents unless the context clearly dictates otherwise. Thus, for example, reference to “an excipient” includes a plurality of excipients. The term “at least one” intends one or more.

Throughout this application, the term “about” is used to indicate that a value includes the standard deviation of error for the device or method being employed to determine the value. The term “about” when used before a numerical designation, e.g., temperature, time, amount, and concentration, including range, indicates approximations which may vary by (+) or (−) by up to 10%.

Unless defined otherwise, all technical and scientific terms used herein have the same meaning as commonly understood by one of ordinary skill in the art to which this disclosure belongs. As used herein the following terms have the following meanings.

As used herein, the term “comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed disclosure, such as compositions for treating or preventing multiple sclerosis. “Consisting of” shall mean excluding more than trace elements of other ingredients and substantial method steps. Embodiments defined by each of these transition terms are within the scope of this disclosure.

By “biocompatible” it is meant that the components of the delivery system will not cause tissue injury or injury to the human biological system. To impart biocompatibility, polymers and excipients that have had history of safe use in humans or with GRAS (Generally Accepted As Safe) status, will be used preferentially. By biocompatibility,” it is meant that the ingredients and excipients used in the composition will ultimately be “bioabsorbed” or cleared by the body with no adverse effects to the body. For a composition to be biocompatible and be regarded as non-toxic it must not cause toxicity to cells. Similarly, the term “bioabsorbable” refers to nanoparticles made from materials that undergo bioabsorption in vivo over a period of time such that long term accumulation of the material in the patient is avoided. In a certain embodiment, the biocompatible nanoparticle is bioabsorbed over a period of less than two years, preferably less than one year and even more preferably less than six months. The rate of bioabsorption is related to the size of the particle, the material used, and other factors well-recognized by the skilled artisan. A mixture of bioabsorbable, biocompatible materials can be used to form the nanoparticle cores used in this disclosure. In one embodiment, iron oxide and a biocompatible, bioabsorbable polymer can be combined. For example, iron oxide and PGLA can be combined to form a nanoparticle.

The term “major histocompatiblility complex” or “MHC” refers to an antigen-presenting molecule on an immune cell that has the ability to associate with the antigen to form an antigen-associated immune cell. In some embodiments, the MHC is a class I or class II molecule. In some embodiments, the MHC comprises, consists of, or consists essentially of classical MHC class I protein, non-classical MHC class I protein, classical MHC class II protein, non-classical MHC class II protein, MHC dimers (Fc fusions), MHC tetramers, or a polymeric form of an MHC protein. The MHC bind cell surface molecules selected from CD4 and CD8.

An polypeptide/antigen-MHC-nanoparticle complex (“NP-complex” or “complex” or “pMHC-NP” or “nanoparticle complex”) refers to presentation of a peptide, carbohydrate, lipid, or other antigenic segment, fragment, or epitope of an antigenic molecule or protein (i.e., self-peptide or autoantigen) presented by a MHC molecule on a surface, such as a nanoparticle core.

The “nanoparticle core” is the nanoparticle substrate that does or does not include layers or coatings. The nanoparticle complex comprises the core with at least the pMHC complex coupled to the core. Nanoparticle cores can be made from any of various materials and can be biocompatible.

As used herein, the term “nuclear factor of activated T-cells” or “NFAT” is a general name applied to a family of transcription factors shown to be important in immune response (e.g., activating T-cell-regulated immune response). The immune system can express one or more members of the NFAT family members, which include but are not limited to NFATc1, NFATc2, NFATc3, NFATc4, and NFAT5. NFATc1 through NFATc4 are regulated by calcium signaling. Calcium signaling is critical to NFAT activation because calmodulin (CaM), a well-known calcium sensor protein, activates the serine/threonine phosphatase calcineurin (CN). Nuclear import of NFAT proteins is opposed by maintenance kinases in the cytoplasm and export kinases in the nucleus. Export kinases, such as PKA and GSK-30, must be inactivated for NFAT nuclear retention. In one embodiment, NFAT transcription factors enable integration and coincidence detection of calcium signals with other signaling pathways such as ras-MAPK or PKC.

As used herein, the term “T-cell receptor” or “TCR” refers to a molecule capable of recognizing a peptide when presented by an MHC molecule. In some embodiments, a TCR is a heterodimer comprising a T-cell receptor α-chain (TCR α) and T-cell receptor β-chain (TCR β), each chain comprising a variable (V) region and a constant (C) region, transmembrane domain, and cytosolic domain. The V and C regions are generally homologous to immunoglobulin V and C regions and comprise three complementarity-determining regions (CDRs). Both TCR chains are anchored in the plasma membrane of the cell presenting the TCR. In some embodiments, the TCR is a heterodimer comprising TCR γ-chain (TCR γ) and TCR δ-chain (TCR δ). In some embodiments, the TCR is a single chain TCR construct. The non-limiting examples of TCR α can be found at GenBank, e.g., GenBank Accession Nos. AAB31880.1, AAB28318.1, AAB24428.1, and ADW95878.1, and equivalents of each thereof. The non-limiting examples of TCR β can also be found at GenBank, e.g., GenBank Accession Nos. AAB31887.1, AKG65861.1, ADW95908.1, and AAM53411.1, and equivalents of each thereof. In one embodiment, TCR γ-chain comprises one or more sequences found at GenBank, e.g., GenBank Accession Nos. AAM21533.1, DAA30449.1, and ABG91733.1, and equivalents of each thereof. In one embodiment, TCR δ-chain comprises one or more sequences found at GenBank, e.g., GenBank Accession Nos. Q7YRN2.1, AAC48547.1, JC4663, and NP_001009418.1, and equivalents of each thereof. The single chain TCRs are known in the art. Non-limiting examples of single chain TCRs are disclosed in WO1996018105 and US20120252742, each of which is incorporated by reference in its entirety. In one embodiment, the polynucleotide and the polypeptide sequences of TCR β are listed in the Exemplary Sequence Listing provided below and polynucleotides encoding the polypeptides of the TCR β, and equivalents of these polynucleotides.

In some embodiments, a TCR is associated with CD3 and forms a TCR-associated multi-subunit CD3 chain signaling complex (or the TCR/CD3 complex). In these embodiments, the cell is transduced with one or more polynucleotides encoding a TCR/CD3 complex formed by polypeptides comprising, or alternatively consisting essentially of, or yet further consisting of a and R TCR chains, the CD3γ, δ and ε polypeptides, and the ζ chains. Forming in different modules, the TCR/CD3 complex can carry different roles. In one embodiment, the complex is involved in antigen-specific recognition. In these embodiments, the complex is involved in signal transduction primarily through the presence of an immunorecepter tyrosine-based activation motif (“ITAM”) in the cytoplasmic tails of the CD3 and ζ chains. In some embodiments, the TCR/CD3 complex is involved in a TCR signaling pathway stimulated by an antigen, a superantigen, or an antibody (e.g., anti-receptor antibody). In one embodiment, exogenous expression of the TCR/CD3 complex facilitates the TCR signaling pathway in CD3-negative cells. Non-limiting examples of CD3-negative cells include but are not limited to BW5147 (ATCC No. TIB-472), Nk-92 (ATCC No. CRL-2407), Mino (ATCC No. PTS-CRL-3000), and JeKo-1 (ATCC No. CRL-3006).

As used herein, the term “isolated cell” refers to the cell provided to assess the potency of test agents, including the nanoparticles coupled with pMHC. In one embodiment, the cell is a T lineage cell that is selected from JurMA, Jurkat, BW5147, HuT-78, CEM, or Molt-4. They can be of any appropriate species, e.g., animal, mammal, human, canine, feline, equine, bovine or ovine. In another embodiment, the isolated cells are effector cells such as immune cells. In some embodiments, the effector cells express a T cell receptor (TCR), the TCR-associated CD3 multi-unit chain complex, and/or a TCR-pathway-dependent reporter and a CD4 or CD8 receptor. In a further aspect, the cell also expresses a receptor for a co-stimulatory molecule and/or a cytokine. In a certain embodiment, the TCR is murinized (i.e., wherein the TCR is optimized to interact with a murine CD4 molecule).

As used herein, the term “reporter” means an element on or within an isolated cell having a characteristic (e.g., activity, expression, localization, interaction, modification, etc.) which is one or more of: dependent upon, correlates with, or activated by physiological changes or conditions of the cell. For example, “TCR-pathway-dependent reporter” refers to an element on or within the cells, a characteristic of which is activated or dependent upon the activation or modulation of the TCR pathway. In some embodiments, the TCR-pathway-dependent reporter is activated by an upstream transcription factor-binding DNA sequence or promoter (e.g., NFAT transcription factor-binding DNA sequence or promoter, NF-κB transcription factor-binding DNA sequence or promoter, AP1 transcription factor-binding DNA sequence or promoter, and IL-2 transcription factor-binding DNA sequence or promoter). In one embodiment, the report (e.g., TCR-pathway-dependent reporter) comprises, consists essentially of, or yet consists of a gene coding for a protein selected from the group consisting of a luciferase, a beta lactamase, CAT, SEAP, a fluorescent protein, a quantifiable gene product, and/or the combination thereof.

As used herein, the term “CD3” (cluster of differentiation 3) refers to the protein complex associated with the T cell receptor. In some embodiments, antibodies directed against CD3 are able to generate an activation signal in T lymphocytes. Other T cell activation ligands can be used as well, including without limitation CD28, CD134, CD137, and CD27. In some embodiments, the CD3 comprises, or alternatively consists essentially of, or consists of four distinct chains. For mammals, the four distinct chains are: CD3gamma, CD3delta, CD3epsilon and CD3zeta. The non-limiting examples of CD3 chains can be found at GenBank, e.g., GenBank Accession Nos CAA72995.1, AAI45927.1, NP_998940.1, AAB24559.1, NP_000723.1, AEQ93556.1, and EAW67366.1.

As used herein, the term “CD4” (cluster of differentiation 4) refers to a glycoprotein found on surface of immune cells, e.g., T helper cells, monocytes, macrophages, and dendritic cells. In some embodiments, CD4 acts as a co-receptor for the TCR and recruits the tyrosine kinase (e.g., Lck). The non-limiting examples of CD4 can be found at GenBank, e.g., GenBank Accession Nos AAC36010.1, CAA72740.1, AFK73394.1, CAA60883.1, and AAH25782.1. Exemplary polynucleotide and polypeptide sequences of CD4 are listed in the exemplary sequence listing provided below.

The term “ribosome skipping sequence” refers to any sequence that can be introduced between two or more gene sequences under the control of the same promotor so that the gene sequences are translated as separate polypeptides (i.e., translated as biscistronic or multicistronic sequences). Examples of ribosome skipping sequence include but are not limited to 2A peptide sequences. In one embodiment, one ribosome skipping sequence is introduced between the gene sequences. In another embodiment, two or more ribosome skipping sequences are introduced between the gene sequences.

The term “2A ribosome skipping sequence” refers to a peptide sequence comprising the consensus motif of Val/Ile-Glu-X-Asn-Pro-Gly-Pro, wherein X stands for any amino acid (SEQ ID NO:558). In one embodiment, the 2A ribosome skipping sequence comprises, or alternatively consists essentially of, or yet consists of porcine teschovirus-1 2A (P2A); T2A, Thosea asigna virus 2A (T2A); equine rhinitis A virus (ERAV) 2A (E2A); FMDV 2A (F2A), or the combination thereof. Non-limiting examples of 2A peptide sequences, include but are those sequences provided in the exemplary sequence listing provided below.

The 2A ribosome skipping sequences permit expression of multiple genes in one expression vector. For example, an expression vector with the 2A ribosome skipping sequence can express all four proteins that make up the CD3 complex. In one embodiment, the non-limiting exemplary coding region sequence of the expression vector is listed in the Exemplary Sequence Listing provided below as: polynucleotide sequence of murine CD3delta-F2A-gamma-T2A-epsilon-P2A-zeta and polypeptide sequence of murine CD3delta-F2A-gamma-T2A-epsilon-P2A-zeta, and equivalents of each thereof.

In another aspect, an expression vector with the 2A ribosome skipping sequence can express multiple subunits of TCR. In some embodiments, the non-limiting exemplary coding region sequences of the expression vector are provided in the SEQ ID NOs: 527 to 531 (IGRP₁₃₋₂₅ TCR), 533 to 537 (Murinized IGRP₁₃₋₂₅ TCR), 538 to 542 (PPI₇₆₋₉₀ TCR), or 543 to 547 (BDC 2.5 TCR).

The term “IRES sequence” or “internal ribosome entry site sequence” refers to a nucleotide sequence that permits translation initiation in the middle of a RNA sequence. In some embodiments, insertion of an IRES sequence between two gene sequences (e.g., reporter open reading frames) can drive translation of the downstream protein coding region independently of the 5′-cap structure bound to the 5′ end of the mRNA molecule. Suitable IRES sequences are known in the art. In some embodiments, the IRES sequences derive from poliovirus, rhinovirus, encephalomyocarditis virus, foot-and-mouth disease virus, hepatitis A virus, hepatitis C virus, classical swine fever virus, and bovine viral diarrhea virus. Non-limiting examples of IRES sequences can be found at www.iresite.org, which is incorporated by reference in its entirety. The non-limiting examples of IRES sequences are provided in SEQ ID NOs: 524 to 526, and include but are not limited to: EMCV IRES sequence, pBag1 IRES sequence, and synthetic IRES sequence, and equivalents of each thereof.

The term “luciferase” means an protein that can catalyze a bioluminescent reaction. For example, a luciferase as an enzyme can produce a signal when provided with a substrate (e.g., luciferin, longchain aldehyde or colentrazine), an energy source (e.g., ATP), and oxygen. Suitable luciferase sequences for this disclosure are known in the art. In one embodiment, the luciferase gene is from the firefly (e.g., Photinus pyralis). Non-limiting examples of luciferase sequences can be located at GenBank (e.g., GenBank Accession Nos. AAR20792.1, AAL40677.1, AAL40676.1, and AAV35379.1, and equivalents of each thereof. The luciferase reporter system is available commercially (e.g., Promega Cat. #E1500 or E4550). Exemplary polynucleotides encoding a luciferase protein and the polypeptide are provided in SEQ ID NOs: 555 and 556, as provided below.

The term “beta lactamase” refers to an enzyme or protein that can breaks down a beta-lactam ring. In one embodiment, beta lactamase is an enzyme produced by bacteria, which can hydrolyze the beta-lactam ring in a beta-lactam antibiotic, either partially or completely. Non-limiting examples of beta lactamase sequences can be located at GenBank (e.g., GenBank Accession Nos AMM70781.1, CAA54104.1, and AAA23441.1, and equivalents of each thereof), last accessed on Jan. 12, 2017.

The term “chloramphenicol acetyltransferase” or “CAT” refers to an enzyme or protein that can transfer an acetyl group from acetylated co-enzyme A to chloramphenicol or a related derivative. Non-limiting examples of “CAT” can be located at GenBank (e.g., Accession Nos. OCR39292.1, WP_072643749.1, CUB58229.1, and KIX82948.1, and equivalents of each thereof), last accessed on Jan. 12, 2017. The CAT assays are commercially available (e.g., FAST CAT® Chloramphenicol Acetyltransferase Assay Kit (F-2900) from Thermal Fisher).

The term “secreted embryonic alkaline phosphatase” or “SEAP” refers to an enzyme encoded by a SEAP gene (e.g., GenBank Accession No. NP 001623 and equivalents thereof, last accessed on Jan. 12, 2017), which is used as a reporter to study promoter activity or gene expression. Non-limiting examples of SEAP sequences can be located at GenBank (e.g., GenBank Accession Nos. ADV10306.1, AAB64404.1, EEB84921.1, and EFD70636.1, and equivalents of each thereof), last accessed on Jan. 12, 2017. The SEAP activity can be measured by a luminometer (e.g., Turner BioSystems Veritas Microplate Luminometer from Promega).

The term “fluorescent protein” refers to any protein capable of emitting light when excited with appropriate electromagnetic radiation, and which has an amino acid sequence that is either natural or engineered and is derived from the amino acid sequence of Aequorea-related fluorescent protein. The emitting light from the fluorescent protein can be determined by fluorescent readers (e.g., FL600 Fluorescence Microplate reader). Non-limiting examples of fluorescent protein include Green Protein (GFP), Enhanced Green Fluorescent Protein (eGFP), Blue Fluorescent Protein (BFP), Yellow Fluorescent Protein (YFP), Cyan Fluorescent Protein (CFP), Red Fluorescent Protein (RFP), or any other suitable fluorescent protein, or combination thereof, or fluorescent parts or derivatives thereof. The sequences of fluorescent proteins can be located at GenBank (e.g., GenBank Accession Nos. AFA52654.1, ACS44348.1, and AAQ96629.1, and equivalents of each thereof), last accessed on Jan. 12, 2017. The fluorescent protein promoter reporters are commercially available (e.g., TakaRa Cat. #631089).

“Under transcriptional control” is a term well understood in the art and indicates that transcription of a polynucleotide sequence, usually a DNA sequence, depends on its being operatively linked to an element which contributes to the initiation of, or promotes, transcription. “Operatively linked” intends the polynucleotides are arranged in a manner that allows them to function in a cell.

The term “encode” as it is applied to polynucleotides refers to a polynucleotide which is said to “encode” a polypeptide if, in its native state or when manipulated by methods well known to those skilled in the art, it can be transcribed and/or translated to produce the mRNA for the polypeptide and/or a fragment thereof. The antisense strand is the complement of such a nucleic acid, and the encoding sequence can be deduced therefrom.

The term “promoter” refers to a region of DNA that initiates transcription of a particular gene. The promoter includes the core promoter, which is the minimal portion of the promoter required to properly initiate transcription and can also include regulatory elements such as transcription factor binding sites. The regulatory elements may promote transcription or inhibit transcription. Regulatory elements in the promoter can be binding sites for transcriptional activators or transcriptional repressors. A promoter can be constitutive or inducible. A constitutive promoter refers to one that is always active and/or constantly directs transcription of a gene above a basal level of transcription. Non-limiting examples of such include the phosphoglycerate kinase 1 (PGK) promoter; SSFV, CMV, MNDU3, SV40, Ef1a, UBC and CAGG. An inducible promoter is one which is capable of being induced by a molecule or a factor added to the cell or expressed in the cell. An inducible promoter may still produce a basal level of transcription in the absence of induction, but induction typically leads to significantly more production of the protein.

An enhancer is a regulatory element that increases the expression of a target sequence. A “promoter/enhancer” is a polynucleotide that contains sequences capable of providing both promoter and enhancer functions. For example, the long terminal repeats of retroviruses contain both promoter and enhancer functions. The enhancer/promoter may be “endogenous” or “exogenous” or “heterologous.” An “endogenous” enhancer/promoter is one which is naturally linked with a given gene in the genome. An “exogenous” or “heterologous” enhancer/promoter is one which is placed in juxtaposition to a gene by means of genetic manipulation (i.e., molecular biological techniques) such that transcription of that gene is directed by the linked enhancer/promoter. The polynucleotides of this disclosure optionally comprise an enhancer sequence.

As used herein, the term “NFAT promoter,” “NFAT transcription factor-binding DNA sequence” or “nuclear factor of activated T cells promoter” refers to a sequence comprising, consisting essentially of, or yet consisting of one or more NFAT elements. In one embodiment, the binding of an NFAT promoter by an NFAT transcription factor (e.g., NFATc1, NFATc2, NFATc3, NFATc4, or NFAT5) increases or promotes the transcription of downstream sequences (e.g., a reporter). The NFAT promoter sequences are generally in GenBank, which include but are not limited to the following sequences from GenBank at Accession Nos. DQ904462.1, KX591058.1, AF480838.1, and equivalents of each thereof, or a sequence with at least 50%, 60%, 70%, 80%, 90%, 95%, 99%, or 100% identity thereof.

As used herein, the term “AP-1 promoter” or “AP-1 transcription factor-binding DNA sequence” refers to a sequence comprising, or alternatively consisting essentially of, or yet further consisting of one or more AP-1 transcriptional activation elements. In one embodiment, the binding of an AP-1 promoter by an AP-1 transcription factor increases or promotes the transcription of downstream sequences (e.g., a reporter like luciferase or CAT). The AP-1 promoter may derive from human, mouse, rat, zebrafish, flies, or any other species. In one embodiment, the AP-1 promoter has a sequence of ATGAGTCAT, and equivalents thereof, or a sequence with at least 50%, 60%, 70%, 80%, 90%, 95%, 99%, or 100% sequence identity equivalent to ATGAGTCAT.

As used herein, the term “NF-κB promoter” or “NF-κB transcription factor-binding DNA sequence” refers to a sequence comprising, or alternatively consisting essentially of, or yet further consisting of one or more NF-κB elements. In one embodiment, the binding of Rel/NF-kB transcription factors, either as a homodimer or heterodimer, to the NF-κB promoter increases or initiates the transcription of downstream sequences (e.g., a reporter like luciferase or CAT). Some embodiments of the NF-kB promoter or binding site are disclosed in U.S. Pat. No. 8,299,237, which is incorporated by reference in its entirety.

As used herein, the term “IL-2 promoter” or “IL-2 transcription factor-binding DNA sequence” refers to a sequence comprising, or alternatively consisting essentially of, or yet further consisting of one or more IL-2 transcriptional activation elements that respond to T cell simulation. In one embodiment, the binding of transcription factors to the IL-2 promoter increase or initiate the transcription of downstream sequences (e.g., a reporter like luciferase or CAT). In one embodiment, the IL-2 promoter derives from human, mouse, rat, or zebrafish. Some non-limiting exemplary IL-2 promoter sequences are accessible from GenBank at Accession Nos. AJ006884.1, EF397241.1, AB041341.1, KU058846.1, EF457240.1, and HM802330.1, and equivalents of each thereof, last accessed on Jan. 12, 2017.

As used herein, the term “vector” refers to a non-chromosomal nucleic acid comprising an intact replicon such that the vector may be replicated when placed within a cell, for example by a process of transformation. Vectors may be viral or non-viral. Viral vectors include retroviruses, lentivirus, adenoviruses, herpesvirus, bacculoviruses, modified bacculoviruses, papovirus, or otherwise modified naturally occurring viruses. Exemplary non-viral vectors for delivering nucleic acid include naked DNA; DNA complexed with cationic lipids, alone or in combination with cationic polymers; anionic and cationic liposomes; DNA-protein complexes and particles comprising DNA condensed with cationic polymers such as heterogeneous polylysine, defined-length oligopeptides, and polyethylene imine, in some cases contained in liposomes; and the use of ternary complexes comprising a virus and polylysine-DNA.

A “viral vector” is defined as a recombinantly produced virus or viral particle that comprises a polynucleotide to be delivered into a host cell, either in vivo, ex vivo or in vitro. Examples of viral vectors include retroviral vectors, lentiviral vectors, adenovirus vectors, adeno-associated virus vectors, alphavirus vectors and the like. Alphavirus vectors, such as Semliki Forest virus-based vectors and Sindbis virus-based vectors, have also been developed for use in gene therapy and immunotherapy. See, Schlesinger and Dubensky (1999) Curr. Opin. Biotechnol. 5:434-439 and Ying, et al. (1999) Nat. Med. 5(7):823-827.

In aspects where gene transfer is mediated by a lentiviral vector, a vector construct refers to the polynucleotide comprising the lentiviral genome or part thereof, and a therapeutic gene. As used herein, “lentiviral mediated gene transfer” or “lentiviral transduction” carries the same meaning and refers to the process by which a gene or nucleic acid sequences are stably transferred into the host cell by virtue of the virus entering the cell and integrating its genome into the host cell genome. The virus can enter the host cell via its normal mechanism of infection or be modified such that it binds to a different host cell surface receptor or ligand to enter the cell. Retroviruses carry their genetic information in the form of RNA; however, once the virus infects a cell, the RNA is reverse-transcribed into the DNA form which integrates into the genomic DNA of the infected cell. The integrated DNA form is called a provirus. As used herein, lentiviral vector refers to a viral particle capable of introducing exogenous nucleic acid into a cell through a viral or viral-like entry mechanism. A “lentiviral vector” is a type of retroviral vector well-known in the art that has certain advantages in transducing nondividing cells as compared to other retroviral vectors. See, Trono D. (2002) Lentiviral vectors, New York: Spring-Verlag Berlin Heidelberg.

Lentiviral vectors of this invention are based on or derived from oncoretroviruses (the sub-group of retroviruses containing MLV), and lentiviruses (the sub-group of retroviruses containing HIV). Examples include ASLV, SNV and RSV all of which have been split into packaging and vector components for lentiviral vector particle production systems. The lentiviral vector particle according to the invention may be based on a genetically or otherwise (e.g. by specific choice of packaging cell system) altered version of a particular retrovirus.

That the vector particle according to the invention is “based on” a particular retrovirus means that the vector is derived from that particular retrovirus. The genome of the vector particle comprises components from that retrovirus as a backbone. The vector particle contains essential vector components compatible with the RNA genome, including reverse transcription and integration systems. Usually these will include gag and pol proteins derived from the particular retrovirus. Thus, the majority of the structural components of the vector particle will normally be derived from that retrovirus, although they may have been altered genetically or otherwise so as to provide desired useful properties. However, certain structural components and in particular the env proteins, may originate from a different virus. The vector host range and cell types infected or transduced can be altered by using different env genes in the vector particle production system to give the vector particle a different specificity.

As used herein, the term “Jurkat” refers to a human lymphocyte cell line. There are different types of Jurkat cell. In one embodiment, the Jurkat cell is capable of producing IL-2. The Jurkat cell is available commercially or from a cell line repository (e.g., ATCC No. TIB-152), and methods and compositions to culture the cell are described therein.

As used herein, the term “JurMa” or “Jurkat/MA” refers to a Jurkat cell line lacking endogenous TCR expression. One embodiment of the JurMa cells were established by Dr. Erik Hooijberg Vrije at Universiteit Medisch Centrum, Amsterdam (See Asai et al., PLoS One. 8(2): e56820 (2013), last accessed on Jan. 12, 2017.

As used herein, the term “BW5147” refers to a lymphocyte cell line, which can be used to study T-cell function. In some embodiments, BW5147 cells derive from the lymphoma. There are many types of BW5147 cells, which are available commercially or from a cell line repository (e.g., ATCC No. TIB-472), and methods and compositions to culture the cell are described therein.

As used herein, the term “HuT-78” refer to a lymphocyte cell line. In one embodiment, the HuT-78 is a T-cell lymphoma cell line. The HuT-78 cells are available commercially (e.g., Sigma-Aldrich) or from a cell line repository (e.g., ATCC No. TIB-161), and methods and compositions to culture the cell are described therein.

As used herein, the term “CEM” refers to a lymphocyte cell line. In one embodiment, the CEM cell is a peripheral blood lymphoblast cell. The CEM cells are available from a cell line repository (e.g., ATCC Nos. CRL-2265 or CCL-119), and methods and compositions to culture the cell are described therein.

As used herein, the term “Molt-4” refers to a lymphocyte cell line. In one embodiment, the Molt-4 cell is an acute lymphoblastic leukemia cell. The Molt-4 cells are available commercially (e.g., Sigma-Aldrich) or from a cell line repository (e.g., ATCC No. CRL-1582), and methods and compositions to culture the cell are described therein.

“Valency” relates to the number of pMHCs per nanoparticle core, or co-stimulatory per nanoparticle, and/or cytokine per nanoparticle core.

“Density” when referring to pMHC per nanoparticle core, or co-stimulatory per nanoparticle, and/or cytokine per nanoparticle core is calculated as the surface area of the nanoparticle core with outer layers, which can also include linkers. Surface area is the total available surface area of the construct used.

“Antigen” as used herein refers to all, part, fragment, or segment of a molecule that can induce an immune response in a subject or an expansion of an immune cell, preferably a T or B cell. In one aspect, the antigen is a cancer-relevant antigen. In another aspect the antigen is an autoimmune disorder relevant antigen. In a further aspect, the antigen is an allergen.

The term “alkyl” refers to monovalent saturated aliphatic hydrocarbyl groups having from 1 to 10 carbon atoms (i.e., C1-C10 alkyl) or 1 to 6 carbon atoms (i.e., C1-C6 alkyl), or 1 to 4 carbon atoms. This term includes, by way of example, linear and branched hydrocarbyl groups such as methyl (CH3-), ethyl (CH3CH2-), n-propyl (CH3CH2CH2-), isopropyl ((CH3)2CH—), n-butyl (CH3CH2CH2CH2-), isobutyl ((CH3)2CHCH2-), sec-butyl ((CH3)(CH3CH2)CH—), t-butyl ((CH3)3C—), n-pentyl (CH3CH2CH2CH2CH2-), and neopentyl ((CH3)3CCH2-).

The term “alkoxy” refers to —O-alkyl.

A “mimic” is an analog of a given ligand or peptide, wherein the analog is substantially similar to the ligand. “Substantially similar” means that the analog has a binding profile similar to the ligand except that the mimic has one or more functional groups or modifications that collectively account for less than about 50%, less than about 40%, less than about 30%, less than about 20%, less than about 10%, or less than about 5% of the molecular weight of the ligand.

“Immune cells” includes, e.g., white blood cells (leukocytes) that are derived from hematopoietic stem cells (HSC) produced in the bone marrow, lymphocytes (T cells, B cells, natural killer (NK) cells), and myeloid-derived cells (neutrophil, eosinophil, basophil, monocyte, macrophage, dendritic cells). As used herein, the term “B cell” refers to a type of lymphocyte in the humoral immunity of the adaptive immune system. B cells principally function to make antibodies, serve as antigen presenting cells, release cytokines, and develop memory B cells after activation by antigen interaction. B cells are distinguished from other lymphocytes, such as T cells, by the presence of a B-cell receptor on the cell surface. As used herein, the term “T cell” refers to a type of lymphocyte that matures in the thymus. T cells play an important role in cell-mediated immunity and are distinguished from other lymphocytes, such as B cells, by the presence of a T-cell receptor on the cell surface. T-cells may either be isolated or obtained from a commercially available source. “T cell” includes all types of immune cells expressing CD3, including T-helper cells (CD4+ cells), cytotoxic T-cells (CD8+ cells), natural killer T-cells, T-regulatory cells (Treg) and gamma-delta T cells. A “cytotoxic cell” includes CD8+ T cells, natural-killer (NK) cells, and neutrophils, which cells are capable of mediating cytotoxicity responses.

The term “effector T cells,” as used herein, refers to T cells that can specifically bind an antigen and mediate an immune response (effector function) without the need for further differentiation. Examples of effector T cells include CTLs, TH1 cells, TH2 cells, effector memory cells, and T helper cells. In contrast to effector T cells, naïve T cells have not encountered their specific antigen, MHC complex, nor responded to it by proliferation and differentiation into an effector T cell. Effector T cells can be resting (in the GO phase of the cell cycle) or activated (proliferating).

The term “anti-pathogenic autoreactive T cell” refers to a T cell with anti-pathogenic properties (i.e., T cells that counteract an autoimmune disease such as MS, a MS-related disease or disorder, or pre-diabetes). These T cells can include anti-inflammatory T cells, central memory T cells, effector memory T cells, memory T cells, low-avidity T cells, T helper cells, autoregulatory T cells, cytotoxic T cells, natural killer T cells, regulatory T cells, TR1 cells, suppressor T cells, CD4+ T cells, CD8+ T cells, and the like.

The term “anti-inflammatory T cell” refers to a T cell that promotes an anti-inflammatory response. The anti-inflammatory function of the T cell may be accomplished through production and/or secretion of anti-inflammatory proteins, cytokines, chemokines, and the like. Anti-inflammatory proteins are also intended to encompass anti-proliferative signals that suppress immune responses. Anti-inflammatory proteins include IL-4, IL-10, IL-13, IL-21, IL-23, IL-27, IFN-α, TGF-β, IL-Ira, G-CSF, and soluble receptors for TNF and IL-6.

The term “differentiated” refers to when a cell of a first type is induced into developing into a cell of a second type. In some embodiments, a cognate T cell is differentiated into a regulatory TR1 cell. In some embodiments, an activated T cell is differentiated into a TR1 cell. In some embodiments, a memory T cell is differentiated into a TR1 cell. In some embodiments, a B cell is differentiated into a regulatory B cell.

As used herein, “knob-in-hole” refers to a polypeptidyl architecture requiring a protuberance (or “knob”) at an interface of a first polypeptide and a corresponding cavity (or a “hole”) at an interface of a second polypeptide, such that the protuberance can be positioned in the cavity so as to promote heteromultimer formation. Protuberances are constructed by replacing small amino acid side chains from the interface of the first polypeptide with larger side chains (e.g., phenylalanine or tyrosine). Cavities of identical or similar size to the protuberances are created in the interface of the second polypeptide by replacing large amino acid side chains with smaller ones (e.g., alanine or threonine). The protuberances and cavities can be made by synthetic means such as by altering the nucleic acid encoding the polypeptides or by peptide synthesis, using routine methods by one skilled in the art. In some embodiments, the interface of the first polypeptide is located on an Fc domain in the first polypeptide; and the interface of the second polypeptide is located on an Fc domain in the second polypeptide. Knob-in-hole heteromultimers and methods of their preparation and use are disclosed in U.S. Pat. Nos. 5,731,168; 5,807,706; 5,821,333; 7,642,228; 7,695,936; 8,216,805; and 8,679,785, all of which are incorporated by reference herein in their entirety.

As used herein, “MHC-alpha-Fc/MHC-beta-Fc” refers to a heterodimer comprising a first polypeptide and a second polypeptide, wherein the first polypeptide comprises an MHC class II α-chain and an antibody Fc domain; the second polypeptide comprises an MHC class II β-chain and an antibody Fc domain. A knob-in-hole MHC-alpha-Fc/MHC-beta-Fc further requires that the Fc domains of each polypeptide interface with one another through the complementary positioning of a protuberance on one Fc domain within the corresponding cavity on the other Fc domain.

The term “isolated” means separated from constituents, cellular and otherwise, which the polynucleotide, peptide, polypeptide, protein, antibody, or fragment(s) thereof, are normally associated with in nature. For example, with respect to a polynucleotide, an isolated polynucleotide is one that is separated from the 5′ and 3′ sequences with which it is normally associated in the chromosome. As is apparent to those of skill in the art, a non-naturally occurring polynucleotide, peptide, polypeptide, protein, antibody, or fragment(s) thereof, does not require “isolation” to distinguish it from its naturally occurring counterpart. In addition, a “concentrated” “separated,” or “diluted” polynucleotide, peptide, polypeptide, protein, antibody, or fragment(s) thereof, is distinguishable from its naturally occurring counterpart in that the concentration or number of molecules per volume is greater than “concentrated” or less than “separated” than that of its naturally occurring counterpart. A polynucleotide, peptide, polypeptide, protein, antibody, or fragment(s) thereof, which differs from the naturally occurring counterpart in its primary sequence or, for example, by its glycosylation pattern, need not be present in its isolated form since it is distinguishable from its naturally occurring counterpart by its primary sequence, or alternatively, by another characteristic such as its glycosylation pattern. A mammalian cell, such as T cell, is isolated if it is removed from the anatomical site in which it is found in an organism.

An “auto-reactive T cell” is a T cell that recognizes an “auto-antigen”, which is a molecule produced and contained by the same individual that contains the T cell.

A “pathogenic T cell” is a T cell that is harmful to a subject containing the T cell, whereas a non-pathogenic T cell is not substantially harmful to a subject, and an anti-pathogenic T cells reduces, ameliorates, inhibits, or negates the harm of a pathogenic T cell.

As used herein, the terms regulatory B cells or B-regulatory cells (“B-regs”) refer to those cells that are responsible for the anti-inflammatory effect that is characterized by the expression of CD1d and CD5 and the secretion of IL-10. B-regs are also identified by expression of Tim-1 and can be induced through Tim-1 ligation to promote tolerance. The ability of B-regs was shown to be driven by many stimulatory factors such as toll-like receptors, CD40-ligand and others. However, full characterization of B-regs is ongoing. B-regs also express high levels of CD25, CD86, and TGF-β. This subset of B cells is able to suppress Th1 proliferation, thus contributing to the maintenance of self-tolerance. The potentiation of B-reg function should become the aim of many immunomodulatory drugs, contributing to a better control of autoimmune diseases. See, for example: ncbi.nlm.nih.gov/pubmed/23707422, last accessed on Oct. 31, 2013.

Type-1 T Regulatory (TR1) cells are a subset of CD4+ T cells that have regulatory properties and are able to suppress antigen-specific immune responses in vitro and in vivo. These TR1 cells are defined by their unique profile of cytokine production and make high levels of IL-10 and TGF-beta, but no IL-4 or IL-2. The IL-10 and TGF-beta produced by these cells mediate the inhibition of primary naïve T cells in vitro. There is also evidence that TR cells exist in vivo, and the presence of high IL-10-producing CD4(+) T cells in patients with severe combined immunodeficiency who have received allogeneic stem-cell transplants has been documented. TR1 cells are involved in the regulation of peripheral tolerance, and they could potentially be used as a cellular therapy to modulate immune responses in vivo. See, for example: ncbi.nlm.nih.gov/pubmed/10887343, last accessed on Oct. 31, 2013.

TR1 cells are defined by their ability to produce high levels of IL-10 and TGF-beta. Tr1 cells specific for a variety of antigens arise in vivo, but may also differentiate from naïve CD4+ T cells in the presence of IL-10 in vitro. TR1 cells have a low proliferative capacity, which can be overcome by IL-15. TR1 cells suppress naïve and memory T helper type 1 or 2 responses via production of IL-10 and TGF-beta. Further characterization of TR1 cells at the molecular level will define their mechanisms of action and clarify their relationship with other subsets of TR cells. The use of TR1 cells to identify novel targets for the development of new therapeutic agents, and as a cellular therapy to modulate peripheral tolerance, can be foreseen. See, for example, ncbi.nlm.nih.gov/pubmed/11722624, last accessed on Oct. 31, 2013.

An “an effective amount” is an amount sufficient to achieve the intended purpose; non-limiting examples of such include complexing of T cell receptors, initiation of the immune response, modulation of the immune response, suppression of an inflammatory response, and modulation of T cell activity or T cell populations. In one embodiment, the effective amount is one that is sufficient to stimulate TCR-pathway of a target cell. In one aspect, the effective amount is one that functions to achieve a stated therapeutic purpose, i.e., a therapeutically effective amount or to provide a measureable response. As described herein in detail, the effective amount or dosage depends on the purpose and the composition and can be determined according to the present disclosure.

An effective amount of therapeutic composition is determined based on the intended goal. The term “unit dose” or “dosage” refers to physically discrete units suitable for use in a subject, each unit containing a predetermined quantity of the composition calculated to produce the desired responses discussed above in association with its administration, i.e., the appropriate route and regimen. The quantity to be administered, according to both the number of treatments and the unit dose, depends on the result and/or protection desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition. Upon formulation, solutions will be administered in a manner compatible with the dosage formulation and in such amount as is therapeutically or prophylactically effective. The formulations are easily administered in a variety of dosage forms, such as the type of injectable solutions described above.

An “MHC multimer” as the term is used herein means a complex of two or more, usually four, or up to about fifty or more MHC monomers.

As used herein, a “multimer complex” refers to a complex between a target cell population and one or more pMHC complexes, wherein the MHC protein of the pMHC complex comprises multimeric form of the MHC protein. In some embodiments, the multimeric form of the MHC protein includes a dimer, a trimer, a tetramer, a pentamer or a dextramer.

As used herein, the phrase “immune response” or its equivalent “immunological response” refers to the development of a cell-mediated response (mediated by antigen-specific T cells or their secretion products). A cellular immune response is elicited by the presentation of polypeptide epitopes in association with Class I or Class II MHC molecules to treat or prevent a viral infection and/or expand antigen-specific Breg cells, TC1, CD4+T helper cells and/or CD8+ cytotoxic T cells and/or disease generated, autoregulatory T cell and B cell “memory” cells. The response may also involve activation of other components. In some aspects, the term “immune response” may be used to encompass the formation of a regulatory network of immune cells. Thus, the term “regulatory network formation” may refer to an immune response elicited such that an immune cell, preferably a T cell, more preferably a T regulatory cell, triggers further differentiation of other immune cells, including, but not limited to, B cells or antigen-presenting cells, non-limiting examples of which include dendritic cells, monocytes, and macrophages. In certain embodiments, regulatory network formation involves B cells being differentiated into regulatory B cells; in certain embodiments, regulatory network formation involves the formation of tolerogenic antigen-presenting cells.

As used herein, “nanosphere,” “NP,” or “nanoparticle” means a small, discrete particle that is administered singularly or in plural to a subject, cell specimen or tissue specimen as appropriate. In certain embodiments, the term “nanoparticle” as used herein includes any layers around the nanoparticle core and thus includes the core with and without a layer such as a linker layer. In certain embodiments, the nanoparticles are substantially spherical in shape. In certain embodiments, the nanoparticle is not a liposome or a viral particle. In further embodiments, the nanoparticle is comprised of any appropriate material, e.g., a solid, a solid core, a metal, a dendrimer, a polymeric micelle, a metal oxide, or a protein or fragment or combinations thereof. The term “substantially spherical,” as used herein, means that the shape of the particles does not deviate from a sphere by more than about 10%.

The terms “inflammatory response” and “inflammation” as used herein indicate the complex biological response of vascular tissues of an individual to harmful stimuli, such as pathogens, damaged cells, or irritants, and includes secretion of cytokines and, more particularly, of pro-inflammatory cytokines, i.e., cytokines which are produced predominantly by activated immune cells and are involved in the amplification of inflammatory reactions. Exemplary pro-inflammatory cytokines include but are not limited to IL-1, IL-6, IL-10, TNF-α IL-17, IL21, IL23, IL27, and TGF-β. Exemplary inflammations include acute inflammation and chronic inflammation. Acute inflammation indicates a short-term process characterized by the classic signs of inflammation (swelling, redness, pain, heat, and loss of function) due to the infiltration of the tissues by plasma and leukocytes. An acute inflammation typically occurs as long as the injurious stimulus is present and ceases once the stimulus has been removed, broken down, or walled off by scarring (fibrosis). Chronic inflammation indicates a condition characterized by concurrent active inflammation, tissue destruction, and attempts at repair. Chronic inflammation is not characterized by the classic signs of acute inflammation listed above. Instead, chronically inflamed tissue is characterized by the infiltration of mononuclear immune cells (monocytes, macrophages, lymphocytes, and plasma cells), tissue destruction, and attempts at healing, which include angiogenesis and fibrosis. An inflammation can be inhibited in the sense of the present disclosure by affecting and in particular inhibiting any one of the events that form the complex biological response associated with an inflammation in an individual.

As used herein, the term “disease-relevant” antigen refers to an antigen or fragment thereof selected to treat a selected disease and is involved in the disease process. For example, a diabetes-relevant antigen is an antigen or fragment thereof that, when presented, produces an immune response that serves to treat diabetes; thus, a diabetes-relevant antigen producing such an effect is selected to treat diabetes. A multiple sclerosis (MS)-relevant antigen is selected to treat MS. A diabetes-relevant antigen would not be selected to treat MS. Similarly, an autoimmunity-related antigen is an antigen that is relevant to an autoimmune disease and would not be selected for the treatment of a disorder or disease other than autoimmunity, e.g., cancer. Non-limiting, exemplary disease-relevant antigens are disclosed herein and further, such antigens may be determined for a particular disease based on techniques, mechanisms, and methods documented in the literature.

“Autoimmune disease or disorder” includes diseases or disorders arising from and directed against an individual's own tissues or organs or manifestation thereof or a condition resulting there from. In one embodiment, it refers to a condition that results from, or is aggravated by, the production by T cells that are reactive with normal body tissues and antigens. Examples of autoimmune diseases or disorders include, but are not limited to, arthritis (rheumatoid arthritis such as acute arthritis, chronic rheumatoid arthritis, gout or gouty arthritis, acute gouty arthritis, acute immunological arthritis, chronic inflammatory arthritis, degenerative arthritis, type II collagen-induced arthritis, infectious arthritis, Lyme arthritis, proliferative arthritis, psoriatic arthritis, Still's disease, vertebral arthritis, juvenile-onset rheumatoid arthritis, osteoarthritis, arthritis chronica progrediente, arthritis deformans, polyarthritis chronica primaria, reactive arthritis, and ankylosing spondylitis), inflammatory hyperproliferative skin diseases, psoriasis such as plaque psoriasis, guttate psoriasis, pustular psoriasis and psoriasis of the nails, atopy including atopic diseases such as hay fever and Job's syndrome, dermatitis including contact dermatitis, chronic contact dermatitis, exfoliative dermatitis, allergic dermatitis, allergic contact dermatitis, dermatitis herpetiformis, nummular dermatitis, seborrheic dermatitis, non-specific dermatitis, primary irritant contact dermatitis and atopic dermatitis, x-linked hyper IgM syndrome, allergic intraocular inflammatory diseases, urticaria such as chronic allergic urticaria and chronic idiopathic urticaria, including chronic autoimmune urticaria, myositis, polymyositis/dermatomyositis, juvenile dermatomyositis, toxic epidermal necrolysis, scleroderma (including systemic scleroderma), sclerosis such as systemic sclerosis, multiple sclerosis (MS) such as spino-optical MS, primary progressive MS (PPMS), and relapsing remitting MS (RRMS), progressive systemic sclerosis, atherosclerosis, arteriosclerosis, sclerosis disseminata, ataxic sclerosis, neuromyelitis optica spectrum disorder (NMO, also known as Devic's Disease or Devic's Syndrome), inflammatory bowel disease (IBD) (for example, Crohn's disease, autoimmune-mediated gastrointestinal diseases, colitis such as ulcerative colitis, colitis ulcerosa, microscopic colitis, collagenous colitis, colitis polyposa, necrotizing enterocolitis, transmural colitis, and autoimmune inflammatory bowel disease), bowel inflammation, pyoderma gangrenosum, erythema nodosum, primary sclerosing cholangitis, respiratory distress syndrome, including adult or acute respiratory distress syndrome (ARDS), meningitis, inflammation of all or part of the uvea, iritis, choroiditis, an autoimmune hematological disorder, rheumatoid spondylitis, rheumatoid synovitis, hereditary angioedema, cranial nerve damage as in meningitis, herpes gestationis, pemphigoid gestationis, pruritis scroti, autoimmune premature ovarian failure, sudden hearing loss due to an autoimmune condition, IgE-mediated diseases such as anaphylaxis and allergic and atopic rhinitis, encephalitis such as Rasmussen's encephalitis and limbic and/or brainstem encephalitis, uveitis, such as anterior uveitis, acute anterior uveitis, granulomatous uveitis, nongranulomatous uveitis, phacoantigenic uveitis, posterior uveitis, or autoimmune uveitis, glomerulonephritis (GN) with and without nephrotic syndrome such as chronic or acute glomerulonephritis such as primary GN, immune-mediated GN, membranous GN (membranous nephropathy), idiopathic membranous GN or idiopathic membranous nephropathy, membrano- or membranous proliferative GN (MPGN), including Type I and Type II, and rapidly progressive GN, proliferative nephritis, autoimmune polyglandular endocrine failure, balanitis including balanitis circumscripta plasmacellularis, balanoposthitis, erythema annulare centrifugum, erythema dyschromicum perstans, eythema multiform, granuloma annulare, lichen nitidus, lichen sclerosus et atrophicus, lichen simplex chronicus, lichen spinulosus, lichen planus, lamellar ichthyosis, epidermolytic hyperkeratosis, premalignant keratosis, pyoderma gangrenosum, allergic conditions and responses, allergic reaction, eczema including allergic or atopic eczema, asteatotic eczema, dyshidrotic eczema, and vesicular palmoplantar eczema, asthma such as asthma bronchiale, bronchial asthma, and autoimmune asthma, conditions involving infiltration of T cells and chronic inflammatory responses, immune reactions against foreign antigens such as fetal A-B-O blood groups during pregnancy, chronic pulmonary inflammatory disease, autoimmune myocarditis, leukocyte adhesion deficiency, lupus, including lupus nephritis, lupus cerebritis, pediatric lupus, non-renal lupus, extra-renal lupus, discoid lupus and discoid lupus erythematosus, alopecia lupus, systemic lupus erythematosus (SLE) such as cutaneous SLE or subacute cutaneous SLE, neonatal lupus syndrome (NLE), and lupus erythematosus disseminatus, Type I diabetes, Type II diabetes, latent autoimmune diabetes in adults (or Type 1.5 diabetes). Also contemplated are immune responses associated with acute and delayed hypersensitivity mediated by cytokines and T-lymphocytes, sarcoidosis, granulomatosis including lymphomatoid granulomatosis, Wegener's granulomatosis, agranulocytosis, vasculitides, including vasculitis, large-vessel vasculitis (including polymyalgia rheumatica and giant T cell (Takayasu's) arteritis), medium-vessel vasculitis (including Kawasaki's disease and polyarteritis nodosa/periarteritis nodosa), microscopic polyarteritis, immunovasculitis, CNS vasculitis, cutaneous vasculitis, hypersensitivity vasculitis, necrotizing vasculitis such as systemic necrotizing vasculitis, and ANCA-associated vasculitis, such as Churg-Strauss vasculitis or syndrome (CSS) and ANCA-associated small-vessel vasculitis, temporal arteritis, aplastic anemia, autoimmune aplastic anemia, Coombs positive anemia, Diamond Blackfan anemia, hemolytic anemia or immune hemolytic anemia including autoimmune hemolytic anemia (AIHA), Addison's disease, autoimmune neutropenia, pancytopenia, leukopenia, diseases involving leukocyte diapedesis, CNS inflammatory disorders, Alzheimer's disease, Parkinson's disease, multiple organ injury syndrome such as those secondary to septicemia, trauma or hemorrhage, antigen-antibody complex-mediated diseases, anti-glomerular basement membrane disease, anti-phospholipid antibody syndrome, anti-phospholipid syndrome, allergic neuritis, Behcet's disease/syndrome, Castleman's syndrome, Goodpasture's syndrome, Reynaud's syndrome, Sjogren's syndrome, Stevens-Johnson syndrome, pemphigoid such as pemphigoid bullous and skin pemphigoid, pemphigus (including pemphigus vulgaris, pemphigus foliaceus, pemphigus mucus-membrane pemphigoid, and pemphigus erythematosus), autoimmune polyendocrinopathies, Reiter's disease or syndrome, thermal injury, preeclampsia, an immune complex disorder such as immune complex nephritis, antibody-mediated nephritis, polyneuropathies, chronic neuropathy such as IgM polyneuropathies or IgM-mediated neuropathy, autoimmune or immune-mediated thrombocytopenia such as idiopathic thrombocytopenic purpura (ITP) including chronic or acute ITP, acquired thrombocytopenic purpura, scleritis such as idiopathic cerato-scleritis, episcleritis, autoimmune disease of the testis and ovary including autoimmune orchitis and oophoritis, primary hypothyroidism, hypoparathyroidism, autoimmune endocrine diseases including thyroiditis such as autoimmune thyroiditis, Hashimoto's disease, chronic thyroiditis (Hashimoto's thyroiditis), or subacute thyroiditis, autoimmune thyroid disease, idiopathic hypothyroidism, Grave's disease, polyglandular syndromes such as autoimmune polyglandular syndromes (or polyglandular endocrinopathy syndromes), paraneoplastic syndromes, including neurologic paraneoplastic syndromes such as Lambert-Eaton myasthenic syndrome or Eaton-Lambert syndrome, stiff-man or stiff-person syndrome, encephalomyelitis such as allergic encephalomyelitis or encephalomyelitis allergica and experimental allergic encephalomyelitis (EAE), myasthenia gravis such as thymoma-associated myasthenia gravis, cerebellar degeneration, neuromyotonia, opsoclonus or opsoclonus myoclonus syndrome (OMS), and sensory neuropathy, multifocal motor neuropathy, Sheehan's syndrome, autoimmune hepatitis, chronic hepatitis, lupoid hepatitis, giant T cell hepatitis, chronic active hepatitis or autoimmune chronic active hepatitis, lymphoid interstitial pneumonitis (LIP), bronchiolitis obliterans (non-transplant) vs NSIP, Guillain-Barre syndrome, Berger's disease (IgA nephropathy), idiopathic IgA nephropathy, linear IgA dermatosis, acute febrile neutrophilic dermatosis, subcorneal pustular dermatosis, transient acantholytic dermatosis, cirrhosis such as primary biliary cirrhosis and pneumonocirrhosis, autoimmune enteropathy syndrome, Celiac or Coeliac disease, celiac sprue (gluten enteropathy), refractory sprue, idiopathic sprue, cryoglobulinemia, amylotrophic lateral sclerosis (ALS; Lou Gehrig's disease), coronary artery disease, autoimmune ear disease such as autoimmune inner ear disease (AIED), autoimmune hearing loss, polychondritis such as refractory or relapsed or relapsing polychondritis, pulmonary alveolar proteinosis, Cogan's syndrome/nonsyphilitic interstitial keratitis, Bell's palsy, Sweet's disease/syndrome, rosacea autoimmune, zoster-associated pain, amyloidosis, a non-cancerous lymphocytosis, a primary lymphocytosis, which includes monoclonal B cell lymphocytosis (e.g., benign monoclonal gammopathy and monoclonal gammopathy of undetermined significance, MGUS), peripheral neuropathy, paraneoplastic syndrome, channelopathies such as epilepsy, migraine, arrhythmia, muscular disorders, deafness, blindness, periodic paralysis, and channelopathies of the CNS, autism, inflammatory myopathy, focal or segmental or focal segmental glomerulosclerosis (FSGS), endocrine ophthalmopathy, uveoretinitis, chorioretinitis, autoimmune hepatological disorder, fibromyalgia, multiple endocrine failure, Schmidt's syndrome, adrenalitis, gastric atrophy, presenile dementia, demyelinating diseases such as autoimmune demyelinating diseases and chronic inflammatory demyelinating polyneuropathy, Dressler's syndrome, alopecia greata, alopecia totalis, CREST syndrome (calcinosis, Raynaud's phenomenon, esophageal dysmotility, sclerodactyly, and telangiectasia), male and female autoimmune infertility, e.g., due to anti-spermatozoan antibodies, mixed connective tissue disease, Chagas' disease, rheumatic fever, recurrent abortion, farmer's lung, erythema multiforme, post-cardiotomy syndrome, Cushing's syndrome, bird-fancier's lung, allergic granulomatous angiitis, benign lymphocytic angiitis, Alport's syndrome, alveolitis such as allergic alveolitis and fibrosing alveolitis, interstitial lung disease, transfusion reaction, leprosy, malaria, parasitic diseases such as leishmaniasis, kypanosomiasis, schistosomiasis, ascariasis, aspergillosis, Sampter's syndrome, Caplan's syndrome, dengue, endocarditis, endomyocardial fibrosis, diffuse interstitial pulmonary fibrosis, interstitial lung fibrosis, pulmonary fibrosis, idiopathic pulmonary fibrosis, cystic fibrosis, endophthalmitis, erythema elevatum et diutinum, erythroblastosis fetalis, eosinophilic faciitis, Shulman's syndrome, Felty's syndrome, flariasis, cyclitis such as chronic cyclitis, heterochronic cyclitis, iridocyclitis (acute or chronic), or Fuch's cyclitis, Henoch-Schonlein purpura, human immunodeficiency virus (HIV) infection, SCID, acquired immune deficiency syndrome (AIDS), echovirus infection, sepsis, endotoxemia, pancreatitis, thyroxicosis, parvovirus infection, rubella virus infection, post-vaccination syndromes, congenital rubella infection, Epstein-Barr virus infection, mumps, Evan's syndrome, autoimmune gonadal failure, Sydenham's chorea, post-streptococcal nephritis, thromboangitis ubiterans, thyrotoxicosis, tabes dorsalis, chorioiditis, giant T cell polymyalgia, chronic hypersensitivity pneumonitis, keratoconjunctivitis sicca, epidemic keratoconjunctivitis, idiopathic nephritic syndrome, minimal change nephropathy, benign familial and ischemia-reperfusion injury, transplant organ reperfusion, retinal autoimmunity, joint inflammation, bronchitis, chronic obstructive airway/pulmonary disease, silicosis, aphthae, aphthous stomatitis, arteriosclerotic disorders, asperniogenese, autoimmune hemolysis, Boeck's disease, cryoglobulinemia, Dupuytren's contracture, endophthalmia phacoanaphylactica, enteritis allergica, erythema nodosum leprosum, idiopathic facial paralysis, chronic fatigue syndrome, febris rheumatica, Hamman-Rich's disease, sensoneural hearing loss, haemoglobinuria paroxysmatica, hypogonadism, ileitis regionalis, leucopenia, mononucleosis infectiosa, traverse myelitis, primary idiopathic myxedema, nephrosis, ophthalmia symphatica, orchitis granulomatosa, pancreatitis, polyradiculitis acuta, pyoderma gangrenosum, Quervain's thyreoiditis, acquired spenic atrophy, non-malignant thymoma, vitiligo, toxic-shock syndrome, food poisoning, conditions involving infiltration of T cells, leukocyte-adhesion deficiency, immune responses associated with acute and delayed hypersensitivity mediated by cytokines and T-lymphocytes, diseases involving leukocyte diapedesis, multiple organ injury syndrome, antigen-antibody complex-mediated diseases, antiglomerular basement membrane disease, allergic neuritis, autoimmune polyendocrinopathies, oophoritis, primary myxedema, autoimmune atrophic gastritis, sympathetic ophthalmia, rheumatic diseases, mixed connective tissue disease, nephrotic syndrome, insulitis, polyendocrine failure, autoimmune polyglandular syndrome type I, adult-onset idiopathic hypoparathyroidism (AOIH), cardiomyopathy such as dilated cardiomyopathy, epidermolisis bullosa acquisita (EBA), hemochromatosis, myocarditis, nephrotic syndrome, primary sclerosing cholangitis, purulent or nonpurulent sinusitis, acute or chronic sinusitis, ethmoid, frontal, maxillary, or sphenoid sinusitis, an eosinophil-related disorder such as eosinophilia, pulmonary infiltration eosinophilia, eosinophilia-myalgia syndrome, Loffler's syndrome, chronic eosinophilic pneumonia, tropical pulmonary eosinophilia, bronchopneumonic aspergillosis, aspergilloma, or granulomas containing eosinophils, anaphylaxis, seronegative spondyloarthritides, polyendocrine autoimmune disease, sclerosing cholangitis, sclera, episclera, chronic mucocutaneous candidiasis, Bruton's syndrome, transient hypogammaglobulinemia of infancy, Wiskott-Aldrich syndrome, ataxia telangiectasia syndrome, angiectasis, autoimmune disorders associated with collagen disease, rheumatism, neurological disease, lymphadenitis, reduction in blood pressure response, vascular dysfunction, tissue injury, cardiovascular ischemia, hyperalgesia, renal ischemia, cerebral ischemia, and disease accompanying vascularization, allergic hypersensitivity disorders, glomerulonephritides, reperfusion injury, ischemic re-perfusion disorder, reperfusion injury of myocardial or other tissues, lymphomatous tracheobronchitis, inflammatory dermatoses, dermatoses with acute inflammatory components, multiple organ failure, bullous diseases, renal cortical necrosis, acute purulent meningitis or other central nervous system inflammatory disorders, ocular and orbital inflammatory disorders, granulocyte transfusion-associated syndromes, cytokine-induced toxicity, narcolepsy, acute serious inflammation, chronic intractable inflammation, pyelitis, endarterial hyperplasia, peptic ulcer, valvulitis, emphysema, alopecia areata, adipose tissue inflammation/diabetes type II, obesity associated adipose tissue inflammation/insulin resistance, and endometriosis.

In some embodiments, the autoimmune disorder or disease may include, but is not limited to, diabetes mellitus Type I and Type II, pre-diabetes, transplantation rejection, multiple sclerosis, a multiple-sclerosis related disorder, premature ovarian failure, scleroderma, Sjogren's disease/syndrome, lupus, vitiligo, alopecia (baldness), polyglandular failure, Grave's disease, hypothyroidism, polymyositis, pemphigus, Crohn's disease, colitis, autoimmune hepatitis, hypopituitarism, myocarditis, Addison's disease, autoimmune skin diseases, uveitis, pernicious anemia, hypoparathyroidism, and/or rheumatoid arthritis. Other indications of interest include, but are not limited to, asthma, allergic asthma, primary biliary cirrhosis, cirrhosis, Neuromyelitis Optica Spectrum Disorder (Devic's disease, opticospinal multiple sclerosis (OSMS)), Pemphigus vulgaris, inflammatory bowel disease (IBD), arthritis, Rheumatoid arthritis, systemic lupus erythematosus (SLE), Celiac disease, psoriasis, autoimmune cardiomyopathy, idiopathic dilated cardiomyopathy (IDCM), a Myasthenia Gravis, Uveitis, Ankylosing Spondylitis, Immune Mediated Myopathies, prostate cancer, anti-phospholipid syndrome (ANCA+), atherosclerosis, dermatomyositis, chronic obstructive pulmonary disease (COPD), emphysema, spinal cord injury, traumatic injury, tobacco-induced lung destruction, ANCA-associated vasculitis, psoriasis, sclerosing cholangitis, primary sclerosing cholangitis, and diseases of the central and peripheral nervous systems.

In some embodiments, the autoimmune disorder or disease may include, but is not limited to, diabetes, multiple sclerosis, Celiac Disease, primary biliary cirrhosis, pemphigus, pemphigus foliaceus, pemphigus vulgaris, neuromyelitis optica spectrum disorder, arthritis (including rheumatoid arthritis), allergic asthma, inflammatory bowel disease (including Crohn's disease and ulcerative colitis), systemic lupus erythematosus, atherosclerosis, chronic obstructive pulmonary disease, emphysema, psoriasis, autoimmune hepatitis, uveitis, Sjogren's Syndrome, scleroderma, anti-phospholipid syndrome, ANCA-associated vasculitis, and Stiff Man Syndrome.

Multiple sclerosis (MS) is also known as “disseminated sclerosis,” “encephalomyelitis disseminate,” or “allergic encephalomyelitis.” MS is an inflammatory disease in which the fatty myelin sheaths around the axons of the brain and spinal cord are damaged, leading to demyelination and scarring as well as a broad spectrum of signs and symptoms. Multiple sclerosis-related disorders include, for example, neuromyelitis optica spectrum disorder (NMO), uveitis, neuropathic pain, and the like.

“Myelin Oligodendrocyte Glycoprotein” (MOG) is a glycoprotein believed to be important in the process of myelination of nerves in the central nervous system (CNS). In humans this protein is encoded by the MOG gene. It is speculated to serve as a necessary “adhesion molecule” to provide structural integrity to the myelin sheath and is known to develop late on the oligodendrocyte. The GenBank accession numbers NM_001008228.2 and NP_001008229.1 represent the mRNA and protein sequence, respectively, of the MOG gene. The sequence associated with each of these GenBank accession numbers is incorporated by reference for all purposes.

As used herein, the terms “cancer” and “cancerous” refer to or describe the physiological condition in mammals that is typically characterized by unregulated cell growth. Examples of cancer include, but are not limited to, carcinoma, lymphoma, blastoma, sarcoma, and leukemia and metastases thereof. The term “metastasis” refers to the transference of disease-producing organisms or of malignant or cancerous cells to other parts of the body by way of the blood or lymphatic vessels or membranous surfaces. Non-limiting examples of such cancers include squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, prostate cancer, vulval cancer, thyroid cancer, hepatic carcinoma and various types of head and neck cancer. Table 2 is an exemplary non-limiting list of cancer-relevant antigens for use in this disclosure.

As used herein, the term “co-stimulatory” intends molecules that produce a secondary signal in vivo that serves to activate naïve T cells into antigen-specific T cells capable of producing an immune response to cells possessing said specific antigen. The present disclosure is not limited to any specific co-stimulatory molecule. The various co-stimulatory molecules are well-known in the art. Some non-limiting examples of co-stimulatory molecules are 4-IBBL, OX40L, CD40, IL-15/IL-15Ra, CD28, CD80, CD86, CD30L, and ICOSL as are their respective receptors and polynucleotides encoding them. In specific embodiments, the co-stimulatory molecules of the present disclosure may be any one or more of the following ligands and their respective receptors: B7-1/CD80, BTLA, B7-2/CD86, CD28, B7-H1/PD-L1, CTLA-4, B7-H2, Gi24/VISTA/B7-H5, B7-H3, ICOS, B7-H4, PD-1, B7-H6, PD-L2/B7-DC, B7-H7, PDCD6, LILRA3/CD85e, LILRB2/CD85d/ILT4, LILRA4/CD85g/ILT7, LILRB3/CD85a/ILT5, LILRB1/CD85j/ILT2, LILRB4/CD85k/ILT3, 4-1BB/TNFRSF9/CD137, GITR Ligand/TNFSF18, 4-1BB Ligand/TNFSF9, HVEM/TNFRSF14, BAFF/BLyS/TNFSF13B, LIGHT/TNFSF14, BAFF R/TNFRSF13C, Lymphotoxin-alpha/TNF-beta, CD27/TNFRSF7, OX40/TNFRSF4, CD27 Ligand/TNFSF7, OX40 Ligand/TNFSF4, CD30/TNFRSF8, RELT/TNFRSF19L, CD30 Ligand/TNFSF8, TACI/TNFRSF13B, CD40/TNFRSF5, TL1A/TNFSF15, CD40 Ligand/TNFSF5, TNF-alpha, DR3/TNFRSF25, TNF RII/TNFRSF1B, GITR/TNFRSF18, 2B4/CD244/SLAMF4, CD84/SLAMF5, BLAME/SLAMF8, CD229/SLAMF3, CD2, CRACC/SLAMF7, CD2F-10/SLAMF9, NTB-A/SLAMF6, CD48/SLAMF2, SLAM/CD150, CD58/LFA-3, CD7, DPPIV/CD26, CD96, EphB6, CD160, Integrin alpha 4 beta 1, CD200, Integrin alpha 4 beta 7/LPAM-1, CD300a/LMIR1, LAG-3, CRTAM, TIM-1/KIM-1/HAVCR, DAP12, TIM-4, Dectin-1/CLEC7A, TSLP R, ICOSL, and/or biological equivalents of each thereof.

As used herein, the term “co-stimulatory ligand” intends cell surface molecules that interact with co-stimulatory molecules.

As used herein, the term “cytokine” intends low molecular weight proteins secreted by various cells in the immune system that act as signaling molecules for regulating a broad range of biological processes within the body at the molecular and cellular levels. “Cytokines” include individual immunomodulating proteins that fall within the class of lymphokines, interleukins, or chemokines.

As used herein, the term “diabetes” refers to a variable disorder of carbohydrate metabolism caused by a combination of hereditary and environmental factors and is usually characterized by inadequate secretion or utilization of insulin, by excessive urine production, by excessive amounts of sugar in the blood and urine, and by thirst, hunger, and loss of weight. Diabetes is characterized by Type 1 Diabetes and Type 2 Diabetes. The non-obese diabetic (“NOD”) mouse is an accepted animal model for the study and treatment of diabetes. Type 1 Diabetes (T1D) in mice is associated with autoreactive CD8+ T cells. Non-obese diabetic (NOD) mice develop a form of T1D, closely resembling human T1D that results from selective destruction of pancreatic R cells by T cells recognizing a growing list of autoantigens. Although initiation of T1D clearly requires the contribution of CD4+ cells, there is compelling evidence that T1D is CD8+ T-cell-dependent. It has been discovered that a significant fraction of islet-associated CD8+ cells in NOD mice use CDR3-invariant Vα17-Jα42+ TCRs, referred to as “8.3-TCR-like.” These cells, which recognize the mimotope NRP-A7 (defined using combinatorial peptide libraries) in the context of the MHC molecule K^{d}, are already a significant component of the earliest NOD islet CD8+ infiltrates, are diabetogenic, and target a peptide from islet-specific glucose-6-phosphatase catalytic subunit-related protein (IGRP), a protein of unknown function. The CD8+ cells that recognize this peptide (IGRP206-214, similar to NRP-A7) are unusually frequent in the circulation (> 1/200 CD8+ cells). Notably, progression of insulitis to diabetes in NOD mice is invariably accompanied by cyclic expansion of the circulating IGRP206-214-reactive CD8+ pool, and by avid maturation of its islet-associated counterpart. More recently, it has been shown that islet-associated CD8+ cells in NOD mice recognize multiple IGRP epitopes, indicating that IGRP is a dominant autoantigen for CD8+ cells, at least in murine T1D. NOD islet-associated CD8+ cells, particularly those found early on in the disease process also recognize an insulin epitope (Ins B15-23).

As used herein, the term “pre-diabetes” intends an asymptomatic period preceding a diabetic condition characterized by subclinical beta cell damage wherein the patient exhibits normal plasma glucose levels. It is also characterized by the presence of islet cell autoantibodies (ICAs) and, when close to the onset of clinical symptoms, may be accompanied by intolerance to glucose.

As used herein, the term “multiple sclerosis-related disorder” intends a disorder that co-presents with a susceptibility to MS or with MS. Non-limiting examples of such include neuromyelitis optica spectrum disorder (NMO), uveitis, neuropathic pain sclerosis, atherosclerosis, arteriosclerosis, sclerosis disseminata, systemic sclerosis, spino-optical MS, primary progressive MS (PPMS) and relapsing remitting MS (RRMS), progressive systemic sclerosis, and ataxic sclerosis.

The terms “epitope” and “antigenic determinant” are used interchangeably to refer to a site on an antigen to which B and/or T cells respond or recognize. B cell epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually at least 5 or 8-20, amino acids in a unique spatial conformation. Methods of determining spatial conformation of epitopes include, for example, x-ray crystallography and 2-dimensional nuclear magnetic resonance. See, e.g., Glenn E. Morris, Epitope Mapping Protocols (1996). T cells recognize continuous epitopes of about nine amino acids for CD8 cells or about 9-20 amino acids for CD4 cells. T cells that recognize the epitope can be identified by in vitro assays that measure antigen-dependent proliferation, as determined by 3H-thymidine incorporation by primed T cells in response to an epitope (Burke et al., J. Inf. Dis., 170:1110-1119, 1994), by antigen-dependent killing (cytotoxic T lymphocyte assay, Tigges et al., J. Immunol., 156(10):3901-3910, 1996) or by cytokine secretion. The presence of a cell-mediated immunological response can be determined by proliferation assays (CD4+ T cells) or CTL (cytotoxic T lymphocyte) assays.

Optionally, an antigen or preferably an epitope of an antigen, can be chemically conjugated to or expressed as a fusion protein with other proteins, such as MHC and MHC related proteins.

As used herein, the terms “individual,” “patient,” and “subject” are used synonymously and refer to a mammal. In some embodiments, the individual is a human. In other embodiments, the individual is a mammal in need of veterinary medicine or is a mammal commonly used in a laboratory. In some embodiments, the mammal is a mouse, rat, simian, canine, feline, bovine, equine, or ovine.

As used in this disclosure, the term “polynucleotide” refers to a nucleic acid molecule that either is recombinant or has been isolated free of total genomic nucleic acid. Included within the term “polynucleotide” are oligonucleotides (nucleic acids that are 100 residues or fewer in length), recombinant vectors, including, for example, plasmids, cosmids, phage, viruses, and the like. Polynucleotides include, in certain aspects, regulatory sequences, isolated substantially away from their naturally occurring genes or protein encoding sequences. Polynucleotides may be RNA, DNA, analogs thereof, or a combination thereof. A nucleic acid encoding all or part of a polypeptide may contain a contiguous nucleic acid sequence encoding all or a portion of such a polypeptide of the following lengths: 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, 1095, 1100, 1500, 2000, 2500, 3000, 3500, 4000, 4500, 5000, 5500, 6000, 6500, 7000, 7500, 8000, 9000, 10000, or more nucleotides, nucleosides, or base pairs. It is also contemplated that a particular polypeptide from a given species may be encoded by nucleic acids containing natural variations that have slightly different nucleic acid sequences but, nonetheless, encode the same or substantially similar protein, polypeptide, or peptide.

A polynucleotide is composed of a specific sequence of five nucleotide bases: adenine (A); cytosine (C); guanine (G); thymine (T); and uracil (U) for thymine when the polynucleotide is RNA. Thus, the term “polynucleotide sequence” is the alphabetical representation of a polynucleotide molecule. This alphabetical representation can be input into databases in a computer having a central processing unit and used for bioinformatics applications such as functional genomics and homology searching.

The terms “isolated” or “recombinant” as used herein with respect to nucleic acids, such as DNA or RNA, refer to molecules separated from other DNAs or RNAs, respectively that are present in the natural source of the macromolecule as well as polypeptides. The term “isolated or recombinant nucleic acid” is meant to include nucleic acid fragments which are not naturally occurring as fragments and would not be found in the natural state. The term “isolated” is also used herein to refer to polynucleotides, polypeptides, and proteins that are isolated from other cellular proteins and is meant to encompass both purified and recombinant polypeptides. In other embodiments, the term “isolated or recombinant” means separated from constituents, cellular and otherwise, in which the cell, tissue, polynucleotide, peptide, polypeptide, protein, antibody or fragment(s) thereof, are normally associated in nature. For example, an isolated cell is a cell that is separated from tissue or cells of dissimilar phenotype or genotype. An isolated polynucleotide is separated from the 3′ and 5′ contiguous nucleotides with which it is normally associated in its native or natural environment, e.g., on the chromosome. As is apparent to those of skill in the art, a non-naturally occurring polynucleotide, peptide, polypeptide, protein, antibody, or fragment(s) thereof does not require “isolation” to distinguish it from its naturally occurring counterpart.

“Exogenous” with respect to a nucleic acid or polynucleotide indicates that the nucleic acid is part of a recombinant nucleic acid construct, or is not in its natural environment. For example, an exogenous nucleic acid can be a sequence from one species introduced into another species, i.e., a heterologous nucleic acid. Typically, such an exogenous nucleic acid is introduced into the other species via a recombinant nucleic acid construct. An exogenous nucleic acid also can be a sequence that is native to an organism and that has been reintroduced into cells of that organism. An exogenous nucleic acid that includes a native sequence can often be distinguished from the naturally occurring sequence by the presence of non-natural sequences linked to the exogenous nucleic acid, e.g., non-native regulatory sequences (promoters, enhancers, transcriptional terminators, IRES, ribosome skipping sequences) flanking a native sequence in a recombinant nucleic acid construct, or lack of intron sequences. In addition, stably transformed exogenous nucleic acids typically are integrated at positions other than the position where the native sequence is naturally found. The exogenous elements may be added to a construct, for example using genetic recombination.

As used herein, the terms “homologous,” “homology,” or “percent homology” when used herein to describe a nucleic acid sequence, relative to a reference sequence, can be determined using the formula described by Karlin and Altschul (Proc. Natl. Acad. Sci. USA 87: 2264-2268, 1990, modified as in Proc. Natl. Acad. Sci. USA 90:5873-5877, 1993). Such a formula is incorporated into the basic local alignment search tool (BLAST) programs of Altschul et al. (J. Mol. Biol. 215: 403-410, 1990). Percent homology of sequences can be determined using the most recent version of BLAST, as of the filing date of this application.

Percent (%) sequence identity with respect to a reference polypeptide sequence is the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are known for instance, using publicly available computer software such as BLAST, BLAST-2, ALIGN or Megalign (DNASTAR) software. Appropriate parameters for aligning sequences are able to be determined, including algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

A “composition” is intended to mean a combination of active agent and another compound or composition, inert (for example, a detectable agent or label) or active, such as an adjuvant. In certain embodiments, the composition does not contain an adjuvant.

A “pharmaceutical composition” is intended to include the combination of an active agent with a carrier, inert or active, making the composition suitable for diagnostic or therapeutic use in vitro, in vivo, or ex vivo.

As used herein, a “protein” or “polypeptide” or “peptide” refers to a molecule comprising at least five amino acid residues.

Other objects, features, and advantages of the present disclosure will become apparent from the following detailed description. Additional definitions are also provided therein. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

This disclosure provides a novel assay and compositions necessary to conduct the assay. One such composition is an isolated cell comprising an exogenously introduced recombinant T cell receptor (TCR), a TCR-pathway-dependent reporter, and a co-receptor that binds a class I or class II major histocompatibility complex (MHC) complex. In a further aspect, the isolated cell comprises an exogenously introduced a TCR-associated multi-subunit CD3 chain signaling complex. In a yet further aspect, the isolated cell comprises an exogenously introduced receptor for a co-stimulatory molecule and/or a cytokine receptor.

### Cells

The cell to be utilized in a potency assay described herein is a eukaryotic cell. The cell minimally expresses: 1) a TCR, recombinant or natural, that specifically binds a peptide-MHC that is coupled to the pMHC-NP to be assayed; 2) a CD3 signaling complex 3) a TCR-pathway-dependent reporter; and 4) an MHC co-receptor. Some cells or cell lines may naturally express a CD3 signaling complex and an MHC co-receptor (e.g., CD4 or CD8) at levels sufficient to carry out the assay described herein. However, based on the specific cell or cell line an MHC co-receptor, or one or more polypeptides of the CD3 signaling complex can be introduced by an exogenous polynucleotide to increase signal or regulate signal in a homogenous manner. The cell can be a primary cell or a cell line that has been engineered to express one or more of a recombinant T cell receptor (TCR), a TCR-pathway-dependent reporter, an MHC co-receptor, or one or more polypeptides of the CD3 signaling complex. Non-limiting examples of suitable cell lines that can be engineered include JurMA, Jurkat, BW5147, HuT-78, CEM, Molt-4, or the combination thereof. If the cell does not endogenously express any of a recombinant T cell receptor (TCR), a TCR-pathway-dependent reporter, an MHC co-receptor, or one or more polypeptides of the CD3 signaling complex, then that component can be expressed from a polynucleotide introduced into the cell or cell line. In certain embodiments, the cell does not endogenously express a CD3 signaling complex. In certain embodiments, the cell does not endogenously express an MHC co-receptor. In one aspect, the cell endogenously expresses a receptor for a co-stimulatory molecule and/or a cytokine. In certain embodiments, the cell expresses at low level or does not express a receptor for a co-stimulatory molecule and/or a cytokine, but the expression is upregulated when T-cells are activated. The cell can comprise an addition of any one or more of an exogenous polynucleotide encoding a MHC co-receptor, a polypeptide that is part of a CD3 signaling complex. In certain embodiments, the polypeptide that is part of a CD3 signaling complex comprises an amino acid sequence at least 80%, 90%, 95%, 97%, 98%, 99% or 100% identical to SEQ ID NO: 553. In certain embodiments, the polypeptide that is part of a CD3 signaling complex is encoded by polynucleotide at least 80%, 90%, 95%, 97%, 98%, 99% or 100% homologous to SEQ ID NO: 554. In certain embodiments, the MHC co-receptor comprises an amino acid sequence at least 80%, 90%, 95%, 97%, 98%, 99% or 100% identical to SEQ ID NO: 549 or 551. In certain embodiments, the MHC co-receptor is encoded by polynucleotide at least 80%, 90%, 95%, 97%, 98%, 99% or 100% homologous to SEQ ID NO: 550 or 552.

### T Cell Receptor (TCR)

The cells or cell lines utilized for the potency assay described herein express a recombinant T cell receptor (TCR). A recombinant T cell receptor is one that is encoded by a polynucleotide lacking one or more of a 3′ UTR, a 5′UTR, an intron sequence, or native promoter or enhancer elements. This recombinant TCR can be encoded by an exogenous polynucleotide introduced by transduction, transfection, or infection. In certain embodiments, the exogenous polynucleotide is integrated into the genome of the cell or cell line. Non-limiting examples of T cell receptors include, without limitation, a heterodimer comprising a TCR α and TCR β, a heterodimer comprising TCR γ and TCR δ, and a single chain TCR construct. In a certain embodiment, the TCR is murinized (i.e., wherein the TCR is optimized to interact with a murine CD4 molecule). Non-limiting examples of TCR α can be found at GenBank, e.g., GenBank Accession Nos. AAB31880.1, AAB28318.1, AAB24428.1, and ADW95878.1, and equivalents of each thereof. Polynucleotides encoding these proteins are introduced into the cell using methods known in the art and that may further comprise operably coupled regulatory signals for expression on the cell surface, enhancers, as well as vectors for transduction and expression.

Non-limiting examples of TCR β can also be found at GenBank Accession Nos. AAB31887.1, AKG65861.1, ADW95908.1, and AAM53411.1, and equivalents of each thereof. Polynucleotides encoding these proteins are transduced into the cell using methods known in the art. The polynucleotides can be operably coupled regulatory signals for expression on the cell surface, enhancers, and as well as vectors for transduction and expression. In one embodiment, TCR γ-chain comprises one or more sequences found at GenBank, e.g., GenBank Accession Nos. AAM21533.1, DAA30449.1, and ABG91733.1 and equivalents of each thereof. Polynucleotides encoding these polypeptide can be transduced into the cell. The polynucleotides can be operably coupled regulatory signals for expression on the cell surface, enhancers, and as well as vectors for transduction and expression. In one embodiment, TCR δ-chain comprises one or more sequences found at GenBank, e.g., GenBank Accession Nos. Q7YRN2.1, AAC48547.1, JC4663, and NP_001009418.1, and equivalents of each thereof. Polynucleotides encoding these polypeptide can be transduced into the cell. The polynucleotides can be operably coupled regulatory signals for expression on the cell surface, enhancers, and as well as vectors for transduction and expression. The single chain TCRs are known in the art. Non-limiting examples of single chain TCRs are disclosed in WO1996018105 and US20120252742, and equivalents of each thereof, each of which is incorporated by reference in its entirety. Polynucleotides encoding these proteins are transduced into the cell using methods known in the art and further comprising operably coupled regulatory signals for expression on the cell surface, enhancers, as well as vectors for transduction and expression.

In one aspect, the TCR is a single chain TCR as disclosed in WO 1996018105 and US 2012/02522742. Polynucleotides encoding these polypeptides can be transduced into the cell. The polynucleotides can be operably coupled regulatory signals for expression on the cell surface, enhancers, and as well as vectors for transduction and expression.

In certain embodiments, the recombinant TCRs for use with the methods and cell lines described herein comprise a TCR alpha chain and a TCR beta chain. In certain embodiments, the TCR alpha chain and the TCR beta chain are separately translated. In certain embodiments, the TCR alpha chain and the TCR beta chain are translated as a single polypeptide. In certain embodiments, the TCR alpha chain and the TCR beta chain are translated as a single polypeptide as a single chain TCR. In certain embodiments, the TCR alpha chain and the TCR beta chain are translated as a single polypeptide that comprises a cleavage site between the TCR alpha chain and the TCR beta chain. In certain embodiments, the cleavage site comprises a ribosome skipping sequence. In certain embodiments, the TCR alpha chain and the TCR beta chain are expressed on the surface of the cell in a mature (secretory leader sequence cleaved) form.

In certain embodiments, the TCR alpha chain is at least 80%, 90%, 95%, 97%, 98%, 99% or 100% identical to any one of SEQ ID NOs: 528, 530, 534, 536 539, 541, 544, or 546 and the TCR beta chain is at least 80%, 90%, 95%, 97%, 98%, 99% or 100% identical to any one of SEQ ID NOs: 529, 531, 535, 537, 540, 542, 545, or 547. In certain embodiments, the TCR alpha chain is at least 80%, 90%, 95%, 97%, 98%, 99% or 100% identical to any one of SEQ ID NOs: 528, 530, 534, 536 539, or 541 and the TCR beta chain is at least 80%, 90%, 95%, 97%, 98%, 99% or 100% identical to any one of SEQ ID NOs: 529, 531, 535, 537, 540, or 542.

In certain embodiments, the TCR is specific for human islet-specific glucose-6-phosphatase catalytic subunit-related protein (IGRP) amino acids 13 to 25 (QHLQKDYRAYYTF, SEQ ID NO:9) bound to DRB1*0301/DRA*0101. In certain embodiments, the TCR alpha chain is at least 80%, 90%, 95%, 97%, 98%, 99% or 100% identical to any one of SEQ ID NOs: 528, or 530 the TCR beta chain is at least 80%, 90%, 95%, 97%, 98%, 99% or 100% identical to any one of SEQ ID NOs: 529 or 531. In certain embodiments, the TCR alpha chain is at least 80%, 90%, 95%, 97%, 98%, 99% or 100% identical to any one of SEQ ID NOs: 534 or 536 the TCR beta chain is at least 80%, 90%, 95%, 97%, 98%, 99% or 100% identical to any one of SEQ ID NOs: 535 or 537.

In certain embodiments, the TCR is specific for human preproinsulin amino acids 76 to 90 (SLQPLALEGSLQKRG, SEQ ID NO:17) bound to DRB1*0401/DRA*0101. In certain embodiments, the TCR alpha chain is at least 80%, 90%, 95%, 97%, 98%, 99% or 100% identical to any one of SEQ ID NOs: 539 or 541 the TCR beta chain is at least 80%, 90%, 95%, 97%, 98%, 99% or 100% identical to any one of SEQ ID NOs: 540 or 542.

In a further aspect the polynucleotide encoding TCRα and TCRβ further encodes a ribosome skipping sequence, non-limiting examples of which include but are not limited to a 2A ribosome skipping sequence (e.g., P2A, E2A, F2A, or T2A) or comprises an IRES sequence. Therefore, in one aspect, the ribosome skipping sequence comprises a P2A, E2A, F2A, or T2A ribosome skipping sequence. In some embodiments, the 2A ribosome skipping sequence comprises the consensus motif of Val/Ile-Glu-X-Asn-Pro-Gly-Pro (SEQ ID NO:558), wherein X stands for any amino acid. Non-limiting examples of 2A peptide sequences are provided in the Exemplary Sequence Listing. The polynucleotides can further comprise a promoter and/or an enhancer sequence. Examples of ribosomal skipping sequences can be found in WO 2013/057586 which is incorporated by reference.

Non limiting examples of IRES sequences and ribosome skipping sequences are provided in Tables 3 and 4.

In certain embodiments the TCR alpha chain and TCR beta chain are produced as a single polypeptide, and the TCR alpha chain and TCR beta chain are separated by a ribosome skipping sequence with an amino acid sequence set forth in any one of SEQ ID NOs: 456 to 523. In certain embodiments, the single polypeptide comprises an amino acid sequence at least 80%, 90%, 95%, 97%, 98%, 99% or 100% identical to any one of SEQ ID NOs: 524, 526, or 543. In certain embodiments, the single polypeptide comprises an amino acid sequence at least 80%, 90%, 95%, 97%, 98%, 99% or 100% identical to any one of SEQ ID NOs: 524, 526. In certain embodiments, the single polypeptide comprises an amino acid sequence at least 80%, 90%, 95%, 97%, 98%, 99% or 100% identical to any one of SEQ ID NOs: 524. In certain embodiments, the single polypeptide comprises an amino acid sequence at least 80%, 90%, 95%, 97%, 98%, 99% or 100% identical to any one of SEQ ID NOs: 526.

In certain embodiments the, TCR alpha chain and TCR beta chain are encoded by a single polynucleotide and the polynucleotide comprises an IRES sequence between the TCR alpha chain and the TCR beta chain. In certain embodiments, the IRES sequence comprises a nucleotide sequence set forth in any one of SEQ ID NOs: 524 to 526. In certain embodiments, the TCR alpha chain and/or the TCR beta chain are encoded by a poly nucleotide at least 80%, 90%, 95%, 97%, 98%, 99% or 100% homologous to SEQ ID NO: 532 or 557. This poly nucleotide can be stably integrated into the genome of the cell.

The TCR expressed by the cell utilized in the potency assay described herein can be specific for an autoimmune or inflammatory disease-relevant antigen. In certain embodiments, the autoimmune or disease-relevant antigen is a polypeptide bound to an MHC molecule. In certain embodiments, the autoimmune or disease-relevant antigen is a polypeptide bound to an MHC class I molecule. In certain embodiments, the autoimmune or disease-relevant antigen is a polypeptide bound to an MHC Class II molecule. In certain embodiments, the TCR binds to any polypeptide antigen set forth in Table 1.

The TCR expressed by the cell utilized in the potency assay described herein can be specific for a cancer antigen. In certain embodiments, the cancer antigen is a polypeptide bound to an MHC molecule. In certain embodiments, the cancer antigen is a polypeptide bound to an MHC class I molecule. In certain embodiments, the cancer antigen is a polypeptide bound to an MHC Class II molecule. In certain embodiments, the cancer antigen is a polypeptide set forth in Table 2.

### TCR-Dependent Reporter

In some embodiments, the TCR-pathway-dependent reporter is a reporter of TCR activation or TCR pathway activation. In one embodiment, the reporter provides one or more of cellular concentration, expression, activity, localization, protein modification, or protein-protein interactions. In some embodiments, the TCR-pathway-dependent reporter comprises, consists essentially of, or yet consists of a luciferase, a beta lactamase, chloramphenicol acetyltransferase (CAT), secreted embryonic alkaline phosphatase (SEAP), a fluorescent protein, or the combination thereof. In some embodiments, the TCR-pathway-dependent reporter comprises, consists essentially of, or yet consists of a nuclear factor of activated T cells (NFAT) transcription factor-binding DNA sequence or promoter, a NF-κB transcription factor-binding DNA sequence or promoter, an AP-1 transcription factor-binding DNA sequence or promoter, or an IL-2 transcription factor-binding DNA sequence or promoter. In certain embodiments, the luciferase comprises an amino acid sequence at least 80%, 90%, 95%, 97%, 98%, 99% or 100% identical to SEQ ID NO: 555. In certain embodiments, the luciferase is encoded by polynucleotide at least 80%, 90%, 95%, 97%, 98%, 99% or 100% homologous to SEQ ID NO: 556.

The TCR-dependent reporter is activated by an upstream promoter. Non-limiting examples of promoters are described herein and include without limitation NFAT transcription factor-binding DNA sequence or promoter, NF-κB transcription factor-binding DNA sequence or promoter, AP1 transcription factor-binding DNA sequence or promoter, and IL-2 transcription factor-binding DNA sequence or promoter. Additional examples are provided in the Exemplary Sequence Listing. In a further aspect, the polynucleotide further comprises an enhancer sequence.

In another aspect, the TCR-dependent reporter comprises, or alternatively consists essentially of, or yet further consists of a quantifiable gene product reporter Non-limiting examples of the quantifiable gene product reporters include but are not limited to a luciferase, a beta lactamase, CAT, SEAP, a fluorescent protein, or the combination thereof. Non-limiting examples of luciferase sequences for incorporation as a reporter can be located at GenBank (e.g., GenBank Accession Nos. AAR20792.1, AAL40677.1, AAL40676.1, and AAV35379.1, and equivalents of each thereof), last accessed on Jan. 12, 2017. The luciferase reporter system is available commercially (e.g., Promega Cat. #E1500 or E4550). Additional examples are provided in the Exemplary Sequence Listing. Non-limiting examples of beta lactamase sequences can be located at GenBank (e.g., GenBank Accession Nos AMM70781.1, CAA54104.1, and AAA23441.1, and equivalents of each thereof), last accessed on Jan. 12, 2017. Non-limiting examples of “CAT” can be located at GenBank (e.g., Accession Nos. OCR39292.1, WP_072643749.1, CUB58229.1, and KIX82948.1, and equivalents of each thereof), last accessed on Jan. 12, 2017. Polynucleotides encoding these polypeptide can be transduced into the cell. The CAT assays are commercially available (e.g., FAST CAT® Chloramphenicol Acetyltransferase Assay Kit (F-2900) from Thermal Fisher). Non-limiting examples of SEAP sequences can be located at GenBank (e.g., GenBank Accession Nos.

ADV10306.1, AAB64404.1, EEB84921.1, and EFD 70636.1, and equivalents of each thereof), last accessed on Jan. 12, 2017. Polynucleotides encoding these polypeptide can be transduced into the cell. The SEAP activity can be measured by a luminometer (e.g., Turner BioSystems Veritas Microplate Luminometer from Promega). Non-limiting examples of fluorescent protein include Green Fluorescent Protein (GFP), Enhanced Green Fluorescent Protein (eGFP), Blue Fluorekent Protein (BFP), Yellow Fluorescent Protein (YFP), Cyan Fluorescent Protein (CFP), Red Fluorescent Protein (RFP), or any other suitable fluorescent protein, or combination thereof, or fluorescent parts or derivatives thereof. The sequences of fluorescent proteins can be located at GenBank (e.g., GenBank Accession Nos. AFA52654.1, ACS44348.1, and AAQ96629.1, and equivalents of each thereof) last accessed on Jan. 12, 2017. Polynucleotides encoding these polypeptide can be transduced into the cell. The fluorescent protein promoter reporters are commercially available (e.g., TakaRa Cat. #631089).

### MHC Co-Receptors

The transformed cells also express a MHC co-receptor that binds a MHC ligand, e.g., class I and class II MHC ligands. In some embodiments, the MHC ligands comprise, consist of, or consist essentially of classical MHC class I protein, non-classical MHC class I protein, classical MHC class II protein, non-classical MHC class II protein, MHC dimers (Fc fusions), MHC tetramers, MHC multimers, or a polymeric form of an MHC protein.

In one aspect the MHC class I co-receptor comprises a CD8 complex. Exemplary sequences of CD8 can be located at GenBank (e.g., GenBank Accession Nos. AAA92533.1, AJP16706.1, AAA79217.1, and 1203216A, and equivalents of each thereof), last accessed on Jan. 19, 2017. Polynucleotides encoding these proteins are transduced into the cell using methods known in the art. The polynucleotides can be operably coupled regulatory signals for expression on the cell surface, enhancers, and as well as vectors for transduction and expression.

In another aspect, the MHC class II co-receptor comprises a CD4 molecule. Exemplary CD4 protein sequences can be located at GenBank (e.g., GenBank Accession Nos. CAA72740.1, AMR44293.1, ACG76115.1, AAC36010.1, and AAB 51309.1, and equivalents of each thereof), last accessed on Jan. 19, 2017. Polynucleotides encoding these proteins are transduced into the cell using methods known in the art. The polynucleotides can be operably coupled regulatory signals for expression on the cell surface and as well as vectors for transduction and expression.

### CD3

In a further aspect, a polynucleotide encoding “CD3” (cluster of differentiation 3) molecules is transduced into the cell and the cell, lacking endogenous CD3, now expresses the protein(s). In some embodiments, the CD3 comprises, or alternatively consists essentially of, or consists of four distinct chains. The non-limiting examples of CD3 chains can be found at GenBank, e.g., GenBank Accession Nos CAA72995.1, AAI45927.1, NP_998940.1, AAB24559.1, NP_000723.1, AEQ93556.1, and EAW67366.1 and equivalents thereof, are useful in this disclosure. As is apparent to the skilled artisan, the polynucleotide encoding CD3 may be operatively linked to regulatory elements for the expression of CD3 on the cell surface, optionally an enhancer, and included within a vector for expression of the polynucleotides. Polynucleotides encoding these proteins are transduced into the cell using methods known in the art.

In one embodiment, a TCR-associated multi-subunit CD3 chain signaling complex comprises, or alternatively consists essentially of, or yet further consists of a polypeptide or polypeptides of α and β TCR chains, the CD3γ, δ, and ε polypeptides, and the ζ chains. Forming in different modules, the TCR/CD3 complex can carry different roles. In one embodiment, the complex is involved in antigen-specific recognition. In some embodiments, the complex is involved in signal transduction primarily through the presence of immunorecepter tyrosine-based activation motif (“ITAM”) in the cytoplasmic tails of the CD3 chains. In some embodiments, the TCR/CD3 complex is involved in TCR signaling pathway stimulated by an antigen, a superantigen, or an antibody (e.g., receptor antibody). In one embodiment, exogenous expression of the TCR/CD3 complex facilitates the TCR signaling pathway in CD3-negative cells.

Co-Stimulatory Receptor(s) and/or Cytokine(s)

In a further aspect, the cell is transduced with a polynucleotide encoding a receptor for the selected co-stimulatory or cytokine molecule. Non-limiting examples of co-stimulatory and cytokine molecules are provided herein.

### Vectors

Vectors or other gene delivery systems can be used to transduce the cells with the polynucleotides as described above. In one aspect, the term “vector” intends a recombinant vector that retains the ability to infect and transduce non-dividing and/or slowly-dividing cells and integrate into the target cell's genome. In several aspects, the vector is derived from or based on a wild-type virus or plasmid, e.g., plasmid. In further aspects, the vector is derived from or based on a wild-type lentivirus. Examples of such, include without limitation, human immunodeficiency virus (HIV), equine infectious anemia virus (EIAV), simian immunodeficiency virus (SIV) and feline immunodeficiency virus (FIV). Alternatively, it is contemplated that other retrovirus can be used as a basis for a vector backbone such murine leukemia virus (MLV). It will be evident that a viral vector according to the invention need not be confined to the components of a particular virus. The viral vector may comprise components derived from two or more different viruses, and may also comprise synthetic components. Vector components can be manipulated to obtain desired characteristics, such as target cell specificity.

The recombinant vectors of this disclosure can be derived from primates and non-primates. Examples of primate lentiviruses include the human immunodeficiency virus (HIV), the causative agent of human acquired immunodeficiency syndrome (AIDS), and the simian immunodeficiency virus (SIV). The non-primate lentiviral group includes the prototype “slow virus” visna/maedi virus (VMV), as well as the related caprine arthritis-encephalitis virus (CAEV), equine infectious anemia virus (EIAV) and the more recently described feline immunodeficiency virus (FIV) and bovine immunodeficiency virus (BIV). Prior art recombinant lentiviral vectors are known in the art, e.g., see U.S. Pat. Nos. 6,924,123; 7,056,699; 7,07,993; 7,419,829 and 7,442,551, incorporated herein by reference.

U.S. Pat. No. 6,924,123 discloses that certain retroviral sequence facilitate integration into the target cell genome. This patent teaches that each retroviral genome comprises genes called gag, pol and env which code for virion proteins and enzymes. These genes are flanked at both ends by regions called long terminal repeats (LTRs). The LTRs are responsible for proviral integration, and transcription. They also serve as enhancer-promoter sequences. In other words, the LTRs can control the expression of the viral genes. Encapsidation of the retroviral RNAs occurs by virtue of a psi sequence located at the 5′ end of the viral genome. The LTRs themselves are identical sequences that can be divided into three elements, which are called U3, R and U5. U3 is derived from the sequence unique to the 3′ end of the RNA. R is derived from a sequence repeated at both ends of the RNA, and U5 is derived from the sequence unique to the 5′ end of the RNA. The sizes of the three elements can vary considerably among different retroviruses. For the viral genome. and the site of poly (A) addition (termination) is at the boundary between R and U5 in the right hand side LTR. U3 contains most of the transcriptional control elements of the provirus, which include the promoter and multiple enhancer sequences responsive to cellular and in some cases, viral transcriptional activator proteins.

With regard to the structural genes gag, pol and env themselves, gag encodes the internal structural protein of the virus. Gag protein is proteolytically processed into the mature proteins MA (matrix), CA (capsid) and NC (nucleocapsid). The pol gene encodes the reverse transcriptase (RT), which contains DNA polymerase, associated RNase H and integrase (IN), which mediate replication of the genome.

For the production of viral vector particles, the vector RNA genome is expressed from a DNA construct encoding it, in a host cell. The components of the particles not encoded by the vector genome are provided in trans by additional nucleic acid sequences (the “packaging system”, which usually includes either or both of the gag/pol and env genes) expressed in the host cell. The set of sequences required for the production of the viral vector particles may be introduced into the host cell by transient transfection, or they may be integrated into the host cell genome, or they may be provided in a mixture of ways. The techniques involved are known to those skilled in the art.

In one embodiment, the vector is a viral vector. In a related embodiment, the viral vector is selected from the group consisting of a lentiviral vector, retroviral vector, adenovirus vector, adeno-associated virus vector, and alphavirus vector. In yet a further embodiment, the viral vector is a lentiviral vector.

Non-viral vectors may include a plasmid that comprises a heterologous polynucleotide capable of being delivered to a target cell, either in vitro, in vivo or ex-vivo. The heterologous polynucleotide can comprise a sequence of interest and can be operably linked to one or more regulatory elements and may control the transcription of the nucleic acid sequence of interest. As used herein, a vector need not be capable of replication in the ultimate target cell or subject.

In one embodiment, the additional regulatory elements are promoters, enhancer and/or promoter/enhancer combinations. The promoter that regulates expression of the nucleic acid encoding the VEGF protein can be a constitutive promoter. In one aspect, the promoter that regulates the expression of the suicide gene is a constitutive promoter. Non-limiting examples of constitutive promoters include SFFV, CMV, PKG, MDNU3, SV40, Ef1a, UBC, and CAGG. In one aspect, the enhancer is a Woodchuck post-regulatory element (“WPRE”) (see, e.g., Zufferey, R. et al. (1999) J. Virol. 73(4):2886-2992).

Promoters useful in this disclosure can be constitutive or inducible. Some examples of promoters include SV40 early promoter, mouse mammary tumor virus LTR promoter, adenovirus major late promoter, herpes simplex virus promoter, and the CMV promoter. In one embodiment, the promoter that regulates expression of the tetracycline activator protein is a constitutive promoter. In other embodiments, the promoter is an inducible promoter, a tissue specific promoter, or a promoter that regulates expression temporally. In one embodiment, the promoter is a phosphoglycerate kinase promoter (PGK).

In a further aspect, the vector further comprises a marker or detectable label such as a gene encoding an enhanced green fluorescent protein (EGFP), red fluorescent protein (RFP), green fluorescent protein (GFP) and yellow fluorescent protein (YFP) or the like. These are commercially available and described in the technical art.

Other methods of delivering genes of the current invention include but are not limited to, calcium phosphate transfection, DEAE-dextran transfection, electroporation, microinjection, protoplast fusion, or liposome-mediated transfection. The host cells that are transfected with the vectors of this invention may include (but are not limited to) E. coli or other bacteria, yeast, fungi, insect cells (using, for example, baculoviral vectors for expression in SF9 insect cells), or cells derived from mice, humans, or other animals (e.g., mammals). In vitro expression of a protein, fusion, polypeptide fragment, or mutant encoded by cloned DNA may also be used. Those skilled in the art of molecular biology will understand that a wide variety of expression systems and purification systems may be used to produce recombinant proteins and fragments thereof.

### Cell Populations

In one aspect, the disclosure relates to a population of isolated cells, including but not limited to the cells in this disclosure, e.g., JurMA, Jurkat, BW5147, HuT-78, CEM, Molt-4 that are modified as described herein. In another aspect, the cells are CD3-negative cells. Non-limiting examples of CD3-negative cells include but are not limited to BW5147 (ATCC No. TIB-472), Nk-92 (ATCC No. CRL-2407), Mino (ATCC No. PTS-CRL-3000), and JeKo-1 (ATCC No. CRL-3006). In some embodiments, the population is substantially homogeneous. In another embodiment, the population is substantially heterogeneous. In certain embodiments, a population is a plurality of cells of this disclosure. In certain embodiments a population comprises at least 1×10² to 1×10⁹ cells that are at least 50%, 60%, 70%, 80%, 95, 95%, 98%, or 99% pure.

### Monitoring Expression

As is apparent to the skilled artisan, effective expression of the transduced polypeptide can be determined using methods known in the art, e.g., using detectable labeled antibodies or fragments thereof that can quantitatively or qualitatively monitored after transduction and culturing on the cells and cell populations.

### Methods for Preparing the Cells

In another aspect, the disclosure also relates to methods to prepare the isolated cell, which comprise, or consist essentially of, or yet further consist of transducing an isolated cell with one or more polynucleotides encoding: a recombinant T cell receptor (TCR), a TCR-pathway-dependent reporter, and an MHC co-receptor. In a further aspect, the method further includes transducing the cell with a polynucleotide encoding a TCR-associated multi-subunit CD3 chain signaling complex, and/or a co-stimulatory molecule and/or a cytokine. In some embodiment, the methods further comprise, consist essentially of, or yet consist of culturing the cells under conditions that favor expression of the one or more the transduced polynucleotides, e.g. a polynucleotide encoding the recombinant T cell receptor (TCR), the TCR-pathway-dependent reporter, the co-receptor that binds class I or class II major histocompatibility complex (MHC) ligands, optionally a TCR-associated multi-subunit CD3 chain signaling complex, the co-stimulatory molecule and/or the cytokine. In one embodiment, the methods further comprise, consist essentially of, or yet consist of isolating the cells that express the recombinant T cell receptor (TCR), the TCR-pathway-dependent reporter, the co-receptor that binds class I or class II major histocompatibility complex (MHC) ligands, and/or optionally a TCR-associated multi-subunit CD3 chain signaling complex, and further optionally the co-stimulatory molecule and/or the cytokine. In one embodiment, the cells are isolated by a method comprising flow cytometry. The isolated cells are cultured under conditions for expansion and continued expression of the transduced polynucleotides thereby provided a population of cells.

In certain aspects, the disclosure relates to in vitro methods of measuring the potency of the pMHC molecules that are optionally coupled to nanoparticle cores. The methods comprise, or alternatively consist essentially of, or yet consist of: (a) contacting a transduced cell expressing a T cell receptor (TCR) and a TCR-pathway-dependent reporter and a co-receptor that binds an MHC ligand, with an effective amount of a composition comprising pMHC, and (b) detecting said TCR-pathway-dependent reporter or a signal from said reporter. In a further aspect, the cells further comprise a CD3 complex and/or a co-stimulatory receptor, and/or a cytokine receptor.

In another embodiment, the contacting is in vitro.

In one embodiment, at least one pMHC on the complex interacts with the TCR, wherein the interaction activates the TCR-dependent pathway. In some embodiments, the TCR-pathway-dependent reporter is a reporter of TCR activation or TCR pathway activation. In one embodiment, the characteristic of the reporter comprises cellular concentration, expression, activity, localization, protein modification, or protein-protein interactions. In one embodiment, the reporter is a natural reporter, intrinsic to the effector cell type, having a characteristic that is detectable and correlates to TCR activation, or TCR pathway activation. In some embodiments, the reporter is an artificial reporter, exogenous to the effector cell type, having a characteristic that is detectable and correlates to TCR activation or TCR pathway activation.

In some embodiments, the isolated cells are as described above, e.g., effector cells comprising one or more of JurMA, Jurkat, BW5147, HuT-78, CEM, Molt-4, or primary T cells. Non-limiting examples of CD3-negative cells include but are not limited to BW5147 (ATCC No. TIB-472), Nk-92 (ATCC No. CRL-2407), Mino (ATCC No. PTS-CRL-3000), and JeKo-1 (ATCC No. CRL-3006).

In one embodiment, the TCR-pathway-dependent reporter comprises, consists essentially of, or yet consists of a gene coding for a protein selected from the group consisting of a luciferase (firefly or Renilla), a beta lactamase, CAT, SEAP, a fluorescent protein, and a quantifiable gene product. In some embodiments, the TCR-pathway-dependent reporter comprises, consists essentially of, or yet consists of a nuclear factor of activated T cells (NFAT) transcription factor-binding DNA sequence or promoter, a NF-κB transcription factor-binding DNA sequence or promoter, an AP-1 transcription factor-binding DNA sequence or promoter, or an IL-2 transcription factor-binding DNA sequence or promoter. In one embodiment, the reporter comprises, consists essentially of, or yet consists of a gene, the expression of which is under the control of TCR-pathway-dependent pathway.

In one embodiment, a TCR-associated multi-subunit CD3 chain signaling complex comprises, or alternatively consists essentially of, or yet further consists of a polypeptide or polypeptides of α and β TCR chains, the CD3γ, δ, and ε polypeptides, and the ζ chains. Forming in different modules, the TCR/CD3 complex can carry different roles. In one embodiment, the complex is involved in antigen-specific recognition. In some embodiments, the complex is involved in signal transduction primarily through the presence of immunorecepter tyrosine-based activation motif (“ITAM”) in the cytoplasmic tails of the CD3 chains. In some embodiments, the TCR/CD3 complex is involved in TCR signaling pathway stimulated by an antigen, a superantigen, or an antibody (e.g., anti-receptor antibody). In one embodiment, exogenous expression of the TCR/CD3 complex facilitates the TCR signaling pathway in CD3-negative cells. Non-limiting examples of CD3-negative cells include but are not limited to BW5147 (ATCC No. TIB-472), Nk-92 (ATCC No. CRL-2407), Mino (ATCC No. PTS-CRL-3000), and JeKo-1 (ATCC No. CRL-3006). In a further aspect, the cells endogenously express receptors for a cytokine and/or separately, a co-stimulatory molecule.

### Potency Assay Uses

In one aspect, the potency assay can measure the potency, purity, or activity of pMHC-nanoparticles. The assay can be used as, for example, a quality control step to monitor different batches or lots of pMHC-NP to verify that the lot comprises functioning pMHC able to bind T cells and/or induce the desired immune response. In one aspect, the potency assay can measure the activity of pMHC-nanoparticles, which optionally comprise, or further consist thereof, or alternatively further consist essentially of one or more co-stimulatory molecules and/or one or more cytokines coupled to the nanoparticle core.

For the nanoparticles that can be tested in the assay, the pMHC complexes on each nanoparticle core are the same or different from each other; and/or the MHC of the pMHC complexes on each nanoparticle core are the same or different from each other; and/or the cytokines on each nanoparticle core are the same or different from each other; and/or the costimulatory molecules on each nanoparticle core are the same or different from each other; and/or the diameters of the nanoparticle cores are the same or different from each other; and/or the valency of the pMHC complexes on each nanoparticle core are the same or different from each other; and/or the density of the pMHC complexes on each nanoparticle core are the same or different from each other; and/or the valency and/or the density of the co-stimulatory molecules on each nanoparticle core are the same or different from each other; and/or the valency and/or the density of the cytokines on each nanoparticle core are the same or different from each other. In one aspect, a composition is assayed wherein the composition comprising nanoparticles having a plurality pMHC complexes and then a separate plurality of nanoparticles having co-stimulatory and optionally cytokines. As above, the pMHC complexes on each nanoparticle core are the same or different from each other; and/or the MHC of the pMHC complexes on each nanoparticle core are the same or different from each other; and/or the cytokines on each nanoparticle core are the same or different from each other; and/or the costimulatory molecules on each nanoparticle core are the same or different from each other; and/or the diameters of the nanoparticle cores are the same or different from each other; and/or the valency of the pMHC complexes on each nanoparticle core are the same or different from each other; and/or the density of the pMHC complexes on each nanoparticle core are the same or different from each other; and/or the valency and/or the density of the co-stimulatory molecules on each nanoparticle core are the same or different from each other; and/or the valency and/or the density of the cytokines on each nanoparticle core are the same or different from each other.

In certain aspects, the nanoparticles that can be tested in the assay the nanoparticles are provided in a composition comprising a plurality of the nanoparticle complexes provided herein. In some embodiments, the compositions further comprise a carrier, optionally a pharmaceutical carrier.

The assay can be used to determine the potency of pMHC that are optionally coupled to nanoparticles, e.g., pMHC-nanoparticles. The terms “particle,” “nanoparticle,” “microparticle,” “bead,” “microsphere,” and grammatical equivalents thereof herein applies to small discrete particles that are administrable to a subject. In certain embodiments, the particles are substantially spherical in shape. The term “substantially spherical,” as used herein, means that the shape of the particles does not deviate from a sphere by more than about 10%. Various known antigen or peptide complexes of the disclosure may be applied to the particles.

Peptide MHC nanoparticles that are compatible and able to be analyzed using the potency assay described herein are at least those as described in, by way of non-limiting example, WO 2008/109852, WO 2012/041968, WO 2012/062904, WO 2013144811, WO 2014/050286, WO 2015/063616, WO 2016/198932, or PCT/IB2017/001508, all of which are incorporated by reference herein in their entireties.

The potency assay described herein can be used to quantitate a signal from a cell that has been at least transduced with a recombinant TCR and a pathway dependent reporter. The quantitation of the signal can be performed and utilized in many ways by those skilled in the art. In certain embodiments, the signal can be quantitated and compared to a preset threshold to determine whether a given preparation of a nanomedicine or nanoparticle passes a quality control step. The threshold can be at least about 150%, 200%, 300%, 400%, 500%, 600%, 70%, 800%, 900%, or 1,000%, of the signal quantitated from a negative control. A negative control can be, for example, a cell with a recombinant TCR and lacking a reporter of the kind quantitated; or a nanomedicine or nanoparticle that comprises an irrelevant peptide MHC complex or no peptide MHC complex. In certain embodiments, potency assay can be used to define an IC50 of a particular nanoparticle preparation.

### Nanoparticle Core and Layer Compositions

The nanoparticle core of the pMHC-NP comprises, or consists essentially of, or yet further consists of a core, for example a solid core, a metal core, a dendrimer core, a polymeric micelle nanoparticle core, a nanorod, a fullerene, a nanoshell, a coreshell, a protein-based nanostructure, or a lipid-based nanostructure. In some aspects, the nanoparticle core is bioabsorbable and/or biodegradable. In some aspects, the nanoparticle core is a dendrimer nanoparticle core comprising, or alternatively consisting essentially thereof, or yet further consisting of a highly branched macromolecule having a tree-like structure growing from a core. In further aspects, the dendrimer nanoparticle core may comprise, or alternatively consist essentially thereof, or yet further consist of a poly(amidoamine)-based dendrimer or a poly-L-lysine-based dendrimer. In certain aspects, the nanoparticle core is a polymeric micelle core comprising, or alternatively consisting essentially thereof, or yet further consisting of an amphiphilic block co-polymer assembled into a nano-scaled core-shell structure. In further aspects, the polymeric micelle core comprises, or alternatively consists essentially thereof, or yet further consists of a polymeric micelle produced using polyethylene glycol-diastearoylphosphatidylethanolamine block copolymer. In a further aspect, the nanoparticle core comprises, or alternatively consists essentially of, or yet further consists of a metal. In another aspect, the nanoparticle core is not a liposome. Additional examples of core materials include, but are not limited to, standard and specialty glasses, silica, polystyrene, polyester, polycarbonate, acrylic polymers, polyacrylamide, polyacrylonitrile, polyamide, fluoropolymers, silicone, celluloses, silicon, metals (e.g., iron, gold, silver), minerals (e.g., ruby), nanoparticles (e.g., gold nanoparticles, colloidal particles, metal oxides, metal sulfides, metal selenides, and magnetic materials such as iron oxide), and composites thereof. In some embodiments, an iron oxide nanoparticle core comprises iron (II, III) oxide. The core could be of homogeneous composition, or a composite of two or more classes of material depending on the properties desired. In certain aspects, metal nanoparticles will be used. These metal particles or nanoparticles can be formed from Au, Pt, Pd, Cu, Ag, Co, Fe, Ni, Mn, Sm, Nd, Pr, Gd, Ti, Zr, Si, and In, their precursors, their binary alloys, their ternary alloys, and their intermetallic compounds. See U.S. Pat. No. 6,712,997, which is incorporated herein by reference in its entirety. In certain embodiments, the compositions of the core and layers (described below) may vary provided that the nanoparticles are biocompatible and bioabsorbable. The core could be of homogeneous composition or a composite of two or more classes of material depending on the properties desired. In certain aspects, metal nanospheres will be used. These metal nanoparticles can be formed from Fe, Ca, Ga, and the like. In certain embodiments, the nanoparticle comprises, or alternatively consists essentially of, or yet further consists of a core comprising metal or metal oxide such as gold or iron oxide. In some embodiments, a plurality of co-stimulatory molecules and/or a plurality of cytokines are coupled to a nanoparticle dendrimer core or polymeric micelle core.

The particles typically consist of a substantially spherical core and optionally one or more layers or coatings. The core may vary in size and composition as described herein. In addition to the core, the particle may have one or more layers to provide functionalities appropriate for the applications of interest. The thicknesses of layers, if present, may vary depending on the needs of the specific applications. For example, layers may impart useful optical properties.

Layers may also impart chemical or biological functionalities, referred to herein as chemically active or biologically active layers. These layers typically are applied on the outer surface of the particle and can impart functionalities to the pMHC-NPs. The layer or layers may typically range in thickness from about 0.001 micrometers (1 nanometer) to about 10 micrometers or more (depending on the desired particle diameter) or from about 1 nm to 5 nm, or alternatively from about 1 nm to about 10 nm, or alternatively from about 1 nm to about 40 nm, or from about 15 nm to about 25 nm, or from about 15 nm to about 20 nm, and ranges in between.

The layer or coating may comprise, or alternatively consist essentially of, or yet further consist of a biodegradable sugar or other polymer. Examples of biodegradable layers include but are not limited to dextran; poly(ethylene glycol); poly(ethylene oxide); mannitol; poly(esters) based on polylactide (PLA), polyglycolide (PGA), polycaprolactone (PCL); poly(hydroxalkanoate) of the PHB-PHV class; and other modified poly(saccharides) such as starch, cellulose and chitosan. Additionally, the nanoparticle may include a layer with suitable surfaces for attaching chemical functionalities for chemical binding or coupling sites.

Layers can be produced on the nanoparticles in a variety of ways known to those skilled in the art. Examples include sol-gel chemistry techniques such as described in Iler, Chemistry of Silica, John Wiley & Sons, 1979; Brinker and Scherer, Sol-gel Science, Academic Press, (1990). Additional approaches to producing layers on nanoparticles include surface chemistry and encapsulation techniques such as described in Partch and Brown, J. Adhesion, 67:259-276, 1998; Pekarek et al., Nature, 367:258, (1994); Hanprasopwattana, Langmuir, 12:3173-3179, (1996); Davies, Advanced Materials, 10:1264-1270, (1998); and references therein. Vapor deposition techniques may also be used; see, for example, Golman and Shinohara, Trends Chem. Engin., 6:1-6, (2000); and U.S. Pat. No. 6,387,498. Still other approaches include layer-by-layer self-assembly techniques such as described in Sukhorukov et al., Polymers Adv. Tech., 9(10-11):759-767, (1998); Caruso et al., Macromolecules, 32(7):2317-2328, (1998); Caruso et al., J. Amer. Chem. Soc., 121(25):6039-6046, (1999); U.S. Pat. No. 6,103,379 and references cited therein.

The nanoparticles can comprise, consist essentially of, or yet further consist of a nanoparticle core coupled to a plurality of disease-relevant antigen-MHC complexes that are useful for expanding and differentiating T cell populations and treating disease when administered in an effective amount to a subject. In some aspects, the number of pMHCs per nanoparticle core (referred to herein as the “valency” of the nanoparticle complex) having a variety of ranges as described above and incorporated by reference herein.

In some aspects, the nanoparticle core is a dendrimer nanoparticle core comprising, or alternatively consisting essentially thereof, or yet further consisting of a highly branched macromolecule having a tree-like structure growing from a core. In further aspects, the dendrimer nanoparticle may comprise, or alternatively consist essentially thereof, or yet further consist of a poly(amidoamine)-based dendrimer or a poly-L-lysine-based dendrimer. In certain aspects, the nanoparticle core is a polymeric micelle core comprising, or alternatively consisting essentially thereof, or yet further consisting of an amphiphilic block co-polymer assembled into a nano-scaled core-shell structure. In further aspects, the polymeric micelle core may comprise, or alternatively consist essentially thereof, or yet further consist of a polymeric micelle produced using polyethylene glycol-diastearoylphosphatidylethanolamine block copolymer. The dendrimer core or polymeric micelle core may further comprise an outer coating or layer as described herein.

In certain embodiments, specific means of synthesis of dendrimer nanoparticles or nanoparticles with a dendrimer nanoparticle core may require that metal ions are extracted into the interior of dendrimers and then subsequently chemically reduced to yield nearly size-monodispersed particles having dimensions of less than 3 nm, such as the method disclosed in Crooks et al., “Synthesis, Characterization, and Applications of Dendrimer-Encapsulated Nanoparticles.” The Journal of Physical Chemistry B (109): 692-704 (2005), wherein the resulting dendrimer core component serves not only as a template for preparing the nanoparticle but also to stabilize the nanoparticle, making it possible to tune solubility, and provides a means for immobilization of the nanoparticle on solid supports. In some embodiments, a plurality of co-stimulatory molecules and/or a plurality of cytokines are coupled to a nanoparticle dendrimer core or polymeric micelle core.

The size of the nanoparticle core can range from about 1 nm to about 1 μm. In certain embodiments, the nanoparticle core is less than about 1 μm in diameter. In other embodiments, the nanoparticle core is less than about 500 nm, less than about 400 nm, less than about 300 nm, less than about 200 nm, less than about 100 nm, or less than about 50 nm in diameter. In further embodiments, the nanoparticle core is from about 1 nm to about 10 nm, 15 nm, 20 nm, 25 nm, 30 nm, 40 nm, 50 nm, 75 nm, or 100 nm in diameter. In specific embodiments, the nanoparticle core has a diameter of from about 1 nm to about 100 nm; from about 1 nm to about 75 nm; from about 1 nm to about 50 nm; from about 1 nm to about 25 nm; from about 1 nm to about 25 nm; from about 5 nm to about 100 nm; from about 5 nm to about 50 nm; from about 5 nm to about 25 nm: from about 15 nm to about 25 nm; or about 20 nm. In some embodiments, the nanoparticle core has a diameter of from about 25 nm to about 60 nm, or from about 25 nm to about 50 nm, or from about 20 nm to about 40 nm, or from about 15 nm to about 50 nm, or from about 15 nm to about 40 nm, or from about 15 nm to about 35 nm, or from about 15 nm to about 30 nm, or from about 15 nm to about 25 nm, or alternatively about 15 nm, or about 20 nm, or about 25 nm, or about 30 nm, or about 35 nm, or about 40 nm.

The size of the pMHC-NP, with or without the layer, can range from about 5 nm to about 1 μm in diameter. In certain embodiments, the pMHC-NP complex is less than about 1 μm or alternatively less than 100 nm in diameter. In other embodiments, the pMHC-NP complex is less than about 500 nm, less than about 400 nm, less than about 300 nm, less than about 200 nm, less than about 100 nm, or less than about 50 nm in diameter. In further embodiments, the complex is from about 5 nm or 10 nm to about 50 nm, or from about 5 nm to about 75 nm, or from about 5 nm to about 50 nm, or from about 5 nm to about 60 nm, or from about 10 nm to about 50 nm, or from about 10 nm to about 60 nm, or from about 10 nm to about 70 nm, or from about 10 nm to about 75 nm, or from about 20 nm to about 50 nm, or from about 20 nm to about 60 nm, or from about 20 nm to about 70 nm, or from about 20 nm to about 75 nm, or from about 30 nm to about 50 nm, or from about 30 nm to about 60 nm, or from about 30 nm to about 70 nm, or from about 30 nm to about 75 nm, or in one aspect about 55 nm in diameter. In specific embodiments, the pMHC-NP complex is from about 35 nm to about 60 nm, from about 35 nm to about 70 nm, or from about 35 nm to about 75 nm in diameter. In one aspect, the pMHC-NP complex is from about 30 nm to about 50 nm in diameter.

### Antigen-MHC Complexes

The nanoparticles comprise a nanoparticle core, with or without a layer, coupled to an antigen-MHC (pMHC) complex. The antigens are selected for the treatment of the particular autoimmune disorder, allergen, infectious disease or cancer.

The individual polypeptide (e.g., MHC) and the antigenic (e.g., peptide) components form a complex through covalent or non-covalent binding (e.g., through hydrogen bonds, ionic bonds, or hydrophobic bonds). The preparation of such complexes may require varying degrees of manipulation and such methods are well-known in the literature. In some aspects, antigenic components can be associated non-covalently with the pocket portion of the MHC component by, for instance, mixing the MHC and antigenic components; this relies on the natural binding affinity between an MHC and an antigen. Alternatively, in some aspects, the MHC component may be covalently bound to the antigenic component using standard procedures, including, but not limited to, the introduction of known coupling agents or photo affinity labelling (see e.g., Hall et al., Biochemistry 24:5702-5711 (1985)). In certain aspects, an antigenic component may be operatively coupled to the MHC component via peptide linkages or other methods discussed in the literature, including, but not limited to, attachment via carbohydrate groups on the glycoproteins, including, e.g., the carbohydrate moieties of the alpha- and/or beta-chains. In particular embodiments, the antigenic component may be attached to the N-terminal or C-terminal end of an appropriate MHC molecule. Alternatively, in certain embodiments, the MHC complex may be recombinantly formed by incorporating the sequence of the antigenic component into a sequence encoding an MHC, such that both retain their functional properties.

Multiple antigen-MHC complexes may be coupled to the same nanoparticle core; these complexes, MHCs, and/or antigens may be the same or different from one another and the number of pMHCs per nanoparticle core (referred to herein as the “valency” of the nanoparticle complex) having a variety of ranges as described herein. The valency may range between about 1 pMHC complex to 1 nanoparticle core (1:1) to about 6000 pMHC complexes to 1 nanoparticle core (6000:1), or alternatively between about 8:1 to about 6000:1, or alternatively between about 10:1 to 6000:1; or alternatively from about 11:1 to about 6000:1, or alternatively between about 12:1 to about 6000:1, or alternatively at least 2:1, or alternatively at least 8:1, or alternatively at least 9:1, or alternatively at least 10:1, or alternatively at least 11:1, or alternatively at least 12:1. In some aspects, the valency is from about 10:1 to about 6000:1, or from about 20:1 to about 5500:1, or alternatively from about 10:1 to about 5000:1, or alternatively from about 10:1 to about 4000:1, or alternatively from about 10:1 to about 3500:1, or alternatively from about 10:1 to about 3000:1, or alternatively from about 10:1 to about 2500:1, or alternatively from about 10:1 to about 2000:1, or alternatively from about 10:1 to about 1500:1, or alternatively from about 10:1 to 1000:1, or alternatively from about 10:1 to about 500:1, or alternatively from about 10:1 to about 100:1, or alternatively from about 20:1 to about 50:1, or alternatively from about 25:1 to about 60:1, or alternatively from about 30:1 to about 50:1, or alternatively from about 35:1 to about 45:1, or alternatively about 40:1. In another aspect, the valency of the pMHC complexes per nanoparticle core is from about 10:1 to about 100:1, or alternatively from about 10:1 to about 1000:1, or alternatively from 8:1 to 10:1, or alternatively from 13:1 to 50:1.

Applicant also has discovered that pMHC density on the nanoparticle regulates the ability of the pMHC-NPs to trigger or differentiate TR1 cell formation in a dose-independent manner. Density is calculated as the number of complexes per unit surface area of the nanoparticle. The surface area of the nanoparticle may be determined with or without the layers, including, but not limited to, linkers that conjugate the pMHC complex to the nanoparticle. For the purposes of calculating density, the relevant surface area value is based on the final diameter of the particle construct without the pMHC complex, with or without the outer layer on the nanoparticle core.

In these aspects, the pMHC density per nanoparticle is from about 0.025 pMHC/100 nm² to about 100 pMHC/100 nm² of the surface area of the nanoparticle core, or alternatively from about 0.406 pMHC/100 nm² to about 50 pMHC/100 nm², or alternatively from about 0.05 pMHC/100 nm² to about 25 pMHC/100 nm². In certain aspects, the pMHC density per nanoparticle is from about 0.2 pMHC/100 nm² to about 25 pMHC/100 nm², or from about 0.4 pMHC/100 nm² to about 20 pMHC/100 nm², or from about 0.4 pMHC/100 nm² to about 15 pMHC/100 nm², or from about 0.4 pMHC/100 nm² to about 14 pMHC/100 nm², or from about 0.4 pMHC/100 nm² to about 13 pMHC/100 nm², or from about 0.4 pMHC/100 nm² to about 12 pMHC/100 nm², or from about 0.4 p MHC/100 nm² to about 11.6 pMHC/100 nm², or from about 0.4 pMHC/100 nm² to about 11.5 pMHC/100 nm², or from about 0.4 pMHC/100 nm² to about 11 pMHC/100 nm², or from about 0.4 pMHC/100 nm² to about 10 pMHC/100 nm², or from about 0.4 pMHC/100 nm² to about 9 pMHC/100 nm², or from about 0.4 pMHC/100 nm² to about 8 pMHC/100 nm², or from about 0.4 pMHC/100 nm² to about 7 pMHC/100 nm², or from about 0.4 pMHC/100 nm² to about 6 pMHC/100 nm², or from about 0.4 pMHC/100 nm² to about 5 pMHC/100 nm², or from about 0.4 pMHC/100 nm² to about 4 pMHC/100 nm², or from about 0.4 pMHC/100 nm² to about 3 pMHC/100 nm², or from about 0.4 pMHC/100 nm² to about 2.5 pMHC/100 nm², or from about 0.4 pMHC/100 nm² to about 2 pMHC/100 nm², or from about 0.4 pMHC/100 nm² to about 1.5 pMHC/100 nm².

In yet another aspect, the nanoparticle has a pMHC density as defined herein of from about 0.4 pMHC/100 nm² to about 1.3 pMHC/100 nm², or alternatively from about 0.5 pMHC/100 nm² to about 0.9 pMHC/100 nm², or alternatively from about 0.6 pMHC/100 nm² to about 0.8 pMHC/100 nm², and further wherein the nanoparticle core has a diameter from about from about 25 nm to about 60 nm, or from about 25 nm to about 50 nm, or from about 20 nm to about 40 nm, or from about 15 nm to about 50 nm, or from about 15 nm to about 40 nm, or from about 15 nm to about 35 nm, or from about 15 nm to about 30 nm, or from about 15 nm to about 25 nm, or alternatively about 15 nm, or about 20 nm, or about 25 nm, or about 30 nm, or about 35 nm, or about 40 nm. In one embodiment, the density of the pMHC complexes per nanoparticle comprises about 0.2 pMHC/100 nm² of surface area of the nanoparticle to about 0.8 or 10 pMHC/100 nm² of surface area of the nanoparticle. In another aspect, the density of the pMHC complexes per nanoparticle is about 0.65 pMHC/100 nm² of surface area of the nanoparticle to about 12 pMHC/100 nm² of surface area of the nanoparticle, as well as additional density ranges disclosed herein and incorporated herein by reference.

In some aspects, the intermolecular distance of the pMHC complexes is from about 4 nm to about 300 nm, or alternatively about 10 nm to about 250 nm, or alternatively about 10 nm to about 200 nm, or alternatively about 10 to about 150 nm, or alternatively about 10 nm to about 100 nm, or alternatively about 10 nm to about 50 nm, or alternatively about 12 nm to about 30 nm, or alternatively about 12 nm to about 20 nm. In some embodiments, the intermolecular distance of the pMHC complexes is from about 15 nm to about 20 nm.

In some aspects, provided herein is a complex comprising a nanoparticle core, wherein a plurality of disease-relevant antigen-MHC (pMHC) complexes are coupled to the core; the diameter of the core is from about 15 nm to about 25 nm; and wherein the pMHC density on the nanoparticle is from about 0.4 pMHC/100 nm² to about 6 pMHC/100 nm² of the surface area of the nanoparticle. In some embodiments, the complex further comprises an outer layer on the nanoparticle core, wherein the pMHC complex is coupled to the nanoparticle core and/or the outer layer, and wherein the diameter of the nanoparticle core and the outer layer is from about 35 nm to about 75 nm, or alternatively from about 35 nm to about 70 nm, or about 35 nm to about 65 nm.

The term “operatively coupled” or “coated” as used herein, refers to a situation where individual polypeptide (e.g., MHC) and antigenic (e.g., peptide) components are combined to form the active complex prior to binding at the target site, for example, an immune cell. This includes the situation where the individual polypeptide complex components are synthesized or recombinantly expressed and subsequently isolated and combined to form a complex, in vitro, prior to administration to a subject; the situation where a chimeric or fusion polypeptide (i.e., each discrete protein component of the complex is contained in a single polypeptide chain) is synthesized or recombinantly expressed as an intact complex. Typically, polypeptide complexes are added to the nanoparticles to yield nanoparticles with adsorbed or coupled polypeptide complexes having a ratio of number of molecules:number of nanoparticle from about, at least about or at most about 0.1, 0.5, 1, 3, 5, 7, 10, 15, 20, 25, 30, 35, 40, 50, 100, 125, 150, 175, 200, 225, 250, 275, 300, 325, 350, 375, 400, 425, 450, 475, 500, 600, 700, 800, 900, 1000, 1500 or more to:1, more typically 0.1:1, 1:1 to 50:1 or 300:1, and ranges there between where the ratios provide the selected endpoints of each range. The polypeptide content of the nanoparticles can be determined using standard techniques.

### The MHC of the Antigen/MHC

As used herein and unless specifically noted, the term MHC in the context of an pMHC complex intends a classical or a non-classical MHC class I protein and/or or classical or non-classical MHC class II protein, any loci of HLA DR, HLA DQ, HLA DP, HLA-A, HLA-B, HLA-C, HLA-E, CD1d, or a fragment or biological equivalent thereof, dual or single chain constructs, dimers (Fc fusions), tetramers, multimeric forms, and a polymeric form of MHCI or MHCII. In some embodiments, the pMHC can be a single chain construct. In some embodiments, the pMHC can be a dual-chain construct.

In some embodiments, the MHC protein can be a dimer or a multimer.

In some embodiments, the MHC protein may comprise a knob-in-hole-based MHC-alpha-Fc/MHC-beta-Fc heterodimer or multimer.

As noted above, “knob-in-hole” is a polypeptidyl architecture requiring a protuberance (or “knob”) at an interface of a first polypeptide and a corresponding cavity (or a “hole”) at an interface of a second polypeptide, such that the protuberance can be positioned in the cavity so as to promote heteromultimer formation. Protuberances are constructed by replacing small amino acid side chains from the interface of the first polypeptide with larger side chains (e.g., phenylalanine or tyrosine). Cavities of identical or similar size to the protuberances are created in the interface of the second polypeptide by replacing large amino acid side chains with smaller ones (e.g., alanine or threonine). The protuberances and cavities can be made by synthetic means such as by altering the nucleic acid encoding the polypeptides or by peptide synthesis, using routine methods for one skilled in the art. In some embodiments, the interface of the first polypeptide is located on an Fc domain in the first polypeptide; the interface of the second polypeptide is located on an Fe domain on the second polypeptide.

As noted above, “MHC-alpha-Fc/MHC-beta-Fc” is a heterodimer comprising a first polypeptide and a second polypeptide, wherein the first polypeptide comprises an MHC class II α-chain and an antibody Fc domain; the second polypeptide comprises an MHC class II β-chain and an antibody Fc domain. A knob-in-hole MHC-alpha-Fc/MHC-beta-Fc further requires that the Fc domains of each polypeptide interface with one another through the complementary positioning of a protuberance on one Fc domain within the corresponding cavity on the other Fc domain.

In certain embodiments of the disclosure, a particular antigen is identified and presented in the antigen-MHC-nanoparticle complex in the context of an appropriate MHC class I or II polypeptide. Presentation of antigens to T cells is mediated by two distinct classes of molecules, MHC class I (MHC-I) and MHC class II (MHC-II), which utilize distinct antigen processing pathways. Peptides derived from intracellular antigens are presented to CD8+ T cells by MHC class I molecules, which are expressed on virtually all cells, while extracellular antigen-derived peptides are presented to CD4⁺ T cells by MHC-II molecules. However, there are certain exceptions to this dichotomy. Several studies have shown that peptides generated from endocytosed particulate or soluble proteins are presented on MHC-I molecules in macrophages as well as in dendritic cells. In certain aspects, the genetic makeup of a subject may be assessed to determine which MHC polypeptide is to be used for a particular patient and a particular set of peptides. In certain embodiments, the MHC class 1 component may comprise, consist essentially of, or alternatively further consist thereof all or part of a HLA-A, HLA-B, HLA-C, HLA-E, HLA-F, HLA-G or CD-1 molecule. In embodiments wherein the MHC component is an MHC class II component, the MHC class II component may comprise, consist essentially of, or alternatively further consist thereof all or a part of a HLA-DR, HLA-DQ, or HLA-DP. In certain embodiments, the MHC may comprise HLA DRB1, HLA DRB3, HLA DRB4, HLA DRB5, HLA DQB1, HLA DQA1, IAg₇, I-Ab, I-Ad, HLA-DQ, HLA-DP, HLA-A, HLA-B, HLA-C, HLA-E or CD1d.

Non-classical MHC molecules are also contemplated for use in MHC complexes of the disclosure. In some embodiments, non-classical MHC molecules are non-polymorphic, conserved among species, and possess narrow, deep, hydrophobic ligand-binding pockets. These binding pockets are capable of presenting glycolipids and phospholipids to Natural Killer T (NKT) cells. NKT cells represent a unique lymphocyte population that co-express NK cell markers and a semi-invariant T cell receptor (TCR). They are implicated in the regulation of immune responses associated with a broad range of diseases.

As noted above, the term “MHC” may be used interchangeably with the term “human leukocyte antigen” (HLA) when used in reference to human MHC; thus, MHC refers to all HLA subtypes including, but not limited to, the classical MHC genes disclosed above: HLA-A, HLA-B, HLA-C, HLA-DP, HLA-DQ, and HLA-DR, in addition to all variants, isoforms, isotypes, and other biological equivalents thereof.

MHCs for use according to the present disclosure may be produced, isolated, or purified through techniques known in the art. Common protocols for obtaining MHCs involve steps including, but not limited to, electrophoresis or other techniques of charge or size-based separation, biotinylation or other tagging methods and purification, or transfection and induction of vector constructs expressing MHC proteins. Purified animal antibodies are also available through commercially available sources, including retailers such as eBioscience, Biolegend, and Tonbo Biosciences.

In certain embodiments, the MHC of the antigen-MHC complexes may be classical MHCI, non-classical MHCI, classical MHCII, non-classical MHCII, dimers (Fc fusions), MHC tetramers, multimers or a polymeric form of MHC. In some embodiments, MHC multimers are generated according to methods well-documented in the art, see, e.g., Bakker et al. “MHC Multimer Technology: Current Status and Future Prospects,” Current Opinion in Immunology 17(4):428-433 (2005) and references cited therein. Non-limiting exemplary methods include the use of a biotinylating agent such as streptavidin or avidin to bind MHC monomers, creating a multimeric structure with the agent as a backbone. MHC dimers, specifically, may alternatively be produced through fusion with antibody constant regions or Fc regions; this may be accomplished through operative coupling directly or through a linker, e.g., a cysteine linker.

The Antigens of the pMHC

Although specific examples of antigens and antigenic components are disclosed herein, the disclosure is not so limited. Unless specifically stated otherwise, included herein are equivalents of the isolated or purified polypeptide antigens that comprise, or consist essentially of, or yet further consist of the amino acid sequences as described herein, or a polypeptide having at least about 80% sequence identity, or alternatively at least 85%, or alternatively at least 90%, or alternatively at least 95%, or alternatively at least 98% sequence identity to the amino acid sequences of the antigens, or polypeptides encoded by polynucleotides having at about 80% sequence identity, or alternatively at least 85%, or alternatively at least 90%, or alternatively at least 95%, or alternatively at least 98% sequence identity to the polynucleotide encoding the amino acid sequences of the antigen, or its complement, or a polypeptide encoded by a polynucleotide that hybridizes under conditions of moderate to high stringency to a polynucleotide encoding the amino acid sequence of the antigens, or its complement. Also provided are isolated and purified polynucleotides encoding the antigen polypeptides disclosed herein, or amino acids having at least about 80% sequence identity thereto, or alternatively at least 85%, or alternatively at least 90%, or alternatively at least 95%, or alternatively at least 98% sequence identity to the disclosed sequences, or an equivalent, or a polynucleotide that hybridizes under stringent conditions to the polynucleotide, its equivalent, or its complement, and isolated or purified polypeptides encoded by these polynucleotides. The polypeptides and polynucleotides can be combined with non-naturally occurring substances with which they are not associated with in nature, e.g., carriers, pharmaceutically acceptable carriers, vectors, and MHC molecules. In addition to the antigens disclosed herein, the antigens disclosed in Applicant's WO 2016/198932, incorporated herein by reference.

### Modified Peptides and Equivalents Thereto

The antigenic polypeptides, proteins, and fragments thereof may be modified by various amino acid deletions, insertions, and/or substitutions. In particular embodiments, modified polypeptides and/or peptides are capable of modulating an immune response in a subject. As used herein, a “protein” or “polypeptide” or “peptide” refers to a molecule comprising at least five amino acid residues. In some embodiments, a wild-type version of a protein or peptide is employed; however, in many embodiments of the disclosure, a modified protein or polypeptide is employed to generate a peptide/MHC/nanoparticle complex. A peptide/MHC/nanoparticle complex can be used to generate an immune response and/or to modify the T cell population of the immune system (i.e., re-educate the immune system). The terms described above may be used interchangeably herein. A “modified protein” or “modified polypeptide” or “modified peptide” refers to a protein or polypeptide whose chemical structure, particularly its amino acid sequence, is altered with respect to the wild-type protein or polypeptide. In some embodiments, a modified protein or polypeptide or peptide has at least one modified activity or function (recognizing that proteins or polypeptides or peptides may have multiple activities or functions). It is specifically contemplated that a modified protein or polypeptide or peptide may be altered with respect to one activity or function yet retain a wild-type activity or function in other respects, such as immunogenicity or ability to interact with other cells of the immune system when in the context of an MHC/nanoparticle complex.

Proteins of the disclosure may be recombinant or synthesized in vitro. Alternatively, a recombinant protein may be isolated from bacteria or other host cell.

It also will be understood that amino acid and nucleic acid sequences may include additional residues, such as additional N- or C-terminal amino acids or 5′ or 3′ nucleic acid sequences, respectively, and yet still be essentially as set forth in one of the sequences disclosed herein, so long as the sequence meets the criteria set forth above, including the maintenance of biological protein activity (e.g., immunogenicity). The addition of terminal sequences particularly applies to nucleic acid sequences that may, for example, include various non-coding sequences flanking either of the 5′ or 3′ portions of the coding region.

### Disease-Relevant Antigens

The nanoparticles are useful in the therapeutic methods as described herein. The pMHC complex of the pMHC-NP is selected for use based on the disease to be treated. For example, a diabetes-relevant antigen is an antigen or fragment thereof that is expressed in the cell, tissue, or organ targeted in that autoimmune disease, is exposed to the immune system upon cell, tissue, or organ damage caused by the autoimmune response, even if the antigen is not the trigger of the disease process or a key player in its pathogenesis, and, when presented, produces an immune response that serves to treat diabetes; thus, a diabetes-relevant antigen meeting this definition is selected to treat diabetes. A MS-relevant antigen is selected to treat MS. A diabetes-relevant antigen would not be selected to treat MS. Non-limiting, exemplary disease-relevant antigens are disclosed herein and further, such antigens may be determined for a particular disease based on techniques, mechanisms, and methods well-documented in the literature.

Non-limiting examples of diseases of interest include, but are not limited to, asthma, diabetes mellitus Type I and Type II, pre-diabetes, multiple sclerosis, peripheral neuropathy, allergic asthma, primary biliary cirrhosis, cirrhosis, Neuromyelitis optica spectrum disorder, Autoantibody-associated neurological syndromes such as Stiff Person syndrome, Autoimmune Encephalitis, Narcolepsy, Pemphigus vulgaris, Pemphigus foliaceous, Psoriasis, Sjogren's disease/syndrome, Inflammatory bowel disease (IBD), arthritis, Rheumatoid arthritis, Systemic Lupus Erythematosus (SLE), Scleroderma, ANCA-associated Vasculitis, Goodpasture Syndrome, Kawasaki's Disease, Celiac disease, autoimmune cardiomyopathy, idiopathic dilated cardiomyopathy (IDCM), Myasthyenia Gravis, Autoimmune Uveitis, Ankylosing Spondylitis, Grave's Disease, Immune Mediated Myopathies, anti-phospholipid syndrome (ANCA+), atherosclerosis, Autoimmune Hepatitis, Sclerosing Cholangitis, Primary Sclerosing Cholangitis, Dermatomyositis, Chronic Obstructive Pulmonary Disease, Spinal Cord Injury, traumatic injury, tobacco-induced lung destruction, emphysema, pemphigus, uveitis, any other relevant cancer and/or diseases of the central and peripheral nervous systems.

Exemplary antigens or antigenic components include but are not limited to those disclosed in U.S. Application Ser. No. 15/348,959, which is incorporated herein by reference in its entirety.

### Diabetes-Relevant Antigens

Diabetes-relevant antigens include but are not limited to those derived from PPI, IGRP, GAD, islet cell autoantigen-2 (ICA2), and/or insulin. Autoreactive, diabetes-relevant antigenic peptides include, but are not limited to, hInsB₁₀₋₁₈ (HLVEALYLV, SEQ ID NO:4), hIGRP₂₂₈₋₂₃₆ (LNIDLLWSV, SEQ ID NO:5), hIGRP₂₆₅₋₂₇₃ (VLFGLGFAI, SEQ ID NO:6), IGRP₂₀₆₋₂₁₄ (VYLKTNVFL, SEQ ID NO:8), hIGRP₂₀₆₋₂₁₄ (VYLKTNLFL, SEQ ID NO:7), NRP-A7 (KYNKANAFL, SEQ ID NO:11), NRP-I4 (KYNIANVFL, SEQ ID NO:12), NRP-V7 (KYNKANVFL, SEQ ID NO:13), YAI/D^{b} (FQDENYLYL, SEQ ID NO:14) INS B₁₅₋₂₃ (LYLVCGERG, SEQ ID NO:15), PPI_{76-90 (K88S)} (SLQPLALEGSLQSRG, SEQ ID NO:18), IGRP₁₃₋₂₅ (QHLQKDYRAYYTF, SEQ ID NO:9), GAD₅₅₅₋₅₆₇ (NFFRMVISNPAAT, SEQ ID NO:19), GAD_{555-567(557I)}: (NFIRMVISNPAAT, SEQ ID NO:20), IGRP₂₃₋₃₅ (YTFLNFMSNVGDP, SEQ ID NO:10), B₂₄-C₃₆ (FFYTPKTRREAED, SEQ ID NO:21), PPI₇₆₋₉₀ (SLQPLALEGSLQKRG, SEQ ID NO:17), as well as peptides and proteins disclosed in U.S. Publication 2005/0202032, which is incorporated herein by reference in its entirety. Other peptides that may be used in conjunction with this disclosure as autoreactive peptides or as control peptides include, but are not limited to, INS-I9 (LYLVCGERI, SEQ ID NO:16), TUM (KYQAVTTTL, SEQ ID NO:22), and G6Pase (KYCLITIFL, SEQ ID NO:23), as well as equivalents of each thereof. Additional examples include Pro-insulin_{L2-10}, ALWMRLLPL (SEQ ID NO:24); Pro-insulin_{L3-11}, LWMRLLPLL (SEQ ID NO:25); Pro-insulin_{L6-14}, RLLPLLALL (SEQ ID NO:26); Pro-insulin_{B5-14}, HLCGSHLVEA (SEQ ID NO:27); Pro-insulinB₁₀₋₁₈, HLVEALYLV (SEQ ID NO:28); Pro-insulin_{B14-22}, ALYLVCGER (SEQ ID NO:29); Pro-insulin_{B15-24}, LYLVCGERGF (SEQ ID NO:30); Pro-insulin_{B17-25}, LVCGERGFF (SEQ ID NO:31); Pro-insulin_{B18-27}, VCGERGFFYT (SEQ ID NO:32); Pro-insulin_{B20-27}, GERGFFYT (SEQ ID NO:33); Pro-insulin_{B21-29}, ERGFFYTPK (SEQ ID NO:34); Pro-insulin_{B25-C1}, FYTPKTRRE (SEQ ID NO:35); Pro-insulin_{B27-C5}, TPKTRREAEDL (SEQ ID NO:36); Pro-insulin_{C20-28}, SLQPLALEG (SEQ ID NO:37); Pro-insulinc₂₅₋₃₃, ALEGSLQKR (SEQ ID NO:38); Pro-insulin_{C29-A5}, SLQKRGIVEQ (SEQ ID NO:39); Pro-insulin_{A1-10}, GIVEQCCTSI (SEQ ID NO:40); Pro-insulin_{A2-10}, IVEQCCTSI (SEQ ID NO:41); Pro-insulin_{A12-20}, SLYQLENYC (SEQ ID NO:42) or equivalents and/or combinations thereof. Antigens relevant to diabetes include but are not limited to those listed in Table 1, as well as equivalents and combinations thereof.

### MS-Relevant Antigens

Antigens of the disclosure include antigens related to multiple sclerosis. Such antigens include, for example, those disclosed in U.S. Patent Application Publication No. 2012/0077686, and antigens derived from myelin basic protein, myelin associated glycoprotein, myelin oligodendrocyte protein, proteolipid protein, oligodendrocyte myelin oligoprotein, myelin associated oligodendrocyte basic protein, oligodendrocyte specific protein, heat shock proteins, oligodendrocyte specific proteins NOGO A, glycoprotein Po, peripheral myelin protein 22, and 2′3′-cyclic nucleotide 3-phosphodiesterase. In certain embodiments, the antigen is derived from Myelin Oligodendrocyte Glycoprotein (MOG).

In still further aspects, peptide antigens for the treatment of MS and MS-related disorders include without limitation: MOG₃₅₋₅₅, MEVGWYRSPFSRVVHLYRNGK (SEQ ID NO:43): MOG₃₆₋₅₅, EVGWYRSPFSRVVIHLYRNGK (SEQ ID NO:45); MAG₂₈₇₋₂₉₅, SLLLELEEV (SEQ ID NO:60); MAG₅₀₉₋₅₁₇, LMWAKIGPV (SEQ ID NO:61 or 63); MAG₅₅₆₋₅₆₄, VLFSSDFRI (SEQ ID NO:62 or 64); MBP₁₁₀₋₁₁₈, SLSRFSWGA (SEQ ID NO:65); MOG₁₁₄₋₁₂₂, KVEDPFYWV (SEQ ID NO:46); MOG₁₆₆₋₁₇₅, RTFDPHFLRV (SEQ ID NO:47); MOG₁₇₂₋₁₈₀, FLRVPCWKI (SEQ ID NO:48); MOG₁₇₉₋₁₈₈, KITLFVIVPV (SEQ ID NO:49); MOG₁₈₈₋₁₉₆, VLGPLVALI (SEQ ID NO:50); MOG₁₈₁₋₁₈₉, TLFVIVPVL (SEQ ID NO:51); MOG₂₀₅₋₂₁₄, RLAGQFLEEL (SEQ ID NO:52); PLP₈₀₋₈₈, FLYGALLLA (SEQ ID NO:76); MOG₉₇₋₁₀₉ (TCFFRDHSYQEEA, SEQ ID NO:53), MOG₉₇₋₁₀₉ (E107S) (TCFFRDHSYQSEA, SEQ ID NO:54), MBP₈₉₋₁₀₁ (VHFFKNIVTPRTP, SEQ ID NO:71), PLP₁₇₅₋₁₉₂ (YIYFNTWTTCQSIAFPSK, SEQ ID NO:73), PLP₉₄₋₁₀₈ (GAVRQIFGDYKTTIC, SEQ ID NO:74), MBP₈₆₋₉₈ (PVVHFFKNIVTPR, SEQ ID NO:72)-HLA-DRB1*1501 (13mer peptide), PLP₅₄₋₆₈ (NYQDYEYLINVIHAF, SEQ ID NO:75), PLP₂₄₉₋₂₆₃ (ATLVSLLTFMIAATY, SEQ ID NO:77), MOG₁₅₆₋₁₇₀ (LVLLAVLPVLLLQIT, SEQ ID NO:56), MOG₂₀₁₋₂₁₅ (FLRVPCWKITLFVIV, SEQ ID NO:57), and equivalents and/or combinations thereof. Antigens relevant to Multiple Sclerosis include but are not limited to those listed in Table 1, as well as equivalents and combinations thereof.

### Celiac Disease (CD) Relevant Antigens

Antigens relevant to celiac disease include, but are not limited to, those derived from aGlia. Non-limiting celiac disease-relevant antigens include gliadin. Other non-limiting exemplary celiac disease-relevant antigens include: aGlias₅₇₋₆₈: QLQPFPQPELPY (12mer peptide, SEQ ID NO:81); aGlia₆₂₋₇₂: PQPELPYPQPE (11mer peptide, SEQ ID NO:82); aGlia₂₁₇₋₂₁₉; and SGEGSFQPSQQNP (13mer peptide, SEQ ID NO:83), equivalents and combinations thereof. Antigens relevant to Celeriac Disease include but are not limited to those listed in Table 1, as well as equivalents and combinations thereof.

### Primary Biliary Cirrhosis (PBC) Relevant Antigens

Antigens relevant to primary biliary cirrhosis include, but are not limited to, those derived from PDC-E2. Non-limiting examples of exemplary antigens include: PDC-E2₁₂₂₋₁₃₅: GDLIAEVETDKATV (14mer peptide, SEQ ID NO:84); PDC-E2₂₄₉₋₂₆₂: GDLLAEIETDKATI (14mer peptide, SEQ ID NO:85); PDC-E2₂₄₉₋₂₆₃: GDLLAEIETDKATIG (15mer peptide, SEQ ID NO:86); and PDC-E2₆₂₉₋₆₄₃: AQWLAEFRKYLEKPI (15mer peptide, SEQ ID NO:87 or 91), equivalents and combinations thereof. Antigens relevant to Primary Biliary Cirrhosis include but are not limited to those listed in Table 1, as well as equivalents and combinations thereof.

### Pemphigus Folliaceus (PF) and Pemphigus Vulgaris (PV) Relevant Antigens

Antigens relevant to PF and PV include, but are not limited to, those derived from DG1EC2, desmoglein 3, (DG3 or DSG3), and/or desmoglein 1 (DG1 or DSG1). Non-limiting examples include: DG1EC2₂₁₆₋₂₃₅: GEIRTMNNFLDREI (14mer peptide, SEQ ID NO:96); DG3₉₇₋₁₁₁: FGIFVVDKNTGDINI (15mer peptide, SEQ ID NO:106); and DG3₂₅₁₋₂₆₅: CECNIKVKDVNDNFP (15mer peptide, SEQ ID NO:107), equivalents and combinations thereof. Antigens relevant to Pemphigus Folliaceus and Pemphigus Vulgaris include but are not limited to those listed in Table 1, as well as equivalents and combinations thereof.

### Neuromyelitis Optica (NMO) Relevant Antigens

Antigens relevant to NMO include, but are not limited to, those derived from AQP4 or aquaporina 4. Non-limiting examples include: AQP4₁₂₉₋₁₄₃: GAGILYLVTPPSVVG (15mer peptide, SEQ ID NO:117 or SEQ ID NO:120); AQP4₂₈₄₋₂₉₈: RSQVETDDLILKPGV (15mer peptide, SEQ ID NO:118); AQP4₆₃₋₇₆: EKPLPVDMVLISLC (14mer peptide, SEQ ID NO:119); and AQP4₃₉₋₅₃: TAEFLAMLIFVLLSL (15mer peptide, SEQ ID NO:121), equivalents and combinations thereof. Antigens relevant to NMO include but are not limited to those listed in Table 1, as well as equivalents and combinations thereof.

### Collagen-Induced Arthritis Relevant Antigens

Antigens relevant to collagen-induced arthritis include, but are not limited to, those derived from CII. Non-limiting examples include: cCII₂₃₀₋₂₄₄: APGFPGPRGPPGPQG (15mer peptide, SEQ ID NO:1); cCII₆₃₂₋₆₄₆: PAGFAGPPGADGQPG (15mer peptide, SEQ ID NO:2); and CII₂₅₉₋₂₇₃: GIAGFKGDQGPKGET (15mer peptide, SEQ ID NO:3), or equivalents and combinations thereof. Antigens relevant to arthritis include but are not limited to those listed in Table 1, as well as equivalents and combinations thereof.

### Allergic Asthma Relevant Antigens

Antigens relevant to allergic asthma include, but are not limited to, those derived from DERP1 and DERP2. Antigens relevant to allergic asthma include but are not limited to those listed in Table 1, as well as equivalents and combinations thereof.

### Colitis-Relevant Antigens

Antigens relevant to experimental colitis include, but are not limited to, those derived from bacteroides integrase, Fla-2/Fla-X, and YIDX. Antigens relevant to colitis include but are not limited to those listed in Table 1, as well as equivalents and combinations thereof.

### Systemic Lupus Erythematosus (SLE) Relevant Antigens

Antigens relevant to SLE include, but are not limited to, those derived from H4, H2B, H1′, dsDNA, RNP, Smith (Sm), SSA/Ro, SSB/La (SS-B), and/or histones. Non-limiting examples include the following segments of each protein: H4₇₁₋₉₄: TYTEHAKRKTVTAMDVVYALKRQG (SEQ ID NO:153), H4₇₄₋₈₈: EHAKRKTVTAMDVVY (15mer peptide, SEQ ID NO:154); H4₇₆₋₉₀: AKRKTVTAMDVVYAL (15mer peptide, SEQ ID NO:155); H4₇₅₋₈₉: HAKRKTVTAMDVVYA (15mer peptide, SEQ ID NO:156); H4₇₈₋₉₂: RKTVTAMDVVYALKR (15mer peptide, SEQ ID NO:157); H4₈₀₋₉₄: TVTAMDVVYALKRQ (15mer peptide, SEQ ID NO:158); H2B₁₀₋₂₄: PKKGSKKAVTKAQKK (15mer peptide, SEQ ID NO:159); and H2B₁₆₋₃₀: KAVTKAQKKDGKKRK (15mer peptide, SEQ ID NO:160), H1′₂₂₋₄₂: STDHPKYSDMIVAAIQAEKNR (SEQ ID NO:161); and H1′₂₇₋₄₁: KYSDMIVAAIQAEKN (SEQ ID NO:162), as well as equivalents and combinations thereof. Antigens relevant SLE include but are not limited to those listed in Table 1, as well as equivalents and combinations thereof.

### High-Fat Diet-Induced Atherosclerosis Relevant Antigens

Antigens relevant to high-fat diet-induced atherosclerosis include, but are not limited to, those derived from ApoB. Non-limiting examples include the following segments of each protein: ApoB₃₅₀₁₋₃₅₁₆: SQEYSGSVANEANVY (15mer peptide, SEQ ID NO:163); ApoB₁₉₅₂₋₁₉₆₆: SHSLPYESSISTALE (15mer peptide, SEQ ID NO:164); ApoB₉₇₈₋₉₉₃: TGAYSNASSTESASY (15mer peptide, SEQ ID NO:165); ApoB₃₄₉₈₋₃₅₁₃: SFLSQEYSGSVANEA (15mer peptide, SEQ ID NO:166); ApoB_{210A}: KTTKQSFDLSVKAQYKKNKH (20mer peptide, SEQ ID NO:167); ApoB_{210B}: KTTKQSFDLSVKAQY (15mer peptide, SEQ ID NO:168); and ApoB_{210C}: TTKQSFDLSVKAQYK (15mer peptide, SEQ ID NO:169) as well as equivalents and combinations thereof. Antigens relevant to atherosclerosis include but are not limited to those listed in Table 1, as well as equivalents and combinations thereof.

### COPD and Emphysema Relevant Antigens

Antigens relevant to COPD and/or emphysema include, but are not limited to, those derived from elastin. Non-limiting examples include the following segments of elastin. Antigens relevant to COPD and/or emphysema include but are not limited to those listed in Table 1, as well as equivalents and combinations thereof.

### Psoriasis-Relevant Antigens

Antigens relevant to psoriasis include but are not limited to those listed in Table 1, as well as equivalents and combinations thereof. Other non-limiting exemplary psoriasis-relevant antigens include human adamis-like protein 5 (ATL5), cathelicidin antimicrobial peptide (CAP18), and/or ADAMTS-like protein 5 (ADMTSL5).

### Autoimmune Hepatitis-Relevant Antigens

Autoimmune hepatitis-relevant antigens include but are not limited to those disclosed in Table 1, as well as equivalents and combinations thereof. Other non-limiting exemplary autoimmune hepatitis-relevant antigens include cytochrome P450 2D6 (CYP2D6) and/or soluble liver antigen (SLA).

### Uveitis-Relevant Antigens

Uveitis-relevant antigens include but are not limited to those disclosed in Table 1, as well as equivalents and combinations thereof. Other non-limiting exemplary uveitis-relevant antigens include arrestin, S-arrestin, human retinal S-antigen, and/or interphotoreceptor retinoid-binding protein (IRBP).

### Sjögren's Syndrome-Relevant Antigens

Sjögren's Syndrome-relevant antigens include but are not limited to those disclosed in Table las well as equivalents and combinations thereof. Other non-limiting exemplary Sjögren's Syndrome-relevant antigens include SSA/Ro (TROVE), SSB/La, and/or muscarinic receptor 3 (MR3).

### Scleroderma-Relevant Antigens

Scleroderma-relevant antigens include but are not limited to centromere autoantigen centromere protein C (CENP-C), DNA topoisomerase I (TOP1), and/or RNA polymerase III.

### Anti-Phospholipid Syndrome-Relevant Antigens

Anti-phospholipid syndrome relevant antigens include but are not limited to those disclosed in Table 1, as well as equivalents and combinations thereof. Non-limiting exemplary anti-phospholipid syndrome-relevant antigens include beta-2-glycoprotein 1 (BG2P1 or APOH).

### ANCA-Associated Vasculitis-Relevant Antigens

ANCA-associated vasculitis-relevant antigens include but are not limited to those disclosed in Table 1, as well as equivalents and combinations thereof. Non-limiting exemplary ANCA-associated vasculitis-relevant antigens include myeloperoxidase (MPO), proteinase (PR3), or bacterial permeability increasing factor (BPI).

TABLE 1
Disease-related Antigens

| Disease | | | SEQ |
|---|---|---|---|
| Antigen | Antigen Peptides | Amino Acid Sequence | ID No. |
| Collagen- | cCII(230-244) | APGFPGPRGPPGPQG | 1 |
| induced | cCII(632-646) | PAGFAGPPGADGQPG | 2 |
| arthritis | CII(259-273) | GIAGFKGDQGPKGET | 3 |
| relevant | | | |
| antigens | | | |
| Diabetes | hInsB(10-18) | HLVEALYLV | 4 |
| Relevant | hIGRP(228-236) | LNIDLLWSV | 5 |
| Antigenic | hIGRP(265-273) | VLFGLGFAI | 6 |
| Peptides | hIGRP(206-214) | VYLKTNLFL | 7 |
| | IGRP(206-214) | VYLKTNVFL | 8 |
| | IGRP(13-25) | QHLQKDYRAYYTF | 9 |
| | IGRP(23-35) | YTFLNFMSNVGDP | 10 |
| | NRP-A7 | KYNKANAFL | 11 |
| | NRP-I4 | KYNIANVFL | 12 |
| | NRP-V7 | KYNKANVFL | 13 |
| | YAI/D^{b} | FQDENYLYL | 14 |
| | INS B(15-23) | LYLVCGERG | 15 |
| | INS-I9 | LYLVCGERI | 16 |
| | PPI(76-90) | SLQPLALEGSLQKRG | 17 |
| | PPI(76-90)(88S) | SLQPLALEGSLQSRG | 18 |
| | GAD(555-567) | NFFRMVISNPAAT | 19 |
| | GAD(555-567)(557I) | NFIRMVISNPAAT | 20 |
| | Pro-insulin(B24-C36) | FFYTPKTRREAED | 21 |
| | TUM | KYQAVTTTL | 22 |
| | G6Pase | KYCLITIFL | 23 |
| | Pro-insulin(L2-10) | ALWMRLLPL | 24 |
| | Pro-insulin(L3-11) | LWMRLLPLL | 25 |
| | Pro-insulin(L6-14 | RLLPLLALL | 26 |
| | Pro-insulin(B5-14) | HLCGSHLVEA | 27 |
| | Pro-insulin(B10-18) | HLVEALYLV | 28 |
| | Pro-insulin(B14-22) | ALYLVCGER | 29 |
| | Pro-insulin(B15-24) | LYLVCGERGF | 30 |
| | Pro-insulin(B17-25) | LVCGERGFF | 31 |
| | Pro-insulin(B18-27) | VCGERGFFYT | 32 |
| | Pro-insulin(B20-27) | GERGFFYT | 33 |
| | Pro-insulin(B21-29) | ERGFFYTPK | 34 |
| | Pro-insulin(B25-C1) | FYTPKTRRE | 35 |
| | Pro-insulin(B27-C5) | TPKTRREAEDL | 36 |
| | Pro-insulin(C20-28) | SLQPLALEG | 37 |
| | Pro-insulin(C25-33) | ALEGSLQKR | 38 |
| | Pro-insulin(C29-A5) | SLQKRGIVEQ | 39 |
| | Pro-insulin(A1-10) | GIVEQCCTSI | 40 |
| | Pro-insulin(A2-10) | IVEQCCTSI | 41 |
| | Pro-insulin(A12-20) | SLYQLENYC | 42 |
| Multiple | MOG(35-55) | MEVGWYRSPFSRVVHLYRNGK | 43 |
| Sclerosis | MOG(6-20) | IGPRHPIRALVGDEV | 44 |
| Related | MOG(36-55) | EVGWYRSPFSRVVHLYRNGK | 45 |
| Antigens | MOG(114-122) | KVEDPFYWV | 46 |
| | MOG(166-175) | RTFDPHFLRV | 47 |
| | MOG(172-180) | FLRVPCWKI | 48 |
| | MOG(179-188) | KITLFVIVPV | 49 |
| | MOG(188-196) | VLGPLVALI | 50 |
| | MOG(181-189) | TLFVIVPVL | 51 |
| | MOG(205-214) | RLAGQFLEEL | 52 |
| | MOG(97-109) | TCFFRDHSYQEEA | 53 |
| | MOG(97-109)(E107S) | TCFFRDHSYQSEA | 54 |
| | MOG(223-237) | ALIICYNWLHRRLAG | 55 |
| | MOG(156-170) | LVLLAVLPVLLLQIT | 56 |
| | MOG(201-215) | FLRVPCWKITLFVIV | 57 |
| | MOG(38-52) | RHPIRALVGDEVELP | 58 |
| | MOG(203-217) | RVPCWKITLFVIVPV | 59 |
| | MAG(287-295) | SLLLELEEV | 60 |
| | MAG(509-517) | LMWAKIGPV | 61 |
| | MAG(556-564) | VLFSSDFRI | 62 |
| | MAG(509-517) | LMWAKIGPV | 63 |
| | MAG(556-564) | VLFSSDFRI | 64 |
| | MBP(110-118) | SLSRFSWGA | 65 |
| | MBP(13-32) | KYLATASTMDHARHGFLPRH | 66 |
| | MBP(83-99) | ENPVVHFFKNIVTPRTP | 67 |
| | MBP(111-129) | LSRFSWGAEGQRPGFGYGG | 68 |
| | MBP(146-170) | AQGTLSKIFKLGGRDSRSGSPMARR | 69 |
| | MBP(85-97) | NPVVHFFKNIVTP | 70 |
| | MBP(89-101) | VHFFKNIVTPRTP | 71 |
| | MBP(86-98) | PVVHFFKNIVTPR | 72 |
| | PLP(175-192) | YIYFNTWTTCQSIAFPSK | 73 |
| | PLP(94-108) | GAVRQIFGDYKTTIC | 74 |
| | PLP(54-68) | NYQDYEYLINVIHAF | 75 |
| | PLP(80-88) | FLYGALLLA | 76 |
| | PLP(249-263) | ATLVSLLTFMIAATY | 77 |
| | PLP(250-264) | TLVSLLTFMIAATYN | 78 |
| | PLP(88-102) | AEGFYTTGAVRQIFG | 79 |
| | PLP(139-154) | HCLGKWLGHPDKFVGI | 80 |

*MBP Sequences for MBP Isoform 6

| | | | |
|---|---|---|---|
| Celiac Disease | aGlia(57-68) | QLQPFPQPELPY | 81 |
| (CD) relevant | aGlia(62-72) | PQPELPYPQPE | 82 |
| antigens | aGlia(217-229) | SGEGSFQPSQQNP | 83 |
| Primary | PDC-E2(122-135) | GDLIAEVETDKATV | 84 |
| Biliary | PDC-E2(249-262) | GDLLAEIETDKATI | 85 |
| Cirrhosis | PDC-E2(249-263) | GDLLAEIETDKATIG | 86 |
| (PBC) | PDC-E2(629-643) | AQWLAEFRKYLEKPI | 87 |
| relevant | PDC-E2(72-86) | RLLLQLLGSPGRRYY | 88 |
| antigens | PDC-E2(353-367) | GRVFVSPLAKKLAVE | 89 |
| | PDC-E2(422-436) | DIPISNIRRVIAQRL | 90 |
| | PDC-E2(629-643) | AQWLAEFRKYLEKPI | 91 |
| | PDC-E2(80-94) | SPGRRYYSLPPHQKV | 92 |
| | PDC-E2(353-367) | GRVFVSPLAKKLAVE | 93 |
| | PDC-E2(535-549) | ETIANDVVSLATKAR | 94 |
| Pemphigus | DSG1(216-229) | GEIRTMNNFLDREQ | 95 |
| Folliaceus (PF) | DSG1(216-229; 229I) | GEIRTMNNFLDREI | 96 |
| and Pemphigus | DSG1(48-62) | KREWIKFAAACREGE | 97 |
| Vulgaris (PV) | DSG1(206-222) | MFIINRNTGEIRTMN | 98 |
| relevant | DSG1(363-377) | SQYKLKASAISVTVL | 99 |
| antigens | DSG1(3-17) | WSFFRVVAMLFIFLV | 100 |
| | DSG1(192-206) | SKIAFKIIRQEPSDS | 101 |
| | DSG1(326-340) | TNVGILKVVKPLDYE | 102 |
| | DSG1(1-15) | MDWSFFRVVAMLFIF | 103 |
| | DSG1(35-49) | KNGTIKWHSIRRQKR | 104 |
| | DSG1(325-339) | RTNVGILKVVKPLDY | 105 |
| | DSG3(97-111) | FGIFVVDKNTGDINI | 106 |
| | DSG3(251-265) | CECNIKVKDVNDNFP | 107 |
| | DSG3(351-365) | NKAEFHQSVISRYRV | 108 |
| | DSG3(453-467) | DSTFIVNKTITAEVL | 109 |
| | DSG3(540-554) | SITTLNATSALLRAQ | 110 |
| | DSG3(280-294) | ILSSELLRFQVTDLD | 111 |
| | DSG3(326-340) | EGILKVVKALDYEQL | 112 |
| | DSG3(367-381) | STPVTIQVINVREGI | 113 |
| | DSG3(13-27) | AIFVVVILVHGELRI | 114 |
| | DSG3(323-337) | RTNEGILKVVKALDY | 115 |
| | DSG3438-452) | DSKTAEIKFVKNMNR | 116 |
| Neuromyelitis | AQP4(129-143) | GAGILYLVTPPSVVG | 117 |
| optica spectrum | AQP4(284-298) | RSQVETDDLILKPGV | 118 |
| disorder (NMO) | AQP4(63-76) | EKPLPVDMVLISLC | 119 |
| relevant | AQP4(129-143) | GAGILYLVTPPSVVG | 120 |
| antigens | AQP4(39-53) | TAEFLAMLIFVLLSL | 121 |
| Allergic asthma | DERP-1(16-30) | LRQMRTVTPIRMQGG | 122 |
| relevant | DERP-1(171-185) | AVNIVGYSNAQGVDY | 123 |
| antigens | DERP-1(110-124) | RFGISNYCQIYPPNV | 124 |
| | DERP-2(26-40) | PCIIHRGKPFQLEAV | 125 |
| | DERP-2(107-121) | TVKVMGDDGVLACAI | 126 |
| Inflammatory | bacteroides integrase | EAINQGYMHADAYPF | 127 |
| Bowel Disease- | antigen(183-197) | | |
| or colitis- | bacteroides integrase | KDLTYTFLRDFEQYL | 128 |
| relevant | antigen(146-160) | | |
| antigens | bacteroides integrase | RQLRTLVNEAINQGY | 129 |
| | antigen(175-189) | | |
| | bacteroides integrase | MDKIRYRLVYNRQNT | 130 |
| | antigen(1-15) | | |
| | bacteroides integrase | LNQRKIYLKTNVYLK | 131 |
| | antigen(30-44) | | |
| | bacteroides integrase | EYILYLQGIELGYWK | 132 |
| | antigen(70-84) | | |
| | bacteroides integrase | TCATLLIHQGVAITT | 133 |
| | antigen(337-351) | | |
| | bacteroides integrase | AKHMRQLRTLVNEAI | 134 |
| | antigen(171-185) | | |
| | bacteroides integrase | IRYRLVYNRQNTLNR | 135 |
| | antigen(4-18) | | |
| | bacteroides integrase | ENFIRINGKRWLYFK | 136 |
| | antigen(256-270) | | |
| | Fla-2/Fla-X(366-380) | TGAAATYAIDSIADA | 137 |
| | Fla-2/Fla-X(164-178) | NATFSMDQLKFGDTI | 138 |
| | Fla-2/Fla-X(261-275) | DRTVVSSIGAYKLIQ | 139 |
| | Fla-2/Fla-X(1-15) | MVVQHNLRAMNSNRM | 140 |
| | Fla-2/Fla-X(51-65) | KMRKQIRGLSQASLN | 141 |
| | Fla-2/Fla-X(269-283) | GAYKLIQKELGLASS | 142 |
| | Fla-2/Fla-X(4-18) | QHNLRAMNSNRMLGI | 143 |
| | Fla-2/Fla-X(271-285) | YKLIQKELGLASSIG | 144 |
| | YIDX(93-107) | HNIQVADDARFVLNA | 145 |
| | YIDX(98-112) | ADDARFVLNAGKKKF | 146 |
| | YIDX(23-37) | GCISYALVSHTAKGS | 147 |
| | YIDX(78-92) | ADDIVKMLNDPALNR | 148 |
| | YIDX(195-209) | LPVTVTLDIITAPLQ | 149 |
| | YIDX(22-36) | SGCISYALVSHTAKG | 150 |
| | YIDX(80-94) | DIVKMLNDPALNRHN | 151 |
| | YIDX(101-115) | ARFVLNAGKKKFTGT | 152 |
| Systemic Lupus | H4(71 94) | TYTEHAKRKTVTAMDVVYALKRQG | 153 |
| Erythematosus | H4(74-88) | EHAKRKTVTAMDVVY | 154 |
| (SLE) relevant | H4(76-90) | AKRKTVTAMDVVYAL | 155 |
| antigens | H4(75-89) | HAKRKTVTAMDVVYA | 156 |
| | H4(78-92) | RKTVTAMDVVYALKR | 157 |
| | H4(80-94) | TVTAMDVVYALKRQ | 158 |
| | H2B(10-24) | PKKGSKKAVTKAQKK | 159 |
| | H2B(16-30) | KAVTKAQKKDGKKRK | 160 |
| | H1′(22-42) | STDHPKYSDMIVAAIQAEKNR | 161 |
| | H1′(27-41) | KYSDMIVAAIQAEKN | 162 |
| Atherosclerosis | ApoB(3501-3516) | SQEYSGSVANEANVY | 163 |
| relevant | ApoB(1952-1966) | SHSLPYESSISTALE | 164 |
| antigens | ApoB(978-993) | TGAYSNASSTESASY | 165 |
| | ApoB(3498-3513) | SFLSQEYSGSVANEA | 166 |
| | ApoB(210A) | KTTKQSFDLSVKAQYKKNKH | 167 |
| | ApoB(210B) | KTTKQSFDLSVKAQY | 168 |
| | ApoB(210C) | TTKQSFDLSVKAQYK | 169 |
| Chronic | ELN(89-103) | GALVPGGVADAAAAY | 170 |
| Obstructive | ELN(698-712) | AAQFGLVGAAGLGGL | 171 |
| Pulmonary | ELN(8-22) | APRPGVLLLLLSILH | 172 |
| Disease | ELN(94-108) | GGVADAAAAYKAAKA | 173 |
| (COPD) and/or | ELN(13-27) | VLLLLLSILHPSRPG | 174 |
| Emphysema | ELN(695-709) | AAKAAQFGLVGAAGL | 175 |
| relevant | ELN(563-577) | VAAKAQLRAAAGLGA | 176 |
| antigens | ELN(558-572) | KSAAKVAAKAQLRAA | 177 |
| | ELN(698-712) | AAQFGLVGAAGLGGL | 178 |
| | ELN(566-580) | KAQLRAAAGLGAGIP | 179 |
| | ELN(645-659) | VPGALAAAKAAKYGA | 180 |
| Psoriasis- | CAP18(64-78) | RPTMDGDPDTPKPVS | 181 |
| Relevant | CAP18(34-48) | SYKEAVLRAIDGINQ | 182 |
| Antigens | CAP18(47-61) | NQRSSDANLYRLLDL | 183 |
| | CAP18(151-165) | KRIVQRIKDFLRNLV | 184 |
| | CAP18(149-163) | EFKRIVQRIKDFLRN | 185 |
| | CAP18(152-166) | RIVQRIKDFLRNLVP | 186 |
| | CAP18(131-145) | RFALLGDFFRKSKEK | 187 |
| | CAP18(24-38) | QRIKDFLRNLVPRTE | 188 |
| | ADAMTSL5(245-259) | DGRYVLNGHWVVSPP | 189 |
| | ADAMTSL5(267-281) | THVVYTRDTGPQETL | 190 |
| | ADAMTSL5(372-386) | RLLHYCGSDFVFQAR | 191 |
| | ADAMTSL5(289-303) | HDLLLQVLLQEPNPG | 192 |
| | ADAMTSL5(396-410) | ETRYEVRIQLVYKNR | 193 |
| | ADAMTSL5(433-447) | HRDYLMAVQRLVSPD | 194 |
| | ADAMTSL5(142-156) | EGHAFYHSFGRVLDG | 195 |
| | ADAMTSL5(236-250) | RNHLALMGGDGRYVL | 196 |
| | ADAMTSL5(301-315) | NPGIEFEFWLPRERY | 197 |
| | ADAMTSL5(203-217) | VQRVFRDAGAFAGYW | 198 |
| | ADAMTSL5(404-418) | QLVYKNRSPLRAREY | 199 |
| Autoimmune | CYP2D6(193-207) | RRFEYDDPRFLRLLD | 200 |
| Hepatitis- | CYP2D6(76-90) | TPVVVLNGLAAVREA | 201 |
| Relevant | CYP2D6(293-307) | ENLRIVVADLFSAGM | 202 |
| Antigens | CYP2D6(313-332) | TLAWGLLLMILHPDVQRRVQ | 203 |
| | CYP2D6(393-412) | TTLITNLSSVLKDEAVWEKP | 204 |
| | CYP2D6(199-213) | DPRFLRLLDLAQEGL | 205 |
| | CYP2D6(450-464) | RMELFLFFTSLLQHF | 206 |
| | CYP2D6(301-315) | DLFSAGMVTTSTTLA | 207 |
| | CYP2D6(452-466) | ELFLFFTSLLQHFSF | 208 |
| | CYP2D6(59-73) | DQLRRRFGDVFSLQL | 209 |
| | CYP2D6(130-144) | EQRRFSVSTLRNLGL | 210 |
| | CYP2D6(193-212) | RRFEYDDPRFLRLLDLAQEG | 211 |
| | CYP2D6(305-324) | AGMVTTSTTLAWGLLLMILH | 212 |
| | CYP2D6(305-325) | AGMVTTSTTLAWGLLLMILHP | 213 |
| | CYP2D6(131-145) | QRRFSVSTLRNLGLG | 214 |
| | CYP2D6(216-230) | ESGFLREVLNAVPVL | 215 |
| | CYP2D6(238-252) | GKVLRFQKAFLTQLD | 216 |
| | CYP2D6(199-213) | DPRFLRLLDLAQEGL | 217 |
| | CYP2D6(235-252) | GKVLRFQKAFLTQLD | 218 |
| | CYP2D6(293-307) | ENLRIVVADLFSAGM | 219 |
| | CYP2D6(381-395) | DIEVQGFRIPKGTTL | 220 |
| | CYP2D6(429-443) | KPEAFLPFSAGRRAC | 221 |
| | SLA(334-348) | YKKLLKERKEMFSYL | 222 |
| | SLA(196-210) | DELRTDLKAVEAKVQ | 223 |
| | SLA(115-129) | NKITNSLVLDIIKLA | 224 |
| | SLA(373-386) | NRLDRCLKAVRKER | 225 |
| | SLA(186-197) | LIQQGARVGRID | 226 |
| | SLA(317-331) | SPSLDVLITLLSLGS | 227 |
| | SLA(171-185) | DQKSCFKSMITAGFE | 228 |
| | SLA(417-431) | YTFRGFMSHTNNYPC | 229 |
| | SLA(359-373) | YNERLLHTPHNPISL | 230 |
| | SLA(215-229) | DCILCIHSTTSCFAP | 231 |
| | SLA(111-125) | SSLLNKITNSLVLDI | 232 |
| | SLA(110-124) | GSSLLNKITNSLVLD | 233 |
| | SLA(299-313) | NDSFIQEISKMYPGR | 234 |
| | SLA(342-356) | KEMFSYLSNQIKKLS | 235 |
| | SLA(49-63) | STLELFLHELAIMDS | 236 |
| | SLA(119-133) | NSLVLDIIKLAGVHT | 237 |
| | SLA(260-274) | SKCMHLIQQGARVGR | 238 |
| | SLA(26-40) | RSHEHLIRLLLEKGK | 239 |
| | SLA(86-100) | RRHYRFIHGIGRSGD | 240 |
| | SLA(331-345) | SNGYKKLLKERKEMF | 241 |
| | Autoimmune hepatitis- | VVSSHYSRRFTPEIAKRPKV | 242 |
| | relevant antigen (217- | | |
| | 236) | | |
| Uveitis- | SAG(199-214) | QFFMSDKPLHLAVSLN | 243 |
| Relevant | SAG(199-213) | QFFMSDKPLHLAVSL | 244 |
| Antigens | SAG(77-91) | DVIGLTFRRDLYFSR | 245 |
| | SAG(250-264) | NVVLYSSDYYVKPVA | 246 |
| | SAG(172-186) | SSVRLLIRKVQHAPL | 247 |
| | SAG(354-368) | EVPFRLMHPQPEDPA | 248 |
| | SAG(239-253) | KKIKAFVEQVANVVL | 249 |
| | SAG(102-116) | STPTKLQESLLKKLG | 250 |
| | SAG(59-73) | KKVYVTLTCAFRYGQ | 251 |
| | SAG(280-294) | KTLTLLPLLANNRER | 252 |
| | SAG(291-306) | NRERRGIALDGKIKHE | 253 |
| | SAG(195-209) | EAAWQFFMSDKPLHL | 254 |
| | SAG(200-214) | QFFMSDKPLHLAVSL | 255 |
| Sjogren's | TROVE2(127-141) | TFIQFKKDLKESMKC | 256 |
| Relevant | TROVE2(523-537) | DTGALDVIRNFTLDM | 257 |
| Syndrome- | TROVE2(243-257) | EVIHLIEEHRLVREH | 258 |
| Antigens | TROVE2(484-498) | REYRKKMDIPAKLIV | 259 |
| | TROVE2(347-361) | EEILKALDAAFYKTF | 260 |
| | TROVE2(369-383) | KRFLLAVDVSASMNQ | 261 |
| | TROVE2(426-440) | SSA/RO(426-440) | 262 |
| | TROVE2(267-281) | EVWKALLQEMPLTAL | 263 |
| | TROVE2(178-192) | SHKDLLRLSHLKPSS | 264 |
| | TROVE2(358-372) | YKTFKTVEPTGKRFL | 265 |
| | TROVE2(221-235) | ETEKLLKYLEAVEKV | 266 |
| | TROVE2(318-332) | RIHPFHILIALETYK | 267 |
| | TROVE2(407-421) | EKDSYVVAFSDEMVP | 268 |
| | TROVE2(459-473) | TPADVFIVFTDNETF | 269 |
| | TROVE2(51-65) | QKLGLENAEALIRLI | 270 |
| | TROVE2(312-326) | KLLKKARIHPFHILI | 271 |
| | SS-B(241-255) | DDQTCREDLHILFSN | 272 |
| | SS-B(101-115) | TDEYKNDVKNRSVYI | 273 |
| | SS-B(153-167) | SIFVVFDSIESAKKF | 274 |
| | SS-B(178-192) | TDLLILFKDDYFAKK | 275 |
| | SS-B(19-33) | HQIEYYFGDFNLPRD | 276 |
| | SS-B(37-51) | KEQIKLDEGWVPLEI | 277 |
| | SS-B(133-147) | DKGQVLNIQMRRTLH | 278 |
| | SS-B(50-64) | EIMIKFNRLNRLTTD | 279 |
| | SS-B(32-46) | RDKFLKEQIKLDEGW | 280 |
| | SS-B(153-167) | SIFVVFDSIESAKKF | 281 |
| | SS-B(83-97) | SEDKTKIRRSPSKPL | 282 |
| | SS-B(136-150) | QVLNIQMRRTLHKAF | 283 |
| | SS-B(297-311) | RNKEVTWEVLEGEVE | 284 |
| | SS-B(59-73) | NRLTTDFNVIVEALS | 285 |
| | SS-B(151-165) | KGSIFVVFDSIESAK | 286 |
| | SS-B(86-100) | KTKIRRSPSKPLPEV | 287 |
| | SS-B(154-168) | IFVVFDSIESAKKFV | 288 |
| Scleroderma- | TOP1(346-360) | KERIANFKIEPPGLF | 289 |
| Relevant | TOP1(420-434) | QGSIKYIMLNPSSRI | 290 |
| Antigens | TOP1(750-764) | QREKFAWAIDMADED | 291 |
| | TOP1(419-433) | IQGSIKYIMLNPSSR | 292 |
| | TOP1(591-605) | YNASITLQQQLKELT | 293 |
| | TOP1(695-709) | EQLMKLEVQATDREE | 294 |
| | TOP1(305-319) | SQYFKAQTEARKQMS | 295 |
| | TOP1(346-360) | KERIANFKIEPPGLF | 296 |
| | TOP1(419-433) | IQGSIKYIMLNPSSR | 297 |
| | TOP1(425-439) | YIMLNPSSRIKGEKD | 298 |
| | TOP1(614-628) | KILSYNRANRAVAIL | 299 |
| | CENP-C(297-311) | KLIEDEFTIDESDQS | 300 |
| | CENP-C(857-871) | KVYKTLDTPFFSTGK | 301 |
| | CENP-C(887-901) | QDILVFYVNFGDLLC | 302 |
| | CENP-C(212-226) | KVMLKKIEIDNKVSD | 303 |
| | CENP-C(643-657) | EDNIMTAQNVPLKPQ | 304 |
| | CENP-C(832-846) | TREIILMDLVRPQDT | 305 |
| | CENP-C(167-181) | TSVSQNVIPSSAQKR | 306 |
| | CENP-C(246-260) | RIRDSEYEIQRQAKK | 307 |
| | CENP-C(846-860) | TYQFFVKHGELKVYK | 308 |
| | CENP-C(149-163) | DEEFYLSVGSPSVLL | 309 |
| | CENP-C(833-847) | REIILMDLVRPQDTY | 310 |
| | CENP-C(847-861) | YQFFVKHGELKVYKT | 311 |
| Anti- | APOH(235-249) | HDGYSLDGPEEIECT | 312 |
| Phospholipid | APOH(306-320) | KCSYTEDAQCIDGTI | 313 |
| Syndrome- | APOH(237-251) | GYSLDGPEEIECTKL | 314 |
| Relevant | APOH(295-309) | KVSFFCKNKEKKCSY | 315 |
| Antigens | APOH(28-42) | DLPFSTVVPLKTFYE | 316 |
| | APOH(173-187) | ECLPQHAMFGNDTIT | 317 |
| | APOH(264-278) | CKVPVKKATVVYQGE | 318 |
| | APOH(295-309) | KVSFFCKNKEKKCSY | 319 |
| | APOH(49-63) | YSCKPGYVSRGGMRK | 320 |
| | APOH(269-283) | KKATVVYQGERVKIQ | 321 |
| | APOH(295-309) | KVSFFCKNKEKKCSY | 322 |
| | APOH321-355 | EVPKCFKEHSSLAFW | 323 |
| | APOH322-336 | VPKCFKEHSSLAFWK | 324 |
| | APOH324-338 | KCFKEHSSLAFWKTD | 325 |
| ANCA- | MPO(506-520) | QPFMFRLDNRYQPME | 326 |
| Associated | MPO(302-316) | RIKNQADCIPFFRSC | 327 |
| Vasculitis- | MPO(7-21) | SSLRCMVDLGPCWAG | 328 |
| Relevant | MPO(689-703) | QQRQALAQISLPRII | 329 |
| Antigens | MPO(248-262) | RSLMFMQWGQLLDHD | 330 |
| | MPO(444-458) | QEARKIVGAMVQIIT | 331 |
| | MPO(513-527) | DNRYQPMEPNPRVPL | 332 |
| | MPO(97-111) | ELLSYFKQPVAATRT | 333 |
| | MPO(616-630) | QLGTVLRNLKLARKL | 334 |
| | MPO(462-476) | YLPLVLGPTAMRKYL | 335 |
| | MPO(617-631) | LGTVLRNLKLARKLM | 336 |
| | MPO(714-728) | KNNIFMSNSYPRDFV | 337 |
| | PRTN3(44-58) | SLQMRGNPGSHFCGG | 338 |
| | PRTN3(234-248) | TRVALYVDWIRSTLR | 339 |
| | PRTN3(59-73) | TLIHPSFVLTAAHCL | 340 |
| | PRTN3(117-131) | NDVLLIQLSSPANLS | 341 |
| | PRTN3(164-178) | DPPAQVLQELNVTVV | 342 |
| | PRTN3(71-85) | HCLRDIPQRLVNVVL | 343 |
| | PRTN3(241-255) | DWIRSTLRRVEAKGR | 344 |
| | PRTN3(59-73) | TLIHPSFVLTAAHCL | 345 |
| | PRTN3(183-197) | RPHNICTFVPRRKAG | 346 |
| | PRTN3(62-76) | HPSFVLTAAHCLRDI | 347 |
| | PRTN3(118-132) | DVLLIQLSSPANLSA | 348 |
| | PRTN3(239-253) | YVDWIRSTLRRVEAK | 349 |
| Stiff Man | GAD(212-226) | EYVTLKKMREIIGWP | 350 |
| Syndrome- | GAD(555-569) | NFFRMVISNPAATHQ | 351 |
| Relevant | GAD(297-311) | DSVILIKCDERGKMI | 352 |
| Antigens | | | |

### Cancer-Relevant Antigens

In certain aspects, the disease-relevant antigen is a cancer relevant antigen. In further aspects, the cancer is carcinoma, sarcoma, myeloma, leukemia, lymphoma, and/or mixed types of metastases from these or other cancers. Exemplary cancer- or tumor-relevant antigens include but are not limited to those disclosed in Table 2.

TABLE 2
Cancer-related Antigens

| | SEQ ID |
|---|---|
| | NO: |
| Lys Ile Ser Val Ser Leu Pro Leu Ser | 353 |
| Leu Ser Gln Ser Val Cys | |
| Gln Leu Ser Lys Asp Thr Ser Val Leu | 354 |
| Thr Phe Thr Phe Cys | |
| Cys Ser Asp Ala His Pro Gly Asp Ser | 355 |
| Ser Gly Asp Ser Ser Gly Leu Asn | |
| Arg Gly Glu Val Arg Gln Phe Thr Leu | 356 |
| Arg His Trp Leu Lys Val | |
| Gly Asp Tyr Leu Asn Asp Glu Ala Leu | 357 |
| Trp Asn Lys Cys | |
| Gly Lys Val Ile Asp Asp Asn Asp His | 358 |
| Leu Ser Gln Glu Ile Cys | |
| Leu Met Ala Asn Ser Thr Trp Gly Tyr | 359 |
| Pro Phe His Asp Gly | |
| Leu Asn Val Val Pro Trp Asn Leu Thr | 360 |
| Leu Phe Ser Ile Leu | |
| Thr His Ser Phe Thr Ala Phe Lys Arg | 361 |
| His Val Cys | |
| Asn Leu Ser Leu Pro Pro Ser Leu Ser | 362 |
| Leu Ser Ile Cys | |
| Glu Arg Pro Ser Ser Val Leu Thr Ile | 363 |
| Tyr Asp Ile Gly Ile Gln Cys | |
| Cys Tyr Gln Gln Tyr Thr Asn Leu Gln | 364 |
| Glu Arg Pro Ser Ser Val | |
| Thr Val Glu Pro Glu Thr Gly Asp Pro | 365 |
| Val Thr Leu Arg Leu Cys | |
| Cys Ser Arg Lys Lys Arg Ala Asp Lys | 366 |
| Lys Glu Asn Gly Thr Lys Leu Leu | |
| Phe Leu Leu Val Leu Gly Phe Ile Ile | 367 |
| Val Leu Pro Ser Val Ala Met Phe Leu | 368 |
| Leu Val Leu Gly Phe Ile Ile Ala Leu | 369 |
| Lys Val Val Thr Ser Ser Phe Val Val | 370 |
| Leu Val Pro Gly Thr Lys Phe Tyr Ile | 371 |
| Leu Leu Pro Ile Arg Thr Leu Pro Leu | 372 |
| Tyr Leu Val Lys Lys Gly Thr Ala Thr | 373 |
| Ser Leu Phe Ala Glu Thr Ile Trp Val | 374 |
| Met Leu Ile Ala Met Tyr Phe Tyr Thr | 375 |
| Leu Met Trp Thr Leu Pro Val Met Leu | 376 |
| Met Leu Ile Val Tyr Ile Phe Glu Cys | 377 |
| Tyr Ile Phe Glu Cys Ala Ser Cys Ile | 378 |
| Leu Val Leu Met Leu Ile Val Tyr Ile | 379 |
| Ala Leu Cys Arg Arg Arg Ser Met Val | 380 |
| Leu Leu Ser Gly Leu Ser Leu Phe Ala | 381 |
| Phe Leu Leu Val Val Gly Leu Ile Val | 382 |
| Leu Val Val Gly Leu Ile Val Ala Leu | 383 |
| Lys Val Val Lys Ser Asp Phe Val Val | 384 |
| Thr Leu Pro Val Gln Thr Leu Pro Leu | 385 |
| Asp Leu His Val Ile Ser Asn Asp Val | 386 |
| Val Leu Val His Pro Gln Trp Val Leu | 387 |
| Phe Leu Arg Pro Gly Asp Asp Ser Ser | 388 |
| Ala Leu Gly Thr Thr Cys Tyr Ala Ser | 389 |
| Lys Leu Gln Cys Val Asp Leu His Val | 390 |
| Glu Leu Ala His Tyr Asp Val Leu Leu | 391 |
| Asn Leu Asn Gly Ala Gly Asp Pro Leu | 392 |
| Thr Leu Arg Val Asp Cys Thr Pro Leu | 393 |
| Met Met Asn Asp Gln Leu Met Phe Leu | 394 |
| Ala Leu Phe Asp Ile Glu Ser Lys Val | 395 |
| Leu Leu His Glu Thr Asp Ser Ala Val | 396 |
| Val Leu Ala Lys Glu Leu Lys Phe Val | 397 |
| Ile Leu Leu Trp Gln Pro Ile Pro Val | 398 |
| Asp Leu Phe Gly Ile Trp Ser Lys Val | 399 |
| Pro Leu Glu Arg Phe Ala Glu Leu Val | 400 |
| Lys Gln Gly Asn Phe Asn Ala Trp Val | 401 |
| Asn Leu Leu Arg Arg Met Trp Val Thr | 402 |
| Asn Leu Phe Glu Thr Pro Ile Leu Ala | 403 |
| Asn Leu Phe Glu Thr Pro Val Glu Ala | 404 |
| Gly Leu Gln His Trp Val Pro Glu Leu | 405 |
| Val Gln Phe Val Ala Ser Tyr Lys Val | 406 |
| Arg Leu Leu Ala Ala Leu Cys Gly Ala | 407 |
| Leu Leu Leu Leu Thr Val Leu Thr Val | 408 |
| Leu Leu Leu Thr Val Leu Thr Val Val | 409 |
| Phe Leu Ser Phe His Ile Ser Asn Leu | 410 |
| Leu Leu Val Leu Val Cys Val Leu Val | 411 |
| Ala Leu Leu Val Leu Val Cys Val Leu | 412 |
| Ser Leu Ser Tyr Thr Asn Pro Ala Val | 413 |
| Asn Leu Thr Ile Ser Asp Val Ser Val | 414 |
| Ala Leu Ala Ser Thr Ala Pro Pro Val | 415 |
| Ala Ile Leu Cys Trp Thr Phe Trp Val | 416 |
| Phe Ile Leu Met Phe Ile Val Tyr Ala | 417 |
| Leu Thr Ala Glu Cys Ile Phe Phe Val | 418 |
| Met Leu Gln Asp Asn Cys Cys Gly Val | 419 |
| Ile Leu Cys Trp Thr Phe Trp Val Leu | 420 |
| Lys Ile Leu Leu Ala Tyr Phe Ile Leu | 421 |
| Phe Val Gly Ile Cys Leu Phe Cys Leu | 422 |
| Val Leu Leu Ser Val Ala Met Phe Leu | 423 |
| Leu Leu Ser Val Ala Met Phe Leu Leu | 424 |
| Ile Leu Gly Ser Leu Pro Phe Phe Leu | 425 |
| Ile Leu Asn Ala Tyr Leu Val Arg Val | 426 |
| Phe Leu Leu Val Gly Phe Ala Gly Ala | 427 |
| Asn Leu Gln Pro Gln Leu Ala Ser Val | 428 |
| Cys Met Phe Asp Ser Lys Glu Ala Leu | 429 |
| Tyr Leu Tyr Val Leu Val Asp Ser Ala | 430 |
| Tyr Met Asp Gly Thr Met Ser Gln Val | 431 |
| Lys Met Ala Arg Phe Ser Tyr Ser Val | 432 |
| Gly Leu Val Met Asp Glu His Leu Val | 433 |
| Phe Leu Pro Gly Cys Asp Gly Leu Val | 434 |
| Cys Met Leu Gly Ser Phe Cys Ala Cys | 435 |
| Tyr Leu Ala Phe Arg Asp Asp Ser Ile | 436 |
| Trp Leu Pro Lys Lys Cys Ser Leu Cys | 437 |
| Cys Leu Asn Gly Gly Thr Cys Met Leu | 438 |
| Met Leu Val Gly Ile Cys Leu Ser Ile | 439 |
| Phe Glu Leu Gly Leu Val Ala Gly Leu | 440 |
| Lys Met Val Arg Phe Ser Tyr Ser Val | 441 |
| Cys Leu Asn Glu Gly Thr Cys Met Leu | 442 |
| Met Leu Ala Gly Ile Cys Leu Ser Ile | 443 |
| Arg Leu Leu Phe Phe Leu Leu Phe Leu | 444 |
| Thr Leu Ala Tyr Leu Ile Phe Cys Leu | 445 |
| Leu Leu Phe Leu Thr Pro Met Glu Val | 446 |
| Lys Leu Met Ser Pro Lys Leu Tyr Val | 447 |
| Leu Leu Phe Phe Leu Leu Phe Leu Val | 448 |
| Ser Leu Phe Leu Gly Ile Leu Ser Val | 449 |
| Ala Ile Ser Gly Met Ile Leu Ser Ile | 450 |
| Phe Ile Arg Ala His Thr Pro Tyr Ile | 451 |
| Ser Leu Asn Phe Ile Arg Ala His Thr | 452 |
| Leu Lys Met Glu Ser Leu Asn Phe Ile | 453 |
| Ser His Phe Leu Lys Met Glu Ser Leu | 454 |
| Tyr Leu Phe Leu Gly Ile Leu Ser Val | 455 |

Other cancer relevant antigens include those summarized in the Tables in this online database cancerimmunity.org/peptide/ and incorporated herein by reference, last referenced May 6, 2015.

It is contemplated that in compositions of the disclosure, there is between about 0.001 mg and about 10 mg of total protein per ml in the composition. It is also contemplated that an effective dose is from about 0.0004 mg/kg to about 2.027 mg/kg, as measured by pMHC, and ranges in between 0.0004 mg/kg to about 2.027 mg/kg. Thus, the concentration of protein in a composition can be about, at least about or at most about 0.001, 0.010, 0.050, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 50, 100 μg/ml or mg/ml or more (or any range derivable therein). Of this, about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100% may be peptide/MHC/nanoparticle complex.

In addition, U.S. Pat. No. 4,554,101 (Hopp), which is incorporated herein by reference, teaches the identification and preparation of epitopes from primary amino acid sequences on the basis of hydrophilicity. Through the methods disclosed in Hopp, one of skill in the art would be able to identify potential epitopes from within an amino acid sequence and confirm their immunogenicity. Numerous scientific publications have also been devoted to the prediction of secondary structure and to the identification of epitopes, from analyses of amino acid sequences (Chou & Fasman, 1974a,b; 1978a,b; 1979). Any of these may be used, if desired, to supplement the teachings of Hopp in U.S. Pat. No. 4,554,101.

### Cytokines

In certain aspect, the NPs further comprise, or alternatively consist essentially of, or yet further consist of at least one cytokine molecule. As used herein, the term “cytokine” encompasses low molecular weight proteins secreted by various cells in the immune system that act as signaling molecules for regulating a broad range of biological processes within the body at the molecular and cellular levels. “Cytokines” include individual immunomodulating proteins that fall within the class of lymphokines, interleukins, or chemokines.

Non limiting examples are disclosed herein. For instance, IL-1A and IL-1B are two distinct members of the human interleukin-1 (IL-1) family. Mature IL-1A is a 18 kDa protein, also known as fibroblast-activating factor (FAF), lymphocyte-activating factor (LAF), B-cell-activating factor (BAF), leukocyte endogenous mediator (LEM), etc. IL-4 is a cytokine that induces T helper-2 (Th2) cell differentiation, and is closely related to and has similar functions to IL-13. IL-5 is produced by Th2 cells and mast cells. It acts to stimulate B cell growth and increase immunoglobulin secretion. It is also involved in eosinophil activation. IL-6 is an interleukin that can act as either a pro-inflammatory or anti-inflammatory cytokine. It is secreted by T cells and macrophages to stimulate immune response to trauma or other tissue damage leading to inflammation. IL-6 is also produced from muscle in response to muscle contraction. IL-8 is a chemokine produced by macrophages and other cell types such as epithelial cells and endothelial cells and acts as an important mediator of the immune reaction in the innate immune system response. IL-12 is involved in the differentiation of naïve T cells to T helper (Th1 or Th2) cells. As a heterodimeric cytokine, IL-12 is formed after two subunits encoded by two separate genes, IL-12A (p35) and IL-12B (p40), dimerize following protein synthesis. IL-12p70 indicates this heterodimeric composition. IL-13, a cytokine secreted by many cell types, especially Th2 cells, is an important mediator of allergic inflammation and disease. IL-17 is a cytokine produced by T helper cells and is induced by IL-23, resulting in destructive tissue damage in delayed-type reactions. IL-17 functions is a pro-inflammatory cytokine that responds to the invasion of the immune system by extracellular pathogens and induces destruction of the pathogen's cellular matrix. IP-10, or Interferon gamma-induced protein 10, is also known as C-X-C motif chemokine 10 (CXCL10) or small-inducible cytokine B10. As a small cytokine belonging to the CXC chemokine family, IP-10 is secreted by several cell types (including monocytes, endothelial cells, and fibroblasts) in response to IFN-γ. Macrophage Inflammatory Proteins (MIP) belong to the family of chemokines. There are two major forms of human MIP, MIP-1α and MIP-1β, which are also known as chemokine (C-C motif) ligand 3 (CCL3) and CCL4, respectively. Both are produced by macrophages following stimulation with bacterial endotoxins. Granulocyte colony-stimulating factor (G-CSF or GCSF), also known as colony-stimulating factor 3 (CSF 3), is a colony-stimulating factor hormone. G-CSF is a glycoprotein, growth factor, and cytokine produced by a number of different tissues to stimulate the bone marrow to produce granulocytes and stem cells. G-CSF also stimulates the survival, proliferation, differentiation, and function of neutrophil precursors and mature neutrophils. Epidermal growth factor or EGF is a growth factor that plays an important role in the regulation of cell growth, proliferation, and differentiation by binding with high affinity to its receptor EGFR. Vascular endothelial growth factor (VEGF) is a family of growth factors that are important signaling proteins involved in both vasculogenesis (the de novo formation of the embryonic circulatory system) and angiogenesis (the growth of blood vessels from pre-existing vasculature).

The cytokine or cytokines can be coupled to the nanoparticle in the same manner as the pMHC complex. In one embodiment of the present disclosure, the cytokine or cytokines and the pMHC complex are separately attached to the nanoparticle. In another embodiment of the disclosure, the cytokine or cytokines molecule and the pMHC complex are first complexed together and are then subsequently complexed to the nanoparticle. Multiple cytokines may be coupled to the nanoparticle; these may be multiple of the same cytokine or different cytokines.

### Co-Stimulatory Molecule Components

In certain aspects, the NPs additionally comprise, or alternatively consist essentially of, or yet further consist of at least one co-stimulatory molecule. Co-stimulatory molecules are molecules that produce a secondary signal in vivo that serves to activate naïve T cells into antigen-specific T cells capable of producing an immune response to cells possessing said specific antigen. The present disclosure is not limited to any specific co-stimulatory molecule. The various co-stimulatory molecules are well-known in the art. Some non-limiting examples of co-stimulatory molecules are 4-IBBL, OX40L, CD40, IL-15/IL-15Ra, CD28, CD80, CD86, CD30L, and ICOSL. Only one specific co-stimulatory molecule may be coupled to one nanoparticle or a variety of co-stimulatory molecules may be coupled to the same nanoparticle. In certain embodiments, the co-stimulatory molecule is a protein such as an antibody that is capable of agonizing a co-stimulatory receptor on a T cell. In this case, the antibody is capable of inducing a co-stimulatory signal that is necessary to activate naïve T cells and induce an immune response in an antigen-specific manner. Additionally or alternatively, the term “co-stimulatory molecule” as used herein may also refer to an agent capable of generating a co-stimulatory signal by having an agonistic effect on a native co-stimulatory signaling molecule, e.g., anti-CD28 or CD28 ligand generating a CD28 co-stimulatory response. In some aspects, the valency of the co-stimulatory molecules is from about 1 to about 6000, and/or the valency of the co-stimulatory molecules is from about 1 to about 6000, each per nanoparticle core.

### Compositions

In certain aspects, provided herein are compositions comprising a plurality of the complexes provided herein. In some embodiments, the compositions further comprise a carrier, optionally a pharmaceutical carrier. In some embodiments, the compositions provided herein may optionally comprise one or more nanoparticle cores coupled to one or more co-stimulatory molecules and/or cytokines. Accordingly, in some embodiments, the compositions comprise, or alternatively consist essentially of, or yet further consist of: 1) a plurality of nanoparticle cores coupled to a plurality of antigen-MHC complexes wherein at least one portion of the nanoparticle cores further comprises one or more co-stimulatory molecules and/or one or more cytokines, and a second portion of the nanoparticle cores do not further comprise a co-stimulatory molecule and/or a cytokine, and 2) a plurality of nanoparticle cores coupled to one or more co-stimulatory molecules and/or cytokines.

Methods of Making Nanoparticles and pMHC Complexes

pMHC-NPs and nanoparticles can be made by a variety of methods as described in, for example, WO 2008/109852, WO 2012/041968, WO 2012/062904, WO 2013144811, WO 2014/050286, WO 2015/063616, WO 2016/198932, or PCT/IB2017/001508.

### EXAMPLES

The following examples are given for the purpose of illustrating various embodiments of the disclosure and are not meant to limit the present disclosure in any fashion. One skilled in the art will readily appreciate that the present disclosure is well-adapted to carry out the objects and obtain the ends and advantages mentioned, as well as those objects, ends and advantages inherent herein. The present examples, along with the methods described herein are presently representative of embodiments and are exemplary, and are not intended as limitations on the scope of the disclosure. Changes therein and other uses which are encompassed within the spirit of the disclosure as defined by the scope of the claims will occur to those skilled in the art.

### Methods

Mice. NOD/Lt mice were from the Jackson Lab (Bar Harbor, ME). 17.4α/8.3β (8.3-NOD) and BDC2.5-NOD mice (expressing transgenic T-cell receptors for IGRP₂₀₆₋₂₁₄ or NRP-V7/K^{d} and 2.5mi/IA^{g7}, respectively) have been described^{19, 20, 21}.

pMHC production. Two different methods were used to express recombinant pMHC class I complexes. The first involved re-folding MHC class I heavy and light chains expressed in bacteria in the presence of peptide, followed by purification via gel filtration and anion exchange chromatography^{22, 23}. The second involved expressing MHC class I complexes at high yields in mycoplasma-free lentiviral-transduced freestyle Chinese hamster ovary (CHO) cells as single chain constructs in which the peptide-coding sequence, the MHC class I light and heavy chains are sequentially tethered with flexible Glycine-Serine (GS) linkers²⁴ followed by a carboxyterminal linker encoding a BirA site, and 6×His and strep tags ending with a free cysteine. The secreted proteins were purified from culture supernatants using strep tag and/or nickel columns and used directly for NP coating or were biotinylated to produce pMHC tetramers using fluorochrome-conjugated streptavidin.

Recombinant pMHC class II monomers were produced in freestyle CHO cells transduced with lentiviruses encoding a monocistronic message in which the peptide-MHCα and MHCβ chains of the complex were separated by the ribosome skipping P2A sequence. A linker encoding a BirA site, a strep and/or 6×His tags, and a free Cys was added to the carboxyterminal end of the construct. The self-assembled pMHC class II complexes were purified from the culture supernatants by nickel affinity chromatography and used for coating onto NPs or processed for biotinylation and tetramer formation as described above.

NP synthesis. Gold nanoparticles (GNPs) were synthesized by chemical reduction of chloroauric acid (HAuCl₄) with sodium citrate as described (Perrault, S. D. et al. (2009) Nano Lett. 9(5):1909-1915). Briefly, 2 mL of 1% of HAuCl₄ (Sigma Aldrich, Oakville, ON) was added to 100 mL H₂O under vigorous stirring and the solution heated in an oil bath. Six (for 14 nm GNPs) or two mL (for 40 nm GNPs) of 1% sodium citrate were added to the boiling HAuCl₄ solution, which was stirred for an additional 10 min and then cooled down to room temperature. GNPs were stabilized by the addition of 1 uM of thiol-polyethylene glycol (thiol-PEG) linkers (Nanocs, MA) functionalized with carboxyl (—COOH) or primary amine (—NH₂) groups as acceptors of pMHC. Pegylated GNPs were washed with water to remove free thiol-PEG, concentrated and stored in water for further analysis. NP density was calculated from spectrophotometry measurements according to Beer's law.

The SFP series iron oxide (Fe₃O₄) NPs were produced by thermal decomposition of iron acetylacetonate in organic solvents in the presence of surfactants, then rendered solvent in aqueous buffers by pegylation (Xie, J. et al. (2007) Adv Materials 19(20):3163-3166; Xie, J. P. S. et al. (2006) Pure Appl Chem 78(5):1003-1014; Xu, C. et al. (2007) Polymer International 56(7):821-82). Briefly, 2 mmol Fe(acac)3 (Sigma Aldrich) were dissolved in a mixture of 10 mL benzyl ether and oleylamine and heated to 100° C. for 1 hour followed by 300° C. for 2 hours with reflux under the protection of a nitrogen blanket. Synthesized NPs were precipitated by addition of ethanol and resuspended in hexane. For pegylation of the iron-oxide NPs, 100 mg of different dopamine-conjugated PEG (DPA-PEG, 3.5 kDa) linkers (Jenkem Tech USA) were dissolved in a mixture of chloroform and dimethylformamide (DMF). The NP solution (20 mg Fe) was then added to the DPA-PEG solution and stirred for 4 hr at room temperature. Pegylated SFP NPs were precipitated overnight by addition of hexane and resuspended in water. Trace amounts of aggregates were removed by high-speed centrifugation (20,000×g, 30 min). The monodisperse SFP NPs were stored in water for pMHC conjugation. The concentration of iron was determined spectrophotometrically at 410 nm in 2N hydrochloric acid (HCl). Based on the molecular structure and diameter of SFP NPs (Fe₃O₄; 8+1 nm diameter) (Xie, J. et al. (2007) Adv Materials 19(20):3163-3166; Xie, J. P. S. et al. (2006) Pure Appl Chem 78(5):1003-1014), Applicant estimated that SFP solutions containing 1 mg of iron contain 5×10¹⁴ NPs.

Applicant subsequently developed a new iron-oxide NP design that allowed the formation, also by thermal decomposition but in a single step, of pegylated iron-oxide NPs in the complete absence of surfactants (PF series iron-oxide NPs). In this design, PEG molecules were used as in situ surface-coating agent. In a typical reaction, 3 g PEG (2 kDa MW) were melted slowly in a 50 mL round bottom boiling flask at 100° C. and then mixed with 7 mL of benzyl ether and 2 mmol Fe(acac)3. The reaction was vigorously stirred for 1 hr and heated to 260° C. with reflux for an additional 2 hr. The reaction mixture was cooled down to room temperature, transferred to a centrifugation tube and mixed with 30 mL water. Insoluble materials were removed by centrifugation at 2,000×g for 30 min. The free PEG molecules were removed by ultrafiltration through Amicon-15 filters (MWCO 100 kDa, Millipore, Billerica, MA). Iron oxide NPs were generated with most, albeit not all of the PEG molecules tested. The sizes of the iron oxide NPs varied depending on the functional groups of the PEG linkers used in the thermal decomposition reactions. The NPs could be readily purified using magnetic (MACS) columns (Miltenyi Biotec, Auburn, CA) or an IMag cell separation system (BD BioSciences, Mississauga, ON). The purified iron oxide NPs were stored in water at room temperature or 4° C. without any detectable aggregation. NP density was calculated as described above for SFP NPs.

pMHC conjugation to NPs. pMHC conjugation to NPs produced with PEG linkers carrying distal primary amine (—NH₂) or carboxyl (—COOH) groups was achieved via the formation of amide bonds in the presence of 1-Ethyl-3-[3-dimethylaminopropyl]carbodiimide hydrochloride (EDC). NPs (GNP-C, SFP-C and PF-C) with —COOH groups were first dissolved in 20 mM 2-(N-morpholino) ethanesulfonic acid (MES) buffer, pH 5.5. N-hydroxysulfosuccinimide sodium salt (sulpho-NHS; 10 mM) and EDC (1 mM) (Thermo scientific, Waltham, MA) were then added to the NP solution. After 20 min of stirring at room temperature, the NP solution was added drop-wise to the pMHC monomer solution (in 20 mM borate buffer, pH 8.2). The mixture was stirred for additional 4 hr. To conjugate pMHCs to NH₂-functionalized NPs (GNP-N, SFP-N and PF-N), pMHC complexes were first dissolved in 20 mM MES buffer, pH 5.5, containing 100 mM sodium chloride (NaCl). Sulpho-NHS (10 mM) and EDC (5 mM) were then added to the pMHC solution. The activated pMHC molecules were added to the NP solution in 20 mM borate buffer (pH 8.2), and stirred for 4 hr at room temperature.

To conjugate pMHC to maleimide-functionalized NPs (SFP-M and PF-M), pMHC molecules engineered to encode a free C-terminal Cys were mixed with NPs in 40 mM phosphate buffer, pH 6.0, containing 2 mM ethylenediaminetetraacetic acid (EDTA), 150 mM NaCl, and incubated overnight at room temperature. pMHCs were covalently bound with NPs via the formation of a carbon-sulfur bond between maleimide groups and the Cys residue.

Click chemistry was used to conjugate pMHC to NPs functionalized with azide groups (SFP-Z). For this reaction, pMHC molecules were first incubated with dibenzocyclooctyne-N-hydroxysuccinimidyl ester (DBCO-NHS, Click Chemistry Tools, Scottdale, AZ) for 2 hr at room temperature. Free DBCO molecules were removed by dialysis overnight. pMHC-DBCO conjugates were then incubated with SFP-Z for 2 hr, resulting in formation of triazole bonds between pMHCs molecules and NPs.

Unconjugated pMHC complexes in the different pMHC-NP conjugating reactions were removed by extensive dialysis against PBS, pH 7.4, at 4° C. though 300 kDa molecular weight cut off membranes (Spectrum labs). Alternatively, pMHC-conjugated iron oxide NPs were purified by magnetic separation. The conjugated NPs were concentrated by ultrafiltration through Amicon Ultra-15 units (100 kDa MWCO) and stored in PBS.

NP characterization. The core size and dispersity of unconjugated and pMHC-conjugated NPs were first assessed via transmission electron microscopy (TEM, Hitachi H7650). Dynamic light scattering (DLS, Zetasizer, Malvern, UK) was used to determine the NPs' and pMHC-NPs' hydrodynamic size. The chemical nature of the iron oxide core of the PF series of NPs was evaluated using small angle electron beam diffraction (SEBD). The surface chemical properties were evaluated using Fourier transform infrared spectroscopy (FTIR). pMHC conjugated NPs were analyzed via native- and denaturing PAGE, Bradford assay, amino acid analysis and dot-enzyme-linked immunosorbent assay (dot-ELISA).

Fourier transform infrared spectroscopy (FTIR). The surface chemical properties of the PF-series iron oxide NP designs were evaluated using Fourier Transformation Infrared spectroscopy (FTIR). The FTIR spectra of control PEG and PEG anchored on the PF-NP surface were obtained using a Nicolet FTIR spectrophotometer on an ATR (attenuated total reflection) mode. Each of the spectra was recorded as the average of 256 scans at 4 cm¹ spectral resolution. The molecular vibration signatures of the PEG backbone (represented by C—H asymmetric stretching vibration, C—O—C vibration and CH₂ rocking vibration) and their distal pMHC-acceptor functional groups were identified.

Agarose gel electrophoresis. To quickly evaluate changes on the NP charge as a function of pegylation or pMHC coating, NPs were subjected to electrophoresis on 0.8% agarose gels. Pegylated NPs migrated to negative or positive poles depending on the overall surface charge.

Native and denaturing polyacrylamide gel electrophoresis. pMHC conjugated NPs were subjected to native-PAGE and SDS-PAGE (10%) analyses to confirm absence of free (unconjugated pMHC) in the pMHC-NP preparations and to confirm presence of intact trimolecular pMHC complexes on the NP's surface.

pMHC valency measurements. To evaluate the number of pMHC monomers conjugated onto individual NPs (pMHC valency), Applicant measured the pMHC concentration of the pMHC-NP preps using different approaches, including Bradford assay (Thermo Scientific), amino acid analysis (HPLC-based quantification of 17 different amino acids in hydrolyzed pMHC-NP preparations) (University of Toronto) and dot-enzyme-linked immunosorbent assay (dot-ELISA), and the values converted to ratios of pMHC molecular number to NP number. Briefly, in the “dot-ELISA” approach, pMHC-conjugated and unconjugated NPs and pMHC monomer solutions (as standards) were serially diluted in PBS and absorbed to a polyvinylidene fluoride (PVDF) membrane in a multiwell filter plate (PALL Corporation). The plate was allowed to semi-dry at room temperature and then incubated with pMHC-specific primary antibodies (i.e., anti-β2M and anti-K^{d} antibodies for pMHC class I-coated NPs, clones 2M2 and SF1-1.1, respectively; BioLegend, San Diego, CA), followed by HRP- or AP-conjugated secondary antibodies. Upon development of the enzymatic color reactions, the contents of the wells were transferred to wells of a conventional ELISA plate and their absorbance measured at 450 nm using a plate reader. Since the values generated by these different methods were similar, the Bradford assay (using unconjugated NPs as blanks) became the method of choice for ease and simplicity.

TCR signaling in TCR/mCDA-transfected JurMA cells. The TCRα and TCRβ cDNAs encoding the BDC2.5-TCR were generated from BDC2.5-CD4+ T-cell-derived mRNA using the 5′ RACE System for Rapid Amplification of cDNA Ends, version 2.0 kit (Thermo Fisher Scientific, Waltham, USA) and TCRα or TCRβ-specific oligonucleotide primers. The resulting PCR products were cloned into the pCR8 plasmid and sequenced. The full-length cDNAs were then subcloned into a retroviral vector upstream of an IRES-eGFP cassette, as a single open reading frame in which the TCRα and TCRβ cDNAs were separated by a P2A ribosome skipping sequence.

The polypeptide sequence of the TCRα-P2A-TCRβ fusion protein is provided in the Exemplary Sequence Listing provided below.

The sequence of polynucleotide encoding the TCRα-P2A-TCRβ fusion protein is provided in the Exemplary Sequence Listing provided below.

The human CD3+/TCR β-JurMA reporter cell line (engineered to express NFAT-driven luciferase) was transduced with a retrovirus encoding murine CD4 by coculture with the retrovirus-producing GP+envAm12 cell line. Transduced cells were expanded, stained with Pacific Blue-conjugated anti-mCD4 (GK1.5) (BioLegend, San Diego, CA) and sorted with a BD FACSAria II (BD Biosciences, NJ). The CD4+ Jurkat/MA cells were then transduced with a retrovirus encoding the BDC2.5-TCRαβ and IRES-eGFP. eGFP and mCD4 double-positive cells were sorted by flow cytometry and stained with PE-labeled BDC2.5/IAg7 pMHC tetramers to confirm their specificity.

To measure NFAT-driven expression of luciferase, wild-type and BDC2.5/mCD4⁺ JurMA cells were plated in a 48-well plate at 500,000 cells/well in 200 μl of DMEM (Sigma-Aldrich, St. Louis, MO) supplemented with 10% FBS (Sigma-Aldrich), 20 mM L-glutamine (Sigma-Aldrich), 10 mM sodium pyruvate (Thermo Fisher Scientific, Waltham, MA), and antibiotics, in the presence or absence of 20 ng/ml PMA (Sigma-Aldrich) plus 0.5 μM Ionomycin (Sigma-Aldrich), 10 μg/mL of anti-hCD3ε mAb (OKT3, BD Biosciences) or 12.5 μg/mL of BDC2.5/IA^{g7}-coated PF-M. Cells were collected from the wells at different times after stimulation, transferred to a 96-well plate, and washed 3 times with PBS. 105 cells were transferred to a new 96-well plate, lysed in 20 μl Cell Culture Lysis Reagent (Promega, Madison, WI) and incubated with 100 μl of Luciferase Assay Reagent (Promega) in opaque white plates (Greiner Bio One International GmbH, Kremsmünster, Austria) using a Veritas™ Microplate Luminometer (Promega) with injectors. Luciferase activity was expressed as relative luminescence units (RLUs), normalized to the luciferase activity of non-stimulated cells.

Agonistic activity of pMHC-NPs in vitro. FACS-sorted splenic CD8+ or CD4+ cells from TCR-transgenic mice (2.5×105 cells/mL) were incubated with a range of pMHC-conjugated or control NP concentrations for 24-48 h at 37° C. The supernatants were assayed for IFNγ by ELISA.

Responsiveness of human T-cell clones to agonistic mAbs and pMHC-coated NPs was assessed by culturing 5×10⁵ clonal T-cells in 48-well plates, in 500 μl of complete RPMI-1640 media containing anti-CD3/anti-CD28 mAb-coated beads (Life Technologies; at a bead-to-cell ratio of 1:1), PPI_{76-90(88S)}/DRB1*0401-coated PF-M (50 μg of peptide/MHC/ml) or an identical number of control, Cys-coated PF-M. On day 2, supernatants were collected for cytokine content analyses by Luminex and cell pellets harvested for RNA extraction. In other experiments, T-cell clones were incubated with PPI_{76-90(88S)}/DRB1*0401-coated PF-M or Cys-coated PF-M for up to 5 days. Cells were collected on days 0, 2, 3, 4 and 5 and used for RNA extraction.

Transmission electron microscopy (TEM) of pMHC-NP/cell conjugates. BDC2.5-CD4⁺ and 8.3-CD8⁺ T-cells (5×10⁶/mL), isolated from TCR-transgenic animals using biotin-streptavidin CD4⁺ or CD8⁺ T-lymphocyte enrichment kits (BDC ImagTM, BD Biosciences), were incubated with 2.5mi/IA^{g7}- and NRP-V7/K^{d}-coated PF-M NPs for 30 min at 4° C. (15-20 μg/mL of pMHC). The cultures were further incubated at 37° C. for the indicated lengths of time, washed with cold PBS to remove unbound PF-M NPs, fixed and sectioned (70 nm) for TEM imaging with a Hitachi H7650.

Super-resolution microscopy. Purified 8.3-CD8⁺ T-cells were incubated with NRP-V7/K^{d}-PF-M-Alexa-647 NPs at 4° C. for 30 min or at 37° C. for another hr. Cells were washed three times with cold PBS pH 7.4, then fixed in 2% PFA for 15 min on ice. After washing, cells were stained by 1 μg/mL DAPI at RT for 5 min, mounted and observed under a Super-Resolution Microscope (ELYRA 131, Zeiss). Image processing and quantitative analysis of cluster diameter were done with ZEN 2012 software (n=100).

Scanning Electron Microscopy (SEM) and X-ray spectrometry of pMHC-NP/cell conjugates. Thioglycollate-induced peritoneal macrophages and bone marrow-derived DCs were prepared as described above. BDC2.5-CD4⁺ and 8.3-CD8⁺ T-cells were negatively selected from BDC2.5-NOD or 8.3-NOD mouse spleens using biotin-streptavidin CD4⁺ or CD8⁺ T-lymphocyte enrichment kits (BD ImagTM, BD Biosciences). The cells were plated on a coverslip and incubated with unconjugated or Cys-conjugated PF-M, BDC2.5mi/IA^{g7}-PF-M or NRP-V7/Kd-PF-M at 4° C. for 30 min with/without additional 60 or 180 min incubations at 37° C. After incubation, cells were washed with 0.05 M cacodylate buffer (CB) pH 7.4, then fixed with 2.5% glutaraldehyde at 4° C. overnight. The specimens were subjected to sequential dehydration in graded ethanol and immersed in hexamethyldisilazane for 3 min for drying. The samples were observed under XL30 SEM (Philips, Netherlands) by gold coating. Element analysis was carried out using energy-dispersive X-ray spectrometry (EDS).

### Example 1-Molecular pMHC Density on the Nanoparticle (NP) Surface Versus the Biological Activity of pMHC-Based Nanomedicines

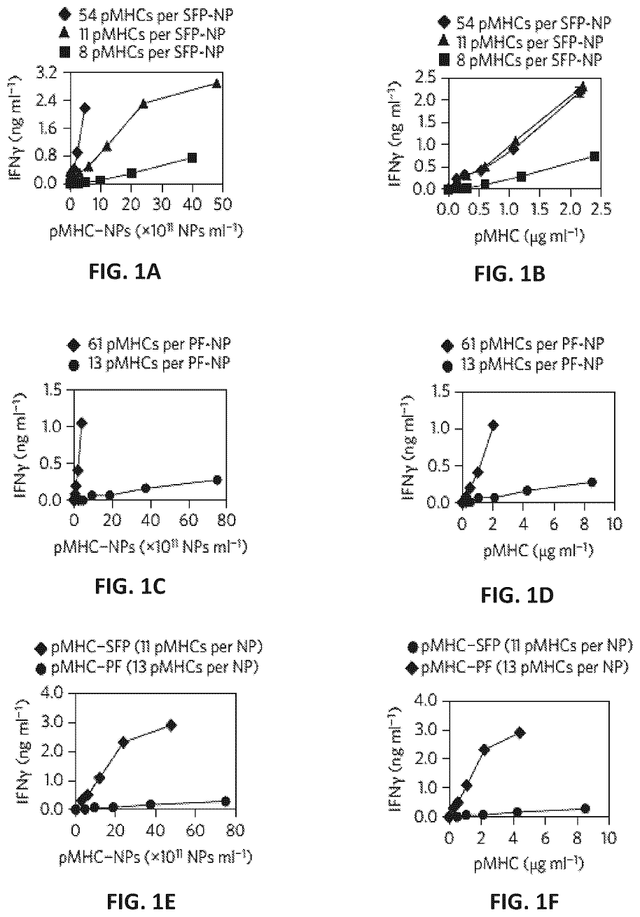

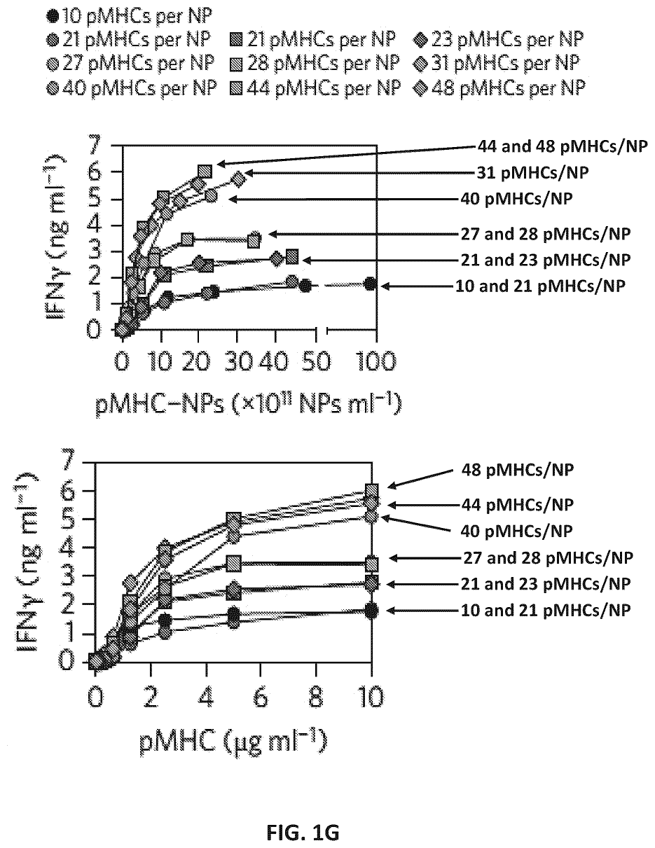

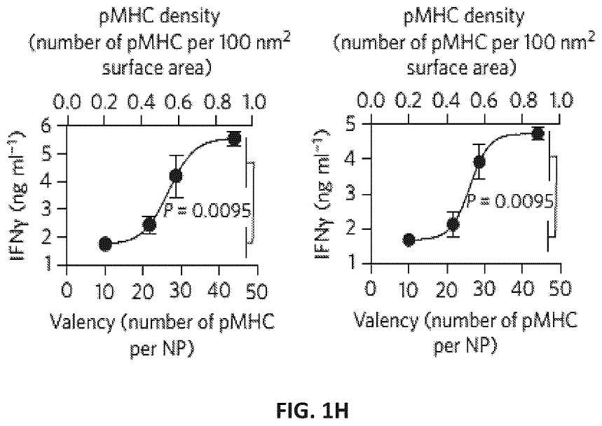

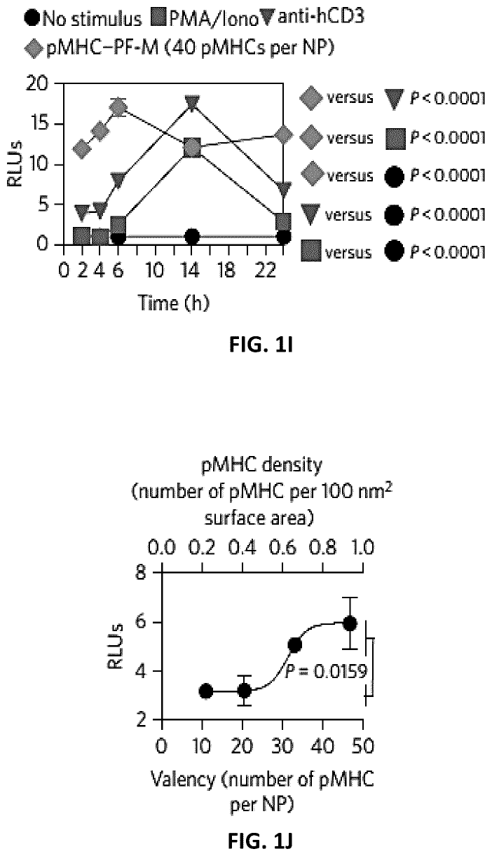

To understand how the valency of peptide-major histocompatibility complexes (pMHC) and pMHC-nanoparticle (NP) concentration contributes to the biological activity of these compounds, Applicant compared the ability of various NRP-V7/Kd-NP preparations to transiently activate cognate (NRP-V7/Kd/IGRP206-214-specific) CD8⁺ T-cells from T-cell-receptor (TCR)-transgenic 8.3-NOD mice. As shown in FIG. 1A, 8.3-CD8⁺ T-cells produced small amounts of interferon gamma (IFNγ) when cultured in the presence of SFP-NPs coated with 8 pMHCs/NP but substantially higher amounts of IFNγ in response to NPs coated with higher pMHC valencies, even as low as 11 pMHCs/NP, over a broad range of pMHC-NP or pMHC concentrations. This observation suggested that there is a threshold of pMHC valency for agonistic activity of SFP-NPs, lying between 9 and 11 pMHCs/NP (FIGS. 1A and 1B). Without being bound by theory, increases in pMHC-NP concentrations can enhance the agonistic properties of pMHC-NPs carrying “threshold” or “supra-threshold” pMHC valencies.

To confirm this observation, Applicant next used PF-NPs, which are larger than SFP-NPs and thus, have greater pMHC-coating capacity. pMHC-PF NPs carrying 13 or fewer pMHCs/NP had very weak or no biological activity up to ˜8×10¹² NPs/mL, as compared to PF-NPs displaying a much higher pMHC valency (61 pMHCs/NP, FIGS. 1C and 1D, and data not shown). This supported the idea that the threshold of pMHC required for agonistic activity increases with NP size (i.e., from >8 pMHCs for ˜8 nm SFP-NPs to >13 pMHCs for ˜20 nm PF-NPs). The inverse effects of NP size and pMHC valency on agonistic activity suggested a role for pMHC density (pMHCs/surface area of NP). This is further illustrated in FIGS. 1E and 1F, where Applicant compared the biological activity of SFP- and PF-NPs coated with a similar number of pMHCs over a range of NP or pMHC concentrations (to compensate for absolute differences in total pMHC ‘load’ when using identical concentrations of NPs of different size).

### Example 2—Rapid Increases in Biological Activity Above Threshold pMHC Densities

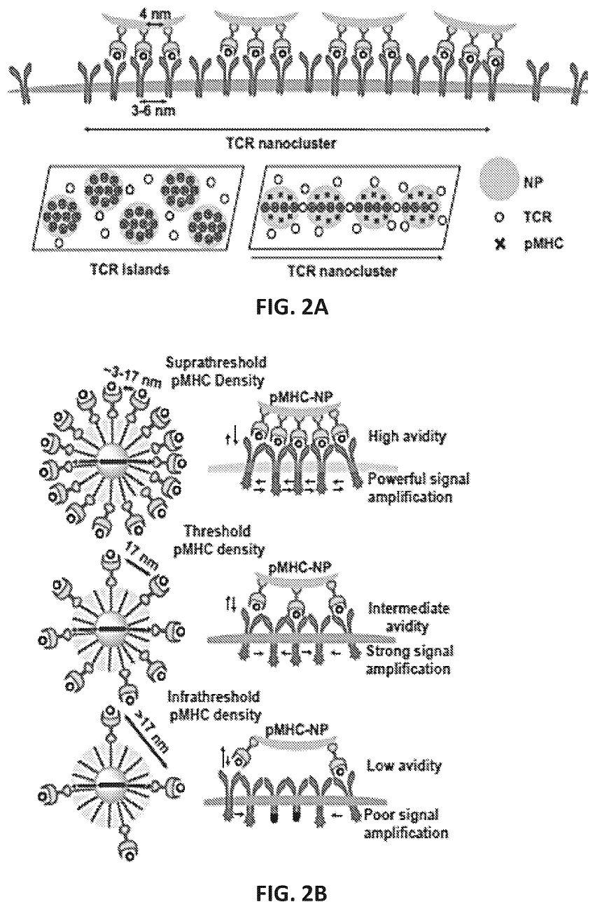

These data suggested that the biological activity threshold is defined by a constant that corresponds to the distance separating individual pMHC monomers on the NP. Applicant compared the maximum and predicted threshold binding capacities of NPs of different sizes, to identify a pMHC-density threshold. The theoretical pMHC density threshold lies at 0.004468 pMHCs/nm², corresponding to 11 pMHCs for an 8 nm NP or 22 pMHCs for a 20 nm NP. These values correspond to a calculated intermolecular distance of ˜16.88 nm. The T-cell antigen receptor (TCR) complex is thought to contain up to two TCRαβ heterodimers within a CD3γ-CD3ε-TCRαβ-CD3ζ-CD3ζ-TCRαβ-CD3δ-CD3ε complex (Rojo, J. M. ET AL. (1991) Immunol Today 12(10):377-378; Fernandez-Miguel, G. et al. (1999) Proc Natl Acad Sci USA 96(4):1547-1552). This structure is compatible with the estimated width of the TCR complex based on 3D reconstruction (12 nm) (Arechaga, I. et al. (2010) Int Immunol 22(11):897-903), and consistent with the calculated inter-pMHC distance of 16.88 nm to reach the agonistic threshold. Applicant calculated the minimum possible inter-molecular distance at ˜3.62 nm, which bodes well with the estimated 3-6 nm distance spanning individual TCRs within TCRαβ nanoclusters; this distance would allow a near-perfect alignment of pMHC on the NPs and cognate TCRs on T-cells (FIG. 2A). pMHC-NPs capable of ligating contiguous TCR heterodimers in these clusters are efficient in eliciting TCR signaling. These models explain why small NPs coated with closely apposed pMHCs have optimal immunological properties. pMHC density controls T_{reg} cell conversion because it can promote the sustained assembly of large TCR microclusters, leading to rapid, robust and prolonged TCR signaling (FIG. 2B).

The hypothesis based on data generated using pMHC class I-coated NPs were tested by comparing the TCR triggering potency of PF-NPs coated with pMHC class II monomers, over a broad range of valencies. CD4⁺ T-cells isolated from BDC2.5-TCR-transgenic NOD mice produced small amounts of IFNγ in response to PF-M NPs coated with up to 22 cognate (BDC2.5mi/IAg7) pMHC complexes (0.0045 pMHCs/nm², FIG. 1G). Remarkably, by plotting the IFNγ secretion data obtained at 10 and 5 μg of pMHC/mL (the concentrations at which the dose-response effect plateaus), the magnitude of IFNγ secretion increases exponentially in response to relatively small increases in pMHC valency, starting at ˜22 pMHCs (the predicted threshold valency) and ending at ˜32 pMHCs/NP (0.0065 pMHCs/nm2, herein referred to as the “minimal optimal valency”) (FIG. 1H). Substantial increases in pMHC valency/density above this minimal optimal valency do not result in significantly higher potency (FIG. 1H).

### Example 3-pMHC Density Controls the Magnitude of pMHC-NP-Induced TCR Signaling

To ascertain if these biological effects could be accounted for by pMHC density-dependent differences in the efficiency of TCR signaling, Applicant transduced the Jurkat/MA (JurMA) human T-cell line (lacking endogenous TCRβ chain expression and carrying a luciferase reporter driven by nuclear factor of activated T-cells (NFAT) transcription factor-binding DNA sequences) (Scholten, K. B. et al. (2005) Clin Immunol 114(2):119-129) with lentiviruses encoding the BDC2.5 TCRαβ heterodimer and the murine CD4 co-receptor. As shown in FIG. 1I, BDC2.5-TCR/mCD4-JurMA cells responded rapidly (within 2h), vigorously and for a sustained period of time (>24h) to BDC2.5mi/IA^{g7}-coated PF-M, as compared to optimal concentrations of an agonistic anti-human CD3ε mAb or PMA/ionomycin, which triggered a much slower response that peaked at 14h and progressively decreased afterwards. Notably, experiments using PF-M NPs coated with a broad range of BDC2.5mi/IA^{g7} valencies indicated that the magnitude of luciferase expression (a direct read-out of TCR signaling) followed kinetics remarkably similar to those seen with primary BDC2.5-CD4⁺ T-cells, indicating that threshold and supra-threshold pMHC densities somehow promote cooperative TCR signaling (FIG. 1J).

Also it is unexpected to observe the assay of this disclosure presenting a sigmoidal curve (FIG. 1J) that closely mimics the pMHC-density-response curve when using naive primary TCR-transgenic T-cells, both in terms of (a) shape consistent with cooperative signaling effects, and (b) the specific pMHC valencies/densities that define threshold and minimal optimal densities. This is completely surprising for a transfected cell line that over expresses the exogenous TCR/co-receptor pairs. One of skill in the art would have expected linear (as opposed to sigmoidal shaped curve) responses from the transfected cells given the difficulty to match the molecular number and precise stoichiometry of the transfected murine TCR and CD4 molecules, and considering that the host cell line is of human origin (including its CD3 chain components), whereas the pMHC and TCR/CD4 molecules tested were murine.

### Example 4-pMHC-NPs Trigger Antigen Receptor Clustering on Murine Cognate T-Cells

Applicant has shown that pMHC-NPs promote T_{reg} cell conversion by directly ligating TCRs on cognate T-cells, rather than by delivering pMHCs to these T-cells via a professional antigen-presenting cell (APC) (Clemente-Casares, X. et al. (2016) Nature 530(7591):434-440). The pMHC density effect revealed by the above experiments, coupled to the rapid and sustained production of NFAT-driven luciferase in pMHC-NP-challenged TCR/mCD4 transfected JurMA cells as compared to other stimuli (FIG. 1J), suggested that pMHC-NPs might operate by inducing prolonged TCR ligation (as opposed to the transient nature of low-affinity monomeric pMHC/TCR interactions).

TCRs are organized, on the surface of naïve T-cells, as linear clusters (Schamel, W. W. et al. (2013) Immunol Rev 251(1):13-20) or non-linear assemblies (Lillemeier, B. F. et al. (2010) Nat Immunol 11(1):90-96) of up to ˜200 nm in diameter/length and composed of up to 30 closely associated TCRs (nanoclusters) (Zhong, L. et al. (2009) PLoS One 4(6):e5945). The nanocluster architecture of these TCR assemblies is thought to increase the physical avidity, hence functional sensitivity, of T-cells for cognate pMHC on professional APCs and promote cooperative intracellular signaling among the closely apposed TCR units. There is evidence that TCR nanocluster formation is constitutive and predates the TCR microcluster formation (leading to sustained TCR signaling) that results from pMHC ligation (which generally range from 300-800 nm in size and contain up to 70 TCRs) (Lillemeier, B. F. et al. (2010) Nat Immunol 11(1):90-96; Yokosuka, T. et al. (2005) Nat Immunol 6(12):1253-1262; Choudhuri, K. et al. (2010) FEBS Lett 584(24):4823-4831; Sherman, E. et al. (2011) Immunity 35(5):705-720).

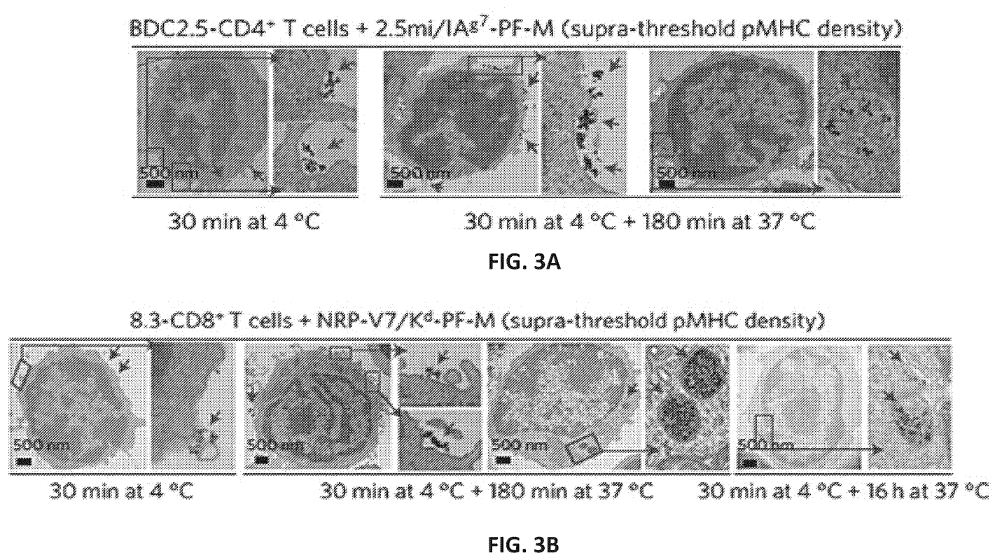

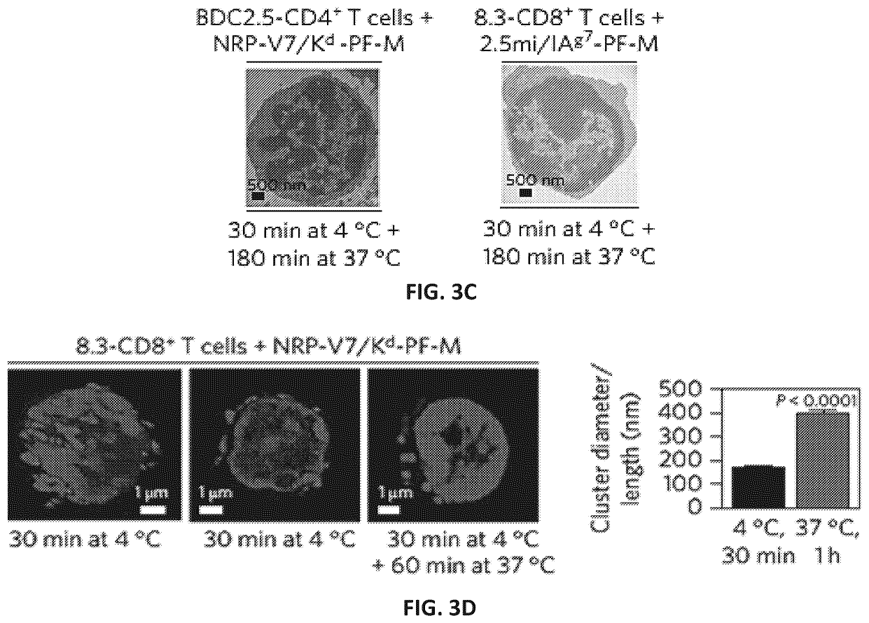

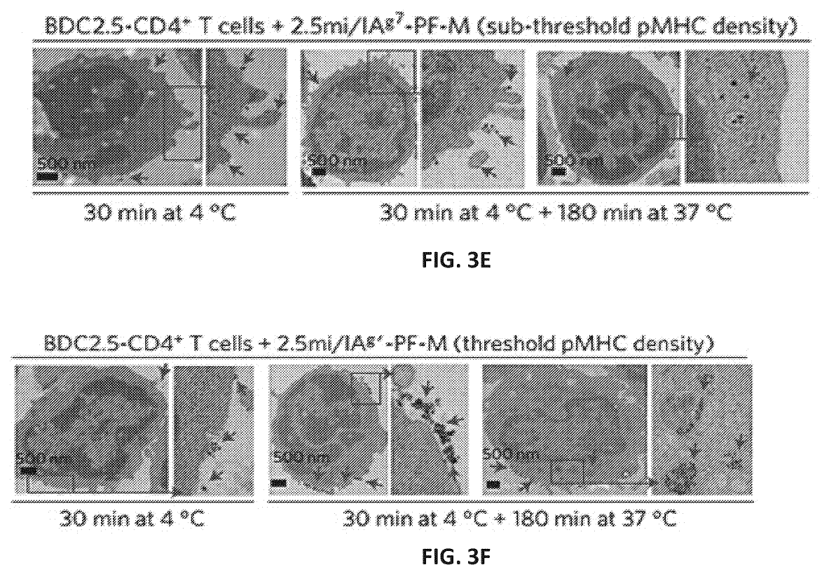

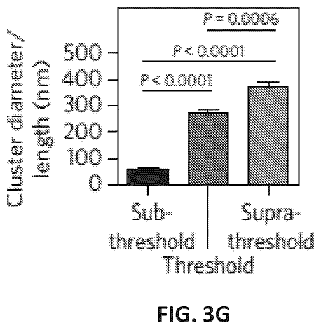

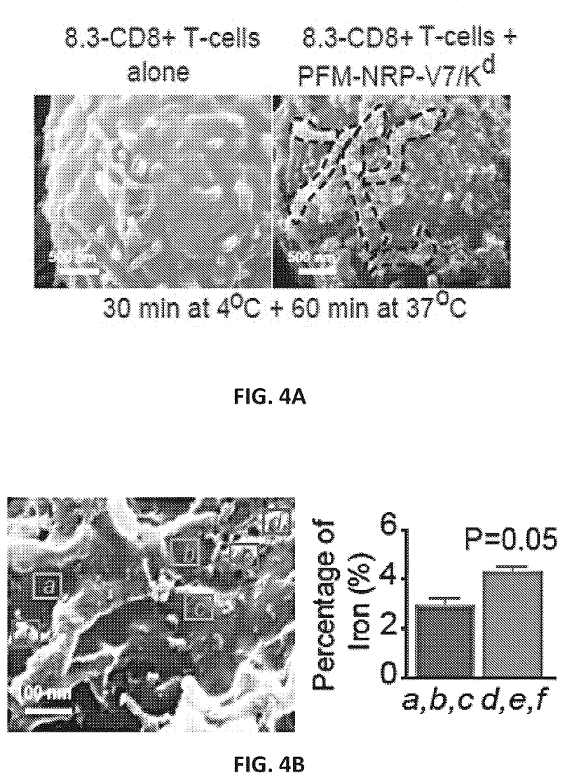

To gain insights into the pMHC density effect described above, Applicant investigated the binding geometry and kinetics of pMHC-coated-NPs (at supra-threshold pMHC densities) to cognate T-cells. TEM studies revealed that pMHC-NPs bind cognate CD8+ or CD4+ T-cells as clusters (islands) of several NPs spanning ˜100-150 nm (FIGS. 3A and 3B). This binding geometry was already seen within 30 min at 4° C., was followed by cluster growth (to diameters/lengths of ˜400 nm) upon incubation at 37° C. (FIGS. 3A, 3B and 3G), and culminated in internalization of the NPs in intracellular vesicles, starting at ˜3 hr after binding (FIGS. 3A and 3B). This clustered engagement was antigen-specific since neither binding nor internalization of NPs were seen when pMHC-NPs were incubated with non-cognate T-cells (FIG. 3C). These results were substantiated by super-resolution microscopy (FIG. 3D) and scanning electron microscopy (SEM) (FIGS. 4A and 4B), confirming the presence of clustered pMHC-NPs on the surface of cognate T cells.

Taken together, these data suggested that pMHC-NPs function as TCR nanocluster-binding and microcluster-triggering devices, raising the possibility that this process might be responsible for, or at least contribute to T_{reg} cell conversion. Since T_{reg} conversion is a direct function of pMHC density, Applicant investigated whether variations in pMHC density had any effects on TCR microcluster formation. Applicant compared BDC2.5mi-IA^{g7}-NP preparations carrying pMHCs at sub-threshold, threshold and supra-threshold densities. Remarkably, NPs coated at sub-threshold densities bound to and were eventually internalized by cognate CD4⁺ T-cells but without forming clusters (FIGS. 3E and 3G). In contrast, NPs coated at threshold densities readily triggered the formation of clusters, and the sizes of these clusters increased using NPs coated at supra-threshold densities (FIGS. 3A, 3F and 3G).

The above data indicate that the binding geometry of pMHC-based nanomedicines to cognate T-cells accounts for the observed pMHC-density effects. Closely apposed pMHC monomers on the NP surface would facilitate the repeated re-engagement of transiently dissociated pMHC monomers on individual NPs, thus delaying TCR internalization and lengthening the t_{1/2s} of individual TCR-pMHC interactions (Zhong, L. et al. (2009) PLoS One 4(6): e5945; Huppa, J. B. et al. (2010) Nature 463(7283):963-967). The cytoskeletal rearrangements triggered by the resulting signaling events would then promote the sustained assembly of proximal pMHC-NP-TCR units into large TCR microclusters (Bunnell, S. C. et al. (2002) J Cell Biol 158(7):1263-1275), further amplifying the duration and magnitude of TCR signaling (Yokosuka, T. et al. (2005) Nat Immunol 6(12):1253-1262). High pMHC densities would also facilitate the cooperative propagation of conformational changes and associated downstream signaling events from pMHC-bound TCRs to their unbound neighbours (Gil, D. et al. (2002) Cell 109(7):901-912; Minguet, S. et al. (2007) Immunity 26(1):43-54), both within and between individual NPs on membrane clusters (Martinez-Martin, N. et al. (2009) Science Signaling 2(83):ra43). This interpretation is compatible with both the kinetic proofreading (McKeithan, T. W. (1995) Proc Natl Acad Sci USA 92(11):5042-5046) and serial TCR engagement models (Valitutti, S. et al. (1995) Nature 375(6527):148-151) of T-cell activation.

The discovery of the unanticipated pMHC-density- and antigen-receptor clustering-dependent signaling properties of these compounds enables the use of antigen receptor-expressing reporter cell lines such as those described herein or similar in potency and batch-release assays.

### Example 5-Example Protocol for a Luciferase Based Potency Assay

An example potency assay for use in determining the potency of any given preparation of pMHC-nanoparticles is detailed in this example. The cells in this case comprise a luciferase gene under control of the NFAT promoter. Murine CD4 is expressed since JurMA cells are a human cell line, and the MHC component of the pMHC assayed in this example is the mouse I-A^{g7}. It is contemplated and shown in subsequent examples that the JurMA cell line works with human MHC as well as mouse (since JurMA cells display endogenous expression of human CD4).
1. In a 96-well Plate (in triplicate), add 500,000 BDC2.5/mCD4+ JurMA cells in 200 μL of Dulbecco's modified eagle's medium (DMEM) (Sigma-Aldrich, catalog #D6429-500ML), supplemented with 10% fetal bovine serum (FBS) (Sigma-Aldrich, Catalog #F6178) in the presence of either: 1) 20 ng/mL PMA (Sigma-Aldrich, catalog #P8139) plus 0.5 μM Ionomycin (Sigma-Aldrich, Catalog #I3909-1ML), 10 μg/mL of anti-hCD3ε mAb (OKT3, BD Biosciences) (as a positive control); 2)12.5 or 5.0 μg/mL or pMHC-coated on PF-M NP of various valencies ranging from 10-48 pMHCs/NP; or 3) cysteine coated NP (as negative control having equivalent iron concentration to pMHC-NP). Incubate overnight in a CO₂ incubator at 37° C. with 9% CO₂ supply. As a control replicate this setup with wild-type JurMA cells.
2. The next day, centrifuge the cells at 1200 rpm 5 mins and remove the medium. After this, add 200 μL PBS and wash the cells 3 times.
3. Resuspend the cell pellet in 100 μL of Lysis Buffer 1× (Cell Culture Lysis Reagent, Promega, Cat. #E1531) and incubate for 30 mins with gentle shaking.
4. Take out 20 μL of the lysate and transfer it to an opaque white 96-well plate (Greiner Bio-one Ref. #655075).
5. Add 100 μL of Luciferase Assay Reagent (Promega, Cat. #E1500) per well, then read it immediately by using a Veritas™ Microplate Luminometer (The injector of this instrument automatically adds 100 μL of Luciferase Assay Reagent per well. The plate is advanced to the next well for a repeat of the inject-then-read process). The light produced is measured for a period of 10 seconds (integration time). The delay time is 2 seconds.

This method is generally applicable to an assay for potency and activity of many different types of nanoparticle compositions, as is shown in the following examples.

### Example 6—Measuring Inter-Assay Variability

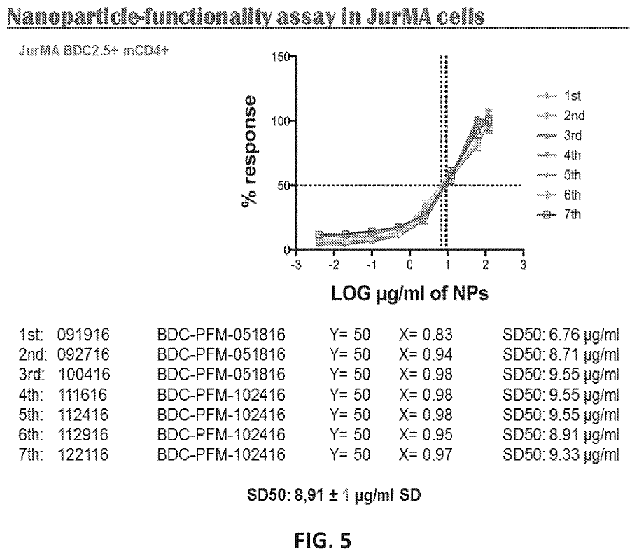

To measure inter-assay variability, Applicant prepared pMHC-NPs having the same specificity (i.e., the same pMHC complexes attached to the core) and assayed for SD50 (concentration that yields half-maximum activity, as measured in a semilog plot). These experiments utilized JurMA cells transfected with a recombinant TCR specific for GAD₅₂₄₋₅₄₃ bound to I-Ag7 (BDC 2.5mi) and a recombinant mouse CD4. The results are summarized in FIG. 5 and show that current data with 7 experiments is 8.91 plus/minus 1 microgram/mL (mean plus/minus standard deviation of the mean). The reproducibility tested and observed with Applicant's assay is unexpected because the reporter is not really a TCR proximal signaling event, and one of skill in the art would expect more assay to assay variability than shown. However, this data calculation shows tight responses and establishes that such a quantitative assay is much preferred than conventional but less quantitative or semi-quantitative assays (e.g., measurements of the intensity of phosphorylation of signaling intermediates upstream of the TCR signaling reporter). The advantage of quantitation, coupled with low inter-experimental variability and faithful reproduction of pMHC density thresholds responsible for biological activity, can provide excellent and highly sensitive batch-to-batch comparison of composition and quality of this disclosure.

### Example 7—Cell-Based Potency Assay to Assess Potential Effects of Anti-Navacim Antibodies on Navacim In Vitro T Cell Stimulatory Function

Post-in vivo delivery, the potential exists for an immunocompetent host to generate a humoral response against various components of pMHC-NPs. These include protein purification tags such as the 6×His tag present within the pMHC monomers coated on their surface, as well as PEG which is a structural component of the pMHC-NP. This example gauges whether antibodies directed against the various components of pMHC-NPs (pMHC, PEG, His tag) have an appreciable effect on the ability of pMHC-NP to engage and induce TCR signaling in T cells. Previous results have demonstrated the ability of human serum exposure to pMHC-NPs to block binding of anti-PEG (AGP4) and anti-His (6G2A9) antibodies to the particles. Therefore, this assay will test both human serum pre-exposed and non-exposed particles for their ability to stimulate cognate JurMA T cells after exposure to anti-His, anti-PEG, or anti-MHC monoclonal antibodies or rabbit hyperimmune serum.

### Reagents and Experimental Layout

Anti-pMHC mAbs
Purified anti-mouse/rat MHC Class II RT1B mAb (clone OX-6) (1 mg/mL in PBS, Bio-Rad Catalog #MCA46R)
Purified anti-PEG mAb (clone AGP4) (1.4 mg/mL in PBS, Anti-PEG, Catalog #AGP4-PABM-A)
Purified anti-His tag mAb (clone 6G2A9) (0.5 mg/mL in PBS, Genscript, Catalog #A00186)
Purified Mouse IgG (clone MOPC21) (0.5 mg/mL in PBS, BD Biosciences Catalog #554121)

### Serum

Anti-PEG hyperimmune rabbit serum
Anti-BDC2.5mi pMHC hyperimmune rabbit serum
Pre-immune rabbit serum
Human serum (Sigma, Cat #H4522)
   pMHC-NPs
BDC2.5mi-PFM-112017 (Fe: 2.15 mg/mL, pMHC: 0.97 mg/mL, Valency 42 pMHC/NP)
Cys-PFM-111417 (Fe: 1.52 mg/mL)

### Luciferase Detection Method:

Promega firefly luciferase assay kit with cell culture lysis buffer (Promega Catalog #E1500)
Spectramax i3x plate reading luminometer with reagent injectors
The potency assay was performed following Example 5-Example protocol for a luciferase based potency assay).

### Results

With or without human serum pre-exposure, as expected anti-MHC-II (anti-BDC2.5mi/IAg7) directed mAb or antisera were able to markedly inhibit Navacim activity in the in vitro potency assay in a titer-dependent manner. These treatments were included as positive inhibition controls to help validate the assay.

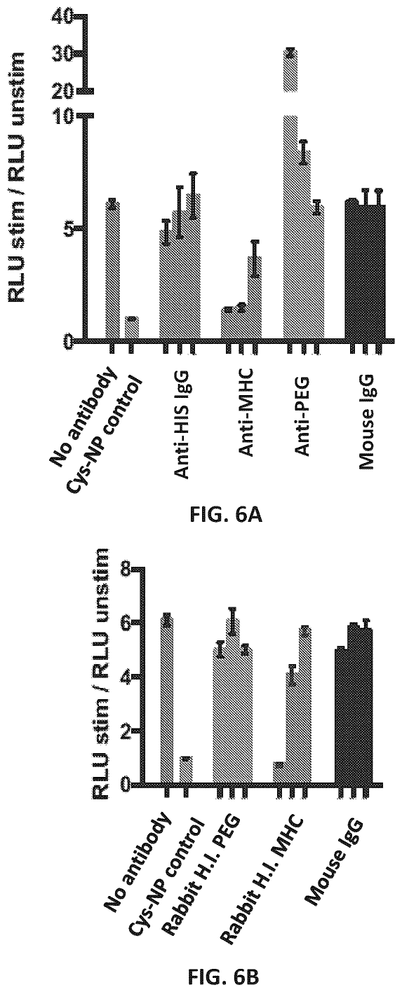

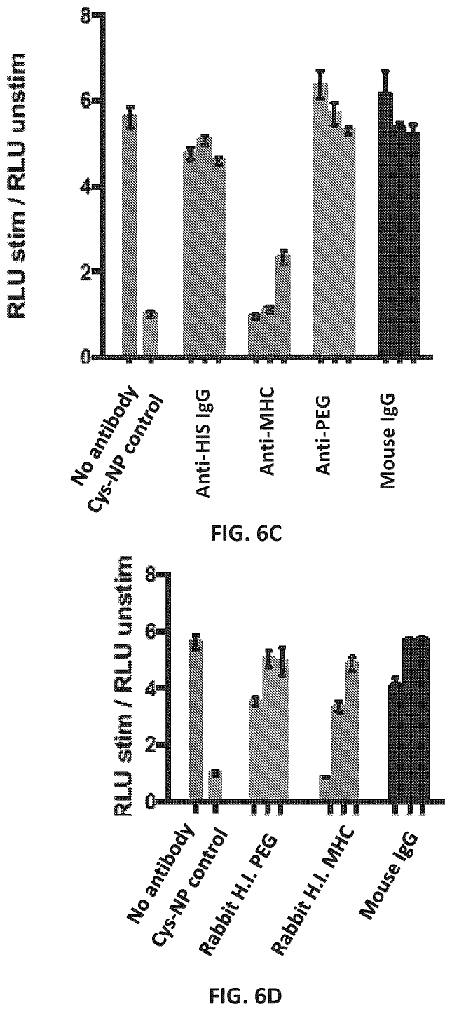

As shown in FIG. 6A to 6D, no inhibition of pMHC-NP activity was seen with anti-His tag, anti-PEG mAbs or rabbit anti-PEG hyperimmune sera compared with the negative controls (Mouse IgG or rabbit pre-immune serum), either with or without human serum pre-exposure. In the absence of human serum, pre-exposure, anti-PEG mAbs actually showed a strong potentiating effect on pMHC-NP T lymphocyte stimulation (possibly due to cross-linking by the pentameric structure of the pMHC-NP complex, as this is not seen with anti-PEG hyperimmune serum). This effect was blocked by pre-exposure of Navacims to human serum, consistent with our previous findings that serum exposure blocked anti-PEG antibody binding. Thus, exposure of Navacims to human serum does not reduce their potency in the JurMA assay

### Example 8-IGRP13-25/DR3 pMHC Heterodimers Bind to Engineered Cell Lines Expressing Cognate TCR

The ability of cys-trapped, zipperless, knob-in-hole IGRP₁₃₋₂₅ pMHC-DR3 heterodimers to bind a T-cell receptor was tested. For this, a reporter cell line expressing the alpha and beta chain from a human T-cell receptor specific for IGRP₁₃₋₂₅ pMHC-DR3 was used.

Transduction Protocol of the JURMA-hCD4 Cell Line with Retrovirus Encoding IGRP-TCR

Generation of the GP+EnvAM12 packaging cell line. We transfected 293T cells with a retrovirus expressing IGRP-TCR and a GFP reporter, along with gag/pol and VSV packaging constructs. Three days after VSV-pseudotyped enriched supernatants were harvested, aliquoted and frozen. These aliquots were used to transduce the amphotrophic packaging cell line GP+envAm12 (ATCC CRL-9641) by spin infection (2700 rpm 1 h). After 5 spin infections, transduced GP+envAm12 were sorted for expression of GFP if needed.

Transduction of JURMA-hCD4 Cell Line with Retrovirus Encoding IGRP-TCR

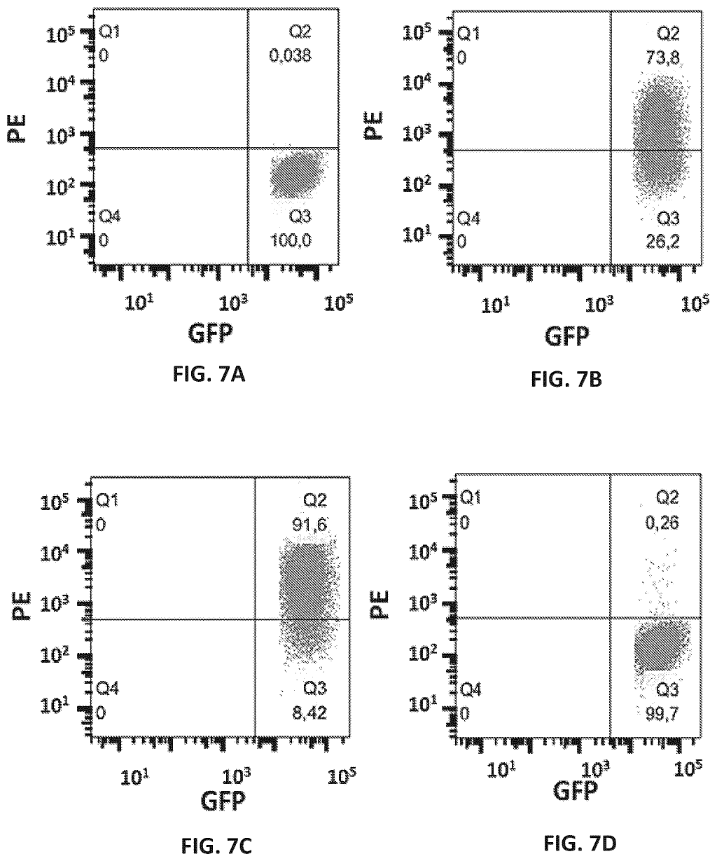

Three million transduced and sorted GP+envAm12 were plated per well of a 6 well plate in a final volume of 3 ml. Next day 100,000 JURMA-hCD4 were co-cultured with the pre-plated transduced GP+envAm12 in a final volume of 3 ml supplemented with 8 ug/ml of polybrene. This co-culture was maintained during two weeks changing the media every 2 or 3 days. After co-culture, JURMA-hCD4 cells were harvested analyzed by flow cytometry and sorted for high transgene expression. Cells were then stained with PE labeled heterodimers. FIG. 7A depicts unstained cells as a negative control, FIG. 7D depicts cells stained with irrelevant tetramer, FIG. 7B depicts staining with tetramers made from heterodimers expressed using cys-trap and leucine zipper technology, FIG. 7C depicts tetramers made from heterodimers expressed using cys, trap and knob-in-hole technology, without a leucine zipper. The staining between heterodimers made using either technology was robust. These data demonstrate that heterodimers made using a zipperless, cys-trapped knob-in hole technology are able to bind T cell receptor.

### Example 9—IGRP13-25/DR3 Knob-In-Hole pMHC Heterodimers Stimulate Reporter Cell Lines In Vitro

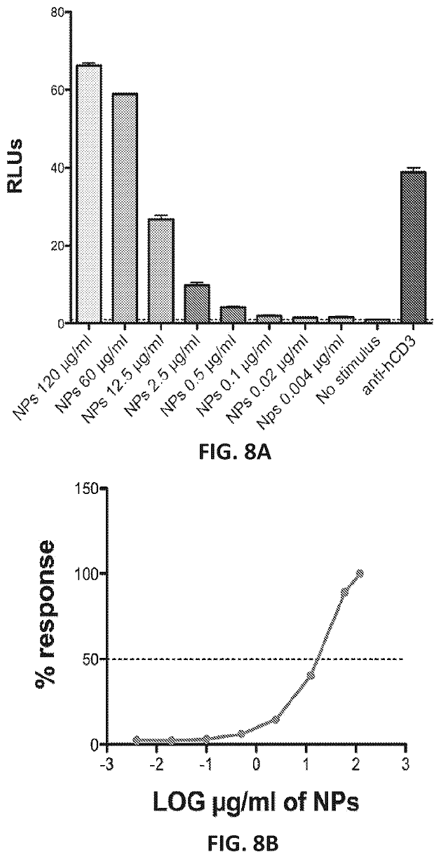

The ability of cys-trapped, knob-in-hole stabilized heterodimers when attached to iron oxide nanoparticles to stimulate T cell signaling was tested using JurMA cells expressing a human IGRP₁₃₋₂₅ TCR and luciferase under the control of the NFAT promoter. These results shown in FIG. 8A and FIG. 8B indicate that cys-trapped, knob-in-hole stabilized heterodimers, when attached to iron oxide nanoparticles, are capable of inducing T-cell signaling.

It should be understood that although the present disclosure has been specifically disclosed by certain embodiments and optional features, modification, improvement and variation of the disclosures embodied disclosed herein may be resorted to by those skilled in the art, and that such modifications, improvements and variations are considered to be within the scope of this disclosure. The materials, methods, and examples provided here are representative of certain embodiments, are exemplary, and are not intended as limitations on the scope of the disclosure.

The disclosure has been described broadly and generically herein. Each of the narrower species and subgeneric groupings falling within the generic disclosure also form part of the disclosure. This includes the generic description of the disclosure with a proviso or negative limitation removing any subject matter from the genus, regardless of whether or not the excised material is specifically recited herein.

In addition, where features or aspects of the disclosure are described in terms of Markush groups, those skilled in the art will recognize that the disclosure is also thereby described in terms of any individual member or subgroup of members of the Markush group.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Throughout this disclosure, various publications, patents and published patent specifications are referenced by an identifying citation. All publications, patent applications, patents, and other references mentioned herein are expressly incorporated by reference in their entirety, to the same extent as if each were incorporated by reference individually. In case of conflict, the present specification, including definitions, will control.

### EXEMPLARY SEQUENCE LISTINGS

TABLE 3
Ribosome skipping sequences

| | | | SEQ |
|---|---|---|---|
| Source | | Sequence | ID NO: |
| Picornaviruses | EMC-B-119 aa | GIFNAHYAGYFADLLIHDIETNPG | 456 |
| | EMC-D | GIFNAHYAGYFADLLIHDIETNPGP | 457 |
| | EMC-PV21 | RIFNAHYAGYFADLLIHDIETNPGP | 458 |
| | MENGO | HVFETHYAGYFSDLLIHDVETNPGP | 459 |
| | TME-GD7-109 aa- | KAVRGYHADYYKQRLIHDVEMNPGP | 460 |
| | TME-DA | RAVRAYHADYYKQRLIHDVEMNPGP | 461 |
| | TME-BEAN | KAVRGYHADYYRQRLIHDVETNPGP | 462 |
| | Theiler's-Like Virus | KHVREYHAAYYKQRLMHDVETNPGP | 463 |
| | Ljungan virus (174F) | MHSDEMDFAGGKFLNQCGDVETNPGP | 464 |
| | Ljungan virus (145SL) | MHNDEMDYSGGKFLNQCGDVESNPGP | 465 |
| | Ljungan virus (87-012) | MHSDEMDFAGGKFLNQCGDVETNPGP | 466 |
| | Ljungan virus (M1 146) | YHDKDMDYAGGKFLNQCGDVETNPGP | 467 |
| | FMD-A10 | LLNFDLLKLAGDVESNPGP | 468 |
| | FMD-A12 | LLNFDLLKLAGDVESNPGP | 469 |
| | FMD-C1 | LLNFDLLKLAGDVESNPGP | 470 |
| | FMD-OIG | LLNFDLLKLAGDMESNPGP | 471 |
| | FMD-OIK | LTNFDLLKLAGDVESNPGP | 472 |
| | FMD-0 (Taiwan) | LLNFDLLKLAGDVESNPGP | 473 |
| | FMD-O/SK | LLSFDLLKLAGDVESNPGP | 474 |
| | FMD-SAT3 | MCNFDLLKLAGDVESNPGP | 475 |
| | FMD-SAT2 | LLNFDLLKLAGDVESNPGP | 476 |
| | ERAV | CTNYSLLKLAGDVESNPGP | 477 |
| | ERBV | GATNFSLLKLAGDVELNPGP | 478 |
| | ERV-3 | GATNFDLLKLAGDVESNPGP | 479 |
| | PTV-1 | GPGATNFSLLKQAGDVEENPGP | 480 |
| | PTV-2 | GPGATNFSLLKQAGDVEENPGP | 481 |
| | PTV-3 | GPGASSFSLLKQAGDVEENPGP | 482 |
| | PTV-4 | GPGASNFSLLKQAGDVEENPGP | 483 |
| | PTV-5 | GPGAANFSLLRQAGDVEENPGP | 484 |
| | PTV-6 | GPGATNFSLLKQAGDVEENPGP | 485 |
| | PTV-7 | GPGATNFSLLKQAGDVEENPGP | 486 |
| | PTV-8 | GPGATNFSLLKQAGDIEENPGP | 487 |
| | PTV-9 | GPGATNFSLLKQAGDVEENPGP | 488 |
| | PTV-10 | GPGATNFSLLKQAGDVEENPGP | 489 |
| | PTV-11 | GPGATNFSLLKRAGDVEENPGP | 490 |
| Insect Viruses | CrPV | FLRKRTQLLMSGDVESNPGP | 491 |
| | DCV | EAARQMLLLLSGDVETNPGP | 492 |
| | ABPV | GSWTDILLLLSGDVETNPGP | 493 |
| | ABPV isolate Poland 1 | GSWTDILLLLSGDVETNPGP | 494 |
| | ABPV isolate Hungary 1 | GSWTDILLLWSGDVETNPGP | 495 |
| | IFV | TRAEIEDELIRAGIESNPGP | 496 |
| | TaV | RAEGRGSLLTCGDVEENPGP | 497 |
| | EEV | QGAGRGSLVTCGDVEENPGP | 498 |
| | APV | NYPMPEALQKIIDLESNPPP | 499 |
| | KBV | GTWESVLNLLAGDIELNPGP | 500 |
| | PnPV (a) | AQGWVPDLTVDGDVESNPGP | 501 |
| | PnPV (b) | IGGGQKDLTQDGDIESNPGP | 502 |
| Ectropis | (a) | AQGWAPDLTQDGDVESNPGP | 503 |
| obliqua | (b) | IGGGQRDLTQDGDIESNPGP | 504 |
| picorna-like | | | |
| virus | | | |
| Providence | (a) | VGDRGSLLTCGDVESNPGP | 505 |
| virus | (b) | SGGRGSLLTAGDVEKNPGP | 506 |
| | (c) | GDPIEDLTDDGDIEKNPGP | 507 |
| Type C | Bovine Rotavirus | SKFQIDRILISGDIELNPGP | 508 |
| Rotaviruses | Porcine Rotavirus | AKFQIDKILISGDVELNPGP | 509 |
| | Human Rotavirus | SKFQIDKILISGDIELNPGP | 510 |
| Reovirus | Bombyx mor | FRSNYDLLKLCGDIESNPGP | 511 |
| (cypovirus 1) | Lymantria dispar | FRSNYDLLKLCGDVESNPGP | 512 |
| | Dendrolimus punctatus | FRSNYDLLKLCGDVESNPGP | 513 |
| Tr pansoma | T. brucei TSR1 | SSIIRTKMLVSGDVEENPGP | 514 |
| spp. Repeated | (CAB95325.1) | SSIIRTKMLLSGDVEENPGP | 515 |
| Sequences | (CAB95342.1) | SSIIRTKMLLSGDVEENPGP | 516 |
| | (CAB95559.1) | SSIIRTKILLSGDVEENPGP | 517 |
| | T. cruzi AP | CDAQRQKLLLSGDIEQNPGP | 518 |
| | Endonuclease | | |
| Prokaryotic | T. maritima aguA | YIPDFGGFLVKADSEFNPGPX | 519 |
| Sequences | B. bronchiseptica | VHCAGRGGPVRLLDKEGNPGP | 520 |
| Eukaryotic | Mouse mor-1F | DLELETVGSHQADAETNPGPX | 521 |
| (cellular) | | | |
| Sequences: | | | |
| Eukaryotic | D. melanogaster | TAADKIQGSWKMDTEGNPGPX | 522 |
| (cellular) | mod(mdg4) | | |
| Sequences: | A. nidulans Ca | PITNRPRNSGLIDTEINPGP | 523 |
| | channel MIDI | | |

TABLE 4
IRES Sequences

| | | SEQ |
|---|---|---|
| Source | Sequence | ID NO: |
| EMCV IRES | CCCCTCTCCCTCCCCCCCCCCTAACGTTA | 524 |
| Sequence | CTGGCCGAAGCCGCTTGGAATAAGGCCG | |
| | GTGTGCGTTTGTCTATATGTTATTTTCCA | |
| | CCATATTGCCGTCTTTTGGCAATGTGAGG | |
| | GCCCGGAAACCTGGCCCTGTCTTCTTGA | |
| | CGAGCATTCCTAGGGGTCTTTCCCCTCTC | |
| | GCCAAAGGAATGCAAGGTCTGTTGAATG | |
| | TCGTGAAGGAAGCAGTTCCTCTGGAAGC | |
| | TTCTTGAAGACAAACAACGTCTGTAGCG | |
| | ACCCTTTGCAGGCAGCGGAACCCCCCAC | |
| | CTGGCGACAGGTGCCTCTGCGGCCAAAA | |
| | GCCACGTGTATAAGATACACCTGCAAAG | |
| | GCGGCACAACCCCAGTGCCACGTTGTGA | |
| | GTTGGATAGTTGTGGAAAGAGTCAAATG | |
| | GCTCTCCTCAAGCGTATTCAACAAGGGG | |
| | CTGAAGGATGCCCAGAAGGTACCCCATT | |
| | GTATGGGATCTGATCTGGGGCCTCGGTA | |
| | CACATGCTTTACATGTGTTTAGTCGAGGT | |
| | TAAAAAAACGTCTAGGCCCCCCGAACCA | |
| | CGGGGACGTGGTTTTCCTTTGAAAAACA | |
| | CGATGATAATATGGCCAC | |
| pBag1 IRES | CTGGGCGGTCAACAAGTGCGGGCCTGGC | 525 |
| Sequence | TCAGCGCGGGGGGGCGCGGAGACCGCG | |
| | AGGCGACCGGGAGCGGCTGGGTTCCCGG | |
| | CTGCGCGCCCTTCGGCCAGGCCGGGAGC | |
| | CGCGCCAGTCGGAGCCCCCGGCCCAGCG | |
| | TGGTCCGCCTCCCTCTGGGCGTCCACCTG | |
| | CCCGGAGTACTGCCAGCGGGCATGACCG | |
| | ACCCACCAGGGGCGCCGCCGCCGGCGCT | |
| | CGCAGGCCGCGGATGAAGAAGAAAACC | |
| | CGGCGCCGCTCGACCCGGAGCGAGGAGT | |
| | TGACCCGGAGCGAGGAGTTGACCCTGAG | |
| | TGAGGAAGCGACCTGGAGTGAAGAGGC | |
| | GACCCAGAGTGAGGAGGCGACCCAGGG | |
| | CGAAG | |
| Synthetic | AAAAGAAGGAAAAAGAAGGAAAAGAAG | 526 |
| IRES | GAAAAAGAAGGCTGCAGGCGGCTGCAG | |
| Sequence | AAAAGAAGGAAAAAGAAGGAAA | |
| | AGAAGGAAAA AGAAGG | |

TABLE 5
Polypeptide and polynucleotide sequences

| | | SEQ |
|---|---|---|
| Notes | Sequence | ID NO: |
| IGRP TCR | MAMLLGASVLILWLQPDWVNSQQKNDDQQVKQNSPSLSV | 527 |
| | QEGRISILNCDYTNSMFDYFLWYKKYPAEGPTFLISISSIKDK | |
| | NEDGRFTVFLNKSAKHLSLHIVPSQPGDSAVYFCAATNSGG | |
| | SNYKLTFGKGTLLTVNPNIQNPDPAVYQLRDSKSSDKSVCL | |
| | FTDFDSQTNVSQSKDSDVYITDKTVLDMRSMDFKSNSAVA | |
| | WSNKSDFACANAFNNSIIPEDTFFPSPESSCDVKLVEKSFETD | |
| | TNLNFQNLSVIGFRILLLKVAGFNLLMTLRLWSSGSGATNFS | |
| | LLKQAGDVEENPGPMSNQVLCCVVLCFLGANTVDGGITQSP | |
| | KYLFRKEGQNVTLSCEQNLNHDAMYWYRQDPGQGLRLIY | |
| | YSQIVNDFQKGDIAEGYSVSREKKESFPLTVTSAQKNPTAFY | |
| | LCASGGRVYQPQHFGDGTRLSILEDLNKVFPPEVAVFEPSEA | |
| | EISHTQKATLVCLATGFFPDHVELSWWVNGKEVHSGVSTDP | |
| | QPLKEQPALNDSRYCLSSRLRVSATFWQNPRNHFRCQVQFY | |
| | GLSENDEWTQDRAKPVTQIVSAEAWGRADCGFTSVSYQQG | |
| | VLSATILYEILLGKATLYAVLVSALVLMAMVKRKDF | |
| IGRPTCRa | MAMLLGASVLILWLQPDWVNSQQKNDDQQVKQNSPSLSV | 528 |
| | QEGRISILNCDYTNSMFDYFLWYKKYPAEGPTFLISISSIKDK | |
| | NEDGRFTVFLNKSAKHLSLHIVPSQPGDSAVYFCAATNSGG | |
| | SNYKLTFGKGTLLTVNPNIQNPDPAVYQLRDSKSSDKSVCL | |
| | FTDFDSQTNVSQSKDSDVYITDKTVLDMRSMDFKSNSAVA | |
| | WSNKSDFACANAFNNSIIPEDTFFPSPESSCDVKLVEKSFETD | |
| | TNLNFQNLSVIGFRILLLKVAGFNLLMTLRLWSS | |
| IGRP TCRb | MSNQVLCCVVLCFLGANTVDGGITQSPKYLFRKEGQNVTLS | 529 |
| | CEQNLNHDAMYWYRQDPGQGLRLIYYSQIVNDFQKGDIAE | |
| | GYSVSREKKESFPLTVTSAQKNPTAFYLCASGGRVYQPQHF | |
| | GDGTRLSILEDLNKVFPPEVAVFEPSEAEISHTQKATLVCLA | |
| | TGFFPDHVELSWWVNGKEVHSGVSTDPQPLKEQPALNDSR | |
| | YCLSSRLRVSATFWQNPRNHFRCQVQFYGLSENDEWTQDR | |
| | AKPVTQIVSAEAWGRADCGFTSVSYQQGVLSATILYEILLG | |
| | KATLYAVLVSALVLMAMVKRKDF | |
| IGRPTCRa | DQQVKQNSPSLSVQEGRISILNCDYTNSMFDYFLWYKKYPA | 530 |
| no signal | EGPTFLISISSIKDKNEDGRFTVFLNKSAKHLSLHIVPSQPGDS | |
| | AVYFCAATNSGGSNYKLTFGKGTLLTVNPNIQNPDPAVYQL | |
| | RDSKSSDKSVCLFTDFDSQTNVSQSKDSDVYITDKTVLDMR | |
| | SMDFKSNSAVAWSNKSDFACANAFNNSIIPEDTFFPSPESSC | |
| | DVKLVEKSFETDTNLNFQNLSVIGFRILLLKVAGFNLLMTLR | |
| | LWSS | |
| IGRP TCRb | DGGITQSPKYLFRKEGQNVTLSCEQNLNHDAMYWYRQDPG | 531 |
| no signal | QGLRLIYYSQIVNDFQKGDIAEGYSVSREKKESFPLTVTSAQ | |
| | KNPTAFYLCASGGRVYQPQHFGDGTRLSILEDLNKVFPPEV | |
| | AVFEPSEAEISHTQKATLVCLATGFFPDHVELSWWVNGKEV | |
| | HSGVSTDPQPLKEQPALNDSRYCLSSRLRVSATFWQNPRNH | |
| | FRCQVQFYGLSENDEWTQDRAKPVTQIVSAEAWGRADCGF | |
| | TSVSYQQGVLSATILYEILLGKATLYAVLVSALVLMAMVKR | |
| | KDF | |
| Poly- | ATGGCCATGCTCCTGGGGGCATCAGTGCTGATTCTGTGGC | 532 |
| nucleotide | TTCAGCCAGACTGGGTAAACAGTCAACAGAAGAATGATG | |
| sequence of | ACCAGCAAGTTAAGCAAAATTCACCATCCCTGAGCGTCC | |
| IGRP TCR | AGGAAGGAAGAATTTCTATTCTGAACTGTGACTATACTA | |
| | ACAGCATGTTTGATTATTTCCTATGGTACAAAAAATACCC | |
| | TGCTGAAGGTCCTACATTCCTGATATCTATAAGTTCCATT | |
| | AAGGATAAAAATGAAGATGGAAGATTCACTGTCTTCTTA | |
| | AACAAAAGTGCCAAGCACCTCTCTCTGCACATTGTGCCCT | |
| | CCCAGCCTGGAGACTCTGCAGTGTACTTCTGTGCAGCAAC | |
| | AAATAGTGGAGGTAGCAACTATAAACTGACATTTGGAAA | |
| | AGGAACTCTCTTAACCGTGAATCCAAATATCCAGAACCCT | |
| | GACCCTGCCGTGTACCAGCTGAGAGACTCTAAATCCAGT | |
| | GACAAGTCTGTCTGCCTATTCACCGATTTTGATTCTCAAA | |
| | CAAATGTGTCACAAAGTAAGGATTCTGATGTGTATATCAC | |
| | AGACAAAACTGTGCTAGACATGAGGTCTATGGACTTCAA | |
| | GAGCAACAGTGCTGTGGCCTGGAGCAACAAATCTGACTT | |
| | TGCATGTGCAAACGCCTTCAACAACAGCATTATTCCAGA | |
| | AGACACCTTCTTCCCCAGCCCAGAAAGTTCCTGTGATGTC | |
| | AAGCTGGTCGAGAAAAGCTTTGAAACAGATACGAACCTA | |
| | AACTTTCAAAACCTGTCAGTGATTGGGTTCCGAATCCTCC | |
| | TCCTGAAAGTGGCCGGGTTTAATCTGCTCATGACGCTGCG | |
| | GCTGTGGTCCAGCGGCTCCGGAGCCACGAACTTCTCTCTG | |
| | TTAAAGCAAGCAGGAGACGTGGAAGAAAACCCCGGTCCC | |
| | ATGAGCAACCAGGTGCTCTGCTGTGTGGTCCTTTGTTTCC | |
| | TGGGAGCAAACACCGTGGATGGTGGAATCACTCAGTCCC | |
| | CAAAGTACCTGTTCAGAAAGGAAGGACAGAATGTGACCC | |
| | TGAGTTGTGAACAGAATTTGAACCACGATGCCATGTACT | |
| | GGTACCGACAGGACCCAGGGCAAGGGCTGAGATTGATCT | |
| | ACTACTCACAGATAGTAAATGACTTTCAGAAAGGAGATA | |
| | TAGCTGAAGGGTACAGCGTCTCTCGGGAGAAGAAGGAAT | |
| | CCTTTCCTCTCACTGTGACATCGGCCCAAAAGAACCCGAC | |
| | AGCTTTCTATCTCTGTGCCAGTGGGGGACGGGTCTATCAG | |
| | CCCCAGCATTTTGGTGATGGGACTCGACTCTCCATCCTAG | |
| | AGGACCTGAACAAGGTGTTCCCACCCGAGGTCGCTGTGT | |
| | TTGAGCCATCAGAAGCAGAGATCTCCCACACCCAAAAGG | |
| | CCACACTGGTGTGCCTGGCCACAGGCTTCTTCCCTGACCA | |
| | CGTGGAGCTGAGCTGGTGGGTGAATGGGAAGGAGGTGCA | |
| | CAGTGGGGTCAGCACGGACCCGCAGCCCCTCAAGGAGCA | |
| | GCCCGCCCTCAATGACTCCAGATACTGCCTGAGCAGCCG | |
| | CCTGAGGGTCTCGGCCACCTTCTGGCAGAACCCCCGCAA | |
| | CCACTTCCGCTGTCAAGTCCAGTTCTACGGGCTCTCGGAG | |
| | AATGA | |
| IGRP TCR | MAMLLGASVLILWLQPDWVNSQQKNDDQQVKQNSPSLSV | 533 |
| polypeptide | QEGRISILNCDYTNSMFDYFLWYKKYPAEGPTFLISISSIKDK | |
| murinized | NEDGRFTVFLNKSAKHLSLHIVPSQPGDSAVYFCAATNSGG | |
| | SNYKLTFGKGTLLTVNPNIQNPEPAVYQLKDPRSQDSTLCLF | |
| | TDFDSQINVPKTMESGTFITDKTVLDMKAMDSKSNGAIAWS | |
| | NQTSFTCQDIFKETNATYPSSDVPCDATLTEKSFETDMNLNF | |
| | QNLSVMGLRILLLKVAGFNLLMTLRLWSSGSGATNFSLLKQ | |
| | AGDVEENPGPMSNQVLCCVVLCFLGANTVDGGITQSPKYLF | |
| | RKEGQNVTLSCEQNLNHDAMYWYRQDPGQGLRLIYYSQIV | |
| | NDFQKGDIAEGYSVSREKKESFPLTVTSAQKNPTAFYLCAS | |
| | GGRVYQPQHFGDGTRLSILEDLRNVTPPKVSLFEPSKAEIAN | |
| | KQKATLVCLARGFFPDHVELSWWVNGKEVHSGVSTDPQA | |
| | YKESNYSYCLSSRLRVSATFWHNPRNHFRCQVQFHGLSEED | |
| | KWPEGSPKPVTQNISAEAWGRADCGITSASYQQGVLSATIL | |
| | YEILLGKATLYAVLVSTLVVMAMVKRKNS | |
| IGRP | MAMLLGASVLILWLQPDWVNSQQKNDDQQVKQNSPSLSV | 534 |
| TCRa | QEGRISILNCDYTNSMFDYFLWYKKYPAEGPTFLISISSIKDK | |
| murinized | NEDGRFTVFLNKSAKHLSLHIVPSQPGDSAVYFCAATNSGG | |
| | SNYKLTFGKGTLLTVNPNIQNPEPAVYQLKDPRSQDSTLCLF | |
| | TDFDSQINVPKTMESGTFITDKTVLDMKAMDSKSNGAIAWS | |
| | NQTSFTCQDIFKETNATYPSSDVPCDATLTEKSFETDMNLNF | |
| | QNLSVMGLRILLLKVAGFNLLMTLRLWSS | |
| IGRP | MSNQVLCCVVLCFLGANTVDGGITQSPKYLFRKEGQNVTLS | 535 |
| TCRb | CEQNLNHDAMYWYRQDPGQGLRLIYYSQIVNDFQKGDIAE | |
| murinized | GYSVSREKKESFPLTVTSAQKNPTAFYLCASGGRVYQPQHF | |
| | GDGTRLSILEDLRNVTPPKVSLFEPSKAEIANKQKATLVCLA | |
| | RGFFPDHVELSWWVNGKEVHSGVSTDPQAYKESNYSYCLS | |
| | SRLRVSATFWHNPRNHFRCQVQFHGLSEEDKWPEGSPKPVT | |
| | QNISAEAWGRADCGITSASYQQGVLSATILYEILLGKATLYA | |
| | VLVSTLVVMAMVKRKNS | |
| IGRP | DQQVKQNSPSLSVQEGRISILNCDYTNSMFDYFLWYKKYPA | 536 |
| TCRa | EGPTFLISISSIKDKNEDGRFTVFLNKSAKHLSLHIVPSQPGDS | |
| murinized | AVYFCAATNSGGSNYKLTFGKGTLLTVNPNIQNPEPAVYQL | |
| no signal | KDPRSQDSTLCLFTDFDSQINVPKTMESGTFITDKTVLDMKA | |
| | MDSKSNGAIAWSNQTSFTCQDIFKETNATYPSSDVPCDATL | |
| | TEKSFETDMNLNFQNLSVMGLRILLLKVAGFNLLMTLRLWS | |
| | S | |
| IGRP | DGGITQSPKYLFRKEGQNVTLSCEQNLNHDAMYWYRQDPG | 537 |
| TCRb | QGLRLIYYSQIVNDFQKGDIAEGYSVSREKKESFPLTVTSAQ | |
| murinized | KNPTAFYLCASGGRVYQPQHFGDGTRLSILEDLRNVTPPKV | |
| no signal | SLFEPSKAEIANKQKATLVCLARGFFPDHVELSWWVNGKEV | |
| | HSGVSTDPQAYKESNYSYCLSSRLRVSATFWHNPRNHFRCQ | |
| | VQFHGLSEEDKWPEGSPKPVTQNISAEAWGRADCGITSASY | |
| | QQGVLSATILYEILLGKATLYAVLVSTLVVMAMVKRKNS | |
| PPI TCR | METLLGVSLVILWLQLARVNSQQGEEDPQALSIQEGENATM | 538 |
| polypeptide | NCSYKTSINNLQWYRQNSGRGLVHLILIRSNEREKHSGRLR | |
| | VTLDTSKKSSSLLITASRAADTASYFCATGRMDSSYKLIFGS | |
| | GTRLLVRPDIQNPDPAVYQLRDSKSSDKSVCLFTDFDSQTN | |
| | VSQSKDSDVYITDKTVLDMRSMDFKSNSAVAWSNKSDFAC | |
| | ANAFNNSIIPEDTFFPSPESSCDVKLVEKSFETDTNLNFQNLS | |
| | VIGFRILLLKVAGFNLLMTLRLWSSGSGATNFSLLKQAGDV | |
| | EENPGPMDSWTFCCVSLCILVAKHTDAGVIQSPRHEVTEMG | |
| | QEVTLRCKPISGHNSLFWYRQTMMRGLELLIYFNNNVPIDD | |
| | SGMPEDRFSAKMPNASFSTLKIQPSEPRDSAVYFCASSEQLS | |
| | GNTIYFGEGSWLTVVEDLNKVFPPEVAVFEPSEAEISHTQKA | |
| | TLVCLATGFFPDHVELSWWVNGKEVHSGVSTDPQPLKEQP | |
| | ALNDSRYCLSSRLRVSATFWQNPRNHFRCQVQFYGLSENDE | |
| | WTQDRAKPVTQIVSAEAWGRADCGFTSVSYQQGVLSATIL | |
| | YEILLGKATLYAVLVSALVLMAMVKRKDF | |
| PPI TCRa | METLLGVSLVILWLQLARVNSQQGEEDPQALSIQEGENATM | 539 |
| | NCSYKTSINNLQWYRQNSGRGLVHLILIRSNEREKHSGRLR | |
| | VTLDTSKKSSSLLITASRAADTASYFCATGRMDSSYKLIFGS | |
| | GTRLLVRPDIQNPDPAVYQLRDSKSSDKSVCLFTDFDSQTN | |
| | VSQSKDSDVYITDKTVLDMRSMDFKSNSAVAWSNKSDFAC | |
| | ANAFNNSIIPEDTFFPSPESSCDVKLVEKSFETDTNLNFQNLS | |
| | VIGFRILLLKVAGFNLLMTLRLWSS | |
| PPI TCRb | MDSWTFCCVSLCILVAKHTDAGVIQSPRHEVTEMGQEVTLR | 540 |
| | CKPISGHNSLFWYRQTMMRGLELLIYFNNNVPIDDSGMPED | |
| | RFSAKMPNASFSTLKIQPSEPRDSAVYFCASSEQLSGNTIYFG | |
| | EGSWLTVVEDLNKVFPPEVAVFEPSEAEISHTQKATLVCLAT | |
| | GFFPDHVELSWWVNGKEVHSGVSTDPQPLKEQPALNDSRY | |
| | CLSSRLRVSATFWQNPRNHFRCQVQFYGLSENDEWTQDRA | |
| | KPVTQIVSAEAWGRADCGFTSVSYQQGVLSATILYEILLGK | |
| | ATLYAVLVSALVLMAMVKRKDF | |
| PPI TCRa | QQGEEDPQALSIQEGENATMNCSYKTSINNLQWYRQNSGR | 541 |
| no signal | GLVHLILIRSNEREKHSGRLRVTLDTSKKSSSLLITASRAADT | |
| | ASYFCATGRMDSSYKLIFGSGTRLLVRPDIQNPDPAVYQLR | |
| | DSKSSDKSVCLFTDFDSQTNVSQSKDSDVYITDKTVLDMRS | |
| | MDFKSNSAVAWSNKSDFACANAFNNSIIPEDTFFPSPESSCD | |
| | VKLVEKSFETDTNLNFQNLSVIGFRILLLKVAGFNLLMTLRL | |
| | WSS | |
| PPI TCRb | GVIQSPRHEVTEMGQEVTLRCKPISGHNSLFWYRQTMMRG | 542 |
| no signal | LELLIYFNNNVPIDDSGMPEDRFSAKMPNASFSTLKIQPSEPR | |
| | DSAVYFCASSEQLSGNTIYFGEGSWLTVVEDLNKVFPPEVA | |
| | VFEPSEAEISHTQKATLVCLATGFFPDHVELSWWVNGKEVH | |
| | SGVSTDPQPLKEQPALNDSRYCLSSRLRVSATFWQNPRNHF | |
| | RCQVQFYGLSENDEWTQDRAKPVTQIVSAEAWGRADCGFT | |
| | SVSYQQGVLSATILYEILLGKATLYAVLVSALVLMAMVKRK | |
| | DF | |
| BDc2.5 | MHSLHVSLVFLWLQLGGVSSQEKVQQSPESLTVPEGAMAS | 543 |
| TCR | LNCTISDSASQSIWWYQQNPGKGPKALISIFSNGNKKEGRLT | |
| | VYLNRASLHVSLHIRDSHPSDSAVYLCAASLAGSWQLIFGS | |
| | GTQLTVMPDIQNPEPAVYQLKDPRSQDSTLCLFTDFDSQINV | |
| | PKTMESGTFITDKTVLDMKAMDSKSNGAIAWSNQTSFTCQ | |
| | DIFKETNATYPSSDVPCDATLTEKSFETDMNLNFQNLSVMG | |
| | LRILLLKVAGFNLLMTLRLWSSATNFSLLKQAGDVEENPGP | |
| | MGSIFLSCLAVCLLVAGPVDPKIIQKPKYLVAVTGSEKILICE | |
| | QYLGHNAMYWYRQSAKKPLEFMFSYSYQKLMDNQTASSR | |
| | FQPQSSKKNHLDLQITALKPDDSATYFCASSQGGTTNSDYTF | |
| | GSGTRLLVIEDLRNVTPPKVSLFEPSKAEIANKQKATLVCLA | |
| | RGFFPDHVELSWWVNGKEVHSGVSTDPQAYKESNYSYCLS | |
| | SRLRVSATFWHNPRNHFRCQVQFHGLSEEDKWPEGSPKPVT | |
| | QNISAEAWGRADCGITSASYHQGVLSATILYEILLGKATLYA | |
| | VLVSGLVLMAMVKRKNS | |
| BDc2.5 | MHSLHVSLVFLWLQLGGVSSQEKVQQSPESLTVPEGAMAS | 544 |
| TCRa | LNCTISDSASQSIWWYQQNPGKGPKALISIFSNGNKKEGRLT | |
| | VYLNRASLHVSLHIRDSHPSDSAVYLCAASLAGSWQLIFGS | |
| | GTQLTVMPDIQNPEPAVYQLKDPRSQDSTLCLFTDFDSQINV | |
| | PKTMESGTFITDKTVLDMKAMDSKSNGAIAWSNQTSFTCQ | |
| | DIFKETNATYPSSDVPCDATLTEKSFETDMNLNFQNLSVMG | |
| | LRILLLKVAGFNLLMTLRLWSS | |
| BDC2.5 | MGSIFLSCLAVCLLVAGPVDPKIIQKPKYLVAVTGSEKILICE | 545 |
| TCRb | QYLGHNAMYWYRQSAKKPLEFMFSYSYQKLMDNQTASSR | |
| | FQPQSSKKNHLDLQITALKPDDSATYFCASSQGGTTNSDYTF | |
| | GSGTRLLVIEDLRNVTPPKVSLFEPSKAEIANKQKATLVCLA | |
| | RGFFPDHVELSWWVNGKEVHSGVSTDPQAYKESNYSYCLS | |
| | SRLRVSATFWHNPRNHFRCQVQFHGLSEEDKWPEGSPKPVT | |
| | QNISAEAWGRADCGITSASYHQGVLSATILYEILLGKATLYA | |
| | VLVSGLVLMAMVKRKNS | |
| BDc2.5 | QSPESLTVPEGAMASLNCTISDSASQSIWWYQQNPGKGPKA | 546 |
| TCRa no | LISIFSNGNKKEGRLTVYLNRASLHVSLHIRDSHPSDSAVYL | |
| signal | CAASLAGSWQLIFGSGTQLTVMPDIQNPEPAVYQLKDPRSQ | |
| | DSTLCLFTDFDSQINVPKTMESGTFITDKTVLDMKAMDSKS | |
| | NGAIAWSNQTSFTCQDIFKETNATYPSSDVPCDATLTEKSFE | |
| | TDMNLNFQNLSVMGLRILLLKVAGFNLLMTLRLWSS | |
| BDC2.5 | KIIQKPKYLVAVTGSEKILICEQYLGHNAMYWYRQSAKKPL | 547 |
| TCRb no | EFMFSYSYQKLMDNQTASSRFQPQSSKKNHLDLQITALKPD | |
| signal | DSATYFCASSQGGTTNSDYTFGSGTRLLVIEDLRNVTPPKVS | |
| | LFEPSKAEIANKQKATLVCLARGFFPDHVELSWWVNGKEV | |
| | HSGVSTDPQAYKESNYSYCLSSRLRVSATFWHNPRNHFRCQ | |
| | VQFHGLSEEDKWPEGSPKPVTQNISAEAWGRADCGITSASY | |
| | HQGVLSATILYEILLGKATLYAVLVSGLVLMAMVKRKNS | |
| poly- | ATGCATTCCTTACATGTTTCACTAGTGTTCCTCTGGCTTCA | 548 |
| nucleotide | ACTAGGTGGGGTGAGCAGCCAGGAGAAGGTACAGCAGA | |
| sequence of | GCCCAGAATCTCTCACAGTCCCAGAGGGAGCCATGGCCT | |
| BDC2.5mi- | CCCTCAACTGCACTATCAGCGACAGTGCTTCTCAGTCCAT | |
| TCRalpha_ | CTGGTGGTACCAACAGAATCCTGGGAAAGGCCCCAAAGC | |
| P2A- | ACTAATATCCATATTCTCTAATGGCAACAAGAAAGAAGG | |
| TCRbeta | CAGATTGACAGTTTACCTCAATAGAGCCAGCCTGCATGTT | |
| | TCCCTGCACATCAGAGACTCCCATCCCAGTGACTCCGCCG | |
| | TCTACCTCTGTGCAGCGAGCCTTGCGGGCAGCTGGCAACT | |
| | CATCTTTGGATCTGGAACCCAACTGACAGTTATGCCTGAC | |
| | ATCCAGAACCCAGAACCTGCTGTGTACCAGTTAAAAGAT | |
| | CCTCGGTCTCAGGACAGCACCCTCTGCCTGTTCACCGACT | |
| | TTGACTCCCAAATCAATGTGCCGAAAACCATGGAATCTG | |
| | GAACGTTCATCACTGACAAAACTGTGCTGGACATGAAAG | |
| | CTATGGATTCCAAGAGCAATGGGGCCATTGCCTGGAGCA | |
| | ACCAGACAAGCTTCACCTGCCAAGATATCTTCAAAGAGA | |
| | CCAACGCCACCTACCCCAGTTCAGACGTTCCCTGTGATGC | |
| | CACGTTGACCGAGAAAAGCTTTGAAACAGATATGAACCT | |
| | AAACTTTCAAAACCTGTCAGTTATGGGACTCCGAATCCTC | |
| | CTGCTGAAAGTAGCCGGATTTAACCTGCTCATGACGCTGA | |
| | GGCTGTGGTCCAGTGCCACGAACTTCTCTCTGTTAAAGCA | |
| | AGCAGGAGACGTGGAAGAAAACCCCGGTCCCATGGGCTC | |
| | CATTTTCCTCAGTTGCCTGGCCGTTTGTCTCCTGGTGGCA | |
| | GGTCCAGTCGACCCGAAAATTATCCAGAAACCAAAATAT | |
| | CTGGTGGCAGTCACAGGGAGCGAAAAAATCCTGATATGC | |
| | GAACAGTATCTAGGCCACAATGCTATGTATTGGTATAGA | |
| | CAAAGTGCTAAGAAGCCTCTAGAGTTCATGTTTTCCTACA | |
| | GCTATCAAAAACTTATGGACAATCAGACTGCCTCAAGTC | |
| | GCTTCCAACCTCAAAGTTCAAAGAAAAACCATTTAGACC | |
| | TTCAGATCACAGCTCTAAAGCCTGATGACTCGGCCACATA | |
| | CTTCTGTGCCAGCAGCCAAGGGGGGACAACAAACTCCGA | |
| | CTACACCTTCGGCTCAGGGACCAGGCTTTTGGTAATAGAG | |
| | GATCTGAGAAATGTGACTCCACCCAAGGTCTCCTTGTTTG | |
| | AGCCATCAAAAGCAGAGATTGCAAACAAACAAAAGGCT | |
| | ACCCTCGTGTGCTTGGCCAGGGGCTTCTTCCCTGACCACG | |
| | TGGAGCTGAGCTGGTGGGTGAATGGCAAGGAGGTCCACA | |
| | GTGGGGTCAGCACGGACCCTCAGGCCTACAAGGAGAGCA | |
| | ATTATAGCTACTGCCTGAGCAGCCGCCTGAGGGTCTCTGC | |
| | TACCTTCTGGCACAATCCTCGCAACCACTTCCGCTGCCAA | |
| | GTGCAGTTCCATGGGCTTTCAGAGGAGGACAAGTGGCCA | |
| | GAGGGCTCACCCAAACCTGTCACACAGAACATCAGTGCA | |
| | GAGGCCTGGGGCCGAGCAGACTGTGGAATCACTTCAGCA | |
| | TCCTATCATCAGGGGGTTCTGTCTGCAACCATCCTCTATG | |
| | AGATCCTACTGGGGAAGGCCACCCTATATGCTGTGCTGGT | |
| | CAGTGGCCTGGTGCTGATGGCCATGGTCAAGAGAAAAAA | |
| | TTCCTGA | |
| Human | MNRGVPFRHLLLVLQLALLPAATQGKKVVLGKKGDTVELT | 549 |
| CD4 | CTASQKKSIQFHWKNSNQIKILGNQGSFLTKGPSKLNDRADS | |
| polypeptide | RRSLWDQGNFPLIIKNLKIEDSDTYICEVEDQKEEVQLLVFG | |
| sequence | LTANSGTHLLQGQSLTLTLESPPGSSPSVQCRSPRGKNIQGG | |
| | KTLSVSQLELQDSGTWTCTVLQNQKKVEFKIDIVVLAFQKA | |
| | SSIVYKKEGEQVEFSFPLAFTVEKLTGSGELWWQAERASSS | |
| | KSWITFDLKNKEVSVKRVTQDPKLQMGKKLPLHLTLPQALP | |
| | QYAGSGNLTLALEAKTGKLHQEVNLVVMRATQLQKNLTCE | |
| | VWGPTSPKLMLSLKLENKEAKVSKREKAVWVLNPEAGMW | |
| | QCLLSDSGQVLLESNIKVLPTWSTPVQPMALIVLGGVAGLL | |
| | LFIGLGIFFCVRCRHRRRQAERMSQIKRLLSEKKTCQCPHRF | |
| | QKTCSPI | |
| Human | ATGAACCGGGGAGTCCCTTTTAGGCACTTGCTTCTGGTGC | 550 |
| CD4 | TGCAACTGGCGCTCCTCCCAGCAGCCACTCAGGGAAAGA | |
| poly- | AAGTGGTGCTGGGCAAAAAAGGGGATACAGTGGAACTG | |
| nucleotide | ACCTGTACAGCTTCCCAGAAGAAGAGCATACAATTCCAC | |
| sequence | TGGAAAAACTCCAACCAGATAAAGATTCTGGGAAATCAG | |
| | GGCTCCTTCTTAACTAAAGGTCCATCCAAGCTGAATGATC | |
| | GCGCTGACTCAAGAAGAAGCCTTTGGGACCAAGGAAACT | |
| | TTCCCCTGATCATCAAGAATCTTAAGATAGAAGACTCAG | |
| | ATACTTACATCTGTGAAGTGGAGGACCAGAAGGAGGAGG | |
| | TGCAATTGCTAGTGTTCGGATTGACTGCCAACTCTGGTAC | |
| | CCACCTGCTTCAGGGGCAGAGCCTGACCCTGACCTTGGA | |
| | GAGCCCCCCTGGTAGTAGCCCCTCAGTGCAATGTAGGAG | |
| | TCCAAGGGGTAAAAACATACAGGGGGGGAAGACCCTCTC | |
| | CGTGTCTCAGCTGGAGCTCCAGGATAGTGGCACCTGGAC | |
| | GTGCACTGTCTTGCAGAACCAGAAGAAGGTGGAGTTCAA | |
| | AATAGACATCGTGGTGCTAGCTTTCCAGAAGGCCTCCAG | |
| | CATAGTCTATAAGAAAGAGGGGGAACAGGTGGAGTTCTC | |
| | CTTCCCACTCGCCTTTACAGTTGAAAAGCTGACGGGCAGT | |
| | GGCGAGCTGTGGTGGCAGGCGGAGAGGGCTTCCTCCTCC | |
| | AAGTCTTGGATCACCTTTGACCTGAAGAACAAGGAAGTG | |
| | TCTGTAAAACGGGTTACCCAGGACCCTAAGCTCCAGATG | |
| | GGCAAGAAGCTCCCGCTCCACCTCACCCTGCCCCAGGCC | |
| | TTGCCTCAGTATGCTGGCTCTGGAAACCTCACCCTGGCCC | |
| | TTGAAGCGAAAACAGGAAAGTTGCATCAGGAAGTGAACC | |
| | TGGTGGTGATGAGAGCCACTCAGCTCCAGAAAAATTTGA | |
| | CCTGTGAGGTGTGGGGACCCACCTCCCCTAAGCTGATGCT | |
| | GAGCTTGAAACTGGAGAACAAGGAGGCAAAGGTCTCGA | |
| | AGCGGGAGAAGGCGGTGTGGGTGCTGAACCCTGAGGCGG | |
| | GGATGTGGCAGTGTCTGCTGAGTGACTCGGGACAGGTCC | |
| | TGCTGGAATCCAACATCAAGGTTCTGCCCACATGGTCCAC | |
| | CCCGGTGCAGCCAATGGCCCTGATTGTGCTGGGGGGCGT | |
| | CGCCGGCCTCCTGCTTTTCATTGGGCTAGGCATCTTCTTCT | |
| | GTGTCAGGTGCCGGCACCGAAGGCGCCAAGCAGAGCGGA | |
| | TGTCTCAGATCAAGAGACTCCTCAGTGAGAAGAAGACCT | |
| | GCCAGTGCCCTCACCGGTTTCAGAAGACATGTAGCCCCAT | |
| | TTGAGTCGACAAGGGCGAATTAATTCAGATCTTACGTAG | |
| | CTAGCGGATCCCAATTGCTCGAGCGGGATCAATTCCGCCC | |
| | CCCCCCTAACGTTACTGGCCGAAGCCGCTTGGAATAAGG | |
| | CCGGTGTGCGTTTGTCTATATGTTATTTTCCACCATATTGC | |
| | CGTCTTTTGGCAATGTGAGGGCCCGGAAACCTGGCCCTGT | |
| | CTTCTTGACGAGCATTCCTAGGGGTCTTTCCCCTCTCGCC | |
| | AAAGGAATGCAAGGTCTGTTGAATGTCGTGAAGGAAGCA | |
| | GTTCCTCTGG | |
| Mouse | MCRAISLRRLLLLLLQLSQLLAVTQGKTLVLGKEGESAELPC | 551 |
| CD4 | ESSQKKITVFTWKFSDQRKILGQHGKGVLIRGGSPSQFDRFD | |
| polypeptide | SKKGAWEKGSFPLIINKLKMEDSQTYICELENRKEEVELWV | |
| sequence | FKVTFSPGTSLLQGQSLTLTLDSNSKVSNPLTECKHKKGKV | |
| | VSGSKVLSMSNLRVQDSDFWNCTVTLDQKKNWFGMTLSV | |
| | LGFQSTAITAYKSEGESAEFSFPLNFAEENGWGELMWKAEK | |
| | DSFFQPWISFSIKNKEVSVQKSTKDLKLQLKETLPLTLKIPQV | |
| | SLQFAGSGNLTLTLDKGTLHQEVNLVVMKVAQLNNTLTCE | |
| | VMGPTSPKMRLTLKQENQEARVSEEQKVVQVVAPETGLW | |
| | QCLLSEGDKVKMDSRIQVLSRGVNQTVFLACVLGGSFGFLG | |
| | FLGLCILCCVRCRHQQRQAARMSQIKRLLSEKKTCQCPHRM | |
| | QKSHNLI | |
| Mouse | ATGTGCCGAGCCATCTCTCTTAGGCGCTTGCTGCTGCTGC | 552 |
| CD4 | TGCTGCAGCTGTCACAACTCCTAGCTGTCACTCAAGGGAA | |
| poly- | GACGCTGGTGCTGGGGAAGGAAGGGGAATCAGCAGAAC | |
| nucleotide | TGCCCTGCGAGAGTTCCCAGAAGAAGATCACAGTCTTCA | |
| sequence | CCTGGAAGTTCTCTGACCAGAGGAAGATTCTGGGGCAGC | |
| | ATGGCAAAGGTGTATTAATTAGAGGAGGTTCGCCTTCGC | |
| | AGTTTGATCGTTTTGATTCCAAAAAAGGGGCATGGGAGA | |
| | AAGGATCGTTTCCTCTCATCATCAATAAACTTAAGATGGA | |
| | AGACTCTCAGACTTATATCTGTGAGCTGGAGAACAGGAA | |
| | AGAGGAGGTGGAGTTGTGGGTGTTCAAAGTGACCTTCAG | |
| | TCCGGGTACCAGCCTGTTGCAAGGGCAGAGCCTGACCCT | |
| | GACCTTGGATAGCAACTCTAAGGTCTCTAACCCCTTGACA | |
| | GAGTGCAAACACAAAAAGGGTAAAGTTGTCAGTGGTTCC | |
| | AAAGTTCTCTCCATGTCCAACCTAAGGGTTCAGGACAGC | |
| | GACTTCTGGAACTGCACCGTGACCCTGGACCAGAAAAAG | |
| | AACTGGTTCGGCATGACACTCTCAGTGCTGGGTTTTCAGA | |
| | GCACAGCTATCACGGCCTATAAGAGTGAGGGAGAGTCAG | |
| | CGGAGTTCTCCTTCCCACTCAACTTTGCAGAGGAAAACGG | |
| | GTGGGGAGAGCTGATGTGGAAGGCAGAGAAGGATTCTTT | |
| | CTTCCAGCCCTGGATCTCCTTCTCCATAAAGAACAAAGAG | |
| | GTGTCCGTACAAAAGTCCACCAAAGACCTCAAGCTCCAG | |
| | CTGAAGGAAACGCTCCCACTCACCCTCAAGATACCCCAG | |
| | GTCTCGCTTCAGTTTGCTGGTTCTGGCAACCTGACTCTGA | |
| | CTCTGGACAAAGGGACACTGCATCAGGAAGTGAACCTGG | |
| | TGGTGATGAAAGTGGCTCAGCTCAACAATACTTTGACCTG | |
| | TGAGGTGATGGGACCTACCTCTCCCAAGATGAGACTGAC | |
| | CCTGAAGCAGGAGAACCAGGAGGCCAGGGTCTCTGAGGA | |
| | GCAGAAAGTAGTTCAAGTGGTGGCCCCTGAGACAGGGCT | |
| | GTGGCAGTGTCTACTGAGTGAAGGTGATAAGGTCAAGAT | |
| | GGACTCCAGGATCCAGGTTTTATCCAGAGGGGTGAACCA | |
| | GACAGTGTTCCTGGCTTGCGTGCTGGGTGGCTCCTTCGGC | |
| | TTTCTGGGTTTCCTTGGGCTCTGCATCCTCTGCTGTGTCAG | |
| | GTGCCGGCACCAACAGCGCCAGGCAGCACGAATGTCTCA | |
| | GATCAAGAGGCTCCTCAGTGAGAAGAAGACCTGCCAGTG | |
| | CCCCCACCGGATGCAGAAGAGCCATAATCTCATCTGAAG | |
| | CGGCCGCGTCGACTCGAGCGGGATCAATTCCGCCCCCCC | |
| | CCTAACGTTACTGGCCGAAGCCGCTTGGAATAAGGCCGG | |
| | TGTGCGTTTGTCTATATGTTATTTTCCACCATATTGCCGTC | |
| | TTTTGGCAATGTGAGGGCCCGGAAACCTGGCCCTGTCTTC | |
| | TTGACGAGCATTCCTAGGGGTCTTTCCCCTCTCGCCAAAG | |
| | GAATGCAAGGTCTGTTGAATGTCGTGAAGGAAGCAGTTC | |
| | CTCTGG | |
| Polypeptide | MEHSGILASLILIAVLPQGSPFKIQVTEYEDKVFVTCNTSVM | 553 |
| sequence of | HLDGTVEGWFAKNKTLNLGKGVLDPRGIYLCNGTEQLAKV | |
| murine | VSSVQVHYRMCQNCVELDSGTMAGVIFIDLIATLLLALGIYC | |
| CD3delta- | FAGHETGRPSGAAEVQALLKNEQLYQPLRDREDTQYSRLG | |
| F2A- | GNWPRNKKSGPVKQTLNFDLLKLAGDVESNPGPMEQRKGL | |
| gamma- | AGLFLVISLLQGTVAQTNKAKNLVQVDGSRGDGSVLLTCGL | |
| T2A- | TDKTIKWLKDGSIISPLNATKNTWNLGNNAKDPRGTYQCQG | |
| epsilon- | AKETSNPLQVYYRMCENCIELNIGTISGFIFAEVISIFFLALGV | |
| P2A-zeta: | YLIAGQDGVRQSRASDKQTLLQNEQLYQPLKDREYDQYSH | |
| | LQGNQLRKKRSEGRGSLLTCGDVEENPGPMRWNTFWGILC | |
| | LSLLAVGTCQDDAENIEYKVSISGTSVELTCPLDSDENLKWE | |
| | KNGQELPQKHDKHLVLQDFSEVEDSGYYVCYTPASNKNTY | |
| | LYLKARVCEYCVEVDLTAVAIIIIVDICITLGLLMVIYWSKN | |
| | RKAKAKPVTRGTGAGSRPRGQNKERPPPVPNPDYEPIRKGQ | |
| | RDLYSGLNQRAVGSATNFSLLKQAGDVEENPGPMKWKVSV | |
| | LACILHVRFPGAEAQSFGLLDPKLCYLLDGILFIYGVIITALY | |
| | LRAKFSRSAETAANLQDPNQLYNELNLGRREEYDVLEKKR | |
| | ARDPEMGGKQQRRRNPQEGVYNALQKDKMAEAYSEIGTK | |
| | GERRRGKGHDGLYQGLSTATKDTYDALHMQTLAPR | |
| Poly- | ATGGAACACAGCGGGATTCTGGCTAGTCTGATACTGATTGCTG | 554 |
| nucleotide | TTCTCCCCCAAGGGAGCCCCTTCAAGATACAAGTGACCGAATA | |
| sequence of | TGAGGACAAAGTATTTGTGACCTGCAATACCAGCGTCATGCAT | |
| murine | CTAGATGGAACGGTGGAAGGATGGTTTGCAAAGAATAAAACA | |
| CD3delta- | CTCAACTTGGGCAAAGGCGTTCTGGACCCACGAGGGATATATC | |
| F2A-gamma- | TGTGTAATGGGACAGAGCAGCTGGCAAAGGTGGTGTCTTCTGT | |
| T2A- | GCAAGTCCATTACCGAATGTGCCAGAACTGTGTGGAGCTAGAC | |
| epsilon- | TCGGGCACCATGGCTGGTGTCATCTTCATTGACCTCATCGCAAC | |
| P2A-zeta: | TCTGCTCCTGGCTTTGGGCATCTACTGCTTTGCAGGACATGAGA | |
| | CCGGAAGGCCTTCTGGGGCTGCTGAGGTTCAAGCACTGCTGAA | |
| | GAATGAGCAGCTGTATCAGCCTCTTCGAGATCGTGAAGATACC | |
| | CAGTACAGCCGTCTTGGAGGGAACTGGCCCCGGAACAAGAAA | |
| | TCCGGACCGGTGAAACAGACTTTGAATTTTGACCTTCTCAAGTT | |
| | GGCGGGAGACGTGGAGTCCAACCCAGGGCCCATGGAGCAGAG | |
| | GAAGGGTCTGGCTGGCCTCTTCCTGGTGATCTCTCTTCTTCAAG | |
| | GCACTGTAGCCCAGACAAATAAAGCAAAGAATTTGGTACAAG | |
| | TGGATGGCAGCCGAGGAGACGGTTCTGTACTTCTGACTTGTGG | |
| | CTTGACTGACAAGACTATCAAGTGGCTTAAAGACGGGAGCATA | |
| | ATAAGTCCTCTAAATGCAACTAAAAACACATGGAATCTGGGCA | |
| | ACAATGCCAAAGACCCTCGAGGCACGTATCAGTGTCAAGGAG | |
| | CAAAGGAGACGTCAAACCCCCTGCAAGTGTATTACAGAATGTG | |
| | TGAAAACTGCATTGAGCTAAACATAGGCACCATATCCGGCTTT | |
| | ATCTTCGCTGAGGTCATCAGCATCTTCTTCCTTGCTCTTGGTGT | |
| | ATATCTCATTGCGGGACAGGATGGAGTTCGCCAGTCAAGAGCT | |
| | TCAGACAAGCAGACTCTGTTGCAAAATGAACAGCTGTACCAGC | |
| | CCCTCAAGGACCGGGAATATGACCAGTACAGCCATCTCCAAGG | |
| | AAACCAACTGAGGAAGAAGAGATCTGAGGGCAGAGGAAGTCT | |
| | GCTAACATGCGGTGACGTCGAGGAGAATCCTGGCCCAATGCGG | |
| | TGGAACACTTTCTGGGGCATCCTGTGCCTCAGCCTCCTAGCTGT | |
| | TGGCACTTGCCAGGACGATGCCGAGAACATTGAATACAAAGTC | |
| | TCCATCTCAGGAACCAGTGTAGAGTTGACGTGCCCTCTAGACA | |
| | GTGACGAGAACTTAAAATGGGAAAAAAATGGCCAAGAGCTGC | |
| | CTCAGAAGCATGATAAGCACCTGGTGCTCCAGGATTTCTCGGA | |
| | AGTCGAGGACAGTGGCTACTACGTCTGCTACACACCAGCCTCA | |
| | AATAAAAACACGTACTTGTACCTGAAAGCTCGAGTGTGTGAGT | |
| | ACTGTGTGGAGGTGGACCTGACAGCAGTAGCCATAATCATCAT | |
| | TGTTGACATCTGTATCACTCTGGGCTTGCTGATGGTCATTTATT | |
| | ACTGGAGCAAGAATAGGAAGGCCAAGGCCAAGCCTGTGACCC | |
| | GAGGAACCGGTGCTGGTAGCAGGCCCAGAGGGCAAAACAAGG | |
| | AGCGGCCACCACCTGTTCCCAACCCAGACTATGAGCCCATCCG | |
| | CAAAGGCCAGCGGGACCTGTATTCTGGCCTGAATCAGAGAGCA | |
| | GTCGGATCCGCCACGAACTTCTCTCTGTTAAAGCAAGCAGGAG | |
| | ACGTGGAAGAAAACCCCGGTCCCATGAAGTGGAAAGTGTCTGT | |
| | TCTCGCCTGCATCCTCCACGTGCGGTTCCCAGGAGCAGAGGCA | |
| | CAGAGCTTTGGTCTGCTGGACCCCAAACTCTGCTACTTGCTAG | |
| | ATGGAATCCTCTTCATCTACGGAGTCATCATCACAGCCCTGTAC | |
| | CTGAGAGCAAAATTCAGCAGGAGTGCAGAGACTGCTGCCAAC | |
| | CTGCAGGACCCCAACCAGCTCTACAATGAGCTCAATCTAGGGC | |
| | GAAGAGAGGAATATGACGTCTTGGAGAAGAAGCGGGCTCGGG | |
| | ACCCAGAGATGGGAGGCAAACAGCAGAGGAGGAGGAACCCCC | |
| | AGGAAGGCGTATACAATGCACTGCAGAAAGACAAGATGGCAG | |
| | AAGCCTACAGTGAGATCGGCACAAAAGGCGAGAGGCGGAGAG | |
| | GCAAGGGGCACGATGGCCTTTACCAGGGTCTCAGCACTGCCAC | |
| | CAAGGACACCTATGATGCCCTGCATATGCAGACCCTGGCCCCT | |
| | CGCTAA | |
| Polypeptide | MEDAKNIKKGPAPFYPLEDGTAGEQLHKAMKRYALVPGTI | 555 |
| sequence of | AFTDAHIEVNITYAEYFEMSVRLAEAMKRYGLNTNHRIVVC | |
| a luciferase | SENSLQFFMPVLGALFIGVAVAPANDIYNERELLNSMNISQP | |
| protein | TVVFVSKKGLQKILNVQKKLPIIQKIIIMDSKTDYQGFQSMY | |
| | TFVTSHLPPGFNEYDFVPESFDRDKTIALIMNSSGSTGLPKG | |
| | VALPHRTACVRFSHARDPIFGNQIIPDTAILSVVPFHHGFGMF | |
| | TTLGYLICGFRVVLMYRFEEELFLRSLQDYKIQSALLVPTLFS | |
| | FFAKSTLIDKYDLSNLHEIASGGAPLSKEVGEAVAKRFHLPG | |
| | IRQGYGLTETTSAILITPEGDDKPGAVGKVVPFFEAKVVDLD | |
| | TGKTLGVNQRGELCVRGPMIMSGYVNNPEATNALIDKDGW | |
| | LHSGDIAYWDEDEHFFIVDRLKSLIKYKGYQVAPAELESILL | |
| | QHPNIFDAGVAGLPDDDAGELPAAVVVLEHGKTMTEKEIV | |
| | DYVASQVTTAKKLRGGVVFVDEVPKGLTGKLDARKIREILI | |
| | KAKKGGKSKL | |
| Poly- | AAAGGAGGAAAAACTGTTTCATACAGAAGGCGTGGAGG | 556 |
| nucleotide | AAAAACTGTTTCATACAGAAGGCGTGGAGGAAAAACTGT | |
| sequence | TTCATACAGAAGGCGTATTTTGACACCCCCATAATATTTT | |
| encoding a | TCCAGAATTAACAGTATAAATTGCATCTCTTGTTCAAGAG | |
| luciferase | TTCCCTATCACTCTCTTTAATCACTACTCACAGTAACCTCA | |
| protein | ACTCCTGCCACAGGTACCGAGCTCAAGTTTGTACAAAAA | |
| | AGCAGGCTGCCACCATGGAAGACGCCAAAAACATAAAG | |
| | AAAGGCCCGGCGCCATTCTATCCGCTAGAGGATGGAACC | |
| | GCTGGAGAGCAACTGCATAAGGCTATGAAGAGATACGCC | |
| | CTGGTTCCTGGAACAATTGCTTTTACAGATGCACATATCG | |
| | AGGTGAACATCACGTACGCGGAATACTTCGAAATGTCCG | |
| | TTCGGTTGGCAGAAGCTATGAAACGATATGGGCTGAATA | |
| | CAAATCACAGAATCGTCGTATGCAGTGAAAACTCTCTTCA | |
| | ATTCTTTATGCCGGTGTTGGGCGCGTTATTTATCGGAGTT | |
| | GCAGTTGCGCCCGCGAACGACATTTATAATGAACGTGAA | |
| | TTGCTCAACAGTATGAACATTTCGCAGCCTACCGTAGTGT | |
| | TTGTTTCCAAAAAGGGGTTGCAAAAAATTTTGAACGTGC | |
| | AAAAAAAATTACCAATAATCCAGAAAATTATTATCATGG | |
| | ATTCTAAAACGGATTACCAGGGATTTCAGTCGATGTACAC | |
| | GTTCGTCACATCTCATCTACCTCCCGGTTTTAATGAATAC | |
| | GATTTTGTACCAGAGTCCTTTGATCGTGACAAAACAATTG | |
| | CACTGATAATGAACTCCTCTGGATCTACTGGGTTACCTAA | |
| | GGGTGTGGCCCTTCCGCATAGAACTGCCTGCGTCAGATTC | |
| | TCGCATGCCAGAGATCCTATTTTTGGCAATCAAATCATTC | |
| | CGGATACTGCGATTTTAAGTGTTGTTCCATTCCATCACGG | |
| | TTTTGGAATGTTTACTACACTCGGATATTTGATATGTGGA | |
| | TTTCGAGTCGTCTTAATGTATAGATTTGAAGAAGAGCTGT | |
| | TTTTACGATCCCTTCAGGATTACAAAATTCAAAGTGCGTT | |
| | GCTAGTACCAACCCTATTTTCATTCTTCGCCAAAAGCACT | |
| | CTGATTGACAAATACGATTTATCTAATTTACACGAAATTG | |
| | CTTCTGGGGGCGCACCTCTTTCGAAAGAAGTCGGGGAAG | |
| | CGGTTGCAAAACGCTTCCATCTTCCAGGGATACGACAAG | |
| | GATATGGGCTCACTGAGACTACATCAGCTATTCTGATTAC | |
| | ACCCGAGGGGGATGATAAACCGGGCGCGGTCGGTAAAGT | |
| | TGTTCCATTTTTTGAAGCGAAGGTTGTGGATCTGGATACC | |
| | GGGAAAACGCTGGGCGTTAATCAGAGAGGCGAATTATGT | |
| | GTCAGAGGACCTATGATTATGTCCGGTTATGTAAACAATC | |
| | CGGAAGCGACCAACGCCTTGATTGACAAGGATGGATGGC | |
| | TACATTCTGGAGACATAGCTTACTGGGACGAAGACGAAC | |
| | ACTTCTTCATAGTTGACCGCTTGAAGTCTTTAATTAAATA | |
| | CAAAGGATACCAGGTGGCCCCCGCTGAATTGGAGTCGAT | |
| | ATTGTTACAACACCCCAACATCTTCGACGCGGGCGTGGC | |
| | AGGTCTTCCCGACGATGACGCCGGTGAACTTCCCGCCGCC | |
| | GTTGTTGTTTTGGAGCACGGAAAGACGATGACGGAAAAA | |
| | GAGATCGTGGATTACGTCGCCAGTCAAGTAACAACCGCG | |
| | AAAAAGTTGCGCGGAGGAGTTGTGTTTGTGGACGAAGTA | |
| | CCGAAAGGTCTTACCGGAAAACTCGACGCAAGAAAAATC | |
| | AGAGAGATCCTCATAAAGGCCAAGAAGGGCGGAAAGTC | |
| | CAAATTGTAA | |
| Poly- | CTGGGAGCAAACACCGTGGATGGTGGAATCACTCAGTCC | 557 |
| nucleotide | CCAAAGTACCTGTTCAGAAAGGAAGGACAGAATGTGACC | |
| sequence | CTGAGTTGTGAACAGAATTTGAACCACGATGCCATGTACT | |
| encoding | GGTACCGACAGGACCCAGGGCAAGGGCTGAGATTGATCT | |
| IGRP TCR | ACTACTCACAGATAGTAAATGACTTTCAGAAAGGAGATA | |
| murinized | TAGCTGAAGGGTACAGCGTCTCTCGGGAGAAGAAGGAAT | |
| | CCTTTCCTCTCACTGTGACATCGGCCCAAAAGAACCCGAC | |
| | AGCTTTCTATCTCTGTGCCAGTGGGGGACGGGTCTATCAG | |
| | CCCCAGCATTTTGGTGATGGGACTCGACTCTCCATCCTAG | |
| | AGGATCTGAGAAATGTGACTCCACCCAAGGTCTCCTTGTT | |
| | TGAGCCATCAAAAGCAGAGATTGCAAACAAACAAAAGG | |
| | CTACCCTCGTGTGCTTGGCCAGGGGCTTCTTCCCTGACCA | |
| | CGTGGAGCTGAGCTGGTGGGTGAATGGCAAGGAGGTCCA | |
| | CAGTGGGGTCAGCACGGACCCTCAGGCCTACAAGGAGAG | |
| | CAATTATAGCTACTGCCTGAGCAGCCGCCTGAGGGTCTCT | |
| | GCTACCTTCTGGCACAATCCTCGCAACCACTTCCGCTGCC | |
| | AAGTGCAGTTCCATGGGCTTTCAGAGGAGGACAAGTGGC | |
| | CAGAGGGCTCACCCAAACCTGTCACACAGAACATCAGTG | |
| | CAGAGGCCTGGGGCCGAGCAGACTGTGGGATTACCTCAG | |
| | CATCCTATCAACAAGGGGTCTTGTCTGCCACCATCCTCTA | |
| | TGAGATCCTGCTAGGGAAAGCCACCCTGTATGCTGTGCTT | |
| | GTCAGTACACTGGTGGTGATGGCTATGGTCAAAAGAAAG | |
| | AATTCATGA | |

1-48. (canceled)

49. An in vitro method for assaying agonistic activity of a nanomedicine, wherein the nanomedicine comprises a nanoparticle coupled to a construct comprising a disease-relevant antigen bound to a major histocompatibility (MHC) molecule, the method comprising:
a) contacting the nanomedicine with a cell comprising:
a recombinant T cell receptor (TCR) comprising a TCR alpha chain and a TCR beta chain; and
a T cell receptor-pathway-dependent reporter, wherein the recombinant T cell receptor is specific for the disease-relevant antigen bound to the MHC molecule coupled to the nanoparticle; and
b) detecting a signal produced by the T cell receptor-pathway-dependent reporter.

50. The method of claim 49, wherein the nanomedicine comprises a plurality of nanoparticles coupled to a construct comprising a disease-relevant antigen bound to the MHC molecules.

51. The method of claim 50, wherein each nanoparticle of the plurality of nanoparticles comprises a plurality of disease-relevant antigens bound to the MHC molecule coupled to the nanoparticle.

52. The method of claim 51, wherein the disease-relevant antigen is an autoimmune or inflammatory disease-relevant antigen.

53. The method of claim 52, wherein the autoimmune or inflammatory disease-relevant antigen is chosen from a diabetes mellitus Type I antigen, an asthma or allergic asthma antigen, a multiple sclerosis antigen, a peripheral neuropathy antigen, a primary biliary cirrhosis antigen, a neuromyelitis optica spectrum disorder antigen, a stiff-person syndrome antigen, an autoimmune encephalitis antigen, a pemphigus vulgaris antigen, a pemphigus foliaceus antigen, a psoriasis antigen, a Sjogren's disease/syndrome antigen, an inflammatory bowel disease antigen, an arthritis or rheumatoid arthritis antigen, a systemic lupus erythematosus antigen, a scleroderma antigen, an ANCA-associated vasculitis antigen, a Goodpasture syndrome antigen, a Kawasaki's disease antigen, a celiac disease, an autoimmune cardiomyopathy antigen, a myasthenia gravis antigen, an autoimmune uveitis antigen, a Grave's disease antigen, an anti-phospholipid syndrome antigen, an autoimmune hepatitis antigen, a sclerosing cholangitis antigen, a primary sclerosing cholangitis antigen, chronic obstructive pulmonary disease antigen, or a uveitis relevant antigen, and combinations thereof.

54. The method of claim 50, wherein each nanoparticle of the plurality of nanoparticles comprises a diameter of from 1 nanometer to about 100 nanometers.

55. The method of claim 49, further comprising quantifying the T cell receptor-pathway-dependent reporter signal.

56. The method of claim 55, wherein the quantifying step comprises determining a concentration of the nanomedicine that initiates a response that is about 50% of a maximal response, wherein the maximal response is the response initiated at the highest concentration of nanomedicine contacted with the cell or a population of cells when a plurality of concentrations of the nanomedicine are contacted with the cell or population of cells.

57. The method of claim 56, wherein the plurality of the concentrations of the nanomedicine are contacted with the cell or the population of cells in the same assay.

58. The method of claim 55, wherein the quantifying step comprises determining a concentration of the nanomedicine that initiates a response that is at least about 200%, of a negative control, wherein the negative control comprises a nanomedicine that does not specifically interact with the recombinant T cell receptor (TCR) of the cell or a population of cells.

59. The method of claim 49, wherein the signal is produced by an enzyme.

60. The method of claim 59, wherein the enzyme is luciferase or peroxidase.

61. The method of claim 49, wherein the signal is a fluorescent signal.

62. (canceled)

63. The method of claim 49, used as a quality control step in a manufacturing process.

64. The method of claim 49, wherein the T cell receptor-pathway-dependent reporter is actively transcribed.

65. The method of claim 49, wherein the T cell receptor-pathway-dependent reporter activates transcription of a gene chosen from a luciferase gene, a beta lactamase gene, a chloramphenicol acetyltransferase (CAT) gene, a secreted embryonic alkaline phosphatase (SEAP) gene, a fluorescent protein gene, and combinations thereof.

66. The method of claim 49, wherein the T cell receptor-pathway-dependent reporter comprises a polynucleotide sequence chosen from a nuclear factor of activated T cells (NFAT) transcription factor-binding DNA sequence or promoter, an NF-κB transcription factor-binding DNA sequence or promoter, an AP1 transcription factor-binding DNA sequence or promoter, an IL-2 transcription factor-binding DNA sequence or promoter, and combinations thereof.

67. The method of claim 49, wherein the cell is selected from JurMA, Jurkat, BW5147, HuT-78, CEM, or Molt-4.

68. The method of claim 49, wherein the disease-relevant antigen is a polypeptide consisting of any one of SEQ ID NOs: 1 to 352 and combinations thereof.

69. The method of claim 49, wherein the disease-relevant antigen is a polypeptide consisting of any one of SEQ ID NOs: 353 to 455 and combinations thereof.

70. The method of claim 49, wherein the TCR alpha chain and TCR beta chain are translated as a single polypeptide.

71. The method of claim 70, wherein the TCR alpha chain and TCR beta chain of the single polypeptide are separated by a ribosome skipping sequence.

72. The method of claim 71, wherein the ribosome skipping sequence is set forth in any one of SEQ ID NOs: 456 to 523.

73. The method of claim 70, wherein the single polypeptide comprises an amino acid sequence at least 80%, 90%, 95%, or 100% identical to any one of SEQ ID NOs: 527, 533, or 538.

74. The method of claim 49, wherein the TCR alpha chain and TCR beta chain are translated as separate polypeptides.

75. The method of claim 49, wherein the TCR alpha chain comprises an amino acid sequence at least 80%, 90%, 95%, or 100% identical to any one of SEQ ID NOs: 528, 530, 534, 536 539, 541, and the TCR beta chain comprises an amino acid sequence at least 80%, 90%, 95%, or 100% identical to any one of SEQ ID NOs: 529, 531, 535, 537, 540, or 542.

76. The method of claim 49, wherein the TCR alpha chain and TCR beta chain are expressed at the surface of the cell.

77. The method of claim 49, wherein the cell comprises at least one exogenous polynucleotide encoding the TCR alpha chain and the TCR beta chain.

78. The method of claim 77, wherein the at least one exogenous polynucleotide comprises an internal ribosome entry site (IRES) nucleic acid sequence.

79. The method of claim 78, wherein the IRES nucleic acid sequence is set forth in any one of SEQ ID NOs: 524 to 526.

80. The method of claim 79, wherein the at least one exogenous polynucleotide comprises a nucleic acid sequence at least 80%, 90%, 95%, or 100% homologous to that set forth in any one of SEQ ID NOs: 532 or 557.

81. The method of claim 49, wherein the nanomedicine is for use in a human individual.
